(19) United States
(12) Patent Application Publication
(10) Pub. No.: US 2025/0332244 A1
(43) Pub. Date: Oct. 30, 2025

(54) IMMUNOGENIC COMPOSITIONS AGAINST INFLUENZA

(71) Applicant: Pfizer Inc., New York, NY (US)

(72) Inventors: Bogos Agianian, Stratford, CT (US); Pirada Suphaphiphat Allen, Congers, NY (US); Adam Lee Campbell, West Nyack, NY (US); Ye Che, Niantic, CT (US); Liyang Cui, Woodcliff Lake, NJ (US); Ivna Padron De Souza, Nyack, NY (US); KariAnn Sweeney Efferen, Lagrangeville, NY (US); Miguel Angel Garcia, Midland Park, NJ (US); Qianjing He, Nanuet, NY (US); Sue-Jean Hong, Montvale, NJ (US); Shenping Lui, Waterford, CT (US); Aravinda Munasinghe, Groton, CT (US); James Forrest Smith, Gales Ferry, CT (US); Herg Hwa Zhang, Dunellen, NJ (US); Lei Zhang, Bardonia, NY (US)

(21) Appl. No.: 18/882,451

(22) Filed: Sep. 11, 2024

Related U.S. Application Data
(60) Provisional application No. 63/582,513, filed on Sep. 13, 2023.
(60) Provisional application No. 63/611,106, filed on Dec. 15, 2023.
(60) Provisional application No. 63/559,863, filed on Feb. 29, 2024.
(60) Provisional application No. 63/562,257, filed on Mar. 6, 2024.
(60) Provisional application No. 63/568,754, filed on Mar. 22, 2024.
(60) Provisional application No. 63/634,399, filed on Apr. 15, 2024.
(60) Provisional application No. 63/656,098, filed on Jun. 4, 2024.

(51) Int. Cl. A61K 39/145; A61K 9/1271; A61K 9/51; A61K 39/00; C07K 14/005

(52) U.S. Cl. CPC A61K 39/145; A61K 9/1271; A61K 9/5123; C07K 14/005; A61K 2039/53; A61K 2039/55555; A61K 2039/70; C12N 2760/16122; C12N 2760/16134

(57) ABSTRACT

The invention relates to compositions and methods for the preparation, manufacture and therapeutic use ribonucleic acid vaccines comprising polynucleotide molecules encoding one or more influenza antigens, such as hemagglutinin antigens.

### CROSS-REFERENCE TO RELATED APPLICATIONS

This application claims the benefit of U.S. provisional application No. 63/582,513, filed Sep. 13, 2023, U.S. provisional application No. 63/611,106, filed Dec. 15, 2023, U.S. provisional application No. 63/559,863, filed Feb. 29, 2024, U.S. provisional application No. 63/562,257, filed Mar. 6, 2024, U.S. provisional application No. 63/568,754, filed Mar. 22, 2024, U.S. provisional application No. 63/634,399, filed Apr. 15, 2024, and U.S. provisional application No. 63/656,098, filed Jun. 4, 2024, each of which is incorporated by reference herein in its entirety.

### REFERENCE TO SEQUENCE LISTING

This application is being filed electronically via Patent Center and includes an electronically submitted sequence listing in .XML format. The XML file contains a sequence listing entitled, “PC073030A.xml,” created on Sep. 12, 2024, and having a size of 163 KB. The sequence listing contained in this XML file is part of the specification and is incorporated herein by reference in its entirety.

### FIELD

The invention relates to compositions and methods for the preparation, manufacture and therapeutic use of ribonucleic acid vaccines comprising polynucleotide molecules encoding one or more influenza antigens, such as hemagglutinin antigens.

### BACKGROUND

Influenza viruses are members of the orthomyxoviridae family, and are classified into three types (A, B, and C), based on antigenic differences between their nucleoprotein (NP) and matrix (M) protein.

The genome of influenza A virus includes eight molecules (seven for influenza C virus) of linear, negative polarity, single-stranded RNAs, which encode several polypeptides including: the RNA-directed RNA polymerase proteins (PB2, PB1 and PA) and nucleoprotein (NP), which form the nucleocapsid; the matrix proteins (M1, M2, which is also a surface-exposed protein embedded in the virus membrane); two surface glycoproteins, which project from the lipoprotein envelope: hemagglutinin (HA) and neuraminidase (NA); and nonstructural proteins (NS1 and NS2). Hemagglutinin is the major envelope glycoprotein of influenza A and B viruses, and hemagglutinin-esterase (HE) of influenza C viruses is a protein homologous to HA.

A challenge for therapy and prophylaxis against influenza and other infections using traditional vaccines is the limitation of vaccines in breadth, providing protection only against closely related subtypes. In addition, the length of time required to complete current standard influenza virus vaccine production processes inhibits the rapid development and production of an adapted vaccine in a pandemic situation.

There is a need for improved immunogenic compositions against influenza.

### SUMMARY

The unmet needs for improved immunogenic compositions against influenza, among other things, are provided herein. In one aspect, the disclosure relates to an improved polypeptide derived from influenza virus, wherein the polypeptide has mutations in a fusion peptide and fusion peptide proximal regions (FPPR), relative to the corresponding wild-type influenza polypeptide. In preferred embodiments, the polypeptide is derived from an influenza hemagglutinin polypeptide. In some embodiments, the polypeptide is derived from a hemagglutinin of an influenza B virus.

In some embodiments, the influenza hemagglutinin polypeptide may be derived from hemagglutinin of an influenza virus from the B/Yamagata lineage (as represented by B/Yamagata/16/88) or from the B/Victoria lineage (as represented by B/Victoria/2/87). In some embodiments, the polypeptide is derived from B/Brisbane/60/08, B/lowa/06/2017, or B/Lee/40.

In some embodiments, the polypeptide has at least 60%, 70%, 75%, 80%, 90%, 95%, 97%, 98%, 99%, or 100% sequence identify to any one of amino acid sequences SEQ ID NO: 10-SEQ ID NO: 68. In some embodiments, the polypeptide comprises an amino acid sequence selected from any one of SEQ ID NO: 10-SEQ ID NO: 68.

As used herein, the terms “non-natural,” “non-naturally occurring,” and “mutant” are used interchangeably in the context of an organism, polypeptide, or nucleic acid. The terms “non-natural” and “non-naturally occurring” and “mutant” in this context refer to a polypeptide or nucleic acid having at least one variation or mutation at an amino acid position or nucleic acid position as compared to the respective wild-type polypeptide or nucleic acid. Non-limiting examples of the at least one variation are an insertion of one or more amino acids or nucleotides, a deletion of one or more amino acids or nucleotides, or a substitution of one or more amino acids or nucleotides. In some embodiments, the polypeptides and/or nucleic acids of the present disclosure, e.g., polypeptides comprising an amino acid sequence of an influenza B virus hemagglutinin protein or nucleic acids encoding such polypeptides, are non-naturally occurring and include a deletion relative to the respective wild-type sequence at specified positions of the respective wild-type sequence. Further, when referring to “residues X to Y” of a specified sequence herein, one of ordinary skill in the art understands this to mean a contiguous sequence of the indicated amino acid residues in the respective specified sequence. In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acids. In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids. The “percent identity” (% identity) between two sequences is determined when sequences are aligned for maximum homology, and not including gaps or truncations as set forth in the alignment parameters. Exemplary parameters for determining relatedness of two or more amino acid sequences using the BLAST algorithm, for example, can be as provided in BLASTP. Nucleic acid sequence alignments can be performed using BLASTN. Modifications can be made to the alignment parameters to either increase or decrease the stringency of the comparison, for example, for determining the relatedness of two or more sequences. Additional sequences added to a polypeptide sequence, including but not limited to immunodetection tags, purification tags, localization sequences (presence or absence), etc., do not affect the % identity. Algorithms such as Align, BLAST, ClustalW and others can be used to compare and determine a raw sequence's similarity or identity to another sequence, and also determine the presence or significance of gaps in the sequence which can be assigned a weight or score. Such algorithms are similarly applicable for determining nucleotide or amino acid sequence similarity or identity, and can be useful in identifying orthologs of genes of interest. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 45% to 100% sequence identity. Proteins that are unrelated can have an identity which is essentially the same as would be expected to occur by chance if a database of sufficient size is scanned (about 5%). For example, alignment can be performed using the Needleman-Wunsch algorithm implemented through the BALIGN tool. Default parameters may be used for the alignment and BLOSUM62 may be used as the scoring matrix. In some cases, it can be useful to use the BLAST algorithm to understand the sequence identity between an amino acid motif in a template sequence and a target sequence. Therefore, in some embodiments, BLAST is used to identify or understand the identity of a shorter stretch of amino acids (e.g., a sequence motif) between a template and a target protein. BLAST finds similar sequences using a heuristic method that approximates the Smith-Waterman algorithm by locating short matches between the two sequences. The BLAST algorithm can identify library sequences that resemble the query sequence above a certain threshold. As used herein, an amino acid position (or simply, amino acid) “corresponding to” an amino acid position in another polypeptide sequence is the position that is aligned with the referenced amino acid position when the polypeptides are aligned. The polypeptides may be aligned with maximum homology, for example, as determined by BLAST, which allows for gaps in sequence homology within protein sequences to align related sequences and domains. Alternatively, in some instances, when polypeptide sequences are aligned for maximum homology, a corresponding amino acid may be the nearest amino acid to the identified amino acid that is within the same amino acid biochemical grouping—i.e., the nearest acidic amino acid, the nearest basic amino acid, the nearest aromatic amino acid, etc., to the identified amino acid. By “substantially identical,” with reference to a nucleic acid sequence (e.g., a gene, RNA, or cDNA) or amino acid sequence (e.g., a protein or polypeptide) is meant one that has at least 50%, at least 55%, at least 60%, at least 65%, at least 70%, at least 75%, at least 80%, at least 85%, at least 90%, at least 95%, at least 96%, at least 97% at least 98%, or at least 99% nucleotide or amino acid identity, respectively, to a reference sequence.

In one aspect, the disclosure relates to a nucleic acid encoding a polypeptide derived from an influenza polypeptide, preferably a hemagglutinin polypeptide, that comprises a fusion peptide and proximal regions (FPPR), wherein the FPPR comprises a deletion of at least three to seven amino acid residues between amino acid positions 369 and 382, more preferably 352 and 382, corresponding to the amino acid positions of SEQ ID NO: 9. In some preferred embodiments, the disclosure relates to a nucleic acid encoding a polypeptide derived from an influenza polypeptide, preferably a hemagglutinin polypeptide, that comprises a fusion peptide and proximal regions (FPPR), wherein the FPPR comprises a deletion of at least three to seven amino acid residues between amino acid positions 352 and 382, corresponding to the amino acid positions of SEQ ID NO: 9.

In one aspect, the disclosure relates to an immunogenic composition including: (i) a first ribonucleic acid (RNA) polynucleotide having an open reading frame encoding a first antigen, said antigen including at least one influenza virus antigenic polypeptide or an immunogenic fragment thereof, and (ii) a second RNA polynucleotide having an open reading frame encoding a second antigen, said second antigen including at least one influenza virus antigenic polypeptide or an immunogenic fragment thereof, wherein the first and second RNA polynucleotides are formulated in a lipid nanoparticle (LNP). In some embodiments, the first and second antigens include hemagglutinin (HA), or an immunogenic fragment or variant thereof. In some embodiments, the first antigen includes an HA from a different subtype of influenza virus to the influenza virus antigenic polypeptide or an immunogenic fragment thereof of the second antigen. In some embodiments, the composition further includes (iii) a third antigen including at least one influenza virus antigenic polypeptide or an immunogenic fragment thereof, wherein the third antigen is from influenza virus but is from a different strain of influenza virus to both the first and second antigens. In some embodiments, the first, second and third RNA polynucleotides are formulated in a lipid nanoparticle.

In some embodiments, the composition further includes (iv) a fourth RNA polynucleotide having an open reading frame encoding a fourth antigen, said antigen including at least one influenza virus antigenic polypeptide or an immunogenic fragment thereof, wherein the fourth antigen is from influenza virus but is from a different strain of influenza virus to the first, second and third antigens. In some embodiments, the first, second, third, and fourth RNA polynucleotides are formulated in a lipid nanoparticle.

In some embodiments, each RNA polynucleotide includes a modified nucleotide. In some embodiments, the modified nucleotide is selected from the group consisting of pseudouridine, 1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

In some embodiments, each RNA polynucleotide includes a 5′ terminal cap, a 5′ UTR, a 3′UTR, and a 3′ polyadenylation tail. In some embodiments, the 5′ terminal cap includes:

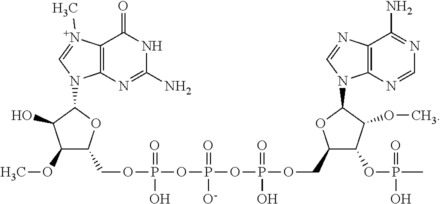

In some embodiments, the 5′ UTR includes SEQ ID NO: 1. In some embodiments, the 3′ UTR includes SEQ ID NO: 2. In some embodiments, the 3′ polyadenylation tail includes SEQ ID NO: 3.

In some embodiments, the RNA polynucleotide has an integrity greater than 85%. In some embodiments, the RNA polynucleotide has a purity of greater than 85%.

In some embodiments, the lipid nanoparticle includes 20-60 mol % ionizable cationic lipid, 5-25 mol % neutral lipid, 25-55 mol % cholesterol, and 0.5-5 mol % PEG-modified lipid.

In some embodiments, the cationic lipid includes:

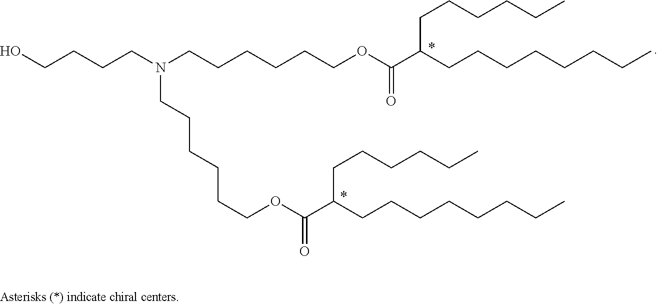 [SMILES: CCCCCCCCC(CCCCCC)C(=O)OCCCCCCN(CCCCO)CCCCCCOC(=O)C(CCCCCC)CCCCCCCC]

In some embodiments, the PEG-modified lipid includes:

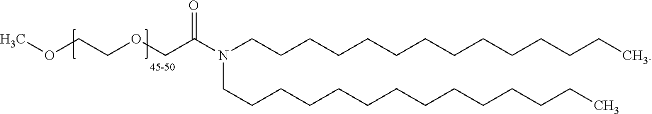

In some embodiments, the first antigen is HA from influenza A subtype H1 or an immunogenic fragment or variant thereof and the second antigen is HA from a different H1 strain to the first antigen or an immunogenic fragment or variant thereof. In some embodiments, the first and second antigens are HA from influenza A subtype H3 or an immunogenic fragment or variant thereof and wherein both antigens are derived from different strains of H3 influenza virus.

In some embodiments, the first and second antigens are HA from influenza A subtype H1 or an immunogenic fragment or variant thereof and the third and fourth antigens are from influenza A subtype H3 or an immunogenic fragment or variant thereof and wherein the first and second antigens are derived from different strains of H1 virus and the third and fourth antigens are from different strains of H3 influenza virus.

In some embodiments, at least the first and second RNA polynucleotides are formulated in a single lipid nanoparticle. In some embodiments, the first and second RNA polynucleotides are formulated in a single lipid nanoparticle. In some embodiments, the first, second, and third RNA polynucleotides are formulated in a single lipid nanoparticle. In some embodiments, the first, second, third, and fourth RNA polynucleotides are formulated in a single LNP.

In some embodiments, each of the RNA polynucleotides is formulated in a single LNP, wherein each single LNP encapsulates the RNA polynucleotide encoding one antigen. In some embodiments, the first RNA polynucleotide is formulated in a first LNP; and the second RNA polynucleotide is formulated in a second LNP. In some embodiments, the first RNA polynucleotide is formulated in a first LNP; the second RNA polynucleotide is formulated in a second LNP; and the third RNA polynucleotide is formulated in a third LNP. In some embodiments, the first RNA polynucleotide is formulated in a first LNP; the second RNA polynucleotide is formulated in a second LNP; the third RNA polynucleotide is formulated in a third LNP; and the fourth RNA polynucleotide is formulated in a fourth LNP.

In another aspect, the disclosure relates to any of the immunogenic compositions described herein, for use in the eliciting an immune response against influenza.

In another aspect, the disclosure relates to a method of eliciting an immune response against influenza disease, including administering an effective amount of any of the immunogenic compositions described herein.

In another aspect, the disclosure relates to a method of purifying an RNA polynucleotide synthesized by in vitro transcription. The method includes ultrafiltration and diafiltration. In some embodiments, the method does not comprise a chromatography step. In some embodiments, the purified RNA polynucleotide is substantially free of contaminants comprising short abortive RNA species, long abortive RNA species, double-stranded RNA (dsRNA), residual plasmid DNA, residual in vitro transcription enzymes, residual solvent and/or residual salt. In some embodiments, the residual plasmid DNA is ≤500 ng DNA/mg RNA. In some embodiments, the yield of the purified mRNA is about 70% to about 99%. In some embodiments, purity of the purified mRNA is between about 60% and about 100%. In some embodiments, purity of the purified mRNA is between about 85%-95%.

In some embodiments, the disclosure provides a nucleic acid encoding a polypeptide described herein. In some embodiments, the disclosure provides an expression construct comprising a nucleic acid described herein. In some embodiments, the disclosure provides a method of inducing an immunological response against an influenza B virus in a subject in need thereof, comprising administering to the subject an immunologically effective amount of a polypeptide or protein trimer described herein, the immunogenic composition described herein, or combination thereof.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-H. Functional Anti-HA Antibodies Elicited by Immunization of Mice With Monovalent or Quadrivalent LNP-Formulated modRNA Encoding Influenza HA as Measured By MNT. FIG. 1A depicts 50% neutralization titers 3 weeks post prime (against A/H1N1); FIG. 1B depicts 50% neutralization titers 2 weeks post boost (against A/H1N1); FIG. 1C depicts 50% neutralization titers 3 weeks post prime (against A/H3N2); FIG. 1D depicts 50% neutralization titers 2 weeks post boost (against A/H3N2); FIG. 1E depicts 50% neutralization titers 3 weeks post prime (against B/Yamagata); FIG. 1F depicts 50% neutralization titers 2 weeks post boost (against B/Yamagata); FIG. 1G depicts 50% neutralization titers 3 weeks post prime (against B/Victoria);

FIG. 1H depicts 50% neutralization titers 2 weeks post boost (against B/Victoria).

FIG. 2. Example of a mutant (#24) (also referred to herein as any one of A355-363 and pBV-024 (SEQ ID NO: 33) shows increased potency when compared to wild type FluB.

FIG. 3. Correlation of In Vitro Expression in Human Hela Cells determined using monoclonal antibody CR8071 vs polycolonal antibody B295-HA variants of Influenza virus. Selected variants are labeled with their SEQ NO IDs.

FIG. 4. Correlation of In Vitro Expression in Human Hela Cells measured using monoclonal antibodies CR8071 and CR9114 of HA variants of Influenza virus. Selected variants are labeled with their SEQ NO IDs. CR8071 and CR9114 recognize different epitopes of the HA.

FIG. 5. Functional Anti-HA Antibodies Elicited by Immunization of Mice With Monovalent or Quadrivalent LNP-Formulated modRNA Encoding Influenza HA as Measured By MNT In vivo immunogenicity felicitated by HA variants of influenzae B virus determined by neutralization antibody titer, 3 weeks post dose 1.

FIG. 6. In vivo immunogenicity felicitated by HA variants of influenzae B virus measured by neutralization antibody titer, 2 weeks post dose 2.

FIG. 7. Correlation of In vivo immunogenicity felicitated by HA variants of influenzae B virus measured by neutralization antibody titer, 2 weeks post dose 2, and their in vitro expression in Heman cells. DS, drug substances; DR, Drug product.

FIG. 8A-C. Same design principle can be applied to HA of other influenza B virus strains to improve the immunogenicity against those virus strains. In this example, the HA variant with the mutation equivalent to SEQ ID NO: 47 was engineered in Washington and Colorado sublineages of Victory strain of influenza B virus, and Phuket sublineages of Yamagata strain. Improvements on immunogenicity measured by neutralization antibody titers were observed.

FIG. 9A-C. CleanCap AG saRNA performed better than enzymatically capped saRNA in THP-1. In the number of cells expressing antigen encoded by saRNA & geometric mean fluorescence intensity (GMFI) of the antigen in saRNA-transfected cells (the antigen copy number). FIG. 9C depicts A/Wisc/588/19 HA expression in THP-1. THP-1 cells were transfected with either saRNA-TC83-A/Wisc/588/19 HA-40A or bicistronic saRNA-TC83-A/Wisc/588/19 HA-NA-80A either with no nucleoside modifications, m5C, Hm5C, or 2′Ome-G incorporation (11-point, 2-fold dilution series starting from 1000 ng). Number of HA expressing cells (% HA+ cells) (FIG. 9A), total HA expression per HA positive cell (Geometric mean fluorescence intensity (GMFI)) (FIG. 9B), and number of live cells (% live cells) (FIG. 9C), were determined by flow cytometry at 22 hrs post transfection. Results are presented as mean±standard deviation for each group from a representative experiment.

FIG. 10A-B. FIG. 10A depicts % of encapsulation and LNP size (d. nm) of LNPs before dialysis and after filtration. FIG. 10B depicts % of positive express in HEK293T cells when comparing LNP formulations in the presence of egg sphingomyelin (ESM) and cholesterol against a benchmark LNP formulation in the absence of ESM and cholesterol.

FIG. 11A-C. FIG. 11A depicts testing various LNP formulations and measuring LNP size (d. nm), wherein the samples tested are described in Table 54. Successful combination of sitosterol and SM can be achieved using PBS as buffer. FIG. 11B depicts fraction of positive expression in HEK293T, testing samples as described respectively in Table 52. FIG. 11C depicts mean fluorescence intensity (MFI) of samples as described respectively in Table 52.

FIG. 12A-B. FIG. 12A depicts LNP size change of samples with various cationic lipid and ESM ratios. Samples tested are respectively described in Table 54. FIG. 12B depicts % encapsulation efficiency (EE) change of samples with various cationic lipid and ESM ratios. Samples tested are respectively described in Table 54.

FIG. 13A-B. FIG. 13A depicts % of positive expression in HEK293T cells of samples with various cationic lipid and ESM ratios. Samples tested are respectively described in Table 55.

FIG. 13B depicts MFI of positive expression cells from samples respectively described in Table 55.

FIG. 14A-G depict various aspects of the immune response measured in the study. FIG. 14A shows HA-specific antibody titers were measured by a hemagglutination inhibition assay (HAI) (FIG. 14A), and FIG. 14B illustrates the results of a microneutralization assay test (MNT) (FIG. 14B). Splenocytes were harvested two weeks after the second immunization, stimulated with peptides spanning the H1N1 HA protein from the vaccine strain (A/Wisconsin/588/2019), and assessed by intracellular cytokine staining for CD4+ T cells expressing IFN-γ, IL-4, IL-2, TNF-α and/or CD154, and CD8+ T cells expressing IFN-γ, TNF-α and/or CD107a. FIG. 14C demonstrates that immunization with two doses of mIRV induced a higher percentage of IFN-γ-producing CD4+ T cells than IL-4-producing CD4+ T cells (FIG. 14C), indicative of a Th1-biased response, whereas FIG. 14D shows that two doses of QIV induced a higher percentage of IL-4+CD4+ T cells than IFN-γ+CD4+ T cells (FIG. 14D), indicative of a Th2-biased response. FIG. 14E highlights results of a strong polyfunctional (IFN-γ+, IL-2+, TNF-α+, CD154+) CD4+ T cell response was also observed with the mIRV vaccine, but not with QIV (FIG. 14E). In addition, FIG. 14F depicts results showing immunization with mlRV induced higher levels of IFN-γ+CD8+ T cells (FIG. 14F) and FIG. 14G shows polyfunctional (IFN-γ+, TNF-α+, CD107a+) CD8+ T cells (FIG. 14G) compared to QIV. Each data point represents one mouse. IM, intramuscular; QIV, quadrivalent influenza vaccine; HAI, hemagglutination inhibition assay; MNT, microneutralization assay; GMT, geometric mean titer; LOD, limit of detection for MNT assay; ICS, intracellular cytokine staining; SEM, standard error of the mean.

FIG. 15A-F. HA-specific antibodies were measured by HAI (FIG. 15A-B). FIG. 15A depicts functional antibody and virus neutralization responses against the mIRV strain A/Wisconsin/588/2019 (H1N1) measured by HAI in Rhesus macaques; FIG. 15B depicts depicts functional antibody and virus neutralization responses against the mIRV strain A/Wisconsin/588/2019 (H1N1) measured by HAI in Cynomologus macaques) and MNT (FIG. 15C-D. FIG. 15C depicts functional antibody and virus neutralization responses against the mIRV strain A/Wisconsin/588/2019 (H1N1) measured by 50% MNT in Rhesus macaques; FIG. 15D depicts functional antibody and virus neutralization responses against the mIRV strain A/Wisconsin/588/2019 (H1N1) measured by 50% MNT in Cynomologus macaques). Each data point represents an individual NHP and the bar depicts the GMT of the 3 NHPs per group with 95% CI. T cell immunity was quantified by measuring cytokine-expressing peripheral CD4+ and CD8+ T cells after ex vivo stimulation of peripheral blood mononuclear cells (PBMCs) with HA peptide pools derived from the H1N1 vaccine strain (FIG. 15E-F), i.e., FIG. 15E depicts ICS assay results plotted as the % of IFN-γ+ cells within CD4+ T cell subsets for rhesus macaques;

FIG. 15F depicts ICS assay results plotted as the % of IFN-γ+ cells within CD4+ T cell subsets for cynomolgus macaques. Each data point represents an individual NHP and the bar depicts the median of the 3 NHPs per group with 95% CI. The connecting line in A-D shows the geomean group kinetics over time, and the line in E-F shows the median group kinetics over time. IM, intramuscular; NHP, nonhuman primate; HAI, hemagglutination inhibition assay; MNT, microneutralization assay; GMT, geometric mean titer; LOD, limit of detection for MNT assay; ICS, intracellular cytokine staining; PMBCs, peripheral mononuclear blood cells.

FIG. 16A-B. Mice were immunized with two doses of quadrivalent modRNA-HA vaccine (qIRV; 0.8 μg (0.2 μg/HA) or licensed adjuvanted QIV (FLUADR; 2.4 μg; 10/group) or saline (10/group) 28 days apart. Two weeks after the second immunization, functional antibodies against each of the four strains encoded by the vaccines were measured by HAI (FIG. 16A) and MNT (FIG. 16B). FIG. 16A depicts functional antibody and virus neutralization responses against each of the four qIRV strains, A/Wisconsin/588/2019 (H1N1), A/Cambodia/e0826360/2020 (H3N2), B/Washington/02/2019 (B/Victoria), and B/Phuket/3073/2013 (B/Yamagata), as measured by HAI. FIG. 16B depicts functional antibody and virus neutralization responses against each of the four qIRV strains, A/Wisconsin/588/2019 (H1N1), A/Cambodia/e0826360/2020 (H3N2), B/Washington/02/2019 (B/Victoria), and B/Phuket/3073/2013 (B/Yamagata), as measured by 50% MNT. All titers are reported as GMT with 95% CI. Each data point represents two paired animals. Statistical comparisons were performed using an independent sample t-test, ***, p<0.0001; ns, not significant. IM, intramuscular; QIV, quadrivalent influenza vaccine; HAI, hemagglutination inhibition assay; MNT, microneutralization assay; GMT, geometric mean titer; LOD, limit of detection for MNT assay.

FIG. 17A-D. Two weeks after the second immunization, virus neutralization titers against the vaccine-matched strains were measured by MNT (FIG. 17). Female naïve BALB/c mice were immunized IM with two doses (prime+boost) of a monovalent (mIRV; 0.2 μg; 10/group), trivalent (tIRV; 0.6 μg (0.2 μg/HA); 10/group), or quadrivalent (qIRV; 0.8 μg (0.2 μg/HA); 10/group) modRNA-HA vaccine, a QIV comparator (Fluad; 2.4 μg; 10/group), or saline (10/group). FIG. 17A depicts virus neutralization responses against the IRV-targeted strain A/Wisconsin/588/2019 (H1N1). FIG. 17B depicts virus neutralization responses against the IRV-targeted strain A/Darwin/6/2021 (H3N2). FIG. 17C depicts virus neutralization responses against the IRV-targeted strain B/Austria/1359417/2021 (B/Victoria). FIG. 17D depicts virus neutralization responses against each of the IRV-targeted strain B/Phuket/3073/2013 (B/Yamagata). Neutralization titers elicited by mIRV, tIRV, or qIRV against the shared vaccine strains (H1N1, H3N2, and B/Vic) were not statistically different (FIG. 17A-C) indicating an absence of interference. Neutralization titers against B/Yamagata elicited by mIRV and qIRV were also not statistically different (FIG. 17D). All titers are reported as GMT with 95% CI. Each data point represents one animal. Statistical comparisons were performed using ANOVA and Tukey's multiple comparisons test, ***, p<0.0001; ns, not significant. IM, intramuscular; QIV, quadrivalent influenza vaccine; MNT, microneutralization assay; GMT, geometric mean titer; LOD, limit of detection for MNT assy.

FIG. 18A-D. Hematology findings were consistent with an inflammatory leukogram and included higher neutrophil counts on Days 3 and 17; a higher incidence of hyper-segmented neutrophils on Day 17; and higher monocytes, eosinophils, and/or large unstained cells on Days 3 and 17 (FIG. 18A). Findings consistent with an acute phase response were also noted for both vaccines and included higher fibrinogen on Day 17; higher globulin and/or lower albumin on Days 3 and 17; and higher alpha-2 macroglobulin (A2M) and alpha-1-acid glycoprotein (A1AGP) on Days 3 and 17 (FIG. 18B-D). Neutrophil counts (A), fibrinogen concentrations (B), A2M concentrations (C), and A1AGP concentrations (D) in male (M) and female (F) Wistar Han rats immunized IM with mIRV (34 μg; 15/sex/group), qIRV (30 μg; 7.5 μg/HA; 15/sex/group), or saline (control). Blood samples were collected on Dosing Phase Days 3, 17, and/or 39 (D3, D17, and D39, respectively). Box-and-whisker plots represent group medians (middle line), 25th and 75th percentiles (box), and min and max (lower and upper whiskers). All saline groups depicted in panels A-D are shown in black. A2M, alpha-2 macroglobulin; A1AGP, alpha-1-acid glycoprotein; IM, intramuscular.

FIG. 19 depicts Mean Influenza Challenge Virus Viral Load by qRT-PCR by Day, Per Protocol Population, wherein treatment groups tested are monovalent modRNA HA (N=55), QIV comparator (N=48), and placebo (N=52).

FIG. 20 depicts Box Plot of Area Under the Curve of Influenza Challenge Virus Viral Load by qRT-PCR by Day, Per Protocol Population. Treatment groups tested are monovalent modRNA HA (N=55), QIV comparator (N=48), and placebo (N=52). Whiskers represent the minimum and maximum, the box represents the interquartile range with the line representing the median, the diamond representing the mean, and the circles showing the values for each individual.

FIG. 21. Forest Plot of Vaccine Efficacy for qRT-PCR Confirmed Moderately Severe Influenza Infection, Per Protocol Population, i.e., QIV comparator and monovalent modRNA HA.

FIG. 22 depicts 50% neutralization titer results 3 weeks post dose 1 against H1N1 A/California strain from drug product formulations respectively described in Table 57 and Table 58.

FIG. 23 depicts 50% neutralization titer results 3 weeks post dose 1 against RSV M37 strain from drug product formulations respectively described in Table 57 and Table 58.

FIG. 24 depicts 50% neutralization titer results 3 weeks post dose 1 against RSV B18537 strain from drug product formulations respectively described in Table 57 and Table 58.

FIG. 25A-D. FIG. 25A depicts 50% neutralization titer results 3 weeks post dose 1 against Influenza B/Colorado strain from Bv/Colorado/06/2017-HA-A370-374 samples respectively described in Table 61. FIG. 25B depicts 50% neutralization titer results 3 weeks post dose 1 against Influenza B/Washington strain from Bv/Washington/02/2019-HA-A369-373 samples respectively described in Table 61. FIG. 25C depicts 50% neutralization titer results 3 weeks post dose 1 against Influenza B/Phuket strain from By/Phuket/3073/2013-HA-A371-375 samples respectively described in Table 61. FIG. 25D depicts 50% neutralization titer results 3 weeks post dose 1 against Influenza B/Austria strain from mutant B/Austria samples respectively described in Table 62.

FIG. 26A-D. FIG. 26A depicts 50% neutralization titer results 2 weeks post dose 2 against Influenza B/Colorado strain from Bv/Colorado/06/2017-HA-A370-374 samples respectively described in Table 61. FIG. 26B depicts 50% neutralization titer results 2 weeks post dose 2 against Influenza B/Washington strain from Bv/Washington/02/2019-HA-A369-373 samples respectively described in Table 61. FIG. 26C depicts 50% neutralization titer results 2 weeks post dose 2 against Influenza B/Phuket strain from By/Phuket/3073/2013-HA-A371-375 samples respectively described in Table 61. FIG. 26D depicts 50% neutralization titer results 2 weeks post dose 2 against Influenza B/Austria strain from mutant B/Austria samples respectively described in Table 62.

FIG. 27 depicts 50% neutralization titer results 2 weeks post dose 2 against Influenza B/Austria strain from mutant B/Austria samples. Flu B modRNA constructs bearing a deletion within the HA fusion peptide, A (369-373), led to higher neutralizing antibody titers in-vivo compared to WT benchmark control.

FIG. 28 depicts 50% neutralization titer results 2 weeks post dose 2 against Influenza B/Austria strain from samples respectively described in Table 60.

FIG. 29 depicts 50% neutralization titer results 2 weeks post dose 2 against Influenza H1N1 A/Wisconsin/67/2022 strain from samples respectively described in Table 63.

FIG. 30A-B. FIG. 30A depicts 50% neutralization titer results 2 weeks post dose 2 against Influenza H1N1 A/Wisconsin/67/2022 strain from samples respectively described in Table 63. FIG. 30B depicts 50% neutralization titer results 2 weeks post dose 2 against Influenza H3N2 A/Darwin/06/2021 strain from samples respectively described in Table 63.

FIG. 31 depicts 50% neutralization titer results 3 weeks post dose 1 against Influenza Bv/Austria strain from samples respectively described in Table 63. The “?” in the FIG. represents “A.”

FIG. 32A-B. FIG. 32A depicts 50% neutralization titer results 3 weeks post dose 1 against Influenza H1N1 A/Wisconsin/67/2022 strain from samples respectively described in Table 63, wherein the “?” represents “A.” FIG. 32B depicts 50% neutralization titer results 3 weeks post dose 1 against Influenza H3N2 A/Darwin/06/2021 strain from samples respectively described in Table 63. The “?” in the FIG. represents “A.”

FIG. 33 depicts 50% neutralization titer results 2 weeks post dose 2 against Influenza Bv/Austria strain from samples respectively described in Table 63. The “?” in the FIG. represents “A.”

FIG. 34A-B. FIG. 34A depicts 50% neutralization titer results 2 weeks post dose 2 against Influenza H1N1 A/Wisconsin/67/2022 strain from samples respectively described in Table 63. The “?” in the FIG. represents “A.” FIG. 34B depicts 50% neutralization titer results 2 weeks post dose 2 against Influenza H3N2 A/Darwin/06/2021 strain from samples respectively described in Table 63. The “?” in the FIG. represents “A.”

### DETAILED DESCRIPTION

Embodiments of the present disclosure provide RNA (e.g., mRNA) vaccines that include polynucleotide encoding an influenza virus antigen. Influenza virus RNA vaccines, as provided herein may be used to induce a balanced immune response, comprising both cellular and humoral immunity, without many of the risks associated with DNA vaccination. In some embodiments, the virus is a strain of Influenza A or Influenza B or combinations thereof.

In one aspect, the disclosure relates to an immunogenic composition including: (i) a first ribonucleic acid (RNA) polynucleotide having an open reading frame encoding a first antigen, said antigen including at least one influenza virus antigenic polypeptide or an immunogenic fragment thereof, and (ii) a second RNA polynucleotide having an open reading frame encoding a second antigen, said second antigen including at least one influenza virus antigenic polypeptide or an immunogenic fragment thereof, wherein the first and second RNA polynucleotides are formulated in a lipid nanoparticle (LNP). In some embodiments, the first and second antigens include hemagglutinin (HA), or an immunogenic fragment or variant thereof. In some embodiments, the first antigen includes an HA from a different subtype of influenza virus to the influenza virus antigenic polypeptide or an immunogenic fragment thereof of the second antigen. In some embodiments, the composition further includes (iii) a third antigen including at least one influenza virus antigenic polypeptide or an immunogenic fragment thereof, wherein the third antigen is from influenza virus but is from a different strain of influenza virus to both the first and second antigens. In some embodiments, the first, second and third RNA polynucleotides are formulated in a lipid nanoparticle.

In some embodiments, the composition further includes (iv) a fourth RNA polynucleotide having an open reading frame encoding a fourth antigen, said antigen including at least one influenza virus antigenic polypeptide or an immunogenic fragment thereof, wherein the fourth antigen is from influenza virus but is from a different strain of influenza virus to the first, second and third antigens. In some embodiments, the first, second, third, and fourth RNA polynucleotides are formulated in a lipid nanoparticle.

In some embodiments, the RNA polynucleotides are mixed in desired ratios in a single vessel and are subsequently formulated into lipid nanoparticles. The inventors surprisingly discovered that the initial input of different RNA polynucleotides at a known ratio to be formulated in a single LNP process surprisingly resulted in LNPs encapsulating the different RNA polynucleotides in about the same ratio as the input ratio. The results were surprising in view of the potential for the manufacturing process to favor one RNA polynucleotide to another when encapsulating the RNA polynucleotides into an LNP. Such embodiments may be referred herein as “pre-mix”. Accordingly, in some embodiments, first and second RNA polynucleotides are formulated in a single lipid nanoparticle. In some embodiments, the first, second, third, and fourth RNA polynucleotides are formulated in a single LNP. In some embodiments, the first, second, third, fourth, and fifth RNA polynucleotides are formulated in a single LNP. In some embodiments, the first, second, third, fourth, fifth, and sixth RNA polynucleotides are formulated in a single LNP. In some embodiments, the first, second, third, fourth, fifth, sixth, and seventh RNA polynucleotides are formulated in a single LNP. In some embodiments, the first, second, third, fourth, fifth, sixth, seventh, and eighth RNA polynucleotides are formulated in a single LNP.

In some embodiments, the molar ratio of the first RNA polynucleotide to the second RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the second RNA polynucleotide is greater than 1:1.

In some embodiments, the molar ratio of the first RNA polynucleotide to the third RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the third RNA polynucleotide is greater than 1:1

In some embodiments, the molar ratio of the first RNA polynucleotide to the fourth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the fourth RNA polynucleotide is greater than 1:1. In some embodiments, the molar ratio of the first RNA polynucleotide to the fifth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the fifth RNA polynucleotide is greater than 1:1. In some embodiments, the molar ratio of the first RNA polynucleotide to the sixth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the sixth RNA polynucleotide is greater than 1:1. In some embodiments, the molar ratio of the first RNA polynucleotide to the seventh RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the seventh RNA polynucleotide is greater than 1:1. In some embodiments, the molar ratio of the first RNA polynucleotide to the eighth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the eighth RNA polynucleotide is greater than 1:1.

Self-Amplifying RNA (saRNA)

In some embodiments, the RNA molecule, such as the first RNA molecule, is an saRNA. “saRNA,” “self-amplifying RNA,” and “replicon” refer to RNA with the ability to replicate itself. Self-amplifying RNA molecules may be produced by using replication elements derived from a virus or viruses, e.g., alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNAs. These daughter RNAs, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the protein of interest, e.g., an antigen. The overall result of this sequence of transcriptions is an amplification in the number of the introduced saRNAs and so the encoded gene of interest, e.g., a viral antigen, can become a major polypeptide product of the cells.

In some embodiments, the self-amplifying RNA includes at least one or more genes selected from any one of viral replicases, viral proteases, viral helicases and other nonstructural viral proteins. In some embodiments, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In some embodiments, the self-amplifying RNA molecule is not encapsulated in a virus-like particle. Self-amplifying RNA molecules described herein may be designed so that the self-amplifying RNA molecule cannot induce production of infectious viral particles. This may be achieved, for example, by omitting one or more viral genes encoding structural proteins that are necessary to produce viral particles in the self-amplifying RNA. For example, when the self-amplifying RNA molecule is based on an alphavirus, such as Sinbis virus (SIN), Semliki forest virus and Venezuelan equine encephalitis virus (VEE), one or more genes encoding viral structural proteins, such as capsid and/or envelope glycoproteins, may be omitted.

In some embodiments, a self-amplifying RNA molecule described herein encodes (i) an RNA-dependent RNA polymerase that may transcribe RNA from the self-amplifying RNA molecule and (ii) a polypeptide of interest, e.g., a viral antigen. In some embodiments, the polymerase may be an alphavirus replicase, e.g., including any one of alphavirus protein nsP1, nsP2, nsP3, nsP4, and any combination thereof. In some embodiments, the self-amplifying RNA molecules described herein may include one or more modified nucleotides (e.g., pseudouridine, N6-methyladenosine, 5-methylcytidine, 5-methyluridine). In some embodiments, the self-amplifying RNA molecules does not include a modified nucleotide (e.g., pseudouridine, N6-methyladenosine, 5-methylcytidine, 5-methyluridine).

The saRNA construct may encode at least one non-structural protein (NSP), disposed 5′ or 3′ of the sequence encoding at least one peptide or polypeptide of interest. In some embodiments, the sequence encoding at least one NSP is disposed 5′ of the sequences encoding the peptide or polypeptide of interest. Thus, the sequence encoding at least one NSP may be disposed at the 5′ end of the RNA construct. In some embodiments, at least one non-structural protein encoded by the RNA construct may be the RNA polymerase nsP4. In some embodiments, the saRNA construct encodes nsP1, nsP2, nsP3 and, nsP4. As is known in the art, nsP1 is the viral capping enzyme and membrane anchor of the replication complex (RC). nsP2 is an RNA helicase and the protease responsible for the ns polyprotein processing. nsP3 interacts with several host proteins and may modulate protein poly- and mono-ADP-ribosylation. nsP4 is the core viral RNA-dependent RNA polymerase. In some embodiments, the polymerase may be an alphavirus replicase, e.g., comprising one or more of alphavirus proteins nsP1, nsP2, nsP3, and nsP4.

Whereas natural alphavirus genomes encode structural virion proteins in addition to the non-structural replicase polypeptide, in some embodiments, the self-amplifying RNA molecules do not encode alphavirus structural proteins. In some embodiments, the self-amplifying RNA may lead to the production of genomic RNA copies of itself in a cell, but not to the production of RNA that includes virions. Without being bound by theory or mechanism, the inability to produce these virions means that, unlike a wild-type alphavirus, the self-amplifying RNA molecule cannot perpetuate itself in infectious form. The alphavirus structural proteins which are necessary for perpetuation in wild-type viruses can be absent from self-amplifying RNAs of the present disclosure and their place can be taken by gene(s) encoding the immunogen of interest, such that the subgenomic transcript encodes the immunogen rather than the structural alphavirus virion proteins.

In some embodiments, the self-amplifying RNA molecule may have two open reading frames. The first (5′) open reading frame can encode a replicase; the second (3′) open reading frame can encode a polypeptide comprising an antigen of interest. In some embodiments the RNA may have additional (e.g., downstream) open reading frames, e.g., to encode further antigens or to encode accessory polypeptides.

In some embodiments, the second RNA or the saRNA molecule further includes (1) an alphavirus 5′ replication recognition sequence, and (2) an alphavirus 3′ replication recognition sequence. In some embodiments, the 5′ sequence of the self-amplifying RNA molecule is selected to ensure compatibility with the encoded replicase.

Optionally, self-amplifying RNA molecules described herein may also be designed to induce production of infectious viral particles that are attenuated or virulent, or to produce viral particles that are capable of a single round of subsequent infection.

In some embodiments, the saRNA molecule is alphavirus-based. Alphaviruses include a set of genetically, structurally, and serologically related arthropod-borne viruses of the Togaviridae family. Exemplary viruses and virus subtypes within the alphavirus genus include Sindbis virus, Semliki Forest virus, Ross River virus, and Venezuelan equine encephalitis virus. As such, the self-amplifying RNA described herein may incorporate an RNA replicase derived from any one of semliki forest virus (SFV), sindbis virus (SIN), Venezuelan equine encephalitis virus (VEE), Ross-River virus (RRV), or other viruses belonging to the alphavirus family. In some embodiments, the self-amplifying RNA described herein may incorporate sequences derived from a mutant or wild-type virus sequence, e.g., the attenuated TC83 mutant of VEEV has been used in saRNAs.

Alphavirus-based saRNAs are (+)-stranded saRNAs that may be translated after delivery to a cell, which leads to translation of a replicase (or replicase-transcriptase). The replicase is translated as a polyprotein which auto-cleaves to provide a replication complex which creates genomic (−)-strand copies of the (+)-strand delivered RNA. These (−)-strand transcripts may themselves be transcribed to give further copies of the (+)-stranded parent RNA and also to give a subgenomic transcript which encodes the desired gene product. Translation of the subgenomic transcript thus leads to in situ expression of the desired gene product by the infected cell. Suitable alphavirus saRNAs may use a replicase from a sindbis virus, a semliki forest virus, an eastern equine encephalitis virus, a Venezuelan equine encephalitis virus, or mutant variants thereof.

In some embodiments, the self-amplifying RNA molecule is derived from or based on a virus other than an alphavirus, such as a positive-stranded RNA virus, and in particular a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375). In some aspects, one or more of the alphaviruses in the list may be excluded.

In some embodiments, the self-amplifying RNA molecules described herein are larger than other types of RNA (e.g., saRNA). Typically, the self-amplifying RNA molecules described herein include at least about 4 kb. For example, the self-amplifying RNA may be equal to any one of, at least any one of, at most any one of, or between any two of 3 kb, 4 kb, 5 kb, 6 kb, 7 kb, 8 kb, 9 kb, 10 kb, 11 kb, 12 kb, 13 kb, 14 kb, 15 kb, 16 kb. In some instances the self-amplifying RNA may include at least about 5 kb, at least about 6 kb, at least about 7 kb, at least about 8 kb, at least about 9 kb, at least about 10 kb, at least about 11 kb, at least about 12 kb, or more than 12 kb. In certain examples, the self-amplifying RNA is about 4 kb to about 12 kb, about 5 kb to about 12 kb, about 6 kb to about 12 kb, about 7 kb to about 12 kb, about 8 kb to about 12 kb, about 9 kb to about 12 kb, about 10 kb to about 12 kb, about 11 kb to about 12 kb, about 5 kb to about 11 kb, about 5 kb to about 10 kb, about 5 kb to about 9 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb.

In some embodiments, the self-amplifying RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-amplifying RNA may then be produced as a fusion polypeptide or engineered in such a manner to result in separate polypeptide or peptide sequences. In some embodiments, the saRNA molecule may encode one polypeptide of interest or more, such as an antigen or more than one antigen, e.g., two, three, four, five, six, seven, eight, nine, ten, or more polypeptides. Alternatively, or in addition, one saRNA molecule may also encode more than one polypeptide of interest or more, such as an antigen, e.g., a bicistronic, or tricistronic RNA molecule that encodes different or identical antigens. An exemplary bicistronic saRNA encoding HA and NA include the sequence set forth in SEQ ID NO: 70 (published as SEQ ID NO: 9 of PCT/IB2023/057034, published as WO2024/013625). Another exemplary bicistronic saRNA includes the the sequence set forth in SEQ ID NO: 71 (published as SEQ ID NO: 10 of PCT/IB2023/057034, published as WO2024/013625). See, for example, International patent application PCT/IB2023/057034, published as WO2024/013625, entitled, “Self-amplifying rna encoding an influenza virus antigen,” (Pfizer Inc.) filed on Jul. 7, 2023, which is incorporated by reference in its entirety and describes saRNA molecules and bicistronic saRNA.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first RNA molecule to a RNA molecule at the 5′-end or the 3′-end, but also includes insertion of the whole first RNA molecule into any two nucleotides in the second RNA molecule. The first second RNA molecule can be linked to a second RNA molecule by a phosphodiester bond or a linker. The linker can be, e.g., a polynucleotide.

In some embodiments, the self-amplifying RNA described herein may encode one or more polypeptide antigens that include a range of epitopes. In some embodiments, the self-amplifying RNA described herein may encode epitopes capable of eliciting either a helper T-cell response or a cytotoxic T-cell response or both.

In some embodiments, the saRNA molecule is purified, e.g., such as by filtration that may occur via, e.g., ultrafiltration, diafiltration, or, e.g., tangential flow ultrafiltration/diafiltration.

Some embodiments of the disclosure are directed to a composition comprising a self-amplifying RNA molecule comprising a 5′ Cap, a 5′ untranslated region, a coding region comprising a sequence encoding an RNA-dependent RNA polymerase (also referred to as a “replicase”), a subgenomic promoter, such as one derived from an alphavirus, an open reading frame encoding a gene of interest (e.g., an antigen derived from influenza virus), a 3′ untranslated region, and a 3′ poly A sequence. In some embodiments, at least 5% of a total population of a particular nucleotide in the saRNA molecule has been replaced with one or more modified or unnatural nucleotides.

In some embodiments, the saRNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine, which is further described below.

In some embodiments, the saRNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine, which is further described below. In some embodiments, the RNA may include a 5′ cap comprising a 7′-methylguanosine, and the first 1, 2 or 3 5′ ribonucleotides may be methylated at the 2′ position of the ribose. In alternative embodiments, each RNA polynucleotide encoding a particular antigen is formulated in an individual LNP, such that each LNP encapsulates an RNA polynucleotide encoding identical antigens. Such embodiments may be referred herein as “post-mix”. Accordingly, in some embodiments, the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; the sixth RNA polynucleotide is formulated in a sixth LNP; the seventh RNA polynucleotide is formulated in a seventh LNP; and the eighth RNA polynucleotide is formulated in an eighth LNP.

In some embodiments, the molar ratio of the first LNP to the second LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the second LNP is greater than 1:1.

In some embodiments, the molar ratio of the first LNP to the third LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the third LNP is greater than 1:1.

In some embodiments, the molar ratio of the first LNP to the fourth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the fourth LNP is greater than 1:1. In some embodiments, the molar ratio of the first LNP to the fifth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the fifth LNP is greater than 1:1. In some embodiments, the molar ratio of the first LNP to the sixth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the sixth LNP is greater than 1:1. In some embodiments, the molar ratio of the first LNP to the seventh LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the seventh LNP is greater than 1:1. In some embodiments, the molar ratio of the first LNP to the eighth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the eighth LNP is greater than 1:1.

Surprisingly, the inventors discovered that regardless of the process, the resulting ratio of RNA polynucleotide was comparable whether the plurality of RNA polynucleotides are mixed prior to formulation in an LNP (pre-mixed) or whether the RNA polynucleotides encoding a particular antigen is formulated in an individual LNP and the plurality of LNPs for different antigens are mixed (post-mixed). As a result of the discovery, there may be an option for medical professionals to mix and administer different ratios of antigens depending on the influenza season, particularly when the individual LNPs encapsulate RNA for a single antigen.

In some embodiments, the antigenic polypeptide encodes a hemagglutinin protein or immunogenic fragment thereof. In some embodiments, the hemagglutinin protein is H1, H2, H3, H4, H5, H6, H7, H8, H9, H10, H11, H12, H13, H14, H15, H16, H17, H18, or an immunogenic fragment thereof. In some embodiments, the hemagglutinin protein does not comprise a head domain. In some embodiments, the hemagglutinin protein comprises a portion of the head domain. In some embodiments, the hemagglutinin protein does not comprise a cytoplasmic domain. In some embodiments, the hemagglutinin protein comprises a portion of the cytoplasmic domain. In some embodiments, the truncated hemagglutinin protein comprises a portion of the transmembrane domain.

Some embodiments provide influenza vaccines comprising one or more RNA polynucleotides having an open reading frame encoding a hemagglutinin protein and a pharmaceutically acceptable carrier or excipient, formulated within a cationic lipid nanoparticle. In some embodiments, the hemagglutinin protein is selected from H1, H7 and H10. In some embodiments, the RNA polynucleotide further encodes neuraminidase (NA) protein. In some embodiments, the hemagglutinin protein is derived from a strain of Influenza A virus or Influenza B virus or combinations thereof. In some embodiments, the Influenza virus is selected from H1N1, H3N2, H7N9, and H10N8.

In some embodiments, the virus is a strain of Influenza A or Influenza B or combinations thereof. In some embodiments, the strain of Influenza A or Influenza B is associated with birds, pigs, horses, dogs, humans, or non-human primates. In some embodiments, the antigenic polypeptide encodes a hemagglutinin protein or fragment thereof. In some embodiments, the hemagglutinin protein is H7 or H10 or a fragment thereof. In some embodiments, the hemagglutinin protein comprises a portion of the head domain (HA1). In some embodiments, the hemagglutinin protein comprises a portion of the cytoplasmic domain. In some embodiments, the truncated hemagglutinin protein. In some embodiments, the protein is a truncated hemagglutinin protein comprises a portion of the transmembrane domain. In some embodiments, the virus is selected from the group consisting of H7N9 and H10N8. Protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length.

In some embodiments, an Influenza RNA composition includes an RNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the influenza antigen. Antigenic fusion proteins, in some embodiments, retain the functional property from each original protein.

Some embodiments provide methods of preventing or treating influenza viral infection comprising administering to a subject any of the vaccines described herein. In some embodiments, the antigen specific immune response comprises a T cell response. In some embodiments, the antigen specific immune response comprises a B cell response. In some embodiments, the antigen specific immune response comprises both a T cell response and a B cell response. In some embodiments, the method of producing an antigen specific immune response involves a single administration of the vaccine. In some embodiments, the vaccine is administered to the subject by intradermal, intramuscular injection, subcutaneous injection, intranasal inoculation, or oral administration.

In some embodiments, the RNA (e.g., mRNA) polynucleotides or portions thereof may encode one or more polypeptides or fragments thereof of an influenza strain as an antigen.

mRNA Vaccines of the Disclosure

The present disclosure relates to mRNA vaccines in general. Several mRNA vaccine platforms are available in the prior art. The basic structure of in vitro transcribed (IVT) mRNA closely resembles “mature” eukaryotic mRNA and includes (i) a protein-encoding open reading frame (ORF), flanked by (ii) 5′ and 3′ untranslated regions (UTRs), and at the end sides (iii) a 7-methyl guanosine 5′ cap structure and (iv) a 3′ poly(A) tail. The non-coding structural features play important roles in the pharmacology of mRNA and can be individually optimized to modulate the mRNA stability, translation efficiency, and immunogenicity. By incorporating modified nucleosides, mRNA transcripts referred to as “nucleoside-modified mRNA” can be produced with reduced immunostimulatory activity, and therefore an improved safety profile can be obtained. In addition, modified nucleosides allow the design of mRNA vaccines with strongly enhanced stability and translation capacity, as they can avoid the direct antiviral pathways that are induced by type IFNs and are programmed to degrade and inhibit invading mRNA. For instance, the replacement of uridine with pseudouridine in IVT mRNA reduces the activity of 2′-5′-oligoadenylate synthetase, which regulates the mRNA cleavage by RNase L. In addition, lower activities are measured for protein kinase R, an enzyme that is associated with the inhibition of the mRNA translation process.

Besides the incorporation of modified nucleotides, other approaches have been validated to increase the translation capacity and stability of mRNA. One example is the development of “sequence-engineered mRNA”. Here, mRNA expression can be strongly increased by sequence optimizations in the ORF and UTRs of mRNA, for instance by enriching the GC content, or by selecting the UTRs of natural long-lived mRNA molecules. Another approach is the design of “self-amplifying mRNA” constructs. These are mostly derived from alphaviruses and contain an ORF that is replaced by the antigen of interest together with an additional ORF encoding viral replicase. The latter drives the intracellular amplification of mRNA and can therefore significantly increase the antigen expression capacity.

Also, several modifications have been implemented at the end structures of mRNA. Anti-reverse cap (ARCA) modifications can ensure the correct cap orientation at the 5′ end, which yields almost complete fractions of mRNA that can efficiently bind the ribosomes. Other cap modifications, such as phosphorothioate cap analogs, can further improve the affinity towards the eukaryotic translation initiation factor 4E, and increase the resistance against the RNA decapping complex.

Conversely, by modifying its structure, the potency of mRNA to trigger innate immune responses can be further improved, but to the detriment of translation capacity. By stabilizing the mRNA with either a phosphorothioate backbone, or by its precipitation with the cationic protein protamine, antigen expression can be diminished, but stronger immune-stimulating capacities can be obtained.

In one aspect the invention relates to an immunogenic composition comprising an mRNA molecule that encodes one or more polypeptides or fragments thereof of an influenza strain as an antigen.

In some embodiments, the mRNA molecule comprises a nucleoside-modified mRNA. mRNA useful in the disclosure typically include a first region of linked nucleosides encoding a polypeptide of interest (e.g., a coding region), a first flanking region located at the 5′-terminus of the first region (e.g., a 5-UTR), a second flanking region located at the 3′-terminus of the first region (e.g., a 3-UTR), at least one 5′-cap region, and a 3′-stabilizing region. In some embodiments, the mRNA of the disclosure further includes a poly-A region or a Kozak sequence (e.g., in the 5′-UTR). In some cases, mRNA of the disclosure may contain one or more intronic nucleotide sequences capable of being excised from the polynucleotide. In some embodiments, mRNA of the disclosure may include a 5′ cap structure, a chain terminating nucleotide, a stem loop, a poly A sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine), and/or a 5-substituted cytidine (e.g., 5-methyl-cytidine).

The compositions described herein comprise at least one RNA polynucleotide, such as a mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide.

In preferred embodiments, the 5′ UTR comprises SEQ ID NO: 1.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

In preferred embodiments, the 3′ UTR comprises SEQ ID NO: 2.

An “open reading frame” is a continuous stretch of DNA beginning with a start codon (e.g., methionine (ATG)), and ending with a stop codon (e.g., TAA, TAG or TGA) and encodes a polypeptide. 20

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In preferred embodiments, the 3′ polyadenylation tail comprises SEQ ID NO: 3.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

In some embodiments, a LNP includes one or more RNAs, and the one or more RNAs, lipids, and amounts thereof may be selected to provide a specific N: P ratio. The N: P ratio of the composition refers to the molar ratio of nitrogen atoms in one or more lipids to the number of phosphate groups in an RNA. In general, a lower N: P ratio is preferred. The one or more RNA, lipids, and amounts thereof may be selected to provide an N: P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N: P ratio may be from about 2:1 to about 8:1. In other embodiments, the N: P ratio is from about 5:1 to about 8:1. For example, the N: P ratio may be about 5.0:1, about 5.5:1, about 5.67:1, about 6.0:1, about 6.5:1, or about 7.0:1. For example, the N: P ratio may be about 5.67:1.

mRNA of the disclosure may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

mRNA of the disclosure may include one or more alternative components, as described herein, which impart useful properties including increased stability and/or the lack of a substantial induction of the innate immune response of a cell into which the polynucleotide is introduced. For example, a modRNA may exhibit reduced degradation in a cell into which the modRNA is introduced, relative to a corresponding unaltered mRNA. These alternative species may enhance the efficiency of protein production, intracellular retention of the polynucleotides, and/or viability of contacted cells, as well as possess reduced immunogenicity.

mRNA of the disclosure may include one or more modified (e.g., altered or alternative) nucleobases, nucleosides, nucleotides, or combinations thereof. The mRNA useful in a LNP can include any useful modification or alteration, such as to the nucleobase, the sugar, or the internucleoside linkage (e.g., to a linking phosphate/to a phosphodiester linkage/to the phosphodiester backbone). In certain embodiments, alterations (e.g., one or more alterations) are present in each of the nucleobase, the sugar, and the internucleoside linkage. Alterations according to the present disclosure may be alterations of ribonucleic acids (RNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

mRNA of the disclosure may or may not be uniformly altered along the entire length of the molecule. For example, one or more or all types of nucleotide (e.g., purine or pyrimidine, or any one or more or all of A, G, U, C) may or may not be uniformly altered in a mRNA, or in a given predetermined sequence region thereof. In some instances, all nucleotides X in a mRNA (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus. The polynucleotide may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some instances, nucleic acids do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc., and/or 3) termination or reduction in protein translation.

In some embodiments, the mRNA comprises one or more alternative nucleoside or nucleotides. The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

In some embodiments, the nucleobase is an alternative uracil. Exemplary nucleobases and nucleosides having an alternative uracil include pseudouridine (w), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (m U), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5s2U), 5-aminomethyl-2-thio-uracil (nmVu), 5-methylaminomethyl-uracil (mnm5U), 5-methylaminomethyl-2-thio-uracil (mnmVu), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnmVu), 5-propynyl-uracil, 1-propynyl-pseudouracil, 5-taurinomethyl-uracil (xm5U), 1-taurinomethyl-pseudouridine, 5-taurinomethyl-2-thio-uracil (xm5s2U), 1-taurinomethyl-4-thio-pseudouridine, 5-methyl-uracil (m5U, i.e., having the nucleobase deoxythymine), 1-methyl-pseudouridine (mV), 5-methyl-2-thio-uracil (m5s2U), I-methyl-4-thio-pseudouridine (m xj/), 4-thio-1-methyl-pseudouridine, 3-methyl-pseudouridine (m \/), 2-thio-1-methyl-pseudouridine, 1-methyl-1-deaza-pseudouri dine, 2-thio-I-methyl-1-deaza-pseudouri dine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl) uracil (acp U), I-methyl-3-(3-amino-3-carboxypropyl) pseudouridine (acp w), 5-(isopentenylaminomethyl) uracil (inm5U), 5-(isopentenylaminomethyl)-2-thio-uracil (inm5s2U), 5,2′-O-dimethyl-uridine (m5Um), 2-thio-2′-O_methyl-uridine (s2Um), 5-methoxycarbonylmethyl-2′-O-methyl-uridine (mem Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m Um), and 5-(isopentenylaminomethyl)-2′-O-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(I-E-propenylamino)]uracil.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocy tidine, 4-thio-1-methy 1-pseudoisocy tidine, 4-thio-1-methyl-1-deaza-pseudoisocytidine, 1-methyl-1-deaza-pseudoisocyti dine, zebularine, 5-aza-zebularine, 5-methy 1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-0-dimethyl-cytidine (m4Cm), 5-formyl-2′-O-methyl-cytidine (f5Cm), N4, N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-azaadenine, 7-deaza-2-amino-purine, 7-deaza-8-aza-2-amino-purine, 7-deaza-2,6-diaminopurine, 7-deaza-8-aza-2,6-diaminopurine, 1-methy 1-adenine (m1 A), 2-methyl-adenine (m2A), N6-methyl-adenine (m6A), 2-methylthio-N6-methyl-adenine (ms2m6A), N6-isopentenyl-adenine (16A), 2-methylthio-N6-isopentenyl-adenine (ms216A), N6-(cis-hydroxyisopentenyl) adenine (io6A), 2-methylthio-N6-(cis-hydroxyisopentenyl) adenine (ms2io6A), N6-glycinylcarbamoyl-adenine (g6A), N6-threonylcarbamoyl-adenine (16A), N6-methyl-N6-threonylcarbamoyl-adenine (m6t6A), 2-methylthio-N6-threonylcarbamoyl-adenine (ms2g6A), N6, N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6, N6,2′-O-trimethyladenosine (m62Am), 1,2′-O-dimethyl-adenosine (m1 Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (mil), wyosine (imG), methylwyosine (mimG), 4-demethyl-wyosine (imG-14), isowyosine (imG2), wybutosine (yW), peroxywybutosine (02yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQI), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2, N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2, N2-dimethyl-6-thio-guanine, N2-methyl-2′-O-methyl-guanosine (m2Gm), N2, N2-dimethyl-2′-O-methyl-guanosine (m22Gm), 1-methyl-2′-O-methyl-guanosine (mlGm), N2,7-dimethyl-2′-O-methyl-guanosine (m2,7Gm), 2′-O-methyl-inosine (Im), 1,2′-O-dimethyl-inosine (mllm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

The mRNA may include a 5′-cap structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing.

Endogenous polynucleotide molecules may be 5′-end capped generating a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-O-methylated. 5′-decapping through hydrolysis and cleavage of the guanylate cap structure may target a polynucleotide molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional alternative guanosine nucleotides may be used such as α-methylphosphonate and seleno-phosphate nucleotides. Additional alterations include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of an mRNA molecule.

Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide. For example, the Anti-Reverse Cap Analog (ARCA) cap contains two guanosines linked by a 5′-5′-triphosphate group, wherein one guanosine contains an N7-methyl group as well as a 3′-O-methyl group (i.e., N7,′-O-dimethyl-guanosine-5′-triphosphate-5 ‘-guanosine, m7G-3′mppp-G, which may equivalently be designated 3’ 0-Me-m7G (5′) ppp (5′) G). The 3′-0 atom of the other, unaltered, guanosine becomes linked to the 5′-terminal nucleotide of the capped polynucleotide (e.g., an mRNA). The N7- and 3′-O-methylated guanosine provides the terminal moiety of the capped polynucleotide (e.g., mRNA). Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference.

Alternatively, a cap analog may be a N7-(4-chlorophenoxy ethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G (5) ppp (5′) G and a N7-(4-chlorophenoxyethyl)-m3′-OG (5) ppp (5′) G cap analog (see, e.g., the various cap analogs and the methods of synthesizing cap analogs described in Kore et al. Bioorganic & Medicinal Chemistry 2013 21:4570-4574; the cap structures of which are herein incorporated by reference). In other instances, a cap analog useful in the polynucleotides of the present disclosure is a 4-chloro/bromophenoxy ethyl analog.

While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the Capl structure. This cap results in a higher translational-competency, cellular stability, and a reduced activation of cellular pro-inflammatory cytokines, as compared, e.g., to other 5 ‘cap analog structures known in the art. Other exemplary cap structures include 7 mG (5’) ppp (5 ‘) N, pN2p (Cap 0), 7 mG (5’) ppp (5 ‘) NImpNp (Cap 1), 7 mG (5’)-ppp (5′) NImpN2mp (Cap 2), and m (7) Gpppm (3) (6,6,2′) Apm (2′) Apm (2′) Cpm (2) (3,2′) Up (Cap 4).

Because the alternative polynucleotides may be capped post-transcriptionally, and because this process is more efficient, nearly 100% of the mRNA may be capped. This is in contrast to −80% when a cap analog is linked to a polynucleotide in the course of an in vitro transcription reaction. 5′-terminal caps may include endogenous caps or cap analogs. A 5′-terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5′-cap. A modification on the 5′-cap may increase the stability of polynucleotide, increase the half-life of the polynucleotide, and could increase the polynucleotide translational efficiency. The modified 5′-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

A 5′-UTR may be provided as a flanking region to the mRNA. A 5′-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization.

In one embodiment, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art-non-limiting examples include services from GeneArt (Life Technologies), DNA2.0 (Menlo Park Calif.) and/or proprietary methods. In some embodiments, the open reading frame (ORF) sequence is optimized using optimization algorithms. To alter one or more properties of an mRNA, 5′-UTRs which are heterologous to the coding region of an mRNA may be engineered. The mRNA may then be administered to cells, tissue or organisms and outcomes such as protein level, localization, and/or half-life may be measured to evaluate the beneficial effects the heterologous 5′-UTR may have on the mRNA. Variants of the 5′-UTRs may be utilized wherein one or more nucleotides are added or removed to the termini, including A, T, C or G. 5′-UTRs may also be codon-optimized, or altered in any manner described herein.

mRNAs may include a stem loop such as, but not limited to, a histone stem loop. The stem loop may be a nucleotide sequence that is about 25 or about 26 nucleotides in length. The histone stem loop may be located 3′-relative to the coding region (e.g., at the 3′-terminus of the coding region). As a non-limiting example, the stem loop may be located at the 3′-end of a polynucleotide described herein. In some cases, an mRNA includes more than one stem loop (e.g., two stem loops). A stem loop may be located in a second terminal region of a polynucleotide. As a non-limiting example, the stem loop may be located within an untranslated region (e.g., 3′-UTR) in a second terminal region. In some cases, a mRNA which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide. In other cases, a mRNA, which includes the histone stem loop may be stabilized by an alteration to the 3′-region of the polynucleotide that can prevent and/or inhibit the addition of oligio (U). In yet other cases, a mRNA, which includes the histone stem loop may be stabilized by the addition of an oligonucleotide that terminates in a 3′-deoxynucleoside, 2′,3′-dideoxynucleoside 3′-O-methylnucleosides, 3-O-ethylnucleosides, 3′-arabinosides, and other alternative nucleosides known in the art and/or described herein. In some instances, the mRNA of the present disclosure may include a histone stem loop, a poly-A region, and/or a 5′-cap structure. The histone stem loop may be before and/or after the poly-A region. The polynucleotides including the histone stem loop and a poly-A region sequence may include a chain terminating nucleoside described herein. In other instances, the polynucleotides of the present disclosure may include a histone stem loop and a 5′-cap structure. The 5′-cap structure may include, but is not limited to, those described herein and/or known in the art. In some cases, the conserved stem loop region may include a miR sequence described herein. As a non-limiting example, the stem loop region may include the seed sequence of a miR sequence described herein. In another non-limiting example, the stem loop region may include a miR-122 seed sequence.

mRNA may include at least one histone stem-loop and a poly-A region or polyadenylation signal. In certain cases, the polynucleotide encoding for a histone stem loop and a poly-A region or a polyadenylation signal may code for a pathogen antigen or fragment thereof. In other cases, the polynucleotide encoding for a histone stem loop and a poly-A region or a polyadenylation signal may code for a therapeutic protein. In some cases, the polynucleotide encoding for a histone stem loop and a poly-A region or a polyadenylation signal may code for a tumor antigen or fragment thereof. In other cases, the polynucleotide encoding for a histone stem loop and a poly-A region or a polyadenylation signal may code for an allergenic antigen or an autoimmune self-antigen.

An mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid. During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the alternative polynucleotides of the present disclosure. Generally, the length of a poly-A region of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides. In some instances, the poly-A region may be 80 nucleotides, 120 nucleotides, 160 nucleotides in length on an alternative polynucleotide molecule described herein. In other instances, the poly-A region may be 20, 40, 80, 100, 120, 140 or 160 nucleotides in length on an alternative polynucleotide molecule described herein. In some cases, the poly-A region is designed relative to the length of the overall alternative polynucleotide. This design may be based on the length of the coding region of the alternative polynucleotide, the length of a particular feature or region of the alternative polynucleotide (such as mRNA) or based on the length of the ultimate product expressed from the alternative polynucleotide. When relative to any feature of the alternative polynucleotide (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the alternative polynucleotide to which it belongs. In this context, the poly-A region may be 10, 20, 30, 40, 50, 60, 70, 80, or 90% or more of the total length of the construct or the total length of the construct minus the poly-A region.

In certain cases, engineered binding sites and/or the conjugation of mRNA for poly-A binding protein may be used to enhance expression. The engineered binding sites may be sensor sequences which can operate as binding sites for ligands of the local microenvironment of the mRNA. As a non-limiting example, the mRNA may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct mRNA may be linked together to the PABP (poly-A binding protein) through the 3′-end using alternative nucleotides at the 3′-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site. In certain cases, a poly-A region may be used to modulate translation initiation. While not wishing to be bound by theory, the poly-A region recruits PABP which in turn can interact with translation initiation complex and thus may be essential for protein synthesis. In some cases, a poly-A region may also be used in the present disclosure to protect against 3′-5′-exonuclease digestion. In some instances, an mRNA may include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant mRNA may be assayed for stability, protein production and other parameters including half-life at various time points. It has been discovered that the polyA-G quartet results in protein production equivalent to at least 75% of that seen using a poly-A region of 120 nucleotides alone. In some cases, mRNA may include a poly-A region and may be stabilized by the addition of a 3′-stabilizing region. The mRNA with a poly-A region may further include a 5′-cap structure. In other cases, mRNA may include a poly-A-G Quartet. The mRNA with a poly-A-G Quartet may further include a 5′-cap structure. In some cases, the 3′-stabilizing region which may be used to stabilize mRNA includes a poly-A region or poly-A-G Quartet. In other cases, the 3′-stabilizing region which may be used with the present disclosure include a chain termination nucleoside such as 3′-deoxyadenosine (cordycepin), 3′-deoxyuridine, 3′-deoxycytosine, 3′-deoxyguanosine, 3′-deoxy thymine, 2′,3′-dideoxynucleosides, such as 2′,3′-dideoxyadenosine, 2',3′-dideoxyuridine, 2',3′-dideoxycytosine, 2', 3′-dideoxyguanosine, 2',3′-dideoxythymine, a 2′-deoxynucleoside, or an O-methylnucleoside. In other cases, mRNA which includes a polyA region or a poly-A-G Quartet may be stabilized by an alteration to the 3′-region of the polynucleotide that can prevent and/or inhibit the addition of oligio (U). In yet other instances, mRNA which includes a poly-A region or a poly-A-G Quartet may be stabilized by the addition of an oligonucleotide that terminates in a 3′-deoxynucleoside, 2′,3′-dideoxynucleoside 3-O-methylnucleosides, 3′-O-ethylnucleosides, 3′-arabinosides, and other alternative nucleosides known in the art and/or described herein.

In an embodiment, the mRNA vaccines of the disclosure comprise lipids. The lipids and modRNA can together form nanoparticles. The lipids can encapsulate the mRNA in the form of a lipid nanoparticle (LNP) to aid cell entry and stability of the RNA/lipid nanoparticles.

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body.

Physiochemical properties of lipid nanoparticles may be altered to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

A LNP may include one or more components described herein. In some embodiments, the LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

In some embodiments, for example, a polymer may be included in and/or used to encapsulate or partially encapsulate a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl (meth) acrylate) (PMMA), poly(ethyl (meth) acrylate), poly(butyl(meth) acrylate), poly(isobutyl (meth) acrylate), poly(hexyl (meth) acrylate), poly(isodecyl(meth) acrylate), poly(lauryl(meth) acrylate), poly(phenyl(meth) acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho) esters, poly(butyric acid), poly(valeric acid), poly(lactide-co-caprolactone), trimethylene carbonate, poly(N-acryloylmorpholine) (PACM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin 34, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN@20], polyoxy ethylene sorbitan [TWEEN® 60], polyoxy ethylene sorbitan monooleate [TWEENR80], sorbitan monopalmitate [SPAN®40], sorbitan monostearate [SPAN@60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN@II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, Tris buffer, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol. The lipid nanoparticle compositions may include one or more structural lipids.

Incorporation of structural lipids in the lipid nanoparticle may help mitigate aggregation of other lipids in the particle. Structural lipids can be selected from the group including but not limited to, cholesterol, fecosterol, sitosterol, β-sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In some embodiments, the structural lipid is a steroid. In some embodiments, the structural lipid is cholesterol. In certain embodiments, the structural lipid is an analog of cholesterol. In some embodiments, the structural lipid is alpha-tocopherol. In some preferred embodiments, the sterol comprises

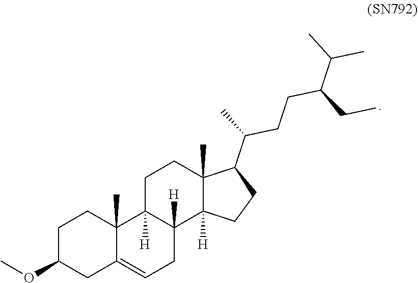

In some preferred embodiments, the sterol comprises

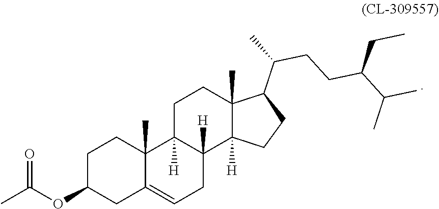

In some preferred embodiments, the sterol comprises stigmasterol, In some preferred embodiments, the sterol comprises sitostanol.

In some embodiments, the structural lipid is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, 45-avenaserol, A7-avenaserol or a A7-stigmasterol, including analogs, salts or esters thereof, alone or in combination. In some embodiments, the sterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol. In some embodiments, the phytosterol is β-sitosterol, campesterol, sigmastanol, or any combination thereof. In some embodiments, the phytosterol is β-sitosterol. In some embodiments, the one or more structural lipids comprises a mixture of β-sitosterol, campesterol, and stigmasterol. In some embodiments, the one or more structural lipids comprises a mixture of β-sitosterol and cholesterol. In one embodiment, the structural lipid is selected from selected from β-sitosterol and cholesterol. In an embodiment, the structural lipid is β-sitosterol. In an embodiment, the structural lipid is cholesterol.

In some embodiments, the one or more structural lipids comprises about 35% to about 85% of β-sitosterol, about 5% to about 35% stigmasterol, and about 5% to about 35% of campesterol. In some embodiments, the one or more structural lipids comprises about 40% to about 80% of β-sitosterol, about 10% to about 30% stigmasterol, and about 10% to about 30% of campesterol. In some embodiments, the one or more structural lipids comprises about 40% to about 70% of β-sitosterol, about 10% to about 25% stigmasterol, and about 10% to about 25% of campesterol. In some embodiments, the one or more structural lipids comprises about 40% to about 70% of β-sitosterol, about 15% to about 25% stigmasterol, and about 15% to about 25% of campesterol. In some embodiments, the one or more structural lipids comprises about 35% to about 45% of β-sitosterol, about 20% to about 30% stigmasterol, and about 20% to about 30% of campesterol. In some embodiments, the one or more structural lipids comprises about 40% to about 50% of β-sitosterol, about 25% to about 35% stigmasterol, and about 25% to about 35% of campesterol. In some embodiments, the one or more structural lipids comprises about 65% to about 75% of β-sitosterol, about 5% to about 15% stigmasterol, and about 5% to about 15% of campesterol. In some embodiments, the one or more structural lipids comprises about 35% to about 85% of β-sitosterol, about 5% to about 35% stigmasterol, and 0% of campesterol. In some embodiments, the one or more structural lipids comprises about 40% to about 80% of β-sitosterol, about 10% to about 30% stigmasterol, and 0% of campesterol. In some embodiments, the one or more structural lipids comprises about 40% to about 70% of β-sitosterol, about 10% to about 25% stigmasterol, and 0% of campesterol. In some embodiments, the one or more structural lipids comprises about 40% to about 70% of β-sitosterol, about 15% to about 25% stigmasterol, and 0% of campesterol. In some embodiments, the one or more structural lipids comprises about 35% to about 45% of β-sitosterol, about 20% to about 30% stigmasterol, and 0% of campesterol. In some embodiments, the one or more structural lipids comprises about 40% to about 50% of β-sitosterol, about 25% to about 35% stigmasterol, and 0% of campesterol. In some embodiments, the one or more structural lipids comprises about 65% to about 75% of β-sitosterol, about 5% to about 15% stigmasterol, and 0% of campesterol. Accordingly, in some preferred embodiments, the composition does not comprise campesterol.

In some embodiments, the composition comprises one or more structural lipids comprises about 10% to about 30% of cholesterol, about 10% to about 30% β-sitosterol, and about 10% to about 30% stigmasterol, and 0% campesterol. See, for example, Table 41. In some embodiments, the composition further comprises about 30-50% cationic lipid and about 5-25% phospholipid.

In some embodiments, the mol % of the one or more structural lipids is between about 1% and 50% of the mol % of the compound having the structure of any of the foregoing compounds present in the lipid nanoparticle. In some embodiments, the mol % of the one or more structural lipids is between about 10% and 40% of the mol % of the compound having the structure of any of the foregoing compounds present in the lipid nanoparticle. In some embodiments, the mol % of the one or more structural lipids is between about 20% and 30% of the mol % of the compound having the structure of any of the foregoing compounds present in the lipid nanoparticle. In some embodiments, the mol % of the one or more structural lipids is about 30% of the mol % of the compound having the structure of any of the foregoing compounds present in the lipid nanoparticle.

In some embodiments, the lipid nanoparticle compositions described herein can comprise about 20 mol % to about 60 mol % structural lipid. In some embodiments, the lipid nanoparticle compositions comprise about 30 mol % to about 50 mol % of structural lipid. In some embodiments, the lipid nanoparticle compositions comprise about 35 mol % to about 45 mol % of structural lipid. In some embodiments, the lipid nanoparticle compositions comprise about 37 mol % to about 42 mol % of structural lipid. In some embodiments, the lipid nanoparticle compositions comprise about 35, about 36, about 37, about 38, about 39, or about 40 mol % of structural lipid. In some embodiments, the nanoparticle comprises about 39 to about 40 mol % structural lipid.

In some embodiments, a LNP of the invention comprises an N: P ratio of from about 2:1 to about 30:1. In some embodiments, a LNP of the invention comprises an N: P ratio of about 6:1. In some embodiments, a LNP of the invention comprises an N: P ratio of about 3:1, 4:1, or 5:1.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin (SM). Further examples of a phospholipid moiety for the lipid nanoparticle include a lipid that is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), monomethyl-phosphatidylethanolamine (such as 16-O-monomethyl PE), dimethyl-phosphatidylethanolamine (such as 16-O-dimethyl PE), 18-1-trans PE, I-stearoyl-2-oleoyl-phosphatidyethanolamine (SOPE), hydrogenated soy phosphatidylcholine (HSPC), egg phosphatidylcholine (EPC), dioleoylphosphatidyl serine (DOPS), sphingomyelin (SM), dimyristoyl phosphatidylcholine (DMPC), dimyristoyl phosphatidylglycerol (DMPG), distearoylphosphatidylglycerol (DSPG), diemcoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, and mixtures thereof. In some embodiments, the lipid nanoparticle includes sphingomyelin. In some embodiments, the nanoparticle composition comprising a plurality of lipid nanoparticles, wherein the lipid nanoparticles comprise: (a) a sphingomyelin of about 5 to 40 mol percent of the total lipid present in the nanoparticle composition; (b) a cationic lipid; (c) a steroid; (d) a polymer conjugated lipid; and (e) a nucleic acid. In some embodiments, the sphingomyelin is about 10 to 40 mol percent of the total lipid present in the nanoparticle composition. In some embodiments, the sphingomyelin is about 10 to 30 mol percent of the total lipid present in the nanoparticle composition. In some embodiments, the sphingomyelin is about 10 to 25 mol percent of the total lipid present in the nanoparticle composition. In some embodiments, the sphingomyelin is about 10 to 20 mol percent of the total lipid present in the nanoparticle composition. In some embodiments, the sphingomyelin is about 10 to 15 mol percent of the total lipid present in the nanoparticle composition. In some embodiments, the sphingomyelin is about 10 mol percent of the total lipid present in the nanoparticle composition. In some embodiments, the sphingomyelin is about 15 mol percent of the total lipid present in the nanoparticle composition. In some embodiments, the sphingomyelin is about 20 mol percent of the total lipid present in the nanoparticle composition. In some embodiments, the sphingomyelin is about 10 to 20 mol percent of the total lipid present in the nanoparticle composition, and wherein the cationic lipid is about 40 to 50 mol percent of the total lipid present in the nanoparticle composition. In some embodiments, the sphingomyelin is about 10 to 15 mol percent of the total lipid present in the nanoparticle composition, and wherein the cationic lipid is about 45 mol percent of the total lipid present in the nanoparticle composition. In some embodiments, the sphingomyelin is about 10 to 15 mol percent of the total lipid present in the nanoparticle composition, and wherein the cationic lipid is about 40 mol percent of the total lipid present in the nanoparticle composition. In some embodiments, the sphingomyelin is about 10 mol percent of the total lipid present in the nanoparticle composition, and wherein the cationic lipid is about 50 mol percent of the total lipid present in the nanoparticle composition. In some embodiments, the sphingomyelin is about 10 mol percent of the total lipid present in the nanoparticle composition, and wherein the cationic lipid is about 45 mol percent of the total lipid present in the nanoparticle composition. In some embodiments, the sphingomyelin is about 15 mol percent of the total lipid present in the nanoparticle composition, and wherein the cationic lipid is about 45 mol percent of the total lipid present in the nanoparticle composition. In some embodiments, the sphingomyelin is a sphingomyelin compound. In some embodiments, the sphingomyelin is selected from SM-01, SM-02, SM-03, SM-04, SM-05, SM-06 and SM-07. In some embodiments, the molar percentage of sphingomyelin in the total lipid present in the nanoparticle composition is the same as the molar percentage of DSPC in the total lipid present in a reference nanoparticle composition. In some embodiments, the sphingomyelin is of about 5 to 40 mol percent of the total lipid present in the composition. In some embodiments, the sphingomyelin is of about 10 to 40 mol percent of the total lipid present in the composition. In some embodiments, the sphingomyelin is of about 10 to 30 mol percent of the total lipid present in the composition. In some embodiments, the sphingomyelin is of about 10 to 25 mol percent of the total lipid present in the composition. In some embodiments, the sphingomyelin is of about 10 to 20 mol percent of the total lipid present in the composition. In some embodiments, the sphingomyelin is of about 10 to 15 mol percent of the total lipid present in the composition. In some embodiments, the sphingomyelin is of about 10 mol percent of the total lipid present in the composition. In some embodiments, the sphingomyelin is of about 15 mol percent of the total lipid present in the composition. In some embodiments, the sphingomyelin is of about 20 mol percent of the total lipid present in the composition. In some embodiments, the sphingomyelin is of about 5 mol percent, about 6 mol percent, about 7 mol percent, about 8 mol percent, about 9 mol percent, about 10 mol percent, about 11 mol percent, about 11.5 mol percent, about 12 mol percent, about 12.5 mol percent, about 13 mol percent, about 13.5 mol percent, about 14 mol percent, about 14.5 mol percent, about 15 mol percent, about 15.5 mol percent, about 16 mol percent, about 16.5 mol percent, about 17 mol percent, about 17.5 mol percent, about 18 mol percent, about 18.5 mol percent, about 19 mol percent, about 19.5 mol percent, about 20 mol percent, about 21 mol percent, about 22 mol percent, about 23 mol percent, about 24 mol percent, about 25 mol percent, about 30 mol percent, about 35 mol percent, or about 40 mol percent of the total lipid present in the composition. In some embodiments, the sphingomyelin in the composition is a sphingomyelin compound having the following structure:

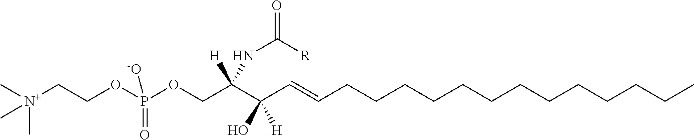

wherein R is an alkyl or alkenyl. In one embodiment, R is a C₁₁-C₂₃ alkyl. In one embodiment, R is a C₁₁-C₁₉ alkyl. In one embodiment, R is a C₁₃-C₁₉ alkyl. In one embodiment, R is a C₁₅-C₁₉ alkyl. In one embodiment, R is a C₁₁ alkyl (e.g., —(CH₂)₁₀—CH₃). In one embodiment, R is a C₁₃ alkyl (e.g., —(CH₂)₁₂—CH₃). In one embodiment, R is a C₁₄ alkyl (e.g., —(CH₂)₁₃—CH₃). In one embodiment, R is a C₁₅ alkyl (e.g., —(CH₂)₁₄—CH₃). In one embodiment, R is a C₁₆ alkyl (e.g., —(CH₂)₁₅—CH₃). In one embodiment, R is a C₁₇ alkyl (e.g., —(CH₂)₁₆—CH₃). In one embodiment, R is a C₁₈ alkyl (e.g., —(CH₂)₁₇-CH₃). In one embodiment, R is a C₁₉ alkyl (e.g., —(CH₂)₁₈—CH₃). In one embodiment, R is a C₂₀ alkyl (e.g., —(CH₂)₁₉—CH₃). In one embodiment, R is a C₂₁ alkyl (e.g., —(CH₂)₂₀—CH₃). In one embodiment, R is a C₂₂ alkyl (e.g., —(CH₂)₂₁—CH₃). In some embodiments, R is a C₂₃ alkyl (e.g., —(CH₂)₂₂—CH₃). In one embodiment, the alkyl is a straight alkyl. In one embodiment, the alkyl is a branched alkyl. In some embodiments, the alkyl is unsubstituted. In some embodiments, the sphingomyelin provided herein is selected from the SM-01, SM-02, SM-03. SM-06 and SM-07 molecules shown in Table 53. In some embodiments, R is a C₁₁-C₂₃ alkenyl. In one embodiment, R is a C₁₃-C₁₉ alkenyl. In one embodiment, R is a C₁₅-C₁₉ alkenyl. In one embodiment, R is a C₁₁ alkenyl. In one embodiment, R is a C₁₃ alkenyl. In one embodiment, R is a C₁₄ alkenyl. In one embodiment, R is a C₁₅ alkenyl. In one embodiment, R is a C₁₆ alkenyl. In one embodiment, R is a C₁₇ alkenyl. In one embodiment, R is a C₁₈ alkenyl. In one embodiment, R is a C₁₉ alkenyl. In one embodiment, R is a C₂₀ alkenyl. In one embodiment, R is a C₂₁ alkenyl. In one embodiment, R is a C₂₂ alkenyl. In one embodiment, R is a C₂₃ alkenyl. In one embodiment, the alkenyl has one double bond. In one embodiment, the double bond has a Z-configuration. In one embodiment, the double bond is at 9-position of the alkenyl R group. In one embodiment, the alkenyl is a straight alkenyl. In one embodiment, the alkenyl is a branched alkenyl. In one embodiment, the alkenyl is unsubstituted.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. Phospholipids also include phosphosphingolipid, such as sphingomyelin. In some embodiments, a phospholipid useful or potentially useful in the present invention is an analog or variant of DSPC.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about-10 mV to about +20 mV, from about-10 mV to about +15 mV, from about-10 mV to about +10 mV, from about-10 mV to about +5 mV, from about-10 mV to about 0 mV, from about-10 mV to about-5 mV, from about-5 mV to about +20 mV, from about-5 mV to about +15 mV, from about-5 mV to about +10 mV, from about-5 mV to about +5 mV, from about-5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity.

The efficacy of the product is dependent on expression of the delivered RNA, which requires a sufficiently intact RNA molecule. RNA integrity is a measure of RNA quality that quantitates intact RNA. The method is also capable of detecting potential degradation products. RNA integrity is preferably determined by capillary gel electrophoresis. The initial specification is set to ensure sufficient RNA integrity in drug product preparations. In some embodiments, the RNA polynucleotide has an integrity of at least about 80%, 85%, 90%, 92%, 94%, 95%, 96%, 97%, 98%, or 99%. In some embodiments, the RNA polynucleotide has an integrity of or greater than about 95%. In some embodiments, the RNA polynucleotide has an integrity of or greater than about 98%. In some embodiments, the RNA polynucleotide has an integrity of or greater than about 99%.

In preferred embodiments, the RNA polynucleotide has a clinical grade purity. In some embodiments, the purity of the RNA polynucleotide is between about 60% and about 100%. In some embodiments, the purity of the RNA polynucleotide is between about 80% and 99%. In some embodiments, the purity of the RNA polynucleotide is between about 90% and about 99%. In some embodiments, wherein the purified mRNA has a clinical grade purity without further purification. In some embodiments, the clinical grade purity is achieved through a method including tangential flow filtration (TFF) purification. In some embodiments, the clinical grade purity is achieved without the further purification selected from high performance liquid chromatography (HPLC) purification, ligand or binding based purification, and/or ion exchange chromatography. In some embodiments, the method of producing the RNA polynucleotides removes long abortive RNA species, double-stranded RNA (dsRNA), residual plasmid DNA residual solvent and/or residual salt. In some embodiments, the short abortive transcript contaminants comprise less than 15 bases. In some embodiments, the short abortive transcript contaminants comprise about 8-12 bases. In some embodiments, the method of the invention also removes RNAse inhibitor.

In some embodiments, the purified RNA polynucleotide comprises 5% or less, 4% or less, 3% or less, 2% or less, 1% or less or is substantially free of protein contaminants as determined by capillary electrophoresis. In some embodiments, the purified RNA polynucleotide comprises less than 5%, less than 4%, less than 3%, less than 2%, less than 1%, or is substantially free of salt contaminants determined by high performance liquid chromatography (HPLC). In some embodiments, the purified RNA polynucleotide comprises 5% or less, 4% or less, 3% or less, 2% or less, 1% or less or is substantially free of short abortive transcript contaminants determined by known methods, such as, e.g., high performance liquid chromatography (HPLC). In some embodiments, the purified RNA polynucleotide has integrity of 60% or greater, 70% or greater, 80% or greater, 81% or greater, 82% or greater, 83% or greater, 84% or greater, 85% or greater, 86% or greater, 87% or greater, 88% or greater, 89% or greater, 90% or greater, 95% or greater, 96% or greater, 97% or greater, 98% or greater, or 99% or greater as determined by a known method, such as, e.g., capillary electrophoresis.

### Modified Nucleobases

Modified nucleobases which may be incorporated into modified nucleosides and nucleotides and be present in the RNA molecules include, for example, m5C (5-methylcytidine), m5U (5-methyluridine), m6A (N6-methyladenosine), s2U (2-thiouridine), Um (2′-O-methyluridine), mIA (1-methyladenosine); m2A (2-methyladenosine); Am (2-1-O-methyladenosine); ms2m6A (2-methylthio-N6-methyladenosine); 16A (N6-isopentenyladenosine); ms2i6A (2-methylthio-N6isopentenyladenosine); io6A (N6-(cis-hydroxyisopentenyl) adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar (p) (2′-O-ribosyladenosine (phosphate)); I (inosine); mil (1-methylinosine); m′1m (1,2′-O-dimethylinosine); m3C (3-methylcytidine); Cm (2T-O-methylcytidine); s2C (2-thiocytidine); ac4C (N4-acetylcytidine); £5C (5-fonnylcytidine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr (p) (2′-O-ribosylguanosine (phosphate)); yW (wybutosine); 02yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl) uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl) uridine)); mchm5U (5-(carboxyhydroxymethyl) uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6, T-O-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); mlGm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-Ce)-alkenyluracil, 5-(C2-C6)-alkynyluracil, 5-(hydroxymethyl) uracil, 5-chlorouracil, 5-fluorouracil, 5-bromouracil, 5-hydroxycytosine, 5-(C1-C₆)-alkylcytosine, 5-methylcytosine, 5-(C2-C₆)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. In some aspects, one or more of the modified nucleosides in the list may be excluded.

Additional exemplary modified nucleotides include any one of N-1-methylpseudouridine; pseudouridine, N6-methyladenosine, 5-methylcytidine, and 5-methyluridine. In some embodiments, the modified nucleotide is N-1-methylpseudouridine.

In some embodiments, the RNA molecule may include phosphoramidate, phosphorothioate, and/or methylphosphonate linkages.

In some embodiments, the RNA molecule includes a modified nucleotide selected from any one of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the modified or unnatural nucleotides are selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. In some embodiments, the modified or unnatural nucleotides are selected from the group consisting of 5-methyluridine, N1-methylpseudouridine, 5-methoxyuridine, and 5-methylcytosine.

In some embodiments, at least 10% of a total population of a particular nucleotide in the saRNA molecule has been replaced with one or more modified or unnatural nucleotides. In some embodiments, at least 25% of a total population of a particular nucleotide in the molecule has been replaced with one or more modified or unnatural nucleotides. In some embodiments, at least 50% of a total population of a particular nucleotide in the molecule has been replaced with one or more modified or unnatural nucleotides. In some embodiments, at least 75% of a total population of a particular nucleotide in the molecule has been replaced with one or more modified or unnatural nucleotides. In some embodiments, essentially all of the particular nucleotide population in the molecule has been replaced with one or more modified or unnatural nucleotides.

In some embodiments, at least a portion, or all of a total population of a particular nucleotide in the saRNA molecule has been replaced with two modified or unnatural nucleotides. In some embodiments, the two modified or unnatural nucleotides are provided in a ratio equal to any one of, at least any one of, at most any one of, or between any two of 1:99 to 99:1, including 1:99; 2:98; 3:97; 4:96; 5:95; 6:94; 7:93; 8:92; 9:91; 10:90; 11:89; 12:88; 13:87; 14:86; 15:85; 16:84; 17:83; 18:82, 19:81; 20:80; 21:79; 22:78; 23:77; 24:76; 25:75; 26:74; 27:73; 28:72; 29:71; 30:70; 31:69; 32:68; 33:67; 34:66; 35:65; 36:64; 37:63; 38:62; 39:61; 40:60; 41:59; 42:58; 43:57; 44:56; 45:55; 46:54; 47:53; 48:52; 49:51; 50:50; 51:49; 52:48; 53:47; 54:46; 55:45; 56:44; 57:43; 58:42; 59:41; 60:40; 61:39; 62:38; 63:37; 64:36; 65:35; 66:34; 67:33; 68:32; 69:31; 70:30; 71:29; 72:28; 73:27; 74:26; 75:25; 76:24; 77:23; 78:22; 79:21; 80:20; 81:19; 82:18; 83:17; 84:16; 85:15; 86:14; 87:13; 88:12; 89:11; 90:10; 91:9; 92:8; 93:7; 94:6; 95:5; 96:4; 97:3; 98:2; and 99:1, or any range derivable therein.

In some embodiments, at least 10% of a total population of a first particular nucleotide in a saRNA molecule as disclosed herein has been replaced with one or more modified or unnatural nucleotides, and at least 10% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and at least 25% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and at least 50% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and at least 25% of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and at least 50% of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, at least 50% of a total population of a first particular nucleotide in the molecule has been replaced with one or more modified or unnatural nucleotides, and at least 50% of a total population of a second particular nucleotide in the molecule has been replaced with one or more modified or unnatural nucleotides. In some embodiments, at least 50% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 50% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, at least 75% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 75% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, essentially all of a total population of a first particular nucleotide in the molecule has been replaced with one or more modified or unnatural nucleotides, and essentially all of a total population of a second particular nucleotide in the molecule has been replaced with one or more modified or unnatural nucleotides.

In some embodiments, at least 25% of a total population of uridine nucleotides in the saRNA molecule has been replaced with N1-methylpseudouridine. In some embodiments, at least 50% of a total population of uridine nucleotides in the molecule has been replaced with N1-methylpseudouridine. In some embodiments, at least 75% of a total population of uridine nucleotides in the molecule has been replaced with N1-methylpseudouridine. In some embodiments, essentially all uridine nucleotides in the molecule have been replaced with N1-methylpseudouridine. In some embodiments, at least 50% of a total population of uridine nucleotides in the molecule has been replaced with 5-methoxyuridine. In some embodiments, essentially all uridine nucleotides in the molecule have been replaced with 5-methoxyuridine. In some embodiments, at least 50% of a total population of uridine nucleotides in the molecule has been replaced with 5-methyluridine. In some embodiments, essentially all uridine nucleotides in the molecule have been replaced with 5-methyluridine. In some embodiments, at least 50% of a total population of cytosine nucleotides in the molecule has been replaced with 5-methylcytosine. In some embodiments, essentially all cytosine nucleotides in the molecule have been replaced with 5-methylcytosine. In some embodiments, at least 50% of a total population of uridine nucleotides in the molecule has been replaced with 2-thiouridine. In some embodiments, essentially all uridine nucleotides in the molecule have been replaced with 2-thiouridine.

In some embodiments, at least 50% of a total population of uridine nucleotides in the molecule has been replaced with N1-methylpseudouridine and essentially all cytosine nucleotides in the molecule have been replaced with 5-methylcytosine. In some embodiments, at least 50% of a total population of uridine nucleotides in the molecule has been replaced with 5-methoxyuridine and essentially all cytosine nucleotides in the molecule have been replaced with 5-methylcytosine. In some embodiments, at least 50% of a total population of uridine nucleotides in the molecule has been replaced with 5-methyluridine and essentially all cytosine nucleotides in the molecule have been replaced with 5-methylcytosine.

In some embodiments, essentially all uridine nucleotides in the molecule have been replaced with about 50% 5-methoxyuridine and about 50% N1-methylpseudouridine. In some embodiments, essentially all uridine nucleotides in the molecule have been replaced with about 75% 5-methoxyuridine and about 25% N1-methylpseudouridine. In some embodiments, essentially all uridine nucleotides in the molecule have been replaced with about 25% 5-methoxyuridine and about 75% N1-methylpseudouridine.

### UTRs

The 5′ untranslated regions (UTR) is a regulatory region of DNA situated at the 5′ end of a protein coding sequence that is transcribed into mRNA but not translated into protein. 5′ UTRs may contain various regulatory elements, e.g., 5′ cap structure, stem-loop structure, and an internal ribosome entry site (IRES), which may play a role in the control of translation initiation. The 3′ UTR, situated downstream of a protein coding sequence, may be involved in regulatory processes including transcript cleavage, stability and polyadenylation, translation, and mRNA localization. In some embodiments, the UTR is derived from an mRNA that is naturally abundant in a specific tissue (e.g., lymphoid tissue), to which the mRNA expression is targeted. In some embodiments, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). According, the UTR sequence may prolong protein synthesis in a tissue-specific manner. In some embodiments, the 5′ UTR and the 3′ UTR sequences are computationally derived. In some embodiments, the 5′ UTR and the 3′ UTRs are derived from a naturally abundant mRNA in a tissue. The tissue may be, for example, liver, a stem cell, or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some embodiments, the 5′ UTR and the 3′ UTR are derived from an alphavirus. In some embodiments, the 5′ UTR and the 3′ UTR are from a wild-type alphavirus. Examples of alphaviruses are described below.

In some embodiments, the first RNA molecule includes a 5′ UTR and the 3′ UTR derived from a naturally abundant mRNA in a tissue. In some embodiments, the first RNA molecule includes a 5′ UTR and the 3′ UTR derived from an alphavirus. In some embodiments, the second RNA or the saRNA molecule includes a 5′ UTR and the 3′ UTR derived from an alphavirus. In some embodiments, the second RNA or the saRNA molecule includes a 5′ UTR and the 3′ UTR from a wild-type alphavirus. In some embodiments, the RNA molecule includes a 5′ cap.

### Open Reading Frame (ORF)

The 5′ and 3′ UTRs may be operably linked to an ORF, which may be a sequence of codons that is capable of being translated into a polypeptide of interest. As stated above, the RNA molecule may include one (monocistronic), two (bicistronic) or more (multicistronic) open reading frames (ORFs).

In some embodiments, the ORF encodes a non-structural viral gene. In some embodiments, the ORF further includes one or more subgenomic promoters. In some embodiments, the RNA molecule includes a subgenomic promoter operably linked to the ORF. In some embodiments, the subgenomic promoter comprises a cis-acting regulatory element. In some embodiments, the cis-acting regulatory element is immediately downstream (5′-3′) of B2. In some embodiments, the cis-acting regulatory element is immediately downstream (5′-3′) of a guanine that is immediately downstream of B2. In some embodiments, the cis-acting regulatory element is an AU-rich element. In some embodiments, the AU-rich element is au, auaaaagau, auaaaaagau, auag, auauauauau, auauauau, auauauauauau, augaugaugau, augau, auaaaagaua, or auaaaagaug. In some embodiments, the second RNA or the saRNA molecule may include (i) an ORF encoding a replicase which may transcribe RNA from the second RNA or the saRNA molecule and (ii) an ORF encoding at least one an antigen or polypeptide of interest. The polymerase may be an alphavirus replicase e.g., including any one of the non-structural alphavirus proteins nsP1, nsP2, nsP3 and nsP4, or a combination thereof. In some embodiments, the RNA molecule includes alphavirus nonstructural protein nsP1. In some embodiments, the RNA molecule includes alphavirus nonstructural protein nsP2. In some embodiments, the RNA molecule includes alphavirus nonstructural protein nsP3. In some embodiments, the RNA molecule includes alphavirus nonstructural protein nsP4. In some embodiments, the RNA molecule includes alphavirus nonstructural proteins nsP1, nsP2, and nsP3. In some embodiments, the RNA molecule includes alphavirus nonstructural proteins nsP1, nsP2, nsP3, and nsP4. In some embodiments, the RNA molecule includes any combination of nsP1, nsP2, nsP3, and nsP4. In some embodiments, the RNA molecule does not include nsP4.

In some embodiments, an open reading frame of an RNA (e.g., saRNA) composition is codon-optimized. In some embodiments, the open reading frame which the influenza polypeptide or fragment thereof is encoded is codon-optimized.

### 5′ Cap

In some embodiments, the saRNA molecule described herein includes a 5′ cap. In some embodiments, the 5′-cap moiety is a natural 5′-cap. A “natural 5′-cap” is defined as a cap that includes 7-methylguanosine connected to the 5′ end of an mRNA molecule through a 5′ to 5′ triphosphate linkage. In some embodiments, the 5′-cap moiety is a 5′-cap analog. In some embodiments, the 5′ end of the RNA is capped with a modified ribonucleotide with the structure m7G (5′) ppp (5′) N (cap 0 structure) or a derivative thereof, which may be incorporated during RNA synthesis (e.g., co-transcriptional capping) or may be enzymatically engineered after RNA transcription (e.g., post-transcriptional capping), wherein “N” is any ribonucleotide. In some embodiments, the 5′ end of the RNA molecule is capped with a modified ribonucleotide via an enzymatic reaction after RNA transcription. In some embodiments, capping is performed after purification, e.g., tangential flow filtration, of the RNA molecule. An exemplary enzymatic reaction for capping may include use of Vaccinia Virus Capping Enzyme (VCE) that includes mRNA triphosphatase, guanylyl-transferase, and guanine-7-methytransferase, which catalyzes the construction of N7-monomethylated cap 0 structures. Cap 0 structure can help maintaining the stability and translational efficacy of the RNA molecule. The 5′ cap of the RNA molecule may be further modified by a 2′-O-Methyltransferase which results in the generation of a cap 1 structure (m7Gppp [m2′-O]N), which may further increase translation efficacy. In some embodiments, the RNA molecule may be enzymatically capped at the 5′ end using Vaccinia guanylyltransferase, guanosine triphosphate, and S-adenosyl-L-methionine to yield cap 0 structure. An inverted 7-methylguanosine cap is added via a 5′ to 5′ triphosphate bridge. Alternatively, use of a 2′O-methyltransferase with Vaccinia guanylyltransferase yields the cap 1 structure where in addition to the cap 0 structure, the 2′OH group is methylated on the penultimate nucleotide. S-adenosyl-L-methionine (SAM) is a cofactor utilized as a methyl transfer reagent. Non-limiting examples of 5′ cap structures are those which, among other things, have enhanced binding of cap binding polypeptides, increased half-life, reduced susceptibility to 5′ endonucleases and/or reduced 5′ decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme may create a canonical 5′-5′-triphosphate linkage between the 5′-terminal nucleotide of an mRNA and a guanine cap nucleotide wherein the cap guanine includes an N7 methylation and the 5′-terminal nucleotide of the mRNA includes a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7 mG (5′) ppp (5′) N, pN2p (cap 0) and 7 mG (5′) ppp (5′) N1mpNp (cap 1). Cap 0 is a N7-methyl guanosine connected to the 5′ nucleotide through a 5′ to 5′ triphosphate linkage, typically referred to as m7G cap or m7Gppp. In the cell, the cap 0 structure can help provide for efficient translation of the mRNA that carries the cap. An additional methylation on the 2′O position of the initiating nucleotide generates Cap 1, or refers to as m7GpppNm-, wherein Nm denotes any nucleotide with a 2′O methylation. In some embodiments, the 5′ terminal cap includes a cap analog, for example, a 5′ terminal cap may include a guanine analog. Exemplary guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine. In some embodiments, the capping region may include a single cap or a series of nucleotides forming the cap. In this embodiment the capping region may be equal to any one of, at least any one of, at most any one of, or between any two of 1, 2, 3, 4, 5, 6, 7, 8, 9, 10, or at least 2, or 10 or fewer nucleotides in length. In some embodiments, the cap is absent. In some embodiments, the first and second operational regions may be equal to any one of, at least any one of, at most any one of, or between any two of 3 to 40, e.g., 5-30, 10-20, 15, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or at least 4, or 30 or fewer nucleotides in length and may comprise, in addition to a Start and/or Stop codon, one or more signal and/or restriction sequences.

In some embodiments, the 5′ Cap is represented by Formula I:

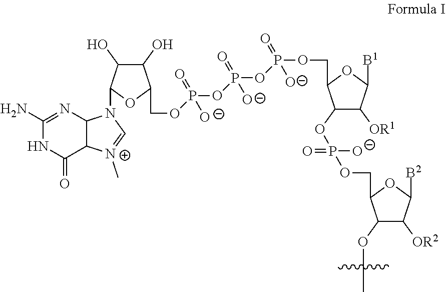

where R¹ and R² are each independently H or Me, and B¹ and B² are each independently guanine, adenine, or uracil. In some embodiments, B¹ and B² are naturally-occurring bases. In some embodiments, R¹ is methyl and R² is hydrogen. In some embodiments, B¹ is guanine. In some embodiments, B¹ is adenine. In some embodiments, B² is adenine. In some embodiments, B² is uracil. In some embodiments, B² is uracil and at least 5% of a total population of uracil nucleotides in the molecule that are downstream of B² have been replaced with one or more modified or unnatural nucleotides.

In some embodiments, the nucleotide immediately downstream (5′ to 3′ direction) of the 5′ Cap comprises guanine. In some embodiments, B¹ is adenine and B² is uracil. In some embodiments, B¹ is adenine, B² is uracil, R¹ is methyl, and R² is hydrogen. In some instances, the saRNA does not comprise a 5′ Cap. In some instances, the 5′ Cap is not represented by Formula I. In some embodiments, the nucleotide immediately downstream (5′ to 3′) of the 5′ Cap comprises guanine, B¹ is adenine, B² is uracil, R¹ is methyl, and R² is hydrogen; this embodiment corresponds to CleanCap AU, and the inclusion of B²=uracil, while optionally subsetting uracil nucleotides downstream of B², has been shown to improve saRNA functionality in some embodiments. In some embodiments, the RNA molecule further comprises: (1) an alphavirus 5′ replication recognition sequence, and (2) an alphavirus 3′ replication recognition sequence. In some embodiments, the RNA molecule encodes at least one antigen. In some embodiments, the RNA molecule comprises at least 7000 nucleotides. In some embodiments, the RNA molecule comprises at least 8000 nucleotides. In some embodiments, at least 80% of the total RNA molecules are full length. In some embodiments, the alphavirus is Venezuelan equine encephalitis virus. In some embodiments, the alphavirus is Semliki Forest virus.

In some embodiments, the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the molecule has been replaced with N1-methylpseudouridine, and essentially all cytosine nucleotides in the molecule have been replaced with 5-methylcytosine. In some embodiments, the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the molecule has been replaced with 5-methoxyuridine, and essentially all cytosine nucleotides in the molecule have been replaced with 5-methylcytosine. In some embodiments, the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the molecule has been replaced with 5-methyluridine, and essentially all cytosine nucleotides in the molecule have been replaced with 5-methylcytosine. In some embodiments, the nucleotide immediately downstream (5′ to 3′) of the 5′ Cap comprises guanine, B¹ is adenine, B² is uracil, R¹ is methyl, and R² is hydrogen, essentially all uridine nucleotides in the molecule have been replaced with about 50% 5-methoxyuridine and about 50% N1-methylpseudouridine. In some embodiments, the nucleotide immediately downstream (5′ to 3′) of the 5′ Cap comprises guanine, B¹ is adenine, B² is uracil, R¹ is methyl, and R² is hydrogen, essentially all uridine nucleotides in the molecule have been replaced with about 75% 5-methoxyuridine and about 25% N1-methylpseudouridine. In some embodiments, the nucleotide immediately downstream (5′ to 3′) of the 5′ Cap comprises guanine, B¹ is adenine, B² is uracil, R¹ is methyl, and R² is hydrogen, essentially all uridine nucleotides in the molecule have been replaced with about 25% 5-methoxyuridine and about 75% N1-methylpseudouridine.

In some embodiments, a 5′ terminal cap is 7 mG (5′) ppp (5′) NImpNp. In some preferred embodiments, the 5′ cap comprises:

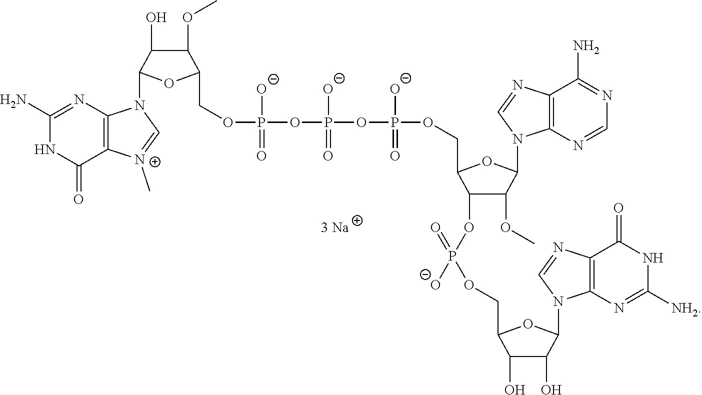 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

In some embodiments, the 5′ cap comprises CLEANCAP® Reagent AG (3′ OMe) for co-transcriptional capping of mRNA, m7 (3′OMeG) (5′) ppp (5′) (2′OMeA) pG,

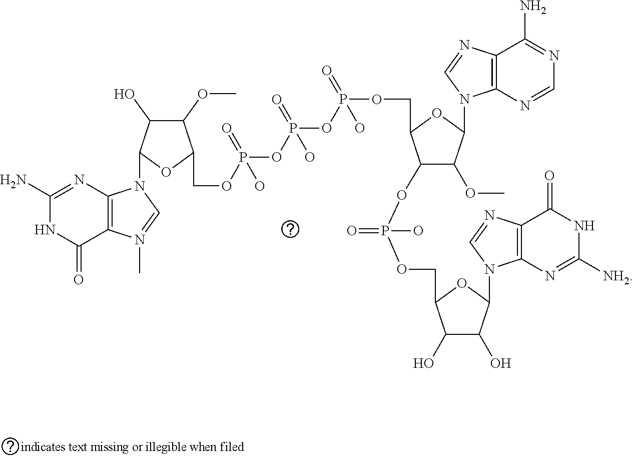

In alternative embodiments, the 5′ cap comprises CLEANCAP® AU for Self-Amplifying mRNA, CLEANCAP® Reagent AU for co-transcriptional capping of mRNA, m7G (5′) ppp (5′) (2′OMeA) pU,

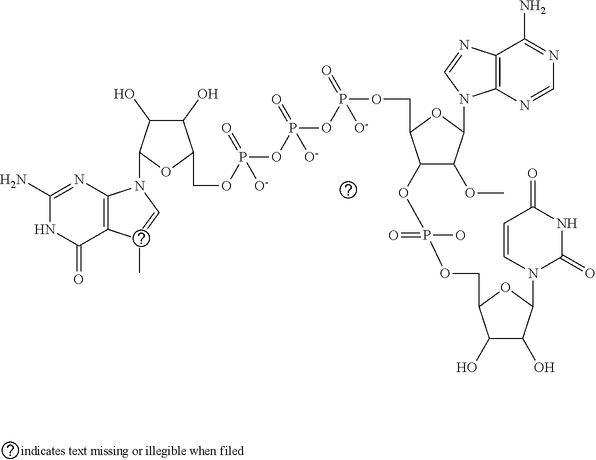 [SMILES: COC1C(OP(=O)(O)OCC2OC(n3ccc(=O)[nH]c3=O)C(O)C2O)C(COP(=O)([O-])OP(=O)([O-])OP(=O)([O-])OCC2OC(n3cc(C)c4c(=O)[nH]c(N)nc43)C(O)C2O)OC1n1cnc2c(N)ncnc21]

### Poly-a Tail

As used herein, “poly A tail” refers to a stretch of consecutive adenine residues, which may be attached to the 3′ end of the RNA molecule. The poly-A tail may increase the half-life of the RNA molecule. Poly-A tails may play key regulatory roles in enhancing translation efficiency and regulating the efficiency of mRNA quality control and degradation. Short sequences or hyperpolyadenylation may signal for RNA degradation. Exemplary designs include a poly-A tails of about 40 adenine residues to about 80 adenine residues. In some embodiments, the RNA molecule further includes an endonuclease recognition site sequence immediately downstream of the poly A tail sequence. In some embodiments, such as for the second RNA or the saRNA molecule, the RNA molecule further includes a poly-A polymerase recognition sequence (e.g., AAUAAA) near its 3′ end. A “full length” RNA molecule is one that includes a 5′-cap and a poly A tail.

In some embodiments, the poly A tail includes 5-400 nucleotides in length. The poly A tail nucleotide length may be equal to any one of, at least any one of, at most any one of, or between any two of 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, and 400. In some embodiments, the RNA molecule includes a poly A tail that includes about 25 to about 400 adenosine nucleotides, a sequence of about 50 to about 400 adenosine nucleotides, a sequence of about 50 to about 300 adenosine nucleotides, a sequence of about 50 to about 250 adenosine nucleotides, a sequence of about 60 to about 250 adenosine nucleotides, or a sequence of about 40 to about 100 adenosine nucleotides. In some embodiments, the RNA molecule includes a poly A tail includes a sequence of greater than 30 adenosine nucleotides (“As”). In some embodiments, the RNA molecule includes a poly A tail that includes about 40 As. In some embodiments, the RNA molecule includes a poly A tail that includes about 80 As. As used herein, the term “about” refers to a deviation of +10% of the value(s) to which it is attached. In some embodiments, the 3′ poly-A tail has a stretch of at least 10 consecutive adenosine residues and at most 300 consecutive adenosine residues. In some embodiments, the RNA molecule includes at least 20 consecutive adenosine residues and at most 40 consecutive adenosine residues. In some embodiments, the RNA molecule includes about 40 consecutive adenosine residues. In some embodiments, the RNA molecule includes about 80 consecutive adenosine residues.

### Composition

In some instances, the compositions described herein include at least one saRNA as described herein. Some embodiments of the present disclosure provide influenza virus (influenza) vaccines (or compositions or immunogenic compositions) that include at least one saRNA polynucleotide having an open reading frame encoding at least one influenza antigenic polypeptide or an immunogenic fragment thereof (e.g., an immunogenic fragment capable of inducing an immune response to influenza).

In some embodiments, equal to any one of, at least any one of, at most any one of, or between any two of 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the total RNA molecules (capped and uncapped) in the composition are capped.

In some embodiments, equal to any one of, at least any one of, at most any one of, or between any two of 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the total RNA molecules in the composition are full length RNA transcripts. Purity may be determined as described herein, e.g., via reverse phase HPLC or Bioanalyzer chip-based electrophoresis and measure by, e.g., peak area of full-length RNA molecule relative to total peak. In some embodiments, a fragment analyzer (FA) may be used to quantify and purify the RNA. The fragment analyzer automates capillary electrophoresis and HPLC.

In some embodiments, the composition is substantially free of one or more impurities or contaminants including the linear DNA template and/or reverse complement transcription products and, for instance, includes RNA molecules that are equal to any one of, at least any one of, at most any one of, or between any two of 90%, 91%, 92%, 93%, 94%, 95%, 96%, 97%, 98%, or 99% pure; at least 98% pure, or at least 99% pure.

In some embodiments, the composition comprises an amount of the first RNA molecule that is greater than the amount of the second RNA molecule. In some embodiments, the composition comprises an amount of the first RNA molecule that is at least about 1 to 2 times greater than the amount of the second RNA molecule. In some embodiments, the composition comprises an amount of the first RNA molecule that is at least about 1 to 100 times greater than the amount of the second RNA molecule.

In some embodiments, the composition further includes a pharmaceutically acceptable carrier. In some embodiments, the composition further includes a pharmaceutically acceptable vehicle.

In some embodiments, the composition further includes a lipid-based delivery system, which delivers an RNA molecule to the interior of a cell, where it can then replicate and/or express the encoded polypeptide of interest. The delivery system may have adjuvant effects which enhance the immunogenicity of an encoded antigen. In some embodiments, the composition further includes neutral lipids, cationic lipids, cholesterol, and polyethylene glycol (PEG), and forms nanoparticles that encompass the RNA molecules. In some embodiments, the composition further includes any one of a cationic lipid, a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, and a cationic nanoemulsion. In some embodiments, the RNA molecule is encapsulated in, bound to or adsorbed on any one of a cationic lipid, a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, and a cationic nanoemulsion, or a combination thereof.

In some instances, the compositions described herein include at least two RNA molecules: a first RNA molecule and a second RNA molecule as described herein. To protect against more than one strain of influenza, a combination vaccine composition may be administered that includes RNA encoding at least one antigenic polypeptide protein (or antigenic portion thereof) of a first influenza virus or organism and further includes a second RNA molecule encoding at least one antigenic polypeptide protein (or antigenic portion thereof) of a second influenza virus or organism. RNA can be co-formulated, for example, in a single lipid nanoparticle (LNP) or can be formulated in separate LNPs for co-administration.

In some embodiments, the second RNA molecule includes any one of a 5′ cap, a 5′ UTR, an open reading frame, a 3′ UTR, and a poly A sequence, or any combination thereof. In some embodiments, the second RNA molecule includes a 5′ cap moiety. In some embodiments, the second RNA molecule includes a 5′ UTR and a 3′UTR. In some embodiments, the second RNA molecule includes a 5′UTR, an open reading frame, a 3′UTR, and does not further include a 5′ cap. In some embodiments, the second RNA molecule includes a 5′ cap moiety, 5′ UTR, coding region, 3′ UTR, and a 3′ poly A sequence. In some embodiments, the second RNA molecule includes a 5′ cap moiety, 5′ UTR, noncoding region, 3′ UTR, and a 3′ poly A sequence. In some embodiments, the second RNA molecule includes a noncoding region and does not further comprise any one of a 5′ cap moiety, 5′ UTR, 3′ UTR, and a 3′ poly A sequence. In some embodiments, the second RNA molecule includes a 5′ cap moiety, a 5′ untranslated region (5′ UTR), a modified nucleotide, an open reading frame, a 3′ untranslated region (3′ UTR), and a 3′ poly A sequence.

Some aspects of the disclosure are directed to a composition comprising (i) first RNA molecule encoding a gene of interest derived from influenza; and (ii) a second RNA molecule comprising a modified or unnatural nucleotide In some instances, the first RNA molecule is any one of the saRNA molecules described herein. In some instances, the first RNA molecule comprises a 5′ Cap, a 5′ untranslated region, a coding region for a nonstructural protein comprising a RNA replicase, a subgenomic promoter, an open reading frame encoding a gene of interest, a 3′ untranslated region, and a 3′ poly A sequence. In some instances, at least 5% of a total population of a particular nucleotide in the first RNA molecule has been replaced with one or more modified or unnatural nucleotides. In some instances, the RNA molecule comprises natural, unmodified nucleotides and does not include a modified or unnatural nucleotide. In some instances, the 5′ Cap is represented by Formula I, where R1 and R2 are each independently H or Me, B1 and B2 are each independently guanine, adenine, or uracil, a 5′ untranslated region, a coding region for a nonstructural protein derived from an alphavirus, a subgenomic promoter, such as one derived from an alphavirus, an open reading frame encoding a gene of interest, a 3′ untranslated region, and a 3′ poly A sequence. In some embodiments, B1 and B2 are naturally-occurring bases. In some embodiments, R¹ is methyl and R2 is hydrogen. In some embodiments, B1 is guanine. In some embodiments, B1 is adenine. In some embodiments, B2 is adenine. In some embodiments, B2 is uracil. In some embodiments, the nucleotide immediately downstream (5′ to 3′ direction) of the 5′ Cap comprises guanine.

In some embodiments, B¹ is adenine and B² is uracil. In some embodiments, B¹ is adenine, B² is uracil, R¹ is methyl, and R² is hydrogen. In some embodiments, the nucleotide immediately downstream (5′ to 3′) of the 5′ Cap comprises guanine, B¹ is adenine, B² is uracil, R¹ is methyl, and R² is hydrogen; this embodiment corresponds to CLEANCAP AU (Trilink), and the inclusion of B²=uracil, while optionally substituting uracil nucleotides downstream of B², which has been shown to provide increased saRNA functionality in some embodiments.

In some embodiments, at least 10% of a total population of a particular nucleotide in the first or second RNA molecule has been replaced with one or more modified or unnatural nucleotides. In some embodiments, at least 25% of a total population of a particular nucleotide in the first or second RNA molecule has been replaced with one or more modified or unnatural nucleotides. In some embodiments, at least 50% of a total population of a particular nucleotide in the first or second RNA molecule has been replaced with one or more modified or unnatural nucleotides. In some embodiments, at least 75% of a total population of a particular nucleotide in the first or second RNA molecule has been replaced with one or more modified or unnatural nucleotides. In some embodiments, essentially all of a particular nucleotide population in the first or second RNA molecule has been replaced with one or more modified or unnatural nucleotides. In some embodiments, the one or more modified or unnatural replacement nucleotides comprise two modified or unnatural nucleotides provided in a ratio ranging from 1:99 to 99:1, or any derivable range therein. In some embodiments, at least 10% of a total population of a first particular nucleotide in the first or second RNA molecule has been replaced with one or more modified or unnatural nucleotides, and at least 10% of a total population of a second particular nucleotide in the first or second RNA molecule has been replaced with one or more modified or unnatural nucleotides. In some embodiments, at least 10% of a total population of a first particular nucleotide in the first or second RNA molecule has been replaced with one or more modified or unnatural nucleotides, and at least 25% of a total population of a second particular nucleotide in the first or second RNA molecule has been replaced with one or more modified or unnatural nucleotides. In some embodiments, at least 10% of a total population of a first particular nucleotide in the first or second RNA molecule has been replaced with one or more modified or unnatural nucleotides, and at least 50% of a total population of a second particular nucleotide in the first or second RNA molecule has been replaced with one or more modified or unnatural nucleotides. In some embodiments, at least 10% of a total population of a first particular nucleotide in the first or second RNA molecule has been replaced with one or more modified or unnatural nucleotides, and at least 75% of a total population of a second particular nucleotide in the first or second RNA molecule has been replaced with one or more modified or unnatural nucleotides. In some embodiments, at least 10% of a total population of a first particular nucleotide in the first or second RNA molecule has been replaced with one or more modified or unnatural nucleotides, and essentially all of a total population of a second particular nucleotide in the first or second RNA molecule has been replaced with one or more modified or unnatural nucleotides. In some embodiments, at least 25% of a total population of a first particular nucleotide in the first or second RNA molecule has been replaced with one or more modified or unnatural nucleotides, and at least 25% of a total population of a second particular nucleotide in the first or second RNA molecule has been replaced with one or more modified or unnatural nucleotides. In some embodiments, at least 25% of a total population of a first particular nucleotide in the first or second RNA molecule has been replaced with one or more modified or unnatural nucleotides, and at least 50% of a total population of a second particular nucleotide in the first or second RNA molecule has been replaced with one or more modified or unnatural nucleotides. In some embodiments, at least 25% of a total population of a first particular nucleotide in the first or second RNA molecule has been replaced with one or more modified or unnatural nucleotides, and at least 75% of a total population of a second particular nucleotide in the first or second RNA molecule has been replaced with one or more modified or unnatural nucleotides. In some embodiments, at least 25% of a total population of a first particular nucleotide in the first or second RNA molecule has been replaced with one or more modified or unnatural nucleotides, and essentially all of a total population of a second particular nucleotide in the first or second RNA molecule has been replaced with one or more modified or unnatural nucleotides. In some embodiments, at least 50% of a total population of a first particular nucleotide in the first or second RNA molecule has been replaced with one or more modified or unnatural nucleotides, and at least 75% of a total population of a second particular nucleotide in the first or second RNA molecule has been replaced with one or more modified or unnatural nucleotides. In some embodiments, at least 50% of a total population of a first particular nucleotide in the first or second RNA molecule has been replaced with one or more modified or unnatural nucleotides, and essentially all of a total population of a second particular nucleotide in the first or second RNA molecule has been replaced with one or more modified or unnatural nucleotides. In some embodiments, at least 75% of a total population of a first particular nucleotide in the first or second RNA molecule has been replaced with one or more modified or unnatural nucleotides, and essentially all of a total population of a second particular nucleotide in the first or second RNA molecule has been replaced with one or more modified or unnatural nucleotides.

In some embodiments, at least 25% of a total population of uridine nucleotides in the first RNA molecule has been replaced with N1-methylpseudouridine. In some embodiments, at least 50% of a total population of uridine nucleotides in the first RNA molecule has been replaced with N1-methylpseudouridine. In some embodiments, at least 75% of a total population of uridine nucleotides in the first RNA molecule has been replaced with N1-methylpseudouridine. In some embodiments, essentially all uridine nucleotides in the first RNA molecule have been replaced with N1-methylpseudouridine. In some embodiments, at least 50% of a total population of uridine nucleotides in the first RNA molecule has been replaced with 5-methoxyuridine. In some embodiments, essentially all uridine nucleotides in the molecule have been replaced with 5-methoxyuridine. In some embodiments, at least 50% of a total population of uridine nucleotides in the first RNA molecule has been replaced with 5-methyluridine. In some embodiments, essentially all uridine nucleotides in the first RNA molecule have been replaced with 5-methyluridine. In some embodiments, at least 50% of a total population of cytosine nucleotides in the first RNA molecule has been replaced with 5-methylcytosine. In some embodiments, essentially all cytosine nucleotides in the first RNA molecule have been replaced with 5-methylcytosine. In some embodiments, at least 50% of a total population of uridine nucleotides in the first RNA molecule has been replaced with 2-thiouridine. In some embodiments, essentially all uridine nucleotides in the first RNA molecule have been replaced with 2-thiouridine.

In some embodiments, at least 25% of a total population of uridine nucleotides in the second RNA molecule has been replaced with N1-methylpseudouridine. In some embodiments, at least 50% of a total population of uridine nucleotides in the second RNA molecule has been replaced with N1-methylpseudouridine. In some embodiments, at least 75% of a total population of uridine nucleotides in the second RNA molecule has been replaced with N1-methylpseudouridine. In some embodiments, essentially all uridine nucleotides in the second RNA molecule have been replaced with N1-methylpseudouridine. In some embodiments, at least 50% of a total population of uridine nucleotides in the second RNA molecule has been replaced with 5-methoxyuridine. In some embodiments, essentially all uridine nucleotides in the second RNA molecule have been replaced with 5-methoxyuridine. In some embodiments, at least 50% of a total population of uridine nucleotides in the second RNA molecule has been replaced with 5-methyluridine. In some embodiments, essentially all uridine nucleotides in the second RNA molecule have been replaced with 5-methyluridine. In some embodiments, at least 50% of a total population of cytosine nucleotides in the second RNA molecule has been replaced with 5-methylcytosine. In some embodiments, essentially all cytosine nucleotides in the second RNA molecule have been replaced with 5-methylcytosine. In some embodiments, at least 50% of a total population of uridine nucleotides in the second RNA molecule has been replaced with 2-thiouridine. In some embodiments, essentially all uridine nucleotides in the second RNA molecule have been replaced with 2-thiouridine.

In some embodiments, at least 50% of a total population of uridine nucleotides in the second RNA molecule has been replaced with N1-methylpseudouridine and essentially all cytosine nucleotides in the second RNA molecule have been replaced with 5-methylcytosine. In some embodiments, at least 50% of a total population of uridine nucleotides in the second RNA molecule has been replaced with 5-methoxyuridine and essentially all cytosine nucleotides in the second RNA molecule have been replaced with 5-methylcytosine. In some embodiments, at least 50% of a total population of uridine nucleotides in the second RNA molecule has been replaced with 5-methyluridine and essentially all cytosine nucleotides in the second RNA molecule have been replaced with 5-methylcytosine.

In some embodiments, essentially all uridine nucleotides in the second RNA molecule have been replaced with about 50% 5-methoxyuridine and about 50% N1-methylpseudouridine. In some embodiments, essentially all uridine nucleotides in the second RNA molecule have been replaced with about 75% 5-methoxyuridine and about 25% N1-methylpseudouridine. In some embodiments, essentially all uridine nucleotides in the second RNA molecule have been replaced with about 25% 5-methoxyuridine and about 75% N1-methylpseudouridine.

In some embodiments, essentially all uridine nucleotides in the first RNA molecule have been replaced with N1-methylpseudouridine and at least 50% of a total population of uridine nucleotides in the second RNA molecule has been replaced with N1-methylpseudouridine. In some embodiments, essentially all uridine nucleotides in the first RNA molecule have been replaced with N1-methylpseudouridine and essentially all uridine nucleotides in the second RNA molecule have been replaced with N1-methylpseudouridine. In some embodiments, essentially all uridine nucleotides in the first RNA molecule have been replaced with N1-methylpseudouridine and at least 50% of a total population of uridine nucleotides in the second RNA molecule has been replaced with 5-methoxyuridine. In some embodiments, essentially all uridine nucleotides in the first RNA molecule have been replaced with N1-methylpseudouridine, at least 50% of a total population of uridine nucleotides in the second RNA molecule has been replaced with 5-methyluridine, and essentially all cytosine nucleotides in the second RNA molecule have been replaced with 5-methylcytosine. In some embodiments, essentially all uridine nucleotides in the first RNA molecule have been replaced with N1-methylpseudouridine and essentially all uridine nucleotides in the second RNA molecule have been replaced with about 50% 5-methoxyuridine and about 50% N1-methylpseudouridine.

### Methods of Use

The RNA compositions may be utilized to treat and/or prevent an influenza virus of various genotypes, strains, and isolates. Some embodiments provide methods of preventing or treating influenza viral infection comprising administering to a subject any of the RNA compositions described herein. In some embodiments, the antigen specific immune response comprises a T cell response. In some embodiments, the antigen specific immune response comprises a B cell response. In some embodiments, the antigen specific immune response comprises both a T cell response and a B cell response. In some embodiments, the method of producing an antigen specific immune response involves a single administration of the RNA composition. In some embodiments, the RNA composition is administered to the subject by intradermal, intramuscular injection, subcutaneous injection, intranasal inoculation, or oral administration. In some embodiments, the nanoparticle has a net neutral charge at a neutral pH value. In some embodiments, the RNA (e.g., mRNA) vaccine is multivalent.

In some embodiments, the RNA polynucleotides or portions thereof may encode one or more polypeptides or fragments thereof of an influenza strain as an antigen.

Some aspects of the disclosure are directed to a method of inducing an immune response in a subject, comprising administering to the subject in need thereof an effective amount of a composition as disclosed herein. Some aspects of the disclosure are directed to a method of vaccinating a subject, comprising administering to the subject in need thereof an effective amount of a composition as disclosed herein. Some aspects of the disclosure are directed to a method comprising administering to the subject in need thereof an effective amount of a composition as disclosed herein. In some embodiments, a composition as disclosed herein elicits an immune response comprising an antibody response. In some embodiments, a composition as disclosed herein elicits an immune response comprising a T cell response.

### Nucleic Acids

In certain embodiments, nucleic acid sequences can exist in a variety of instances such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, saRNA, and complementary sequences of the foregoing described herein. Nucleic acids that encode an epitope to which antibodies may bind. Nucleic acids encoding fusion proteins that include these polypeptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that can be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

In certain embodiments, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some embodiments, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### Lipid Delivery

In some embodiments, the saRNA composition comprises lipids. The lipids and saRNA may together form nanoparticles. In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo % of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the Txo% of the LNP, LNP suspension, lyophilized LNP composition, or LNP formulation of the present disclosure is longer than the Txo % of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more.

In some embodiments, the T1/2 of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the T1/2 of the LNP, LNP suspension, lyophilized LNP composition, or LNP formulation of the present disclosure is longer than the T1/2 of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80%” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T1/2” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation.

Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

The lipid component of a LNP may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the LNP further comprises a phospholipid, a PEG lipid, a structural lipid, or any combination thereof. Suitable phospholipids, PEG lipids, and structural lipids for the methods of the present disclosure are further disclosed herein.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another embodiment, the lipid component includes about 40 mol % said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol. The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA.

The relative amounts of a therapeutic and/or prophylactic (i.e. pharmaceutical substance) and other elements (e.g., lipids) in a LNP may also vary. In some embodiments, the wt/wt ratio of the lipid component to a therapeutic and/or prophylactic in a LNP may be from about 5:1 to about 60:1, such as 5:1, 6:1, 7:1,8:1,9:1, 10:1, 11:1, 12:1, 13:1, 14:1, 15:1, 16:1, 17:1, 18:1, 19:1, 20:1, 25:1,30:1,35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

In some embodiments, the ionizable lipid is a compound of Formula (I):

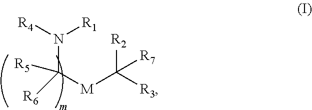

or their N-oxides, or salts or isomers thereof, wherein:

Ri is selected from the group consisting of C5-30 alkyl, C5-20 alkenyl, —R*YR″, —YR″, and —R″M′R′; R2 and R3 are independently selected from the group consisting of H, C1-14 alkyl, C2-14 alkenyl, —R*YR″, —YR″, and —R*OR″, or R2 and R3, together with the atom to which they are attached, form a heterocycle or carbocycle; R4 is selected from the group consisting of hydrogen, a C3-6 carbocycle, —(CH2)nQ, —(CH2)nCHQR, —CHQR, —CQ(R)2, and unsubstituted C1-6 alkyl, where Q is selected from a carbocycle, heterocycle, —OR, —O(CH2)nN(R)2, —C(O) OR, —OC(O)R, —CX3, —CX2H, —CXH2, —CN, —N(R)2, —C(O)N(R)2, —N(R) C(O)R, —N(R)S(O)2R, —N(R)C(O)N(R)2, —N(R)C(S)N(R)2, —N(R)Re, N(R)S(O)2R8, —O(CH2)nOR, —N(R)C(═NR9)N(R)2, —N(R)C(═CHR9)N(R)2, —OC(O)N(R)2J-N(R)C(O)OR, —N(OR)C(O)R, —N(OR)S(O)2R, —N(OR)C(O)OR, —N(OR)C(O)N(R)2, —N(OR)C(S)N(R)2, —N(OR)C(═NR9)N(R)2, —N(OR)C(═CHR9)N(R)2, —C(═NR9)N(R)2, —C(═NR9)R, —C(O)N(R)OR, and —C(R)N(R)2C(O)OR, and each n is independently selected from 1, 2, 3, 4, and 5; each R5 is independently selected from the group consisting of C1-3 alkyl, C2-3 alkenyl, and H; each Re is independently selected from the group consisting of C1-3 alkyl, C2-3 alkenyl, and H; M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)2-, —S—S—, an aryl group, and a heteroaryl group, in which M″ is a bond, C1-13 alkyl or C2-13 alkenyl; R7 is selected from the group consisting of C1-3 alkyl, C2-3 alkenyl, and H; Re is selected from the group consisting of C3-6 carbocycle and heterocycle; R9 is selected from the group consisting of H, CN, NO2, C1-6 alkyl, —OR, —S(O)2R, —S(O)2N(R)2, C2-6 alkenyl, C3-6 carbocycle and heterocycle; each R is independently selected from the group consisting of C1-3 alkyl, C2-3 alkenyl, and H; each R′ is independently selected from the group consisting of Ci- is alkyl, C2- is alkenyl, —R*YR″, —YR″, and H; each R″ is independently selected from the group consisting of C3-15 alkyl and C3-15 alkenyl; each R* is independently selected from the group consisting of Ci-i2 alkyl and C2-12 alkenyl; each Y is independently a C3-6 carbocycle; each X is independently selected from the group consisting of F, C1, Br, and I; and m is selected from 5, 6, 7, 8, 9, 10, 11, 12, and 13; and wherein when R4 is —(CH2)nQ, —(CH2)nCHQR, —CHQR, or —CQ(R)2, then (i) Q is not —N(R) 2 when n is 1, 2, 3, 4 or 5, or (ii) Q is not 5, 6, or 7-membered heterocycloalkyl when n is 1 or 2. In some embodiments, the ionizable lipid is SM-102. In some embodiments, the ionizable lipid is ALC-0315. In some embodiments, the ionizable lipid is:

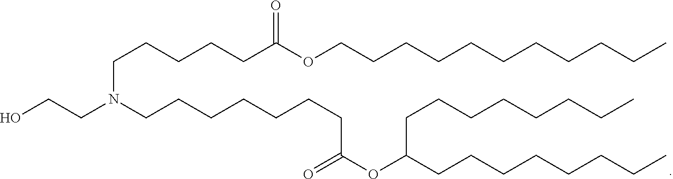 [SMILES: CCCCCCCCCCCOC(=O)CCCCCN(CCO)CCCCCCCC(=O)OC(CCCCCCCC)CCCCCCCC]

In some embodiments, the compounds have the following structure (I):

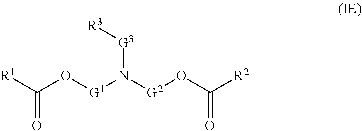

or a pharmaceutically acceptable salt, tautomer, prodrug or stereoisomer thereof, wherein: one of L1 or L2 is —O(C—O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa— or —NRaC (—O)O—, and the other of L1 or L2 is —O(C—O)—, —(C—O)O—, —C(═O)—, —O—, —S(O)x-, —S—S—, —C(—O)S—, SC(—O)—, —NRaC(═O)—, —C(═O)NRa—, NRaC(═O)NRa—, —OC(—O)NRa— or —NRaC(—O)O— or a direct bond; G1 and G2 are each independently unsubstituted C1-C12 alkylene or C1-C12 alkenylene; G3 is C1-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, —C(—O)OR4, —OC(═O)R4 or —NR5C(—O)R4; R4 is C1-C12 alkyl; R5 is H or C1-C6 alkyl; and x is 0, 1 or 2. In a preferred embodiment, the ionizable lipid is:

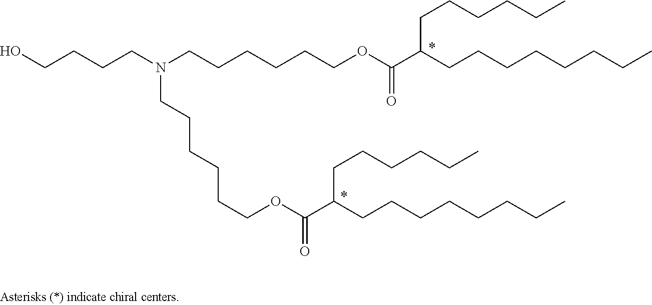 [SMILES: CCCCCCCCC(CCCCCC)C(=O)OCCCCCCN(CCCCO)CCCCCCOC(=O)C(CCCCCC)CCCCCCCC]

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid. As used herein, the term “PEG lipid” refers to polyethylene glycol (PEG)-modified lipids. Non-limiting examples of PEG lipids include PEG-modified phosphatidylethanolamine and phosphatidic acid, PEG-ceramide conjugates (e.g., PEG-CerCI4 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid. In some embodiments, the PEG-modified lipids are a modified form of PEG DMG. In some embodiments, the PEG-modified lipid is PEG lipid with the formula (IV):

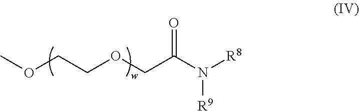

wherein R8 and R9 are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

### Formulation

In one aspect, the disclosure relates to an immunogenic composition including: (i) a first ribonucleic acid (RNA) polynucleotide having an open reading frame encoding a first antigen, said antigen including at least one influenza virus antigenic polypeptide or an immunogenic fragment thereof, and (ii) a second RNA polynucleotide having an open reading frame encoding a second antigen, said second antigen including at least one influenza virus antigenic polypeptide or an immunogenic fragment thereof, wherein the first and second RNA polynucleotides are formulated in a lipid nanoparticle (LNP). In some embodiments, the first and second antigens include hemagglutinin (HA), or an immunogenic fragment or variant thereof. In some embodiments, the first antigen includes an HA from a different subtype of influenza virus to the influenza virus antigenic polypeptide or an immunogenic fragment thereof of the second antigen. In some embodiments, the composition further includes (iii) a third antigen including at least one influenza virus antigenic polypeptide or an immunogenic fragment thereof, wherein the third antigen is from influenza virus but is from a different strain of influenza virus to both the first and second antigens. In some embodiments, the first, second and third RNA polynucleotides are formulated in a lipid nanoparticle.

In some embodiments, the composition comprises an octavalent influenza vaccine comprises RNA encoding an antigenic polypeptide associated with two type A viruses and two type B viruses that are predicted to be prevalent in a relevant jurisdiction. In some embodiments, an octavalent influenza vaccine comprises RNA encoding an antigenic polypeptide derived from HA from an influenza type A virus, RNA encoding an antigenic polypeptide derived from HA from an influenza type A virus, RNA encoding an antigenic polypeptide derived from HA from an influenza type B virus, RNA encoding an antigenic polypeptide derived from HA from an influenza type B virus, RNA encoding an antigenic polypeptide derived from one antigenic polypeptide selected from NA, NP, M1, M2, NS1 and NS2 from an influenza type A virus, RNA encoding an antigenic polypeptide derived from one antigenic polypeptide selected from NA, NP, M1, M2, NS1 and NS2 from an influenza type A virus, RNA encoding an antigenic polypeptide derived from one antigenic polypeptide selected from NA, NP, M1, M2, NS1 and NS2 from an influenza type B virus, and RNA encoding an antigenic polypeptide derived from one antigenic polypeptide selected from NA, NP, M1, M2, NS1 and NS2 from an influenza type B virus. In some embodiments, an octavalent influenza vaccine comprises RNA encoding an antigenic polypeptide derived from HA from an influenza type A virus, RNA encoding an antigenic polypeptide derived from HA from an influenza type A virus, RNA encoding an antigenic polypeptide derived from HA from an influenza type B virus, RNA encoding an antigenic polypeptide derived from HA from an influenza type B virus, RNA encoding an antigenic polypeptide derived from NA from an influenza type A virus, RNA encoding an antigenic polypeptide derived from NA from an influenza type A virus, RNA encoding an antigenic polypeptide derived from NA from an influenza type B virus, and RNA encoding an antigenic polypeptide derived from NA from an influenza type B virus. In some embodiments, an octavalent influenza vaccine comprises RNA encoding an antigenic polypeptide associated with an H1N1 influenza virus, RNA encoding an antigenic polypeptide associated with an H3N2 influenza virus, RNA encoding an antigenic polypeptide associated with a Victoria lineage influenza virus, and RNA encoding an antigenic polypeptide associated with a Yamagata lineage influenza virus. In some embodiments, an octavalent influenza vaccine comprises RNA associated with influenza types that are predicted to be prevalent in a relevant jurisdiction (e.g., HA polypeptides associated with the H1N1, H3N2, B/Victoria, and B/Yamagata influenza viruses that are predicted to be prevalent in a relevant geography). The RNA (e.g., mRNA) vaccines may be utilized in various settings depending on the prevalence of the infection or the degree or level of unmet medical need. The RNA vaccines may be utilized to treat and/or prevent an influenza virus of various genotypes, strains, and isolates. The RNA vaccines typically have superior properties in that they produce much larger antibody titers and produce responses earlier than commercially available anti-viral therapeutic treatments. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

In one aspect, a method of purifying an RNA polynucleotide synthesized by in vitro transcription is provided. The method includes ultrafiltration and diafiltration. In some embodiments, the method does not comprise a chromatography step. In some embodiments, the purified RNA polynucleotide is substantially free of contaminants comprising short abortive RNA species, long abortive RNA species, double-stranded RNA (dsRNA), residual plasmid DNA, residual in vitro transcription enzymes, residual solvent and/or residual salt. In some embodiments, the residual plasmid DNA is ≤500 ng DNA/mg RNA. In some embodiments, purity of the purified mRNA is between about 60% and about 100%. In another aspect, a method of producing an RNA polynucleotide-encapsulated LNP is provided. The method includes buffer exchanging the LNPs. The method further includes concentrating the LNPs via flat sheet cassette membranes. In preferred embodiments, the UFDF process does not utilize hollow fiber membranes.

There may be situations in which persons are at risk for infection with more than one strain of influenza virus. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one strain of influenza, a combination vaccine can be administered that includes RNA (e.g., mRNA) encoding at least one antigenic polypeptide protein (or antigenic portion thereof) of a first influenza virus or organism and further includes RNA encoding at least one antigenic polypeptide protein (or antigenic portion thereof) of a second influenza virus or organism. RNA (e.g., mRNA) can be co-formulated, for example, in a single lipid nanoparticle (LNP) or can be formulated in separate LNPs for co-administration.

Some embodiments of the present disclosure provide influenza virus (influenza) vaccines (or compositions or immunogenic compositions) that include at least one RNA polynucleotide having an open reading frame encoding at least one influenza antigenic polypeptide or an immunogenic fragment thereof (e.g., an immunogenic fragment capable of inducing an immune response to influenza).

In some embodiments, the at least one antigenic polypeptide is one of the defined antigenic subdomains of HA, termed HA1, HA2, or a combination of HA1 and HA2, and at least one antigenic polypeptide selected from neuraminidase (NA), nucleoprotein (NP), matrix protein 1 (M1), matrix protein 2 (M2), non-structural protein 1 (NS1) and non-structural protein 2 (NS2).

In some embodiments, the at least one antigenic polypeptide is HA or derivatives thereof comprising antigenic sequences from HA1 and/or HA2, and at least one antigenic polypeptide selected from HA, NA, NP, M1, M2, NS1 and NS2.

In some embodiments, the at least one antigenic polypeptide is HA or derivatives thereof comprising antigenic sequences from HA1 and/or HA2 and at least two antigenic polypeptides selected from HA, NA, NP, M1, M2, NS1 and NS2.

In some embodiments, a vaccine comprises at least one RNA (e.g., mRNA) polynucleotide having an open reading frame encoding an influenza virus protein, or an immunogenic fragment thereof.

In some embodiments, a vaccine comprises at least one RNA (e.g., mRNA) polynucleotide having an open reading frame encoding multiple influenza virus proteins, or immunogenic fragments thereof.

In some embodiments, a vaccine comprises at least one RNA (e.g., mRNA) polynucleotide having an open reading frame encoding a HA protein, or an immunogenic fragment thereof (e.g., at least one HA1, HA2, or a combination of both).

In some embodiments, a vaccine comprises at least one RNA (e.g., mRNA) polynucleotide having an open reading frame encoding a HA protein, or an immunogenic fragment thereof (e.g., at least one HA1, HA2, or a combination of both, of any one of or a combination of any or all of H1, H2, H3, H4, H5, H6, H7, H8, H9, H10, H11, H12, H13, H14, H15, H16, H17, and/or H18) and at least one other RNA (e.g., mRNA) polynucleotide having an open reading frame encoding a protein selected from a HA protein, NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one any one of or a combination of any or all of H1, H2, H3, H4, H5, H6, H7, H8, H9, H10, H11, H12, H13, H14, H15, H16, H17, and/or H18) and at least two other RNAs (e.g., mRNAs) polynucleotides having two open reading frames encoding two proteins selected from a HA protein, NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least three other RNAs (e.g., mRNAs) polynucleotides having three open reading frames encoding three proteins selected from a HA protein, NP protein, a NA protein, a M protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least four other RNAs (e.g., mRNAs) polynucleotides having four open reading frames encoding four proteins selected from a HA protein, NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least five other RNAs (e.g., mRNAs) polynucleotides having five open reading frames encoding five proteins selected from a HA protein, NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18), HA protein, a NP protein or an immunogenic fragment thereof, a NA protein or an immunogenic fragment thereof, a M1 protein or an immunogenic fragment thereof, a M2 protein or an immunogenic fragment thereof, a NS1 protein or an immunogenic fragment thereof and a NS2 protein or an immunogenic fragment thereof obtained from influenza virus. In some embodiments, an influenza RNA composition includes an saRNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the influenza antigen. Antigenic fusion proteins, in some embodiments, retain the functional property from each original protein.

Some embodiments of the present disclosure provide the following novel influenza virus polypeptide sequences: H1HA 10-Foldon_ANglyl; H1HA 10TM-PR8 (H1 A/Puerto Rico/8/34 HA); H1HA10-PR8-DS (H1 A/Puerto Rico/8/34 HA; pH1HA10-Cal04-DS (H1 A/California/04/2009 HA); Pandemic H1HA 10 from California 04; pH1HA10-ferritin; HA10; Pandemic H1HA10 from California 04; Pandemic H1HA 10 from California 04 strain/without foldon and with K68C/R76C mutation for trimerization; H1HA10 from A/Puerto Rico/8/34 strain, without foldon and with Y94D/N95L mutation for trimerization; H1HA10 from A/Puerto Rico/8/34 strain, without foldon and with K68C/R76C mutation for trimerization; H1N1 A/Viet Nam/850/2009; H3N2 A/Wisconsin/67/2005; H7N9 (A/Anhui/1/2013); H9N2 A/Hong Kong/1073/99; H10N8 A/JX346/2013.

Some embodiments of the present disclosure provide influenza virus (influenza) vaccines that include at least one RNA polynucleotide having an open reading frame encoding at least one influenza antigenic polypeptide or an immunogenic fragment of the novel influenza virus polypeptide sequences described above (e.g., an immunogenic fragment capable of inducing an immune response to influenza). In some embodiments, an influenza vaccine comprises at least one RNA (e.g., mRNA) polynucleotide having an open reading frame encoding at least one influenza antigenic polypeptide comprising a modified sequence that is at least 75% (e.g., any number between 75% and 100%, inclusive, e.g., 70%, 80%, 85%, 90%, 95%, 99%, and 100%) identity to an amino acid sequence of the novel influenza virus sequences described above. The modified sequence can be at least 75% (e.g., any number between 75% and 100%, inclusive, e.g., 70%, 80%, 85%, 90%, 95%, 99%, and 100%) identical to an amino acid sequence of the novel influenza virus sequences described above.

Some embodiments of the present disclosure provide an isolated nucleic acid comprising a sequence encoding the novel influenza virus polypeptide sequences described above; an expression vector comprising the nucleic acid; and a host cell comprising the nucleic acid. The present disclosure also provides a method of producing a polypeptide of any of the novel influenza virus sequences described above. A method may include culturing the host cell in a medium under conditions permitting nucleic acid expression of the novel influenza virus sequences described above, and purifying from the cultured cell or the medium of the cell a novel influenza virus polypeptide. The present disclosure also provides antibody molecules, including full length antibodies and antibody derivatives, directed against the novel influenza virus sequences.

In some embodiments, an open reading frame of a RNA (e.g., mRNA) vaccine is codon-optimized. In some embodiments, the open reading frame which the influenza polypeptide or fragment thereof is encoded is codon-optimized. Some embodiments provide use of an influenza vaccine that includes at least one ribonucleic acid (RNA) polynucleotide having an open reading frame encoding at least one influenza antigenic polypeptide or an immunogenic fragment thereof, wherein at least 80% (e.g., 85%, 90%, 95%, 98%, 99%, 100%) of the uracil in the open reading frame have a chemical modification, optionally wherein the vaccine is formulated in a lipid nanoparticle. In some embodiments, 100% of the uracil in the open reading frame have a chemical modification. In some embodiments, a chemical modification is in the 5-position of the uracil. In some preferred embodiments, a chemical modification is a N1-methyl pseudouridine.

In some embodiments, a RNA (e.g., mRNA) vaccine further comprising an adjuvant.

In some embodiments, at least one RNA polynucleotide encodes at least one influenza antigenic polypeptide that attaches to cell receptors.

In some embodiments, at least one RNA polynucleotide encodes at least one influenza antigenic polypeptide that causes fusion of viral and cellular membranes.

In some embodiments, at least one RNA polynucleotide encodes at least one influenza antigenic polypeptide that is responsible for binding of the virus to a cell being infected.

Some embodiments of the present disclosure provide a vaccine that includes at least one ribonucleic acid (RNA) (e.g., mRNA) polynucleotide having an open reading frame encoding at least one influenza antigenic polypeptide, at least one 5′ terminal cap and at least one chemical modification, formulated within a lipid nanoparticle.

In some embodiments, a 5′ terminal cap is 7 mG (5′) ppp (5′) NImpNp. In some preferred embodiments, the 5′ cap comprises:

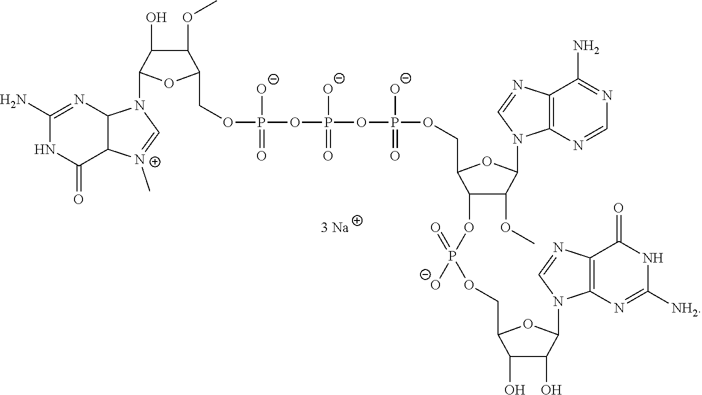 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(N2C=[N+](C)C3C(=O)NC(N)=NC32)C1O]

In some embodiments, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

In some embodiments, a lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino) butanoyl)oxy) heptadecanedioate (L319), (12Z, 15Z)-N,N-dimethyl-2-nonylhenicosa-12,15-dien-1-amine (L608), and N,N-dimethyl-1-[(1S,2R)-2-octylcyclopropyl]heptadecan-8-amine (L530).

Some embodiments of the present disclosure provide a vaccine that includes at least one RNA (e.g., mRNA) polynucleotide having an open reading frame encoding at least one influenza antigenic polypeptide, wherein at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

In some embodiments, an open reading frame of a RNA (e.g., mRNA) polynucleotide encodes at least one influenza antigenic polypeptides. In some embodiments, the open reading frame encodes at least two, at least five, or at least ten antigenic polypeptides. In some embodiments, the open reading frame encodes at least 100 antigenic polypeptides. In some embodiments, the open reading frame encodes 1-100 antigenic polypeptides.

In some embodiments, a vaccine comprises at least two RNA (e.g., mRNA) polynucleotides, each having an open reading frame encoding at least one influenza antigenic polypeptide. In some embodiments, the vaccine comprises at least five or at least ten RNA (e.g., mRNA) polynucleotides, each having an open reading frame encoding at least one antigenic polypeptide or an immunogenic fragment thereof. In some embodiments, the vaccine comprises at least 100 RNA (e.g., mRNA) polynucleotides, each having an open reading frame encoding at least one antigenic polypeptide. In some embodiments, the vaccine comprises 2-100 RNA (e.g., mRNA) polynucleotides, each having an open reading frame encoding at least one antigenic polypeptide.

Also provided herein is an influenza RNA (e.g., mRNA) vaccine of any one of the foregoing paragraphs formulated in a nanoparticle (e.g., a lipid nanoparticle).

In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol.

In some embodiments, the nanoparticle has a polydispersity value of less than 0.4 (e.g., less than 0.3, 0.2 or 0.1).

In some embodiments, the nanoparticle has a net neutral charge at a neutral pH value.

In some embodiments, the RNA (e.g., mRNA) vaccine is multivalent.

Some embodiments of the present disclosure provide methods of inducing an antigen specific immune response in a subject, comprising administering to the subject any of the RNA (e.g., mRNA) vaccine as provided herein in an amount effective to produce an antigen-specific immune response. In some embodiments, the RNA (e.g., mRNA) vaccine is an influenza vaccine. In some embodiments, the RNA (e.g., mRNA) vaccine is a combination vaccine comprising a combination of influenza vaccines (a broad spectrum influenza vaccine).

In some embodiments, an antigen-specific immune response comprises a T cell response or a B cell response.

In some embodiments, a method of producing an antigen-specific immune response comprises administering to a subject a single dose (no booster dose) of an influenza RNA (e.g., mRNA) vaccine of the present disclosure.

In some embodiments, a method further comprises administering to the subject a second (booster) dose of an influenza RNA (e.g., mRNA) vaccine. Additional doses of an influenza RNA (e.g., mRNA) vaccine may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an influenza RNA (e.g., mRNA) vaccine is administered to a subject by intradermal injection, intramuscular injection, or by intranasal administration. In some embodiments, an influenza RNA (e.g., mRNA) vaccine is administered to a subject by intramuscular injection.

Some embodiments, of the present disclosure provide methods of inducing an antigen specific immune response in a subject, including administering to a subject an influenza RNA (e.g., mRNA) vaccine in an effective amount to produce an antigen specific immune response in a subject. Antigen-specific immune responses in a subject may be determined, in some embodiments, by assaying for antibody titer (for titer of an antibody that binds to an influenza antigenic polypeptide) following administration to the subject of any of the influenza RNA (e.g., mRNA) vaccines of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., mRNA) vaccine of the present disclosure. In some embodiments, the control is an anti-antigenic polypeptide antibody titer produced in a subject who has been administered a live attenuated or inactivated influenza, or wherein the control is an anti-antigenic polypeptide antibody titer produced in a subject who has been administered a recombinant or purified influenza protein vaccine. In some embodiments, the control is an anti-antigenic polypeptide antibody titer produced in a subject who has been administered an influenza virus-like particle (VLP) vaccine.

A RNA (e.g., mRNA) vaccine of the present disclosure is administered to a subject in an effective amount (an amount effective to induce an immune response). In some embodiments, the effective amount is a dose equivalent to an at least 2-fold, at least 4-fold, at least 10-fold, at least 100-fold, at least 1000-fold reduction in the standard of care dose of a recombinant influenza protein vaccine, wherein the anti-antigenic polypeptide antibody titer produced in the subject is equivalent to an anti-antigenic polypeptide antibody titer produced in a control subject administered the standard of care dose of a recombinant influenza protein vaccine, a purified influenza protein vaccine, a live attenuated influenza vaccine, an inactivated influenza vaccine, or an influenza VLP vaccine. In some embodiments, the effective amount is a dose equivalent to 2-1000-fold reduction in the standard of care dose of a recombinant influenza protein vaccine, wherein the anti-antigenic polypeptide antibody titer produced in the subject is equivalent to an anti-antigenic polypeptide antibody titer produced in a control subject administered the standard of care dose of a recombinant influenza protein vaccine, a purified influenza protein vaccine, a live attenuated influenza vaccine, an inactivated influenza vaccine, or an influenza VLP vaccine.

In some embodiments, the control is an anti-antigenic polypeptide antibody titer produced in a subject who has been administered a virus-like particle (VLP) vaccine comprising structural proteins of influenza.

In some embodiments, the RNA (e.g., mRNA) vaccine is formulated in an effective amount to produce an antigen specific immune response in a subject.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times. In some embodiments, the effective amount is a total dose of 1 μg to 1000 μg, or 1 μg to 100 μg of saRNA. In some embodiments, the effective amount is a total dose of 30 μg. In some embodiments, the effective amount is a dose of 10 μg administered to the subject a total of two times. In some embodiments, the effective amount is a dose of 10 μg administered to the subject a total of two times. In some embodiments, the effective amount is a dose of 15 μg administered to the subject a total of two times. In some embodiments, the effective amount is a dose of 30 μg administered to the subject a total of two times. In some embodiments, the method includes administering to the subject a saRNA composition described herein at dosage of between 10 μg/kg and 400 μg/kg is administered to the subject.

In some embodiments the dosage of the saRNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the saRNA composition is administered to the subject by intradermal or intramuscular injection. In some embodiments, the saRNA composition is administered to the subject on day zero. In some embodiments, a second dose of the saRNA composition is administered to the subject on day twenty-one.

In some embodiments, the efficacy (or effectiveness) of a RNA (e.g., mRNA) vaccine is greater than 60%. In some embodiments, the RNA (e.g., mRNA) polynucleotide of the vaccine at least one Influenza antigenic polypeptide.

Vaccine efficacy may be assessed using standard analyses. For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:
Efficacy=(ARU−ARV)/ARU×100; and Efficacy=(1−RR)×100.

Likewise, vaccine effectiveness may be assessed using standard analyses. Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination: Effectiveness=(1−OR)×100. In some embodiments, the efficacy (or effectiveness) of a RNA (e.g., mRNA) vaccine is at least 65%, at least 70%, at least 75%, at least 80%, at least 85%, or at least 90%.

In some embodiments, the vaccine immunizes the subject against Influenza for up to 2 years. In some embodiments, the vaccine immunizes the subject against Influenza for more than 2 years, more than 3 years, more than 4 years, or for 5-10 years.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger. In some embodiments, the RNA composition is administered to the subject by intradermal or intramuscular injection. In some embodiments, the RNA composition is administered to the subject on day zero. In some embodiments, a second dose of the RNA composition is administered to the subject on day twenty-one.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject has been exposed to influenza (e.g., C. trachomatis); the subject is infected with influenza (e.g., C. trachomatis); or subject is at risk of infection by influenza (e.g., C. trachomatis).

In some embodiments, the subject has been exposed to betacoronavirus (e.g., SARS-CoV-2); the subject is infected with betacoronavirus (e.g., SARS-COV-2); or subject is at risk of infection by betacoronavirus (e.g., SARS-COV-2).

In some embodiments, the subject has received at least one dose of an immunogenic composition against betacoronavirus (e.g., SARS-COV-2), e.g., selected from any one of COMIRNATY®, the Pfizer-BioNTech COVID-19 vaccine, the Moderna mRNA-1273 COVID-19 vaccine, and the Janssen COVID-19 vaccine; the subject has received at least two doses of an immunogenic composition against betacoronavirus (e.g., SARS-COV-2); the subject is receiving at least one dose of an immunogenic composition against betacoronavirus (e.g., SARS-COV-2), e.g., selected from any one of COMIRNATY®, the Pfizer-BioNTech COVID-19 vaccine, the Moderna mRNA-1273 COVID-19 vaccine, and the Janssen COVID-19 vaccine; or the subject is being administered an immunogenic composition against betacoronavirus (e.g., SARS-COV-2), e.g., selected from any one of COMIRNATY®, the Pfizer-BioNTech COVID-19 vaccine, the Moderna mRNA-1273 COVID-19 vaccine, and the Janssen COVID-19 vaccine at risk of infection by betacoronavirus (e.g., SARS-COV-2) concomitantly, simultaneously, or within 12-48 hours of any one of the immunogenic compositions against influenza disclosed herein.

In some embodiments, the subject is immunocompromised (has an impaired immune system, e.g., has an immune disorder or autoimmune disorder).

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first virus antigenic polypeptide, wherein the RNA polynucleotide does not include a stabilization element, and wherein an adjuvant is not coformulated or co-administered with the vaccine.

In other aspects the invention is a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty-one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100-fold higher level in the local lymph node in comparison with the distal lymph node. In other embodiments the nucleic acid vaccine is chemically modified and in other embodiments the nucleic acid vaccine is not chemically modified.

Aspects of the disclosure provide a nucleic acid vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects of the disclosure provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the disclosure is a neutralizing antibody titer. In some embodiments the neutralizing antibody titer is greater than a protein vaccine. In other embodiments the neutralizing antibody titer produced by the mRNA vaccines of the disclosure is greater than an adjuvanted protein vaccine. In yet other embodiments the neutralizing antibody titer produced by the mRNA vaccines of the disclosure is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

Also provided are nucleic acid vaccines comprising one or more RNA polynucleotides having an open reading frame encoding a first antigenic polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce a neutralizing antibodies within one week of a single administration. In some embodiments, the adjuvant is selected from a cationic peptide and an immunostimulatory nucleic acid. In some embodiments, the cationic peptide is protamine.

Aspects provide nucleic acid vaccines comprising one or more RNA polynucleotides having an open reading frame comprising at least one chemical modification or optionally no modified nucleotides, the open reading frame encoding a first antigenic polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no modified nucleotides, the open reading frame encoding a first antigenic polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Aspects of the disclosure also provide a unit of use vaccine, comprising between 10 μg and 400 μg of one or more RNA polynucleotides having an open reading frame comprising at least one chemical modification or optionally no modified nucleotides, the open reading frame encoding a first antigenic polypeptide, and a pharmaceutically acceptable carrier or excipient, formulated for delivery to a human subject. In some embodiments, the vaccine further comprises a cationic lipid nanoparticle.

Aspects of the disclosure provide methods of creating, maintaining or restoring antigenic memory to a virus strain in an individual or population of individuals comprising administering to said individual or population an antigenic memory booster nucleic acid vaccine comprising (a) at least one RNA polynucleotide, said polynucleotide comprising at least one chemical modification or optionally no modified nucleotides and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration, and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

In some aspects, methods of inducing an antigen specific immune response in a subject are provided. The method includes administering to the subject an influenza RNA composition in an amount effective to produce an antigen specific immune response. In some embodiments, an antigen specific immune response comprises a T cell response or a B cell response. In some embodiments, an antigen specific immune response comprises a T cell response and a B cell response. In some embodiments, a method of producing an antigen specific immune response involves a single administration of the vaccine. In some embodiments, a method further includes administering to the subject a booster dose of the vaccine. In some embodiments, a vaccine is administered to the subject by intradermal or intramuscular injection.

Aspects of the disclosure provide methods of vaccinating a subject comprising administering to the subject a single dosage of between 25 μg/kg and 400 μg/kg of a nucleic acid vaccine comprising one or more RNA polynucleotides having an open reading frame encoding a first antigenic polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Other aspects provide nucleic acid vaccines comprising an LNP formulated RNA polynucleotide having an open reading frame comprising no nucleotide modifications (unmodified), the open reading frame encoding a first antigenic polypeptide, wherein the vaccine has at least 10-fold less RNA polynucleotide than is required for an unmodified mRNA vaccine not formulated in a LNP to produce an equivalent antibody titer. In some embodiments, the RNA polynucleotide is present in a dosage of 25-100 micrograms.

The data presented in the Examples demonstrate significant enhanced immune responses using the formulations of the disclosure. Both chemically modified and unmodified RNA vaccines are useful according to the invention. Surprisingly, in contrast to prior art reports that it was preferable to use chemically unmodified mRNA formulated in a carrier to produce vaccines, it is described herein that chemically modified mRNA-LNP vaccines required a much lower effective mRNA dose than unmodified mRNA, i.e., tenfold less than unmodified mRNA when formulated in carriers other than LNP. Both the chemically modified and unmodified RNA vaccines of the disclosure produce better immune responses than mRNA vaccines formulated in a different lipid carrier.

In other aspects the invention encompasses a method of treating an elderly subject age 60 years or older comprising administering to the subject a nucleic acid vaccine comprising one or more RNA polynucleotides having an open reading frame encoding a virus antigenic polypeptide in an effective amount to vaccinate the subject.

In other aspects the invention encompasses a method of treating a young subject age 17 years or younger comprising administering to the subject a nucleic acid vaccine comprising one or more RNA polynucleotides having an open reading frame encoding a virus antigenic polypeptide in an effective amount to vaccinate the subject.

In other aspects the invention encompasses a method of treating an adult subject comprising administering to the subject a nucleic acid vaccine comprising one or more RNA polynucleotides having an open reading frame encoding a virus antigenic polypeptide in an effective amount to vaccinate the subject.

In some aspects the invention is a method of vaccinating a subject with a combination vaccine including at least two nucleic acid sequences encoding antigens wherein the dosage for the vaccine is a combined therapeutic dosage wherein the dosage of each individual nucleic acid encoding an antigen is a sub therapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject.

In preferred aspects, vaccines of the disclosure (e.g., LNP-encapsulated mRNA vaccines) produce prophylactically- and/or therapeutically efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In exemplary embodiments of the disclosure, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the disclosure, antigen-specific antibodies are measured in units of μg/ml or are measured in units of IU/L (International Units per liter) or mlU/m1 (milli International Units per m1). In exemplary embodiments of the disclosure, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the disclosure, an efficacious vaccine produces >10 mlU/m1, >20 mlU/m1, >50 mlU/m1, >100 mlU/m1, >200 mlU/m1, >500 mlU/m1 or >1000 mlU/m1. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

In some aspects, the disclosure provides a method comprising administering to a human subject a composition comprising: (a) a first messenger ribonucleic acid (mRNA) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid, and wherein the composition comprises 50 or 25 μg to 200 μg of the mRNA in total. In some embodiments, the composition comprises 25 μg of the mRNA in total. In some embodiments, the composition comprises 50 μg of the mRNA in total. In some embodiments, the composition comprises 100 μg of the mRNA in total. In some embodiments, the composition comprises 200 μg of the mRNA in total. In preferred embodiments, the ratio of the first: second: third: fourth mRNA is not 1:1:1:1. In preferred embodiments, the ratio of the first: second: third: fourth mRNA is 1:1: greater than 1: greater than 1, influenza A:A:B:B respectively. In preferred embodiments, the ratio of the first: second: third: fourth mRNA is 1:1:2:2, influenza A:A:B:B respectively. In preferred embodiments. the ratio of the first: second: third: fourth mRNA is 1:1:4:4, influenza A:A:B:B respectively. In some embodiments, each mRNA comprises a 5′ cap analog. In some embodiments, the 5′ cap analog is a 5′ 7 mG (5′) ppp (5′) NImpNp cap. In some embodiments, each mRNA comprises a chemical modification. In some embodiments, the chemical modification is 1-methylpseudouridine. In some embodiments, the lipid nanoparticle comprises 40-50 mol % ionizable amino lipid, 35-45 mol % sterol, 10-15 mol % neutral lipid, and 2-4 mol % PEG-modified lipid. In some embodiments, the lipid nanoparticle comprises 45 mol %, 46 mol %, 47 mol %, 48 mol %, 49 mol %, or 50 mol % ionizable amino lipid. In some embodiments, the sterol is cholesterol. In some embodiments, the neutral lipid is 1,2 distearoyl-sn-glycero-3-phosphocholine (DSPC). In some embodiments, the PEG-modified lipid is 1,2 dimyristoyl-sn-glycerol, methoxypolyethyleneglycol (PEG2000 DMG). In some embodiments, the composition further comprises Tris buffer, sucrose, and sodium acetate. In some embodiments, the composition comprises 10 mM-30 mM Tris buffer, 75 mg/ml-95 mg/mL sucrose, and 5 mM-15 mM sodium acetate, optionally wherein the composition has a pH of 6-8. In some embodiments, the composition comprises 20 mM Tris buffer, 87 mg/ml sucrose, and 10.7 mM sodium acetate, optionally wherein the composition has a pH of 7.5. In some embodiments, the composition comprises 0.5 mg/ml of the mRNA.

In some embodiments, the composition is administered intramuscularly, optionally into a deltoid region of the human subject. In some embodiments, the human subject is 18 to 49 years of age. In some embodiments, the human subject is at least 50 years of age. In some embodiments, the human subject is 50-64 years of age. In some embodiments, the human subject is at least 65 years of age. In some embodiments, the human subject is seropositive for at least one of the HA antigens. In some embodiments, the human subject is seropositive for all of the HA antigens. In some embodiments, the subject is seronegative for all of the HA antigens. In some embodiments, the HA antigens are recommended by or selected according to standardized criteria used by World Health Organization's Global Influenza Surveillance and Response System (GISRS). In some embodiments, the HA antigen are selected using a hemagglutinin inhibition (HAI) assay to identify circulating influenza viruses that are antigenically similar to influenza viruses from a previous season's vaccine, optionally wherein influenza viruses are considered to be antigenically similar if their HAI titers differ by two dilutions or less. In some embodiments, the first mRNA encodes an influenza A HA antigen of the H1 subtype, the second mRNA encodes an influenza A HA antigen of the H3 subtype, the third mRNA encodes an influenza B HA antigen of the B/Yamagata lineage, and the fourth mRNA encodes an influenza B HA antigen of the B/Victoria lineage.

In some embodiments, the mRNA vaccine is administered in an amount effective to induce a neutralizing antibody response against influenza A H1N1, influenza A H3N2, influenza B/Yamagata, and influenza B/Victoria. In some embodiments, the mRNA vaccine is administered in an amount effective to induce a T cell response against influenza A H1N1, influenza A H3N2, influenza B/Yamagata, and influenza B/Victoria. The disclosure, in another aspect, provides a composition comprising: (a) a first messenger ribonucleic acid (mRNA) comprising an open reading frame (ORF) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid; wherein the ratio of the first: second: third: fourth mRNA is 1:1:greater than 1: greater than 1, influenza A:A:B:B strains, respectively.

The disclosure, in another aspect, provides a composition comprising: (a) a first messenger ribonucleic acid (mRNA) comprising an open reading frame (ORF) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid; wherein the ratio of the first: second: third: fourth mRNA is 1:1:greater than 1: greater than 1, influenza A:A:B:B strains, respectively.

Another aspect of the disclosure provides a composition comprising: (a) a first messenger ribonucleic acid (mRNA) comprising an open reading frame (ORF) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid; wherein the ratio of the first: second: third: fourth mRNA is 1:1:greater than 1: greater than 1, influenza A:A:B:B strains, respectively, and wherein the composition comprises 100 μg of the mRNA in total.

The disclosure, in another aspect, provides a composition comprising: (a) a first messenger ribonucleic acid (mRNA) comprising an open reading frame (ORF) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid; wherein the ratio of the first: second: third: fourth mRNA is 1:1:greater than 1: greater than 1, influenza A:A:B:B strains, respectively, and wherein the composition comprises 200 μg of the mRNA in total.

Another aspect of the disclosure includes a method for vaccinating a human subject, comprising administering an mRNA vaccine comprising mRNA encoding at least 4 influenza antigens, wherein the influenza antigens comprise at least 2 hemagglutinin (HA) A antigens, each of a different subtype and at least 2 HA B antigens, each of a different lineage, in an effective amount to produce an antigen specific immune response in the subject.

In some embodiments, the influenza antigens are encoded by one to four mRNAs. In some embodiments, the mRNA comprises a single mRNA encoding the at least 4 influenza antigens. In some embodiments, the mRNA comprises four mRNA each comprising a single open reading frame (ORF) encoding one of the 4 influenza antigens. In some embodiments, the 4 influenza antigens comprise an influenza A HA antigen of the H1 subtype, an influenza A HA antigen of the H3 subtype, an influenza B HA antigen of the B/Yamagata lineage, and an influenza B HA antigen of the B/Victoria lineage.

Another aspect of the disclosure provides a composition comprising an mRNA vaccine comprising mRNA encoding at least 4 influenza antigens, wherein the influenza antigens comprise at least 2 hemagglutinin (HA) A antigens, each of a different subtype and at least 2 HA B antigens, each of a different lineage, wherein the composition further comprises a lipid nanoparticle comprising 40-55 mol % ionizable amino lipid; 5-15 mol % neutral lipid; 35-45 mol % sterol; and 1-5 mol % PEG-modified lipid.

In some embodiments, the influenza antigens are encoded by one to four mRNAs. In some embodiments, the mRNA comprises a single mRNA encoding the at least 4 influenza antigens. In some embodiments, the mRNA comprises four mRNA each comprising a single open reading frame (ORF) encoding one of the 4 influenza antigens. In some embodiments, the 4 influenza antigens comprise an influenza A HA antigen of the H1 subtype, an influenza A HA antigen of the H3 subtype, an influenza B HA antigen of the B/Yamagata lineage, and an influenza B HA antigen of the B/Victoria lineage.

In some embodiments, the 4 mRNA are present in the composition in 1:1:greater than 1: greater than 1 ratio, influenza A:A:B:B strains, respectively. In some embodiments, the composition comprises 25 μg to 200 μg of the mRNA in total. In some embodiments, the percentage of subjects with seroconversion with respect to one of the four influenza antigens after a single dose at Day 29 is at least 40%, at least 50%, at least 60%, at least 70%, at least 80%, at least 90%, at least 95% or 100%. In some embodiments, the percentage of subjects with seroconversion with respect to one of the four influenza antigens after a single dose at Day 29 is 100%.

In some embodiments, the percentage of subjects with seroconversion with respect to two of the four influenza antigens after a single dose at Day 29 is at least 40%, at least 50%, at least 60%, at least 70%, at least 80%, at least 90%, at least 95% or 100%. In some embodiments, the percentage of subjects with seroconversion with respect to two of the four influenza antigens after a single dose at Day 29 is 100%.

In some embodiments, the percentage of subjects with seroconversion with respect to three of the four influenza antigens after a single dose at Day 29 is at least 40%, at least 50%, at least 60%, at least 70%, at least 80%, at least 90%, at least 95% or 100%. In some embodiments, the percentage of subjects with seroconversion with respect to three of the four influenza antigens after a single dose at Day 29 is 100%.

In some embodiments, the percentage of subjects with seroconversion with respect to all four of the influenza antigens after a single dose at Day 29 is at least 40%, at least 50%, at least 60%, at least 70%, at least 80%, at least 90%, at least 95% or 100%. In some embodiments, the percentage of subjects with seroconversion with respect to all four of the influenza antigens after a single dose at Day 29 is 100%.

In some embodiments, the percentage of subjects with at least a 2-fold rise in geometric mean fold rise (GMFR) after a single dose at Day 29 is at least 80%, at least 90%, at least 95% or 100%. In some embodiments, the percentage of subjects with a 2-fold rise in GMFR after a single dose at Day 29 is 100%. In some embodiments, the percentage of subjects with a 4-fold rise in GMFR after a single dose at Day 29 is at least 80%, at least 90%, at least 95% or 100%. In some embodiments, the percentage of subjects with a 4-fold rise in GMFR after a single dose at Day 29 is 100%

In some embodiments, the GMFR comprises the H1N1 HAI titer GMFR. In some embodiments, the GMFR comprises the H3N2 HAI titer GMFR. In some embodiments, the GMFR comprises the B/Yamagata HAI titer GMFR. In some embodiments, the GMFR comprises the B/Victoria HAI titer GMFR.

In some embodiments, the serum antibody titers are increased 4-fold over baseline (Day 0) at Day 29 and Day 57 after a single dose. In some embodiments, the serum antibody titers are decreased at Day 181 over Day 29 titers after a single dose.

In some embodiments, the microneutralization titers are increased 4-fold over baseline (Day 0) at Day 29 and/or Day 57 after a single dose. In some embodiments, the microneutralization titers are decreased at Day 181 over Day 29 titers after a single dose.

In some embodiments, the composition comprises an approximately 25 μg to 250 μg dose of mRNA encoding four influenza HA proteins (e.g., HA proteins associated with the A/H1N1 strain, A/H3N2 strain, B/Victoria lineage and B/Yamagata lineage). In some embodiments, the composition comprises equal amounts of each of the mRNA encoding each of the four HA proteins (e.g., the mRNA are present in the composition at a 1:1:greater than 1: greater than 1 ratio, influenza A:A:B:B strains, respectively). In some embodiments, a composition comprises an approximately 25 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 50 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 100 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 150 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 200 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 250 μg dose of mRNA encoding four influenza HA proteins (e.g., HA proteins associated with the A/H1N1 strain, A/H3N2 strain, B/Victoria lineage and B/Yamagata lineage).

A composition may further comprise a buffer, for example a Tris buffer. For example, a composition may comprise 10 mM-30 mM, 10 mM-20 mM, or 20 mM-30 mM Tris buffer. In some embodiments, a composition comprises 10, 15, 20, 25, or 30 mM Tris buffer. In some embodiments, a composition comprises 20 mM Tris buffer.

In some embodiments, mRNA of a composition is formulated at a concentration of 0.1-1 mg/mL. In some embodiments, mRNA of a composition is formulated at a concentration of 0.1, 0.2, 0.3, 0.4, 0.5, 0.6, 0.7, 0.8, 0.9, or 1 mg/mL. In some embodiments, mRNA of a composition is formulated at a concentration of 0.5 mg/mL.

In some embodiments, a composition comprises sucrose. For example, a composition may comprise 75 mg/mL-95 mg/mL, 75 mg/mL-85 mg/mL, or 85 mg/mL-95 mg/mL sucrose. In some embodiments, a composition comprises 75, 80, 85, 86, 87, 88, 89, 90, or 95 mg/mL sucrose. In some embodiments, a composition comprises 87 mg/mL sucrose. In preferred embodiments, a composition does not include sodium acetate.

A composition may have a pH value of 6-8. In some embodiments, a composition has a pH value of 6, 6.5, 7, 7.5, or 8. In some embodiments, a composition has a pH value of 7.5.

In some embodiments, the composition further comprises a mixture of lipids. The mixture of lipids typically forms a lipid nanoparticle. The mRNA described herein, in some embodiments, is formulated with a lipid nanoparticle (e.g., for administration to a subject). In some embodiments, the lipid mixture, and thus the lipid nanoparticle, comprises: an ionizable amino lipid; a neutral lipid; a sterol; and a PEG-modified lipid.

For example, the lipid mixture/lipid nanoparticle may comprise: 20-60 mol % ionizable amino lipid; 5-25 mol % neutral lipid; 25-55 mol % sterol; and 0.5-15 mol % PEG-modified lipid. In some embodiments, the lipid nanoparticle comprises: 20-60 mol % ionizable amino lipid; 5-25 mol % neutral lipid; 25-55 mol % sterol; and 0.5-15 mol % PEG-modified lipid. In some embodiments, the lipid nanoparticle comprises: 40-55 mol % ionizable amino lipid; 5-15 mol % neutral lipid; 35-45 mol % sterol; and 1-5 mol % PEG-modified lipid. For example, the lipid nanoparticle may comprise: (a) 47 mol % ionizable amino lipid; 11.5 mol % neutral lipid; 38.5 mol % sterol; and 3.0 mol % PEG-modified lipid; (b) 48 mol % ionizable amino lipid; 11 mol % neutral lipid; 38.5 mol % sterol; and 2.5 mol % PEG-modified lipid; (c) 49 mol % ionizable amino lipid; 10.5 mol % neutral lipid; 38.5 mol % sterol; and 2.0 mol % PEG-modified lipid; (d) 50 mol % ionizable amino lipid; 10 mol % neutral lipid; 38.5 mol % sterol; and 1.5 mol % PEG-modified lipid; or (e) 51 mol % ionizable amino lipid; 9.5 mol % neutral lipid; 38.5 mol % sterol; and 1.0 mol % PEG-modified lipid. In some embodiments, the lipid mixture, and thus the lipid nanoparticle, comprises 20-55 mol %, 20-50 mol %, 20-45 mol %, 20-40 mol %, 25-60 mol %, 25-55 mol %, 25-50 mol %, 25-45 mol %, 25-40 mol %, 30-60 mol %, 30-55 mol %, 30-50 mol %, 30-45 mol %, 30-40 mol %, 35-60 mol %, 35-55 mol %, 35-50 mol %, 35-45 mol %, 35-40 mol %, 40-60 mol %, 40-55 mol %, 40-50 mol %, 40-45 mol %, 50-60 mol %, 50-55 mol %, or 55-60 mol % ionizable amino lipid. In some embodiments, the lipid mixture, and thus the lipid nanoparticle, comprises 5-20 mol %, 5-15 mol %, 5-10 mol %, 10-25 mol %, 10-20 mol %, 10-15 mol %, 15-25 mol %, 15-20 mol %, or 20-25 mol % neutral lipid. In some embodiments, the lipid mixture, and thus the lipid nanoparticle, comprises 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid mixture, and thus the lipid nanoparticle, comprises 0.5-10 mol %, 0.5-5 mol %, 0.5-1 mol %, 1-15%, 1-10 mol %, 1-5 mol %, 1.5-15%, 1.5-10 mol %, 1.5-5 mol %, 2-15%, 2-10 mol %, 2-5 mol %, 2.5-15%, 2.5-10 mol %, 2.5-5 mol %, 3-15%, 3-10 mol %, or 3-5 mol %, PEG-modified lipid. In some embodiments, the lipid mixture comprises: 50 mol % ionizable amino lipid; 10 mol % neutral lipid; 38.5 mol % sterol; and 1.5 mol % PEG-modified lipid. In some embodiments, the ionizable amino lipid is heptadecan-9-yl 8 ((2 hydroxyethyl) (6 oxo 6-(undecyloxy) hexyl)amino) octanoate. In some embodiments, the neutral lipid is 1,2 distearoyl sn glycero-3 phosphocholine (DSPC). In some embodiments, the sterol is cholesterol. In some embodiments, the PEG-modified lipid is 1-monomethoxypolyethyleneglycol-2,3-dimyristylglycerol with polyethylene glycol of average molecular weight 2000 (PEG2000 DMG).

A composition may further include a pharmaceutically-acceptable excipient, inert or active. A pharmaceutically acceptable excipient, after administered to a subject, does not cause undesirable physiological effects. The excipient in the pharmaceutical composition must be “acceptable” also in the sense that it is compatible with mRNA and can be capable of stabilizing it. One or more excipients (e.g., solubilizing agents) can be utilized as pharmaceutical carriers for delivery of the mRNA. Examples of a pharmaceutically acceptable excipients include, but are not limited to, biocompatible vehicles (e.g., LNPs), carriers, adjuvants, additives, and diluents to achieve a composition usable as a dosage form. Examples of other excipients include colloidal silicon oxide, magnesium stearate, cellulose, and sodium lauryl sulfate. Additional suitable pharmaceutical excipients, as well as pharmaceutical necessities for their use, are described in Remington's Pharmaceutical Sciences.

In some embodiments, an mRNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof. In some embodiments, a composition comprising mRNA does not include an adjuvant (the composition is adjuvant-free). Compositions may be sterile, pyrogen-free or both sterile and pyrogen-free. General considerations in the formulation and/or manufacture of pharmaceutical agents, such as compositions, may be found, for example, in Remington: The Science and Practice of Pharmacy 21st ed., Lippincott Williams & Wilkins, 2005. Formulations of the compositions described herein may be prepared by any method known or hereafter developed in the art of pharmacology. In general, such preparatory methods include the step of bringing the mRNA into association with an excipient (e.g., a mixture of lipids and/or a lipid nanoparticle), and then, if necessary and/or desirable, dividing, shaping and/or packaging the product into a desired single- or multi-dose unit. Relative amounts of the mRNA, the pharmaceutically-acceptable excipient, and/or any additional ingredients in a composition in accordance with the disclosure may vary, depending upon the identity, size, and/or condition of the subject treated and further depending upon the route by which the composition is to be administered.

SEQUENCES
>B/Austria/1359417/2021-HA| WT
(SEQ ID NO: 9)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-001| Δ(572-582)
(SEQ ID NO: 10)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMV
>pBv-002| V571C
(SEQ ID NO: 11)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMCSRDNVSCSICL
>pBv-003| S577C
(SEQ ID NO: 12)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVCCSICL
>pBv-004| S579C
(SEQ ID NO: 13)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCCICL
>pBv-005| L582C
(SEQ ID NO: 14)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICC
>pBv-006| V571C, S572C
(SEQ ID NO: 15)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMCCRDNVSCSICL
>pBv-007| V571C, S579C
(SEQ ID NO: 16)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMCSRDNVSCCICL
>pBv-008| N575C, V576C
(SEQ ID NO: 17)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDCCSCSICL
>pBv-009| V571C, S572C, S577C
(SEQ ID NO: 18)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMCCRDNVCCSICL
>pBv-010| V571C, S572C, S579C
(SEQ ID NO: 19)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMCCRDNVSCCICL
>pBv-011| R573C, D574C, S579C
(SEQ ID NO: 20)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSCCNVSCCICL
>pBv-012| R573C, D574C, S579C, + KGCCSCGSCC
(SEQ ID NO: 21)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSCCNVSCCICLKGCCSCGSCC
>pBv-013| Δ361, Δ378
(SEQ ID NO: 22)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERG FGAIAGFLEGGWEGMI
GWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-014| Δ(352-357)
(SEQ ID NO: 23)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRP
ERGFFGAIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-015| Δ(352-359)
(SEQ ID NO: 24)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRP
GFFGAIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-016| Δ(352-362)
(SEQ ID NO: 25)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRP
GAIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-017| Δ(352-366)
(SEQ ID NO: 26)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRP
GFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-018| Δ(352-368)
(SEQ ID NO: 27)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRP
LEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-019| Δ(352-370)
(SEQ ID NO: 28)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRP
GGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-020| Δ(354-365)
(SEQ ID NO: 29)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPA
AGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-021| Δ(354-366)
(SEQ ID NO: 30)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPA
GFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-022| Δ(355-358)
(SEQ ID NO: 31)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAK
RGFFGAIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-023| Δ(355-361)
(SEQ ID NO: 32)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAK
FGAIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-024| Δ(355-363)
(SEQ ID NO: 33)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAK
AIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-025| Δ(355-364)
(SEQ ID NO: 34)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAK
IAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-026| Δ(355-367)
(SEQ ID NO: 35)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAK
FLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-027| Δ(356-363)
(SEQ ID NO: 36)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKL
AIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-028| Δ(358-362)
(SEQ ID NO: 37)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLK
GAIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-029| Δ(359-361)
(SEQ ID NO: 38)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKE
FGAIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-030| Δ(359-363)
(SEQ ID NO: 39)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKE
AIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-031| Δ(359-364)
(SEQ ID NO: 40)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKE
IAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-032| Δ(361-364)
(SEQ ID NO: 41)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERG
IAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-033| Δ(361-367)
(SEQ ID NO: 42)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERG
FLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-034| Δ(364-367)
(SEQ ID NO: 43)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFG
FLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-035| Δ(364-369)
(SEQ ID NO: 44)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFG
EGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-036| Δ(364-370)
(SEQ ID NO: 45)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFG
GGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-037| Δ(366-373)
(SEQ ID NO: 46)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAI
EGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-038| Δ(369-373)
(SEQ ID NO: 47)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGF
EGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-039| R359A, G360A
(SEQ ID NO: 48)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKEAAFFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-040| F361A
(SEQ ID NO: 49)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGAFGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-041| F362A
(SEQ ID NO: 50)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFAGAIAGFLEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-042| 1365A
(SEQ ID NO: 51)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAAAGFLEGGWEGMIAGWHG
YTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-043| F368A
(SEQ ID NO: 52)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGALEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-044| L369A
(SEQ ID NO: 53)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGFAEGGWEGMIAGWHGY
TSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-045| G360P, F361P, I365G
(SEQ ID NO: 54)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERPPFGAGAGFLEGGWEGMIAGWHG
YTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICL
>pBv-046| Δ(355-361), S579C
(SEQ ID NO: 55)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAK
FGAIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCCICL
>pBv-047| Δ(361-364), S579C
(SEQ ID NO: 56)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERG
IAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCCICL
>pBv-048| Δ(361-367), S579C
(SEQ ID NO: 57)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERG
FLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCCICL
>pBv-049| Δ(364-367), S579C
(SEQ ID NO: 58)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFG
FLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCCICL
>pBv-050| Δ(366-373), S579C
(SEQ ID NO: 59)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAI
EGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCCICL
>pBv-051| Δ(369-373), S579C
(SEQ ID NO: 60)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGF
EGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCCICL
>pBv-052| Δ(369-373), V571C, S572C
(SEQ ID NO: 61)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGF
EGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMCCRDNVSCSICL
>pBv-053| Δ(369-373), L582C
(SEQ ID NO: 62)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGF
EGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSRDNVSCSICC
>pBv-054| Δ(355-361), R573C, D574C, S579C
(SEQ ID NO: 63)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAK
FGAIAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSCCNVSCCICL
>pBv-055| Δ(361-364), R573C, D574C, S579C
(SEQ ID NO: 64)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERG
IAGFLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSCCNVSCCICL
>pBv-056| Δ(361-367), R573C, D574C, S579C
(SEQ ID NO: 65)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERG
FLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSCCNVSCCICL
>pBv-057| Δ(364-367), R573C, D574C, S579C
(SEQ ID NO: 66)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFG
FLEGGWEGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSCCNVSCCICL
>pBv-058| Δ(366-373), R573C, D574C, S579C
(SEQ ID NO: 67)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAI
EGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSCCNVSCCICL
>pBv-059| Δ(369-373), R573C, D574C, S579C
(SEQ ID NO: 68)
MKAIIVLLMVVTSNADRICTGITSSNSPHVVKTATQGEVNVTGVIPLTTTPTKSHFANLKGTETRG
KLCPKCLNCTDLDV
ALGRPKCTGKIPSARVSILHEVRPVTSGCFPIMHDRTKIRQLPNLLRGYEHVRLSTHNVINTEDA
PGGPYEIGTSGSCLN
ITNGKGFFATMAWAVPKNKTATNPLTIEVPYICTEEEDQITVWGFHSDDETQMARLYGDSKPQK
FTSSANGVTTHYVSQI
GGFPNQTEDGGLPQSGRIVVDYMVQKSGKTGTITYQRGILLPQKVWCASGKSKVIKGSLPLIGE
ADCLHEKYGGLNKSKP
YYTGEHAKAIGNCPIWVKTPLKLANGTKYRPPAKLLKERGFFGAIAGF
EGMIAGWHGYTSHGAHGVAVAADLKST
QEAINKITKNLNSLSELEVKNLQRLSGAMDELHNEILELDEKVDDLRADTISSQIELAVLLSNEGII
NSEDEHLLALERK
LKKMLGPSAVEIGNGCFETKHKCNQTCLDRIAAGTFDAGEFSLPTFDSLNITAASLNDDGLDNH
TILLYYSTAASSLAVT
LMIAIFVVYMVSCCNVSCCICL
>pBv-060|Delta(361-364), L582C
(SEQ ID NO: 69)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
IAGFLEGGWE GMIAGWHGYT SHGAHGVAVA ADLKSTQEAI NKITKNLNSL SELEVKNLQR
LSGAMDELHN EILELDEKVD DLRADTISSQ IELAVLLSNE GIINSEDEHL LALERKLKKM
LGPSAVEIGN GCFETKHKCN QTCLDRIAAG TFDAGEFSLP TFDSLNITAA SLNDDGLDNH
TILLYYSTAA SSLAVTLMIA IFVVYMVSRD NVSCSICC
>pBv-061|Delta(361-364), S579C
(SEQ ID NO: 70)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
IAGFLEGGWE GMIAGWHGYT SHGAHGVAVA ADLKSTQEAI NKITKNLNSL SELEVKNLQR
LSGAMDELHN EILELDEKVD DLRADTISSQ IELAVLLSNE GIINSEDEHL LALERKLKKM
LGPSAVEIGN GCFETKHKCN QTCLDRIAAG TFDAGEFSLP TFDSLNITAA SLNDDGLDNH
TILLYYSTAA SSLAVTLMIA IFVVYMVSRD NVSCCICL
>pBv-062|Delta(355-361), L582C
(SEQ ID NO: 71)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKFGAIAG
FLEGGWEGMI AGWHGYTSHG AHGVAVAADL KSTQEAINKI TKNLNSLSEL EVKNLQRLSG
AMDELHNEIL ELDEKVDDLR ADTISSQIEL AVLLSNEGII NSEDEHLLAL ERKLKKMLGP
SAVEIGNGCF ETKHKCNQTC LDRIAAGTFD AGEFSLPTFD SLNITAASLN DDGLDNHTIL
LYYSTAASSL AVTLMIAIFV VYMVSRDNVS CSICC
>pBv-063|Delta(355-361), S579
(SEQ ID NO: 72)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKFGAIAG
FLEGGWEGMI AGWHGYTSHG AHGVAVAADL KSTQEAINKI TKNLNSLSEL EVKNLQRLSG
AMDELHNEIL ELDEKVDDLR ADTISSQIEL AVLLSNEGII NSEDEHLLAL ERKLKKMLGP
SAVEIGNGCF ETKHKCNQTC LDRIAAGTFD AGEFSLPTFD SLNITAASLN DDGLDNHTIL
LYYSTAASSL AVTLMIAIFV VYMVSRDNVS CCICL
>pBv-064|Delta(355-361), V571C
(SEQ ID NO: 73)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKFGAIAGFLEGGWEGMIAGWHGYTSHGAHGV
AVAADLKSTQEAINKITKNLNSLSELEVKNLQRLSGAMDELHNEILELDEKVDDLRADTISSQIEL
AVLLSNEGIINSEDEHLLALERKLKKMLGPSAVEIGNGCFETKHKCNQTCLDRIAAGTFDAGEFS
LPTFDSLNITAASLNDDGLDNHTILLYYSTAASSLAVTLMIAIFVVYMCSRDNVSCSICL
>pBv-065|Delta(355-363) + V571C
(SEQ ID NO: 74)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKAIAGFLEGGWEGMIAGWHGYTSHGAHGVAVA
ADLKSTQEAINKITKNLNSLSELEVKNLQRLSGAMDELHNEILELDEKVDDLRADTISSQIELAVLL
SNEGIINSEDEHLLALERKLKKMLGPSAVEIGNGCFETKHKCNQTCLDRIAAGTFDAGEFSLPTF
DSLNITAASLNDDGLDNHTILLYYSTAASSLAVTLMIAIFVVYMCSRDNVSCSICL
>pBv-066| Delta(354-366), L582C
(SEQ ID NO: 75)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAGFLEGGW
EGMIAGWHGY TSHGAHGVAV AADLKSTQEA INKITKNLNS LSELEVKNLQ RLSGAMDELH
NEILELDEKV DDLRADTISS QIELAVLLSN EGIINSEDEH LLALERKLKK MLGPSAVEIG
NGCFETKHKC NQTCLDRIAA GTFDAGEFSL PTFDSLNITA ASLNDDGLDN HTILLYYSTA
ASSLAVTLMI AIFVVYMVSR DNVSCSICC
>pBv-067| S579C, L582C
(SEQ ID NO: 76)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCCI CC
>pBv-068|Delta(356-373), L582C
(SEQ ID NO: 77)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLEGMIA
GWHGYTSHGA HGVAVAADLK STQEAINKIT KNLNSLSELE VKNLQRLSGA MDELHNEILE
LDEKVDDLRA DTISSQIELA VLLSNEGIIN SEDEHLLALE RKLKKMLGPS AVEIGNGCFE
TKHKCNQTCL DRIAAGTFDA GEFSLPTFDS LNITAASLND DGLDNHTILL YYSTAASSLA
VTLMIAIFVV YMVSRDNVSC SICC
>pBv-069|Delta(366-373), L582C
(SEQ ID NO: 78)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIEGMIA GWHGYTSHGA HGVAVAADLK STQEAINKIT KNLNSLSELE VKNLQRLSGA
MDELHNEILE LDEKVDDLRA DTISSQIELA VLLSNEGIIN SEDEHLLALE RKLKKMLGPS
AVEIGNGCFE TKHKCNQTCL DRIAAGTFDA GEFSLPTFDS LNITAASLND DGLDNHTILL
YYSTAASSLA VTLMIAIFVV YMVSRDNVSC SICC
>pBv-070|Delta(355-363), L582C
(SEQ ID NO: 79)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKAIAGFL
EGGWEGMIAG WHGYTSHGAH GVAVAADLKS TQEAINKITK NLNSLSELEV KNLQRLSGAM
DELHNEILEL DEKVDDLRAD TISSQIELAV LLSNEGIINS EDEHLLALER KLKKMLGPSA
VEIGNGCFET KHKCNQTCLD RIAAGTFDAG EFSLPTFDSL NITAASLNDD GLDNHTILLY
YSTAASSLAV TLMIAIFVVY MVSRDNVSCS ICC
>pBv-071| G360P, F361P, I365G, L582C
(SEQ NO ID: 80)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERP
PFGAGAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI CC
>pBv-072|Delta361, Delta378, L582C
(SEQ ID NO: 81)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FGAIAGFLEG GWEGMIGWHG YTSHGAHGVA VAADLKSTQE AINKITKNLN SLSELEVKNL
QRLSGAMDEL HNEILELDEK VDDLRADTIS SQIELAVLLS NEGIINSEDE HLLALERKLK
KMLGPSAVEI GNGCFETKHK CNQTCLDRIA AGTFDAGEFS LPTFDSLNIT AASLNDDGLD
NHTILLYYST AASSLAVTLM IAIFVVYMVS RDNVSCSICC
>pBv-073| F368A, L582C
(SEQ ID NO: 82)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIAGALE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI CC
>pBv-074| F368A, S579C
(SEQ ID NO: 83)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIAGALE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCCI CL
>pBv-075| G360P, F361P, I365G, Delta582
(SEQ ID NO: 84)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERP
PFGAGAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI C
>pBv-076| G360P, F361P, I365G, Delta(581-582)
(SEQ ID NO: 85)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERP
PFGAGAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI
>pBv-077|Delta582
(SEQ ID NO: 86)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI C
>pBv-078|Delta(581-582)
(SEQ ID NO: 087)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI
>pBv-079| D471S
(SEQ ID NO: 088)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE SEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI CL
>pBv-080| H473D, A476K
(SEQ ID NO: 089)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEDLLKLERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI CL
>pBv-081| Y321F, S453Y, I451W
(SEQ ID: 090)
MKAIIVLLMV VTSNADRICT GITSSNSPHV VKTATQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP FYTGEHAKAI GNCPIWVKTP LKLANGTKYR PPAKLLKERG
FFGAIAGFLE GGWEGMIAGW HGYTSHGAHG VAVAADLKST QEAINKITKN LNSLSELEVK
NLQRLSGAMD ELHNEILELD EKVDDLRADT WSYQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI CL
>pBV-082| T35C, K406C
(SEQ ID NO: 091)
MKAIIVLLMV VTSNADRICT GITSSNSPHV VKTACQGEVN VTGVIPLTTT PTKSHFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIAGFLE GGWEGMIAGW HGYTSHGAHG VAVAADLKST QEAINCITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI CL
>pBV-083| E63K, H137R, D144K, E195K
(SEQ ID NO: 092)
MKAIIVLLMV VTSNADRICT GITSSNSPHV VKTATQGEVN VTGVIPLTTT PTKSHFANLK
GTKTRGKLCP KCLNCTDLDV ALGRPKCTGK IPSARVSILH EVRPVTSGCF PIMHDRTKIR
QLPNLLRGYE HVRLSTRNVI NTEKAPGGPY EIGTSGSCLN ITNGKGFFAT MAWAVPKNKT
ATNPLTIEVP YICTKEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR PPAKLLKERG
FFGAIAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI CL
>pBv-084| H55C-I302C
(SEQ ID NO: 093)
MKAIIVLLMV VTSNADRICT GITSSNSPHV VKTATQGEVN VTGVIPLTTT PTKSCFANLK
GTETRGKLCP KCLNCTDLDV ALGRPKCTGK IPSARVSILH EVRPVTSGCF PIMHDRTKIR
QLPNLLRGYE HVRLSTHNVI NTEDAPGGPY EIGTSGSCLN ITNGKGFFAT MAWAVPKNKT
ATNPLTIEVP YICTEEEDQI TVWGFHSDDE TQMARLYGDS KPQKFTSSAN GVTTHYVSQI
GGFPNQTEDG GLPQSGRIVV DYMVQKSGKT GTITYQRGIL LPQKVWCASG KSKVIKGSLP
LCGEADCLHE KYGGLNKSKP YYTGEHAKAI GNCPIWVKTP LKLANGTKYR PPAKLLKERG
FFGAIAGFLE GGWEGMIAGW HGYTSHGAHG VAVAADLKST QEAINKITKN LNSLSELEVK
NLQRLSGAMD ELHNEILELD EKVDDLRADT ISSQIELAVL LSNEGIINSE DEHLLALERK
LKKMLGPSAV EIGNGCFETK HKCNQTCLDR IAAGTFDAGE FSLPTFDSLN ITAASLNDDG
LDNHTILLYY STAASSLAVT LMIAIFVVYM VSRDNVSCSI CL
>pBv-085| Q423F
(SEQ ID NO: 094)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
V
RLSTHNVINTEDAPGGPYEIGTSGSCLNITNGKGFFATMAWAVPKNKTATNPLTIEVPYICTEEE
D
QITVWGFHSDDETQMARLYGDSKPQKFTSSANGVTTHYVSQIGGFPNQTEDGGLPQSGRIVV
DYMV
QKSGKTGTITYQRGILLPQKVWCASGKSKVIKGSLPLIGEADCLHEKYGGLNKSKPYYTGEHAK
AI
GNCPIWVKTPLKLANGTKYRPPAKLLKERGFFGAIAGFLEGGWEGMIAGWHGYTSHGAHGVA
VAAD
LKSTQEAINKITKNLNSLSELEVKNLFRLSGAMDELHNEILELDEKVDDLRADTISSQIELAVLLS
NEGIINSEDEHLLALERKLKKMLGPSAVEIGNGCFETKHKCNQTCLDRIAAGTFDAGEFSLPTFD
S
LNITAASLNDDGLDNHTILLYYSTAASSLAVTLMIAIFVVYMVSRDNVSCSICL
>pBv-086| N434P, E435P
(SEQ ID NO: 095)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDELHPPILELDEKVDDLRADT
ISSQIELAVLLSNEGIINSEDEHLLALERKLKKMLGPSAVEIGNGCFETKHKCNQTCLDRIAAGTF
DAGEFSLPTFDSLNITAASLNDDGLDNHTILLYYSTAASSLAVTLMIAIFVVYMVSRDNVSCSICL
>pBv-087| K420C-D449C, K480Y
(SEQ ID NO: 096)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVCNLQRLSGAMDELHNEILELDEKVDDLRAC
TISSQIELAVLLSNEGIINSEDEHLLALERYLKKMLGPSAVEIGNGCFETKHKCNQTCLDRIAAGT
FDAGEFSLPTFDSLNITAASLNDDGLDNHTILLYYSTAASSLAVTLMIAIFVVYMVSRDNVSCSIC
L
>pBv-088| N434P, E435P, K420C-D449C, K480Y
(SEQ ID NO: 097)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVCNLQRLSGAMDELHPPILELDEKVDDLRACT
ISSQIELAVLLSNEGIINSEDEHLLALERYLKKMLGPSAVEIGNGCFETKHKCNQTCLDRIAAGTF
DAGEFSLPTFDSLNITAASLNDDGLDNHTILLYYSTAASSLAVTLMIAIFVVYMVSRDNVSCSICL
>pBv-089| I451W, S453Y, K480Y
(SEQ ID NO: 098)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDELHNEILELDEKVDDLRADT
WSYQIELAVLLSNEGIINSEDEHLLALERYLKKMLGPSAVEIGNGCFETKHKCNQTCLDRIAAGT
FDAGEFSLPTFDSLNITAASLNDDGLDNHTILLYYSTAASSLAVTLMIAIFVVYMVSRDNVSCSIC
L
>pBv-090| H235I, N434P, E435P, H473D, H501F
(SEQ ID: 099)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTIYVSQIGGFPNQTEDGGLPQSGRIV
VDYMVQKSGKTGTITYQRGILLPQKVWCASGKSKVIKGSLPLIGEADCLHEKYGGLNKSKPYYT
GEHAKAIGNCPIWVKTPLKLANGTKYRPPAKLLKERGFFGAIAGFLEGGWEGMIAGWHGYTSH
GAHGVAVAADLKSTQEAINKITKNLNSLSELEVKNLQRLSGAMDELHPPILELDEKVDDLRADTI
SSQIELAVLLSNEGIINSEDEDLLALERKLKKMLGPSAVEIGNGCFETKFKCNQTCLDRIAAGTFD
AGEFSLPTFDSLNITAASLNDDGLDNHTILLYYSTAASSLAVTLMIAIFVVYMVSRDNVSCSICL
>pBv-091| H235I, N434P, E435P, K420C-D449C
(SEQ ID NO: 100)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTIYVSQIGGFPNQTEDGGLPQSGRIV
VDYMVQKSGKTGTITYQRGILLPQKVWCASGKSKVIKGSLPLIGEADCLHEKYGGLNKSKPYYT
GEHAKAIGNCPIWVKTPLKLANGTKYRPPAKLLKERGFFGAIAGFLEGGWEGMIAGWHGYTSH
GAHGVAVAADLKSTQEAINKITKNLNSLSELEVCNLQRLSGAMDELHPPILELDEKVDDLRACTI
SSQIELAVLLSNEGIINSEDEHLLALERKLKKMLGPSAVEIGNGCFETKHKCNQTCLDRIAAGTFD
AGEFSLPTFDSLNITAASLNDDGLDNHTILLYYSTAASSLAVTLMIAIFVVYMVSRDNVSCSICL
>pBv-092| H235I, K420C-D449C, A458I, H501F
(SEQ ID NO: 101)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTIYVSQIGGFPNQTEDGGLPQSGRIV
VDYMVQKSGKTGTITYQRGILLPQKVWCASGKSKVIKGSLPLIGEADCLHEKYGGLNKSKPYYT
GEHAKAIGNCPIWVKTPLKLANGTKYRPPAKLLKERGFFGAIAGFLEGGWEGMIAGWHGYTSH
GAHGVAVAADLKSTQEAINKITKNLNSLSELEVCNLQRLSGAMDELHNEILELDEKVDDLRACTI
SSQIELIVLLSNEGIINSEDEHLLALERKLKKMLGPSAVEIGNGCFETKFKCNQTCLDRIAAGTFD
AGEFSLPTFDSLNITAASLNDDGLDNHTILLYYSTAASSLAVTLMIAIFVVYMVSRDNVSCSICL
>pBV-093| K224Q, N434P, E435P, A458I, H501F
(SEQ ID NO: 102)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQQFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDELHPPILELDEKVDDLRADT
ISSQIELIVLLSNEGIINSEDEHLLALERKLKKMLGPSAVEIGNGCFETKFKCNQTCLDRIAAGTFD
AGEFSLPTFDSLNITAASLNDDGLDNHTILLYYSTAASSLAVTLMIAIFVVYMVSRDNVSCSICL
>pBv-094| H235I, I451W, S453Y, H473D, H501F
(SEQ ID NO: 103)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTIYVSQIGGFPNQTEDGGLPQSGRIV
VDYMVQKSGKTGTITYQRGILLPQKVWCASGKSKVIKGSLPLIGEADCLHEKYGGLNKSKPYYT
GEHAKAIGNCPIWVKTPLKLANGTKYRPPAKLLKERGFFGAIAGFLEGGWEGMIAGWHGYTSH
GAHGVAVAADLKSTQEAINKITKNLNSLSELEVKNLQRLSGAMDELHNEILELDEKVDDLRADT
WSYQIELAVLLSNEGIINSEDEDLLALERKLKKMLGPSAVEIGNGCFETKFKCNQTCLDRIAAGT
FDAGEFSLPTFDSLNITAASLNDDGLDNHTILLYYSTAASSLAVTLMIAIFVVYMVSRDNVSCSIC
L
>pBv-095| H381C-S399C, N434P, E435P, H473D, H501F, K480Y
(SEQ ID NO: 104)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWCGYTS
HGAHGVAVAADLKCTQEAINKITKNLNSLSELEVKNLQRLSGAMDELHPPILELDEKVDDLRADT
ISSQIELAVLLSNEGIINSEDEDLLALERYLKKMLGPSAVEIGNGCFETKFKCNQTCLDRIAAGTF
DAGEFSLPTFDSLNITAASLNDDGLDNHTILLYYSTAASSLAVTLMIAIFVVYMVSRDNVSCSICL
>pBv-096| P487E, P524E, L529R
(SEQ ID NO: 105)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDELHNEILELDEKVDDLRADT
ISSQIELAVLLSNEGIINSEDEHLLALERKLKKMLGESAVEIGNGCFETKHKCNQTCLDRIAAGTF
DAGEFSLETFDSRNITAASLNDDGLDNHTILLYYSTAASSLAVTLMIAIFVVYMVSRDNVSCSICL
>pBv-097| P524D
(SEQ ID NO: 106)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDELHNEILELDEKVDDLRADT
ISSQIELAVLLSNEGIINSEDEHLLALERKLKKMLGPSAVEIGNGCFETKHKCNQTCLDRIAAGTF
DAGEFSLDTFDSLNITAASLNDDGLDNHTILLYYSTAASSLAVTLMIAIFVVYMVSRDNVSCSICL
>pBv-098| Q121C-M429C
(SEQ ID NO: 107)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRCLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGACDELHNEILELDEKVDDLRADT
ISSQIELAVLLSNEGIINSEDEHLLALERKLKKMLGPSAVEIGNGCFETKHKCNQTCLDRIAAGTF
DAGEFSLPTFDSLNITAASLNDDGLDNHTILLYYSTAASSLAVTLMIAIFVVYMVSRDNVSCSICL
>pBv-099| K118C-L432C
(SEQ ID NO: 108)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTCIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDECHNEILELDEKVDDLRAD
TISSQIELAVLLSNEGIINSEDEHLLALERKLKKMLGPSAVEIGNGCFETKHKCNQTCLDRIAAGT
FDAGEFSLPTFDSLNITAASLNDDGLDNHTILLYYSTAASSLAVTLMIAIFVVYMVSRDNVSCSIC
L
>pBv-100| N230C-E431C
(SEQ ID NO: 109)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSACGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDCLHNEILELDEKVDDLRAD
TISSQIELAVLLSNEGIINSEDEHLLALERKLKKMLGPSAVEIGNGCFETKHKCNQTCLDRIAAGT
FDAGEFSLPTFDSLNITAASLNDDGLDNHTILLYYSTAASSLAVTLMIAIFVVYMVSRDNVSCSIC
L
>pBv-101| T233D
(SEQ ID NO: 110)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVDTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDELHNEILELDEKVDDLRADT
ISSQIELAVLLSNEGIINSEDEHLLALERKLKKMLGPSAVEIGNGCFETKHKCNQTCLDRIAAGTF
DAGEFSLPTFDSLNITAASLNDDGLDNHTILLYYSTAASSLAVTLMIAIFVVYMVSRDNVSCSICL
>pBv-102| I335K, p.338K_339TinsQ, T339N, P340T, del.T347, K480Y
(SEQ ID NO: 111)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPKWVKQNTLKLANGKYRPPAKLLKERGFFGAIAGFLEGGWEGMIAGWHGYT
SHGAHGVAVAADLKSTQEAINKITKNLNSLSELEVKNLQRLSGAMDELHNEILELDEKVDDLRA
DTISSQIELAVLLSNEGIINSEDEHLLALERYLKKMLGPSAVEIGNGCFETKHKCNQTCLDRIAAG
TFDAGEFSLPTFDSLNITAASLNDDGLDNHTILLYYSTAASSLAVTLMIAIFVVYMVSRDNVSCSI
CL
>pBv-103| I22C-G372C
(SEQ ID NO: 112)
MKAIIVLLMVVTSNADRICTGCTSSNSPHVVKTATQGEVNVTGVIPLTTTPTKSHFANLKGTETR
GKLCPKCLNCTDLDVALGRPKCTGKIPSARVSILHEVRPVTSGCFPIMHDRTKIRQLPNLLRGYE
HVRLSTHNVINTEDAPGGPYEIGTSGSCLNITNGKGFFATMAWAVPKNKTATNPLTIEVPYICTE
EEDQITVWGFHSDDETQMARLYGDSKPQKFTSSANGVTTHYVSQIGGFPNQTEDGGLPQSGR
IVVDYMVQKSGKTGTITYQRGILLPQKVWCASGKSKVIKGSLPLIGEADCLHEKYGGLNKSKPY
YTGEHAKAIGNCPIWVKTPLKLANGTKYRPPAKLLKERGFFGAIAGFLEGCWEGMIAGWHGYT
SHGAHGVAVAADLKSTQEAINKITKNLNSLSELEVKNLQRLSGAMDELHNEILELDEKVDDLRA
DTISSQIELAVLLSNEGIINSEDEHLLALERKLKKMLGPSAVEIGNGCFETKHKCNQTCLDRIAAG
TFDAGEFSLPTFDSLNITAASLNDDGLDNHTILLYYSTAASSLAVTLMIAIFVVYMVSRDNVSCSI
CL
>pBv-104| S556C, T560C, V571C, N575C
(SEQ ID NO: 113)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDELHNEILELDEKVDDLRADT
ISSQIELAVLLSNEGIINSEDEHLLALERKLKKMLGPSAVEIGNGCFETKHKCNQTCLDRIAAGTF
DAGEFSLPTFDSLNITAASLNDDGLDNHTILLYYSTAASCLAVCLMIAIFVVYMCSRDCVSCSICL
>pBv-105| S556V, T560V, V571C
(SEQ ID NO: 114)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDELHNEILELDEKVDDLRADT
ISSQIELAVLLSNEGIINSEDEHLLALERKLKKMLGPSAVEIGNGCFETKHKCNQTCLDRIAAGTF
DAGEFSLPTFDSLNITAASLNDDGLDNHTILLYYSTAASVLAVCLMIAIFVVYMCSRDNVSCSICL
>pBv-106| S556C, T560C, V571C
(SEQ ID NO: 115)
MKAIIVLLMVVTSNADRICTGITSSNSPHVVKTATQGEVNVTGVIPLTTTPTKSHFANLKGTETRG
KLCPKCLNCTDLDVALGRPKCTGKIPSARVSILHEVRPVTSGCFPIMHDRTKIRQLPNLLRGYEH
VRLSTHNVINTEDAPGGPYEIGTSGSCLNITNGKGFFATMAWAVPKNKTATNPLTIEVPYICTEE
EDQITVWGFHSDDETQMARLYGDSKPQKFTSSANGVTTHYVSQIGGFPNQTEDGGLPQSGRI
VVDYMVQKSGKTGTITYQRGILLPQKVWCASGKSKVIKGSLPLIGEADCLHEKYGGLNKSKPYY
TGEHAKAIGNCPIWVKTPLKLANGTKYRPPAKLLKERGFFGAIAGFLEGGWEGMIAGWHGYTS
HGAHGVAVAADLKSTQEAINKITKNLNSLSELEVKNLQRLSGAMDELHNEILELDEKVDDLRADT
ISSQIELAVLLSNEGIINSEDEHLLALERKLKKMLGPSAVEIGNGCFETKHKCNQTCLDRIAAGTF
DAGEFSLPTFDSLNITAASLNDDGLDNHTILLYYSTAASCLAVCLMIAIFVVYMCSRDNVSCSICL

The immunogenic composition including a lipid-based delivery system may further include one or more salts and/or one or more pharmaceutically acceptable surfactants, preservatives, carriers, diluents, and/or excipients, in some cases. In some aspects, the immunogenic composition including a lipid-based delivery system further include a pharmaceutically acceptable vehicle. In some aspects, each of a buffer, stabilizing agent, and optionally a salt, may be included in the immunogenic composition including a lipid-based delivery system. In other aspects, any one or more of a buffer, stabilizing agent, salt, surfactant, preservative, and excipient may be excluded from the immunogenic composition including a lipid-based delivery system.

In a further aspect, the immunogenic composition including a lipid-based delivery system further comprises a stabilizing agent. In some aspects, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In some aspects, the stabilizing agent is a disaccharide, or sugar. In one aspect, the stabilizing agent is sucrose. In another aspect, the stabilizing agent is trehalose. In a further aspect, the stabilizing agent is a combination of sucrose and trehalose. In some aspects, the total concentration of the stabilizing agent(s) in the composition is about 5% to about 10% w/v. For example, the total concentration of the stabilizing agent may be equal to at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, or 20% w/v or any range or value derivable therein. In some aspects, the stabilizing agent concentration includes, but is not limited to, a concentration of about 10 mg/mL to about 400 mg/mL, about 100 mg/mL to about 200 mg/mL, about 100 mg/mL to about 150 mg/mL, about 100 mg/mL to about 140 mg/mL, about 100 mg/mL to about 130 mg/mL, about 100 mg/mL to about 120 mg/mL, about 100 mg/mL to about 110 mg/mL, or about 100 mg/mL to about 105 mg/mL. In some aspects, the concentration of the stabilizing agent is equal to at least, at most, exactly, or between any two of 10 mg/mL, 20 mg/mL, 50 mg/mL, 100 mg/mL, 101 mg/mL, 102 mg/mL, 103 mg/mL, 104 mg/mL, 105 mg/mL, 106 mg/mL, 107 mg/mL, 108 mg/mL, 109 mg/mL, 110 mg/mL, 150 mg/mL, 200 mg/mL, 300 mg/mL, 400 mg/mL, or more.

In a further aspect, the mass amount of the stabilizing agent and the mass amount of the RNA are in a specific ratio. In one aspect, the ratio of the mass amount of the stabilizing agent and the RNA is no greater than 5000. In another aspect, the ratio of the mass amount of the stabilizing agent and the RNA is no greater than 2000. In another aspect, the ratio of the mass amount of the stabilizing agent and the RNA is no greater than 1000. In another aspect, the ratio of the mass amount of the stabilizing agent and the RNA is no greater than 500. In another aspect, the ratio of the mass amount of the stabilizing agent and the RNA is no greater than 100. In another aspect, the ratio of the mass amount of the stabilizing agent and the pharmaceutical substance is no greater than 50. In another aspect, the ratio of the mass amount of the stabilizing agent and the RNA is no greater than 10. In another aspect, the ratio of the mass amount of the stabilizing agent and the RNA is no greater than 1. In another aspect, the ratio of the mass amount of the stabilizing agent and the RNA is no greater than 0.5. In another aspect, the ratio of the mass amount of the stabilizing agent and the RNA is no greater than 0.1. In another aspect, the stabilizing agent and RNA comprise a mass ratio of about 200-2000 of the stabilizing agent: 1 of the RNA.

In some aspects, the immunogenic composition including a lipid-based delivery system further comprises a buffer. Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, the buffer is a HEPES buffer, a Tris buffer, or a PBS buffer. Accordingly, in some embodiments, the composition further includes an adjuvant, e.g., aluminum-containing compounds, such as, for example, any of the adjuvants listed herein, including aluminum hydroxide and AIPO₄. In one aspect, the buffer is Tris buffer. In another aspect, the buffer is a HEPES buffer. In a further aspect, the buffer is a PBS buffer. For example, the buffer concentration may be equal to at least, at most, exactly, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. The buffer may be at a neutral pH, pH 6.5 to 8.5, pH 7.0 to pH 8.0, or pH 7.2 to pH 7.6. For example, the buffer may be at least, at most, exactly, or between any two of pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

In some aspects, the immunogenic composition including a lipid-based delivery system may further comprise a salt. Examples of salts include but not limited to sodium salts and/or potassium salts. In one aspect, the salt is a sodium salt. In a specific aspect, the sodium salt is sodium chloride. In one aspect, the salt is a potassium salt. In some aspects, the potassium salt comprises potassium chloride. The concentration of the salts in the composition may be about 70 mM to about 140 mM. For example, the salt concentration may be equal to at least, at most, exactly, or between any two of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM.

In some aspects, the salt concentration includes, but is not limited to, a concentration of about 1 mg/mL to about 100 mg/mL, about 1 mg/mL to about 50 mg/mL, about 1 mg/mL to about 40 mg/mL, about 1 mg/mL to about 30 mg/mL, about 1 mg/mL to about 20 mg/mL, about 1 mg/mL to about 10 mg/mL, or about 1 mg/mL to about 15 mg/mL. In some aspects, the concentration of the salt is equal to at least, at most, exactly, or between any two of 1 mg/mL, 2 mg/ml, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, or more. The salt may be at a neutral pH, pH 6.5 to 8.5, pH 7.0 to pH 8.0, or pH 7.2 to pH 7.6. For example, the salt may be at a pH equal to at least, at most, exactly, or between any two of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

In some aspects, the immunogenic composition including a lipid-based delivery system further comprises a surfactant, a preservative, any other excipient, or a combination thereof. As used herein, “any other excipient” includes, but is not limited to, antioxidants, glutathione, EDTA, methionine, desferal, antioxidants, metal scavengers, or free radical scavengers. In one aspect, the surfactant, preservative, excipient or combination thereof is sterile water for injection (sWFI), bacteriostatic water for injection (BWFI), saline, dextrose solution, polysorbates, poloxamers, Triton, divalent cations, Ringer's lactate, amino acids, sugars, polyols, polymers, or cyclodextrins. In some embodiments, the formulations that comprise LNPs described herein excludes Triton X-100.

Examples of excipients, which refer to ingredients in the immunogenic compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, or colorants. Preservatives for use in the compositions disclosed herein include but are not limited to benzalkonium chloride, chlorobutanol, paraben and thimerosal. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, antioxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato; and celluloses such as microcrystalline cellulose. The amount of diluent in the composition may range from about 10% to about 90% by weight of the total composition, about 25% to about 75%, about 30% to about 60% by weight, or about 12% to about 60%.

In some embodiments, the formulation further includes a buffering agent, e.g., a weak base such as a lipid-soluble carboxylic acid salt. In some embodiments, the lipid-soluble carboxylic acid salt is at least one selected from the group consisting of a sodium, potassium, magnesium, and/or calcium salt of caprylic acid, capric acid, lauric acid, stearic acid, myristoleic acid, linoleic acid, linolenic acid, arachidonic acid, eicosenoic acid, palmitoleic acid, sapienic acid, oleic acid, elaidic acid, and/or vaccenic acid. In some preferred embodiments, the formulation includes sodium oleate. In some embodiments, the lipid-soluble carboxylic acid salt (e.g., sodium oleate) content is about 1.1 molar equivalents to about 3 molar equivalents with respect to the compound of formula II or the pharmaceutically acceptable salt thereof. In some preferred embodiments, the composition comprises sodium oleate.

The pH and exact concentration of the various components in the immunogenic composition including a lipid-based delivery system are adjusted according to well-known parameters. The use of such media and agents for pharmaceutical active substances is well known in the art. Except insofar as any conventional media or agent is incompatible with the active ingredients, its use in immunogenic, prophylactic and/or therapeutic compositions is contemplated.

It is contemplated that any embodiment discussed in this specification may be implemented with respect to any method or composition of the disclosure, and vice versa. Furthermore, compositions of the disclosure may be used to achieve methods of the disclosure. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive “or.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The phrase “essentially all” is defined as “at least 95%”; if essentially all members of a group have a certain property, then at least 95% of members of the group have that property. In some instances, essentially all means equal to any one of, at least any one of, or between any two of 95, 96, 97, 98, 99, or 100% of members of the group have that property.

The compositions and methods for their use may “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure.

### EXAMPLES

### Example 1: Drug Product Composition

The drug product composition is an influenza modRNA drug substance targeting the Wisconsin 2021/2022 hemagglutinin.

TABLE 1
Formulation composition of the ready-to-use (RTU)
presentation of Flu vaccine drug product

| Components | Function | Concentration, mg/mL |
|---|---|---|
| PF-07829855 Drug substance (mRNA) | Active | 0.1 |
| ALC-0315 | Functional lipid | 1.43 |
| ALC-0159 | Functional lipid | 0.18 |
| DSPC | Structural lipid | 0.31 |
| Cholesterol | Structural lipid | 0.62 |
| Sucrose | Cryoprotectant/Tonicifier | 1.3 |
| Tris/Tromethamine | Buffer, pH 7.4 | 0.18 |
| Tris HCl | | 1.34 |
| Water for injection | Solvent/Vehicle | q.s. |

In some embodiments, the immunogenic composition comprising one lipid nanoparticle encapsulated mRNA molecule encoding HA is monovalent and has a dose selected from any one of 1 μg mRNA, 2 μg RNA, 5 μg RNA, and 20 μg RNA.

In some embodiments, the immunogenic composition comprising one lipid nanoparticle encapsulated mRNA molecule encoding HA, a second lipid nanoparticle encapsulated mRNA molecule encoding HA, a third lipid nanoparticle encapsulated mRNA molecule encoding NA, and a fourth lipid nanoparticle encapsulated mRNA molecule encoding NA, wherein the total dose is up to 20 μg RNA.

In some embodiments, the subject is aged 30-50 years.

### Example 2: Shipping and Container Closure Information

The Drug Product is shipped frozen on dry ice. The primary container closure is a 2 mL glass Type 1 vial with 13 mm stopper. The drug product should be stored at −60 to −90° C.

### Example3: Dosage Forms

The PF-07252220 influenza modRNA immunogenic composition candidates include one of 3 different dosage forms, selected from 2 monovalent forms and one quadrivalent form, each of which incorporate different constructs of mRNA.

Four Constructs of modRNA:
Wisconsin modRNA (Wisc2019 HA).
Phuket modRNA (Phuk2013 HA).
Washington modRNA (Wash2019 HA).
Cambodia modRNA (Camb2020 HA)

Accordingly, there are 2 monovalent immunogenic compositions (also referred to herein as drug products (DPs)) and one quadrivalent immunogenic composition.
1. Monovalent including Wisconsin modRNA
2. Monovalent including Phuket modRNA
3. Quadrivalent, which includes Wisconsin modRNA, Phuket modRNA, Washington modRNA, and Cambodia modRNA

The immunogenic composition is supplied in a 2 mL glass vial sealed with a chlorobutyl rubber stopper and an aluminum seal with flip-off plastic cap (nominal volume of 0.3 mL).

4.2. Components of the Immunogenic Composition

The immunogenic composition includes modRNA encoding a strain-specific full length, codon-optimized HA envelope glycoprotein which is responsible for viral binding to target cells and mediating cell entry.

The immunogenic composition is a preservative-free, sterile dispersion of LNPs in aqueous cryoprotectant buffer for IM administration. The immunogenic composition is formulated at 0.1 mg/mL RNA in 10 mM Tris buffer, 300 mM sucrose, pH 7.4 as a single-dose vial with 0.5 mL/vial fill volume, and 0.3 mL nominal volume.

4.2.1. Drug Substance

The specific constructs (i.e., Wisconsin modRNA [Wisc2019 HA] and Phuket modRNA [Phuk2013 HA]) or constructs (quadrivalent: Wisconsin modRNA, Phuket modRNA, Washington modRNA, and Cambodia modRNA, in the drug substance (modRNA) are the only active ingredient(s) in the DP. The drug substance is formulated in 10 mM HEPES buffer, 0.1 mM

EDTA at pH 7.0 and stored at 20±5° C. in HDPE bottles EVA flexible containers. In addition to the codon-optimized sequence encoding the antigen, the RNA contains common structural elements optimized for mediating high RNA stability and translational efficiency (5′-cap, 5′UTR, 3′-UTR, poly(A)-tail; see table and sequences below). Furthermore, an intrinsic signal peptide (sec) is part of the open reading frame and is translated as an N-terminal peptide.

The RNA does not contain any uridines; instead of uridine the modified N1-methylpseudouridine is used in RNA synthesis.

The specific constructs each comprise the following elements: 5′-cap analog (m27,3′-OGppp (m12′-0) ApG) for production of RNA containing a cap1 structure is shown below

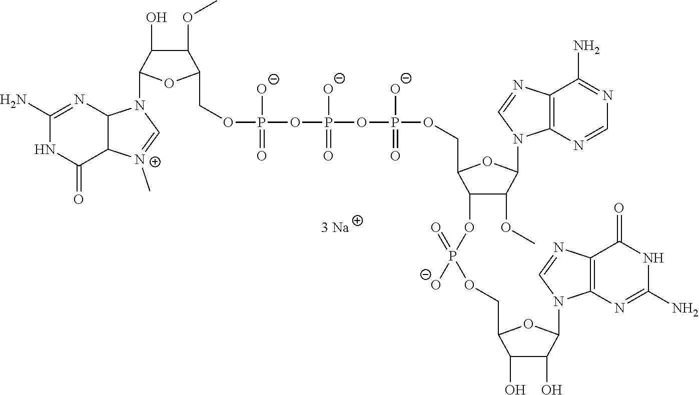 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(N2C=[N+](C)C3C(=O)NC(N)=NC32)C1O]

The cap1 structure (i.e., containing a 2′-O-methyl group on the penultimate nucleoside of the 5′-end of the RNA chain) is incorporated into the drug substance by using a respective cap analog during in vitro transcription. For RNAs with modified uridine nucleotides, the cap1 structure is superior to other cap structures, since cap1 is not recognized by cellular factors such as IFIT1 and, thus, cap1-dependent translation is not inhibited by competition with eukaryotic translation initiation factor 4E. In the context of IFIT1 expression, mRNAs with a cap1 structure give higher protein expression levels.

TABLE 2
Table of elements

| Element | Description | Position |
|---|---|---|
| cap | A modified 5′-cap1 structure (m⁷G⁺m^{3′}-5′-ppp-5′-Am) | 1-2 |
| 5′-UTR | 5′-untranslated region derived from human alpha-globin | 3-54 |
| | RNA with an optimized Kozak sequence | |
| 3′-UTR | The 3′ untranslated region comprises two sequence | 3880-4174 |
| | elements derived from the amino-terminal enhancer of | |
| | split (AES) mRNA and the mitochondrial encoded 12S | |
| | ribosomal RNA to confer RNA stability and high total | |
| | protein expression. | |
| poly(A) | A 110-nucleotide poly(A)-tail consisting of a stretch of 30 | 4175-4284 |
| | adenosine residues, followed by a 10-nucleotide linker | |
| | sequence and another 70 adenosine residues. | |

Sequence
GA

| | |
|---|---|
| GAAΨAAAC ΨAGΨAΨΨCΨΨ CΨGGΨCCCCA CAGACΨCAGA GAGAACCCGC | 50 |
| CACC (SEQ ID NO: 1) | 54 |
| C ΨCGAGCΨGGΨ ACΨGCAΨGCA | 3900 |
| CGCAAΨGCΨA GCΨGCCCCΨΨ ΨCCCGΨCCΨG GGΨACCCCGA GΨCΨCCCCCG | 3950 |
| ACCΨCGGGΨC CCAGGΨAΨGC ΨCCCACCΨCC ACCΨGCCCCA CΨCACCACCΨ | 4000 |
| CΨGCΨAGΨΨC CAGACACCΨC CCAAGCACGC AGCAAΨGCAG CΨCAAAACGC | 4050 |
| ΨΨAGCCΨGC CACACCCCCA CGGGAAACAG CAGΨGAΨΨAA CCΨΨΨAGCAA | 4100 |
| ΨAAACGAAAG ΨΨΨAACΨAAG CΨAΨACΨAAC CCCAGGGΨΨG GΨCAAΨΨΨCG | 4150 |
| ΨGCCAGCCAC ACCCΨGGAGC ΨAGC (SEQ ID NO: 2) | |
| AAAAAA AAAAAAAAAA AAAAAAAAAA | 4200 |
| AAAAGCAΨAΨ GACΨAAAAAA AAAAAAAAAA AAAAAAAAAA AAAAAAAAAA | 4250 |
| AAAAAAAAAA AAAAAAAAAA AAAAAAAAAA AAAA (SEQ ID NO: 3) | 4284 |

Ψ = 1-methyl-3′-pseudouridylyl

The manufacturing process comprises RNA synthesis via in vitro transcription (IVT) step followed by DNase I and proteinase K digestion steps, purification by ultrafiltration/diafiltration (UFDF), and final filtration and dispense. A platform approach to the IVT, digestion, and purification process steps was used in the production of the four modRNA drug substances. The mRNA drug substance clinical batches were prepared at a scale of 37.6 L starting volume for IVT. The primary objective of the DNase I digestion step is to reduce the size of linear DNA template to enable subsequent removal across the ultrafiltration/diafiltration step. A DNase I solution are added at the end of the final IVT incubation. Temperature and agitation rate from IVT step are maintained during this step. The primary objective of the proteinase K digestion step is to reduce the size of proteins in the reaction mixture for subsequent removal across the ultrafiltration/diafiltration step. Proteinase K solution is added to the reaction vessel and incubated for a predetermined amount of time. Temperature and agitation rate implemented during IVT and DNase digestion steps are maintained during this step. All the material is purified by a single 2-stage Ultrafiltration (UF) and diafiltration (DF) (UFDF) to produce the RNA drug substance. Flat sheet cassette membranes were used as part of the UFDF process. Preferably, the UFDF process does not utilize hollow fiber membranes. The UFDF step removes small process-related impurities and concentrates, and buffer exchanges the RNA into the final DS formulation.

Based on the retentate RNA concentration determined after diafiltration 2, the diafiltered retentate is then concentrated, if needed, and recovered through a dual-layer filter into a flexible container. The UFDF system is subsequently rinsed and added to the retentate pool through the same dual-layer filter. Formulation buffer may be added. The final pool is then filtered through a second dual-layer filter into HDPE bottle(s).

TABLE 3
Batch Results for Influenza modRNA Vaccine Wisconsin Drug Substance

| Quality | Analytical | Acceptance | Developmental | Developmental | Clinical Drug |
|---|---|---|---|---|---|
| Attribute | Procedure | Criteria | Material | Material | Substance |
| Appearance | Clarity | ≤6 NTU | NT | ≤3 NTU | ≤1 NTU |
| (Clarity) | | | | | |
| Appearance | Coloration | Not more | NT | ≤B9 | ≤B9 |
| (Coloration) | | intensely | | | |
| | | colored than | | | |
| | | level 7 of the | | | |
| | | brown (B) color | | | |
| | | standard. | | | |
| pH | Potentiometry | 7.0 ± 0.5 | NT | 6.8 | 6.8 |
| Content | UV | 2.25 ± 0.25 | 2.40 mg/mL | 2.28 mg/mL | 2.17 mg/mL |
| (RNA | spectroscopy | mg/mL | | | |
| concentration) | | | | | |
| RT- | Identity of | Identity | NT | Identity | Confirmed |
| PCR | Encoded | confirmed | | confirmed | |
| | RNA | | | | |
| | Sequence | | | | |
| RNA | Capillary | ≥60% | 87% | 86% | 88% |
| integrity | gel | | | | |
| | electrophoresis | | | | |
| RP- | 5′-Cap | Report Results | 82% | 82% | 83% |
| HPLC | | | | | |
| Residual | qPCR | ≤500 ng | NT | 23 ng/mg | 112 ng |
| DNA | | DNA/mg RNA | | | DNA/mg RNA |
| template | | | | | |
| Endotoxin | Endotoxin | ≤12.5 EU/mL | NT | 0.35 EU/mL | NMT 1.00 |
| | (LAL) | | | | EU/mL |
| Bioburden | Bioburden | ≤1 CFU/10 mL | NT | 0 CFU/10 mL | 0 CFU/10mL |

Specifications only apply to clinical supplies
Abbreviations: NTU = nephelometric turbidity units;
NT = not tested;
ddPCR = digital droplet polymerase chain reaction;
RP-HPLC = reversed phased high performance liquid chromatography;
qPCR = quantitative polymerase chain reaction;
LAL = limulus amebocyte lysate;
EU = Endotoxin unit;
CFU = Colony forming unit

TABLE 4
Batch Analyses for Wisconsin Clinical Drug Product

| | | | |
|---|---|---|---|
| | | Acceptance | |
| Analytical Procedure | Quality Attributes | Criteria | Batch Results |

Composition and Strength

| Appearance (Visual) | Appearance | White to off-white | | White to off-white | |
|---|---|---|---|---|---|
| | | suspension | | suspension | |
| Appearance | Appearance (Visible | May contain white to | | Meets | |
| (Particles) | Particulates) | off-white opaque, | | | |
| | | amorphous particles | | | |
| Subvisible particulate | Subvisible particles | Particles ≥10 μm: ≤6000 | | 21 | |
| matter | | per container | | Particles/container | |
| | | Particles ≥25 μm: ≤600 | | 1 Particles/container | |
| | | per container | | | |
| Potentiometry | pH | 7.4 ± 0.5 | | 7.4 | |
| Osmometry | Osmolality | 240-400 | mOsmol/kg | 364 | mOsm/kg |
| Dynamic Light | LNP Size | 40-120 | nm | 67 | nm |
| Scattering (DLS) | LNP Polydispersity | ≤0.3 | | 0.2 | |
| Fluorescence Assay | RNA Content | 0.074-0.126 | mg/mL | 0.104 | mg/mL |
| | RNA Encapsulation | ≥80% | | 97% | |
| HPLC-CAD | ALC-0315 Content | 0.90-1.85 | mg/mL | 1.39 | mg/mL |
| | ALC-0159 Content | 0.11-0.24 | mg/mL | 0.18 | mg/mL |
| | DSPC Content | 0.18-0.41 | mg/mL | 0.32 | mg/mL |
| | Cholesterol Content | 0.36-0.78 | mg/mL | 0.61 | mg/mL |

| | | | |
|---|---|---|---|
| Container content | Vial content (volume) | Not less than 0.30 | Not less than labeled |
| | | mL | volume |

Identity

| HPLC-CAD | Lipid identities | Retention times | Retention times |
|---|---|---|---|
| | | consistent with | consistent with |
| | | references (ALC- | references (ALC- |
| | | 0315, ALC-0159, | 0315, ALC-0159, |
| | | Cholesterol, DSPC) | Cholesterol, DSPC) |
| RT-PCR | Identity of encoded | Identity confirmed | Confirmed |
| | RNA sequence(s) | | |

Purity

| Capillary Gel | RNA Integrity | ≥55% intact RNA | 87% |
|---|---|---|---|
| Electrophoresis | | (release) | |
| | | ≥50% intact RNA | |
| | | (stability) | |

Safety

| | | | | |
|---|---|---|---|---|
| Endotoxin (LAL) | Endotoxin (LAL) | ≤12.5 | EU/mL | NMT 5.0 EU/mL |

| | | | |
|---|---|---|---|
| Sterility | Sterility | No growth detected | No growth detected |

Specifications only apply to clinical supplies
Abbreviations: NTU = nephelometric turbidity units;
NT = not tested;
ddPCR = digital droplet polymerase chain reaction;
RP-HPLC = reversed phased high performance liquid chromatography;
qPCR = quantitative polymerase chain reaction;
LAL = limulus amebocyte lysate;
EU = Endotoxin unit;
CFU = Colony forming unit

TABLE 5
Batch Results for Influenza modRNA Vaccine Phuket Drug Substance

| Quality | Analytical | Acceptance | Developmental | Clinical Drug |
|---|---|---|---|---|
| Attribute | Procedure | Criteria | Material | Substance |
| Appearance | Clarity | ≤6 NTU | NT | ≤0 NTU |
| (Clarity) | | | | |
| Appearance | Coloration | Not more | NT | ≤B9 |
| (Coloration) | | intensely colored | | |
| | | than level 7 of the | | |
| | | brown (B) color | | |
| | | standard. | | |
| pH | Potentiometry | 7.0 ± 0.5 | NT | 6.8 |
| Content (RNA | UV | 2.25 ± 0.25 | 2.42 mg/mL | 2.20 mg/mL |
| concentration) | spectroscopy | mg/mL | | |
| RT-PCR | Identity of | Identity confirmed | NT | Confirmed |
| | Encoded RNA | | | |
| | Sequence | | | |
| RNA integrity | Capillary gel | ≥60% | 88% | 87% |
| | electrophoresis | | | |
| RP-HPLC | 5′-Cap | Report Results | 85% | 88% |
| Residual DNA | qPCR | ≤500 ng DNA/mg | NT | 156 ng DNA/mg |
| template | | RNA | | RNA |
| Endotoxin | Endotoxin | ≤12.5 EU/mL | NT | NMT 1.00 EU/mL |
| | (LAL) | | | |
| Bioburden | Bioburden | ≤1 CFU/10 mL | NT | 0 CFU/10 mL |

Specifications only apply to clinical supplies
Abbreviations: NTU = nephelometric turbidity units;
NT = not tested;
ddPCR = digital droplet polymerase chain reaction;
RP-HPLC = reversed phased high performance liquid chromatography;
qPCR = quantitative polymerase chain reaction;
LAL = limulus amebocyte lysate;
EU = Endotoxin unit;
CFU = Colony forming unit

TABLE 6
Batch Analyses for Phuket Clinical Drug Product

| | | | |
|---|---|---|---|
| Analytical | | | |
| Procedure | Quality Attributes | Acceptance Criteria | Batch Results |

Composition and Strength

| Appearance | Appearance | White to off-white | | White to off-white | |
|---|---|---|---|---|---|
| (Visual) | | suspension | | suspension | |
| Appearance | Appearance (Visible | May contain white to | | Meets | |
| (Particles) | Particulates) | off-white opaque, | | | |
| | | amorphous particles | | | |
| Subvisible | Subvisible particles | Particles ≥10 μm: ≤6000 | | 46 Particles/container | |
| particulate matter | | per container | | <1 Particles/container | |
| | | Particles ≥25 μm: ≤600 | | | |
| | | per container | | | |
| Potentiometry | pH | 7.4 ± 0.5 | | 7.3 | |
| Osmometry | Osmolality | 240-400 mOsmol/kg | | 360 mOsm/kg | |
| Dynamic Light | LNP Size | 40-120 | nm | 80 | nm |
| Scattering (DLS) | LNP Polydispersity | ≤0.3 | | 0.2 | |
| Fluorescence | RNA Content | 0.074-0.126 | mg/mL | 0.086 | mg/mL |
| Assay | RNA Encapsulation | ≥80% | | 94% | |
| HPLC-CAD | ALC-0315 Content | 0.90-1.85 | mg/mL | 1.39 | mg/mL |
| | ALC-0159 Content | 0.11-0.24 | mg/mL | 0.17 | mg/mL |
| | DSPC Content | 0.18-0.41 | mg/mL | 0.29 | mg/mL |
| | Cholesterol Content | 0.36-0.78 | mg/mL | 0.59 | mg/mL |

| Container content | Vial content | Not less than 0.30 mL | Not less than labeled |
|---|---|---|---|
| | (volume) | | volume |

Identity

| HPLC-CAD | Lipid identities | Retention times | Retention times |
|---|---|---|---|
| | | consistent with | consistent with |
| | | references (ALC- | references (ALC-0315, |
| | | 0315, ALC-0159, | ALC-0159, Cholesterol, |
| | | Cholesterol, DSPC) | DSPC) |
| RT-PCR | Identity of encoded | Identity confirmed | Confirmed |
| | RNA sequence(s) | | |

Purity

| Capillary Gel | RNA Integrity | ≥55% intact RNA | 85% |
|---|---|---|---|
| Electrophoresis | | (release) | |

Safety

| | | | |
|---|---|---|---|
| Endotoxin (LAL) | Endotoxin (LAL) | ≤12.5 EU/mL | NMT 5.0 EU/mL |
| Sterility | Sterility | No growth detected | No growth detected |

TABLE 7
Batch Results for Influenza modRNA Vaccine Cambodia Drug Substance

| Quality | Analytical | Acceptance | Developmental | Clinical Drug |
|---|---|---|---|---|
| Attribute | Procedure | Criteria | Material | Substance |
| Appearance | Clarity | ≤6 NTU | NT | ≤1 NTU |
| (Clarity) | | | | |
| Appearance | Coloration | Not more | NT | ≤B9 |
| (Coloration) | | intensely colored | | |
| | | than level 7 of the | | |
| | | brown (B) color | | |
| | | standard. | | |
| pH | Potentiometry | 7.0 ± 0.5 | NT | 6.8 |
| Content (RNA | UV | 2.25 ± 0.25 | 2.31 mg/mL | 2.18 mg/mL |
| concentration) | spectroscopy | mg/mL | | |
| ddPCR | Identity of | Identity confirmed | NT | Confirmed |
| | Encoded RNA | | | |
| | Sequence | | | |
| RNA integrity | Capillary gel | ≥60% | 90% | 75% |
| | electrophoresis | | | |
| RP-HPLC | 5′-Cap | Report Results | 80% | 86% |
| Residual DNA | qPCR | ≤1500 ng | NT | 221 ng DNA/mg |
| template | | DNA/mg RNA | | RNA |
| Endotoxin | Endotoxin | ≤12.5 EU/mL | NT | NMT 1.00 EU/mL |
| | (LAL) | | | |
| Bioburden | Bioburden | ≤1 CFU/10 mL | NT | 0 CFU/10 mL |

Specifications only apply to clinical supplies
Abbreviations: NTU = nephelometric turbidity units; NT = not tested; ddPCR = digital droplet polymerase chain reaction; RP-HPLC = reversed phased high performance liquid chromatography; qPCR = quantitative polymerase chain reaction; LAL = limulus amebocyte lysate; EU = Endotoxin unit; CFU = Colony forming unit

TABLE 8
Batch Results for Influenza modRNA Vaccine Washington Drug Substance

| Quality | Analytical | Acceptance | Developmental | Clinical Drug |
|---|---|---|---|---|
| Attribute | Procedure | Criteria | Material | Substance |
| Appearance | Clarity | ≤6 NTU | NT | ≤1 NTU |
| (Clarity) | | | | |
| Appearance | Coloration | Not more | NT | ≤B9 |
| (Coloration) | | intensely colored | | |
| | | than level 7 of the | | |
| | | brown (B) color | | |
| | | standard. | | |
| pH | Potentiometry | 7.0 ± 0.5 | NT | 6.9 |
| Content (RNA | UV | 2.25 ± 0.25 | 2.41 mg/mL | 2.22 mg/mL |
| concentration) | spectroscopy | mg/mL | | |
| RT-PCR | Identity of | Identity confirmed | NT | Confirmed |
| | Encoded RNA | | | |
| | Sequence | | | |
| RNA integrity | Capillary gel | ≥60% | 87% | 83% |
| | electrophoresis | | | |
| RP-HPLC | 5′-Cap | Report Results | 86% | 87% |
| Residual DNA | qPCR | ≤1500 ng | NT | 364 ng DNA/mg |
| template | | DNA/mg RNA | | RNA |
| Endotoxin | Endotoxin | ≤12.5 EU/mL | NT | NMT 1.00 EU/mL |
| | (LAL) | | | |
| Bioburden | Bioburden | ≤1 CFU/10 mL | NT | 0 CFU/10mL |

Specifications only apply to clinical supplies
Abbreviations: NTU = nephelometric turbidity units; NT = not tested; ddPCR = digital droplet polymerase chain reaction; RP-HPLC = reversed phased high performance liquid chromatography; qPCR = quantitative polymerase chain reaction; LAL = limulus amebocyte lysate; EU = Endotoxin unit; CFU = Colony forming unit

The process parameters for formation and stabilization of lipid nanoparticles are summarized in Table 10.

Table 10. Process Parameters for Formation and Stabilization of LNPs

Process Parameter Acceptable Range

Temperature of aqueous phase 15-25° C.

Temperature of organic phase 15-25° C.

Flow rate ratio of citrate buffer to diluted drug substance for preparation of aqueous phase 4: 1ª

Flow rate ratio of LNP suspension to citrate buffer for stabilization 2: 1ª LNP collection vessel temperature 2-25° C. aTarget set-point during LNP formation. Ratios may be calculated from input flow rates.

Lipid Nanoparticle (LNP) Formation and Stabilization

To form the LNPs, the citrate buffer is combined in-line with the diluted drug substance in a 4:1 flowrate ratio to create the aqueous phase. The organic and aqueous phases are fed into one or more T-mixer(s) to form the LNPs. Post formation of the LNP suspension, the LNPs are stabilized via in-line dilution with citrate buffer in a 2:1 ratio of LNP suspension to citrate buffer and then collected in a vessel which is maintained at 2-25° C.

### Buffer Exchange and Concentration

To prepare for the Buffer Exchange and Concentration operation, the tangential flow filtration (TFF) membranes are flushed with Tris buffer for equilibration.

The LNPs are processed through a tangential flow filtration (TFF) unit operation where they are concentrated and then buffer exchanged with 2 diavolumes of tris buffer to remove ethanol from the suspension. The LNPs are then concentrated further and buffer exchanged with >8 additional diavolumes of Tris buffer.

TABLE 9
In-Process Controls During Drug Product Manufacturing

| Description | In-process control | Acceptance criteria |
|---|---|---|
| LNP formation and | pH of citrate buffer | 4.0 ± 0.1 |
| stabilization | | |
| Buffer exchange, concentration | pH of Tris buffer | 7.5 ± 0.2 |
| and filtration | | |
| Concentration adjustment and | pH of Sucrose/Tris buffer | 7.5 ± 0.2 |
| addition of cryoprotectant | | |
| Concentration adjustment and | RNA content prior to Tris | ≥0.133 mg/mL (Action |
| addition of cryoprotectant | addition | limit) |
| Sterile filtration | Bioburden prior to sterile | ≤2 CFU/20 mL |
| | filtration | |
| Sterile filtration | Filter integrity pre-use/post-use | Pass |
| | sterile filtration | |
| Aseptic filling | Fill weight (measurement) | 0.5 mL (0.52 g) ± 4% |

4.2.2. Excipients

The excipients Tromethamine (Tris base) and Tris Hydrochloride (HCl) present in the LNP drug product are buffer components used in pharmaceuticals and suitable to achieve the desired product pH. Sucrose is also included and was selected for its stabilizing effect to enable storage as a frozen composition prior to distribution and refrigeration at point of use. The 4 lipid excipients in the immunogenic composition are both functional and structural lipids utilized as part of the modRNA platform.

4.3. Dosage and Administration

The immunogenic composition is diluted as needed with normal saline, either by in-vial dilution or syringe to syringe mixing, prior to administration of the monovalent compositions or combination for the bivalent compositions.

For monovalent dosing, the immunogenic composition is dosed in the range of 3.75 to 30 μg per dose with an injection volume of 0.3 mL. Except for the 30-μg dose, dilution with sterile 0.9% sodium chloride (normal saline) is required for dosing. The 4 dose levels are:
3.75 μg mRNA
7.5 μg mRNA
15 μg mRNA
30 μg mRNA

The Wisconsin immunogenic composition is also dosed as a bivalent vaccine in combination with the Phuket immunogenic composition in a total delivered volume of 0.3 mL. The proposed dosing range (total RNA) and ratios of Wisconsin (W) immunogenic composition to Phuket (P) immunogenic composition in the bivalent immunogenic composition are:
15 μg at 1W: 1P (7.5 μg A+7.5 μg B)
30 μg at 1W: 1P (15 μg A+15 μg B)
22.5 μg at 1W: 2P (7.5 μg A+15 μg B)
18.75 μg at 1W: 4P (3.75 μg A+15 μg B)

For quadrivalent dosing, the immunogenic composition is dosed with an injection volume of 0.3 mL containing each of the 4 modRNA sequences for a total dose of up to 30 μg. No dilution is required for administration of the quadrivalent immunogenic composition Container Closure System.

The type I borosilicate glass vials meet USP <660>, Ph. Eur. 3.2.1, and JP 7.01 compendial requirements for hydrolytic resistance for Type I glass containers. The chlorobutyl elastomeric stoppers meet USP <381>, Ph. Eur. 3.2.9 and JP 7.03 compendial chemical testing requirements for elastomeric closures.

4.4. Storage and Transport, Label and Pack of the Drug Product The immunogenic composition is frozen and stored at ultralow temperature (ULT) (−90° C. to 60° C.) for long-term storage.

The influenza modRNA immunogenic composition is comprised of one or more nucleoside-modified mRNAs that encode the full-length HA glycoprotein derived from seasonal human influenza strains. The modRNA is formulated with 2 functional and 2 structural lipids, which protect the modRNA from degradation and enable transfection of the modRNA into host cells after IM injection. Influenza HA is the most abundant envelope glycoprotein on the surface of influenza A and B virions.

The primary pharmacology of the influenza modRNA immunogenic composition was evaluated in nonclinical studies in vitro and in vivo. In vitro and in vivo studies demonstrated the mechanism-of-action for the influenza modRNA immunogenic composition, which is to encode influenza HA that induces an immune response characterized by both a strong functional antibody responses and a Th1-type CD4+ and an IFNg+CD8+ T-cell response. Efficient in vitro expression of the HA glycoprotein from influenza modRNA vaccines was demonstrated in cultured cells. Mouse and rat immunogenicity studies demonstrated that influenza modRNA vaccines elicited strong functional and neutralizing antibody responses and CD4+ and CD8+ T-cell responses. Immunogenicity studies in mice, benchmarked against a licensed, adjuvanted inactivated influenza vaccine, also support the potential use of a multivalent influenza modRNA immunogenic composition formulation to target 4 different influenza virus strains.

A Lipid Nanoparticle Encapsulated RNA immunogenic composition Encoding the Influenza HA as a Vaccine Antigen

The influenza modRNA immunogenic composition is based on a modRNA platform technology. The single-stranded, 5′-capped modRNA contains an open reading frame encoding the HA vaccine antigen and features structural elements optimized for high efficacy of the RNA. The modRNA also contains a substitution of 1-methyl-pseudouridine for each uridine to decrease recognition of the vaccine RNA by innate immune sensors, such as TLRs 7 and 8, resulting in decreased innate immune activation and increased protein translation. The modRNA is encapsulated in a LNP for delivery into target cells. The formulation contains 2 functional lipids, ALC-0315 and ALC-0159, and 2 structural lipids DSPC (1,2-distearoyl-sn-glycero-3-phosphocholine) and cholesterol. The physicochemical properties and the structures of the 4 lipids are shown in the Table below.

TABLE 10
Lipids in the Formulation

| | Molec- | | Physical | |
|---|---|---|---|---|
| | ular | | State and | |
| | Weight | Molecular | Storage | |
| Lipid | [Da] | Formula | Condition | Chemical Name (Synonyms) and Structure |
| ALC-- | 766 | C₄₈H₉₅NO₅ | Liquid | ((4- |
| 0315 | | | (oil) | hydroxybutyl)azanediyl)bis(hexane- |
| | | | −20° C. | 6,1-diyl)bis(2-hexyldecanoate) |
| | | | | 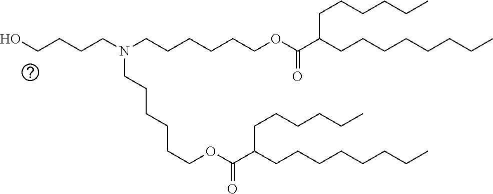 [SMILES: CCCCCCCCC(CCCCCC)C(=O)OCCCCCCN(CCCCO)CCCCCCOC(=O)C(CCCCCC)CCCCCCCC] |
| ALC- | ~2400- | C₃₀H₆₀NO | Solid | 2-[(polyethylene glycol)-2000]-N,N- |
| 0159 | 2600 | (C₂H₄O)ₙ | −20° C. | ditetradecylacetamide |
| | | OCH₃ | | 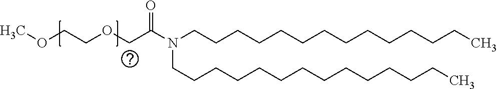 |
| DSPC | 790 | C₄₄H₈₈NO₈P | Solid | 1,2-Distearoyl-sn-glycero-3- |
| | | | −20° C. | phosphocholine |
| | | | | 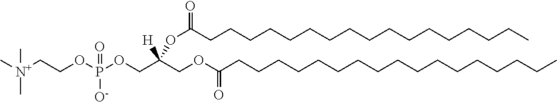 [SMILES: CCCCCCCCCCCCCCCCCC(=O)OC[C@H](COP(=O)([O-])OCC[N+](C)(C)C)OC(=O)CCCCCCCCCCCCCCCCC] |
| Choles- terol | 387 | C₂₇H₄₆O | Solid −20° C. | 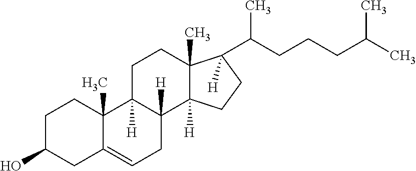 |

CAS = Chemical Abstracts Service;
DSPC = 1,2-Distearoyl-sn-glycero-3-phosphocholine

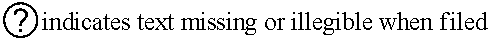

Influenza modRNA vaccine candidates selected for initial clinical testing will contain the full-length, codon-optimized coding sequence for the HA glycoprotein from the 4 cell-based virus strains recommended for use in the 2021-2022 Northern Hemisphere influenza season.
A/Wisconsin/588/2019 (H1N1)
A/Cambodia/e0826360/2020 (H3N2)
B/Phuket/3073/2013 (B Yamagata)
B/Washington/02/2019 (B Victoria)

In another embodiment, PF-07252220 (IRV) vaccine for Suspension for Injection is supplied as a white to off-white sterile frozen liquid, packaged in a 2 mL clear glass vial with a rubber stopper, aluminum overseal and flip off cap. The solution is a white to off-white opalescent liquid which may contain white to off white opaque, amorphous particles. The vial contains 0.5 mL with an extractable volume of 0.3 mL for further dilution via syringe mixing. For in-vial dilution, the vial contents (0.5 mL) should be accounted for the final dosing solution. Each vial includes the 0.1 mg/mL of PF-07252220 in a Lipid Nanoparticle (LNP) construct in 300 mM sucrose and 10 mM Tris, pH 7.4. There is no microbiological growth inhibitor in the formulation. PF-07252220 consists of five variations; four monovalent strain presentations and a quadrivalent strain presentation. The monovalent presentations may be further mixed to bivalent and quadrivalent dosing solutions at the point of use. The stability data presented below applies to all presentations and mixtures.
PF-07836259 (Phuket) Influenza mod RNA Suspension for Injection, 0.1 mg/mL
PF-07829855 (Wisconsin) Influenza mod RNA Suspension for Injection, 0.1 mg/mL
PF-07836261 (Washington) Influenza mod RNA Suspension for Injection, 0.1 mg/ml
PF-07836258 (Cambodia) Influenza mod RNA Suspension for Injection, 0.1 mg/ml
PF-07841697 Quadrivalent Influenza mod RNA Suspension for Injection, 0.1 mg/mL

The active investigational product must be stored at −90 to −60° C. (−130 to −76° F.) prior to use. Vials should be thawed at room temperature (no more than 30° C./86° F.) for approximately 30 minutes and then mixed by gently inverting the vial(s) 10 times. The investigational product will be administered intramuscularly.

TABLE 11
MONOVALENT DOSE PREPARATIONS USING 0.5 ML FILLED VOLUME
VIALS OF MONOVALENT INFLUENZA MOD RNA VACCINE

| | | | | | | | | Final Dosing | | | | Max |
|---|---|---|---|---|---|---|---|---|---|---|---|---|
| | | | | Volume | | Final | | Solution | | | | number |
| | | | | of 0.9% | | Volume | | concentration | | Final | | of Doses |
| | Dilution | Volume of | | Sodium | | of Dosing | | (in Diluted | | Injection | | per DP |
| Dose | Type | PF-07252220 | | Chloride | | solution | | Syringe/Vial) | | Volume | | vial |
| 3.75 mcg | Syringe to | 0.3 | mL | 2.1 | mL | 2.4 | mL | 12.5 | mcg/mL | 0.3 | mL | 5 |
| 7.5 | In-Vial | 0.5 | mL | 1.5 | mL | 2 | mL | 25 | mcg/mL | 0.3 | mL | 4 |
| 15 | In-Vial | 0.5 | mL | 0.5 | mL | 1 | mL | 50 | mcg/mL | 0.3 | mL | 2 |
| 30 | None | 0.5 | mL | 0 | mL | 0.5 | mL | 100 | mcg/mL | 0.3 | mL | 1 |

* Dilutions of Influenza mod RNA PF-07252220 are not limited to the preparations described in this table. The preparation instructions provided in this document are intended to support a specific clinical design, however dose preparation is not limited to these specific instruction sets. Active doses in the verified concentration range are acceptable.

TABLE 12
BIVALENT DOSE PREPARATIONS USING 0.5 ML FILLED VOLUME VIALS OF
MONOVALENT INFLUENZA MOD RNA VACCINE AND SYRINGE TO SYRINGE MIX

| | | | Volume | | Final | Final Dosing | |
|---|---|---|---|---|---|---|---|
| | | | of 0.9% | | Volume of | solution | |
| | DP | Volume of | Sodium | Syringe to | Dosing | Concentration | Final |
| Strain: | Vial | PF-07252220 | Chloride | Syringe Mix | Solution | (total active | Injection |
| Dose (mcg) | Strain | in Vial | into Vial | (1:1) | in Diluted | content) | Volume |
| 1: 7.5 mcg | 1 | 0.5 mL × | 0.5 mL | 0.3 mL | 0.6 mL | 50 mcg/mL | 0.3 mL |
| 2: 7.5 mcg | 2 | 0.5 mL × 1 vial | 0.5 mL | 0.3 mL | | | |
| 1: 15 mcg | 1 | 0.5 mL × | 0 | 0.3 mL | 0.6 mL | 100 mcg/mL | 0.3 mL |
| 2: 15 mcg | 2 | 0.5 mL × 1 vial | 0 | 0.3 mL | | | |
| 1: 30 mcg | 1 | 0.5 mL × 2 vials | 0 | 0.5 mL | 1 mL | 100 mcg/mL | 0.6 mL |
| 2: 30 mcg | 2 | 0.5 mL × 2 vials | 0 | 0.5 mL | | | |

*Bivalent doses can be made of any 2 monovalent strains, designated as Strain 1 and Strain 2

TABLE 13
BIVALENT DOSE PREPARATIONS USING 0.5 ML FILLED VOLUME VIALS
OF MONOVALENT INFLUENZA MOD RNA VACCINE AND IN-VIAL MIX
(VOLUME OF VIAL STRAIN 1 TRANSFERRED TO VIAL STRAIN 2)

| | | | Volume | In- Vial Mix: | | Final Dosing | |
|---|---|---|---|---|---|---|---|
| | | | of 0.9% | Volume of | Final Volume | solution | |
| | DP | Volume of | Sodium | Vial Strain 1 | of Dosing | Concentration | Final |
| Strain: | Vial | PF-07252220 | Chloride | transferred to | Solution in | (total active | Injection |
| Dose (mcg) | Strain | in Vial | into Vial | Vial Strain 2 | Diluted Vial | content) | Volume |
| 1: 7.5 mcg | 1 | 0.5 mL | 0.5 mL | 0.5 mL | 1 mL in | 75 mcg/mL | 0.3 mL |
| 2: 15 mcg | 2 | 0.5 mL | 0 | N/A | Vial 2 | | |
| 1: 3.75 mcg | 1 | 0.5 mL | 1.5 mL | 0.5 mL | 1 mL in | 62.5 mcg/mL | 0.3 mL |
| 2: 15 mcg | 2 | 0.5 mL | 0 | N/A | Vial 2 | | |

*Bivalent doses can be made of any 2 monovalent strains, designated as Strain 1 and Strain 2

TABLE 14
QUADRIVALENT DOSE PREPARATIONS USING 100 MCG/ML
QUADRIVALENT INFLUENZA MOD RNA VACCINE

| | | | Volume | Final Dosing | | Max |
|---|---|---|---|---|---|---|
| | Dilution | Volume of | of 0.9% | Solution | Injection | number |
| Dose | Type | PF-0725222 | Sodium | concentratio | Volum | of |
| 7.5 mcg | None | 0.5 mL | N/A | 100 mcg/mL | 0.3 mL | 1 |
| per strain | | | | | | |
| 15 mcg | None | 0.5 mL × | N/A | 100 mcg/mL | 0.6 mL | 1 |
| per strain | | 2 vials | | | | |

TABLE 15
QUADRIVALENT DOSE PREPARATIONS USING 0.5 ML FILLED VOLUME VIALS OF
MONOVALENT INFLUENZA MOD RNA VACCINE AND SYRINGE TO SYRINGE MIX

| | | | Step 1: | | Step 2: | Final Dosing | |
|---|---|---|---|---|---|---|---|
| | | | Volume from | Volume | Volume from | solution | |
| | DP | Volume of | Vial for | of 0.9% | Step 1 for | Concentration | Final |
| Strain: | Vial | PF-07252220 | syringe to | Sodium | syringe to | (total active | Injection |
| Dose (mcg) | Strain | in Vial | syringe mix | Chloride | syringe mix | content) | Volume |
| 1: 7.5 mcg | 1 | 0.5 mL | 0.3 mL | 1.6 mL | 1.1 mL from | 60 mcg/mL | 1 mL |
| 2: 7.5 mcg | 2 | 0.5 mL | 0.3 mL | | syringe A | | |
| 3: 22.5 | 3 | 0.5 mL × | 0.6 mL | N/A | 0.9 mL from | | |
| 4: 22.5 | | 2 vials | | | syringe B | | |
| | 4 | 0.5 mL × | 0.6 mL | | | | |
| | | 2 vials | | | | | |
| 1: 7.5 mcg | 1 | 0.5 mL | 0.3 mL | 0.4 mL | 0.3 mL from | 90 mcg/mL | 1 mL |
| 2: 7.5 mcg | 2 | 0.5 mL | 0.3 mL | | syringe A | | |
| 3: 37.5 | 3 | 0.5 mL × | 0.6 mL | N/A | 0.9 mL from | | |
| 4: 37.5 | | 2 vials | | | syringe B | | |
| | 4 | 0.5 mL × | 0.6 mL | | | | |
| | | 2 vials | | | | | |

*Quadrivalent doses can be made of any 4 monovalent strains, designated as Strain 1, 2, 3, and 4

### Example 4: Nonclinical Studies

An initial mouse immunogenicity study was conducted using an influenza modRNA immunogenic composition encoding the HA sequence from A/California/07/2009 (H1N1). This HA sequence differs from the H1N1 HA antigen that will be used in the clinical study due to strain differences, but the modRNA was formulated with the same clinical LNP composition and provides supportive data for the platform.

BALB/c mice were immunized IM with 1 μg of the LNP-formulated influenza modRNA vaccine on Days 0 and 28. ELISA of sera obtained on Days 28 and 49 showed high levels of HA-binding IgG. Sera obtained as early as 14 days after the first dose had high neutralization titers against A/California/07/2009 influenza virus, and by Day 49 (21 days after the second dose) serum influenza neutralization titers exceeded 1×104. The HAI titers against A/California/07/2009 measured in sera drawn on Day 49 greatly exceeded the titer of 40 that is generally accepted as protective against influenza in humans. BALB/c mice were immunized twice IM with 1 μg of the vaccine candidate. HA-specific IgG was measured by ELISA. The functionality of the antibodies was measured by influenza virus microneutralization. IFNγ ELISpot using splenocytes harvested on Day 49 and stimulated with antigen-specific peptides showed strong CD4+ and CD8+ T-cell responses. These data confirmed that modRNA formulated with LNPs elicited Th1 phenotype T-cell responses. BALB/c mice received 2 IM immunizations with 1 μg of modRNA encoding influenza HA. The T-cell response was analyzed using antigen-specific peptides to stimulate T cells recovered from the spleen. IFNγ release was measured after peptide stimulation using an ELISpot assay.

The primary serological assay used to measure vaccine-induced immune responses to influenza is the hemagglutinin inhibition assay, or HAI. The HAI quantitatively measures functional antibodies in serum that prevent HA-mediated agglutination of red blood cells in reactions containing receptor-destroying enzyme pretreated serum samples, influenza virus and red blood cells derived from turkey or guinea pig. The HAI titer is the reciprocal of the highest serum dilution resulting in loss of HA activity, visualized as a teardrop shape when the microtiter plate is tilted. Titers from multiple determinations per sample are reported as geometric mean titers (GMT). A HAI titer of ≥1:40 is generally accepted as protective in humans. HAI assays have been developed for each of the 4 influenza strains, A/Wisconsin/588/2019 (H1N1), A/Cambodia/e0826360/2020 (H3N2), B/Phuket/3073/2013 (B Yamagata) and B/Washington/02/2019 (B Victoria).

The influenza virus microneutralization assay, or MNT, quantitatively measures functional antibodies in serum that neutralize influenza virus activity, preventing productive infection of a host cell monolayer. A neutralization reaction occurs when influenza virus is incubated with serum samples; this reaction mixture is then applied to a monolayer of Madin-Darby Canine Kidney (MDCK) cells to measure the extent of neutralization. MNT titers are reported as the reciprocal of the dilution that results in 50% reduction in infection when compared to a no serum control.

Study to evaluate the feasibility of bi-valent modRNA HA flu vaccine with pre-mixed drug substance (RNA) to form an LNP and post-mixed LNP arms

As used herein unless stated otherwise, a “pre-mixed” drug substance refers to a composition wherein RNA expressing either HA or NA is mixed in a desired ratio, followed by a single formulation into an LNP. A “post-mixed” drug product refers to a composition wherein each RNA expressing either HA or NA is encapsulated in an LNP and the resulting RNA-encapsulated LNPs are then mixed in a desired ratio.

Hemagglutination-inhibition (HAI) antibody titers were examined in mice administered with a formulation as described in the following table.

### Study Design Table:

TABLE 16

| | | RNA drug product (DP, i.e., RNA | Dose | Dose Vol/ | Vax | Bleed |
|---|---|---|---|---|---|---|
| Gp# | Mice | encapsulated LNP) Description | (μg) | Route | (Day) | (Day) |
| 1 | 10 | Saline | — | 50 μl / IM | 0, 28 | 21, 42 |
| 2 | 10 | LNP modRNA HA mono-valent - Wisc | 1 | 50 μl / IM | 0, 28 | 21, 42 |
| | | Strain | | | | |
| | | 10 mM Tris and 300 mM Sucrose | | | | |
| 3 | 10 | LNP modRNA HA mono-valent - Wisc | 0.2 | 50 μl / IM | 0, 28 | 21, 42 |
| | | Strain | | | | |
| | | 10 mM Tris and 300 mM Sucrose | | | | |
| 4 | 10 | LNP modRNA HA mono-valent - Phuket | 1 | 50 μl / IM | 0, 28 | 21, 42 |
| | | Strain | | | | |
| | | 10 mM Tris and 300 mM Sucrose | | | | |
| 5 | 10 | LNP modRNA HA mono-valent - Phuket | 0.2 | 50 μl / IM | 0, 28 | 21, 42 |
| | | Strain | | | | |
| | | 10 mM Tris and 300 mM Sucrose | | | | |
| 6 | 10 | LNP modRNA HA pre-mix bi-valent, i.e., | 2 | 50 μl / IM | 0, 28 | 21, 42 |
| | | Bivalent Wisconsin and Phuket (DS mixed | | | | |
| | | prior to LNP formation) | | | | |
| | | 10 mM Tris and 300 mM Sucrose | | | | |
| 7 | 10 | LNP modRNA HA pre-mix bi-valent, i.e., | 0.4 | 50 μl / IM | 0, 28 | 21, 42 |
| | | Bivalent Wisconsin and Phuket (DS mixed | | | | |
| | | prior to LNP formation) | | | | |
| | | 10 mM Tris and 300 mM Sucrose | | | | |
| 8 | 10 | LNP modRNA HA post-mix bi-valent, i.e., | 2 | 50 μl / IM | 0, 28 | 21, 42 |
| | | Bivalent Wisconsin and Phuket (LNP mix) | | | | |
| | | 10 mM Tris and 300 mM Sucrose | | | | |
| 9 | 10 | LNP modRNA HA post-mix bi-valent, i.e., | 0.4 | 50 μl / IM | 0, 28 | 21, 42 |
| | | Bivalent Wisconsin and Phuket (LNP mix) | | | | |
| | | 10 mM Tris and 300 mM Sucrose | | | | |

High HAI Titers were induced by Wisconsin HA modRNA at 3 wks post-dose 1.

Slightly Higher HAI in Bi-Valent Groups.

Higher HAI in pre-mix Formulation for Bi-valent at 0.2 μg Dose. See tables 17-18 below.

TABLE 17
GMTs 3 weeks post-dose 1 (Wisconsin)

| | | | | | |
|---|---|---|---|---|---|
| GMT: | 10 | 422 | 260 | 453 | 130 |
| Sample: | Saline | bi-val. | bi-val. | bi-val. | bi-val. |
| | (Group 1) | pre-mix. | pre-mix. | post- | post- |
| | | | | mix. | mix. |
| RNA Dose | — | 2 | 0.4 | 2 | 0.4 |
| (ug) | | | | | |

TABLE 18
GMTs 3 weeks post-dose 1 (Phuket)

| | | | | | |
|---|---|---|---|---|---|
| GMT: | 10 | 25 | 13 | 28 | 10 |
| Sample: | Saline | bi-val. | bi-val. | bi-val. | bi-val. |
| | (Group 1) | pre-mix. | pre-mix. | post- | post- |
| | | | | mix. | mix. |
| RNA Dose | — | 2 | 0.4 | 2 | 0.4 |
| (ug) | | | | | |

It was also observed that 50% Neutralizing Ab Titers Were Comparable Between Pre-Mix and Post-Mix Drug Product. See Tables 19-22 below.

TABLE 19
at 3 weeks post-dose 1 (against Wisconsin)

| | | | | | |
|---|---|---|---|---|---|
| GMT | 165 | 14319 | 9393 | 24043 | 5221 |
| Sample: | Saline | bi-val. pre- | bi-val. pre- | bi-val. post- | bi-val. post- |
| | | mix. | mix. | mix. | mix. |
| RNA Dose | — | 2 | 0.4 | 2 | 0.4 |
| (ug) | | | | | |

TABLE 20
at 2 weeks post-dose 2 (against Wisconsin)

| | | | | | |
|---|---|---|---|---|---|
| GMT | 169 | 1286052 | 290731 | 1187870 | 278031 |
| Sample: | Saline | bi-val. pre- | bi-val. pre- | bi-val. post- | bi-val. post- |
| | | mix. | mix. | mix. | mix. |
| RNA Dose | — | 2 | 0.4 | 2 | 0.4 |
| (ug) | | | | | |

TABLE 21
at 3 weeks post-dose 1 (against Phuket)

| | | | | | |
|---|---|---|---|---|---|
| GMT | 60 | 730 | 1051 | 1089 | 265 |
| Sample: | Saline | bi-val. pre- | bi-val. pre- | bi-val. post- | bi-val. post- |
| | | mix. | mix. | mix. | mix. |
| RNA Dose | — | 2 | 0.4 | 2 | 0.4 |
| (ug) | | | | | |

TABLE 22
at 2 weeks post-dose 2 (against Phuket)

| | | | | | |
|---|---|---|---|---|---|
| GMT | 103 | 31818 | 6800 | 29035 | 8186 |
| Sample: | Saline | bi-val. pre- | bi-val. pre- | bi-val. post- | bi-val. post- |
| | | mix. | mix. | mix. | mix. |
| RNA Dose | — | 2 | 0.4 | 2 | 0.4 |
| (ug) | | | | | |

HAI Titers Were Comparable Between Pre-Mix Versus Post-Mix Drug Product. See tables 23-26 below.

TABLE 23
at 3 weeks post-dose 1 (against Wisconsin)

| | | | | | |
|---|---|---|---|---|---|
| GMT | 10 | 422 | 260 | 453 | 130 |
| Sample: | Saline | bi-val. pre- | bi-val. pre- | bi-val. post- | bi-val. post- |
| | | mix. | mix. | mix. | mix. |
| RNA Dose | — | 2 | 0.4 | 2 | 0.4 |
| (ug) | | | | | |

TABLE 24
at 2 weeks post-dose 2 (against Wisconsin)

| | | | | | |
|---|---|---|---|---|---|
| GMT | 10 | 2986 | 2389 | 3152 | 2229 |
| Sample: | Saline | bi-val. pre- | bi-val. pre- | bi-val. post- | bi-val. post- |
| | | mix. | mix. | mix. | mix. |
| RNA Dose | — | 2 | 0.4 | 2 | 0.4 |
| (ug) | | | | | |

TABLE 25
at 3 weeks post-dose 1 (against Phuket)

| | | | | | |
|---|---|---|---|---|---|
| GMT | 10 | 25 | 13 | 28 | 10 |
| Sample: | Saline | bi-val. pre- | bi-val. pre- | bi-val. post- | bi-val. post- |
| | | mix. | mix. | mix. | mix. |
| RNA Dose | — | 2 | 0.4 | 2 | 0.4 |
| (ug) | | | | | |

TABLE 26
at 2 weeks post-dose 2 (against Phuket)

| | | | | | |
|---|---|---|---|---|---|
| GMT | 10 | 1040 | 243 | 844 | 184 |
| Sample: | Saline | bi-val. pre- | bi-val. pre- | bi-val. post- | bi-val. post- |
| | | mix. | mix. | mix. | mix. |
| RNA Dose | — | 2 | 0.4 | 2 | 0.4 |
| (ug) | | | | | |

### Example 5: Description of Quadrivalent Drug Product

The quadrivalent drug product is a preservative-free, sterile dispersion of liquid nanoparticles (LNP) in aqueous cryoprotectant buffer for intramuscular administration. The drug product is formulated at 0.1 mg/mL RNA in 10 mM Tris buffer, 300 mM sucrose, pH 7.4.

The drug product is supplied in a 2 mL glass vial sealed with a chlorobutyl rubber stopper and an aluminum seal with flip-off plastic cap (maximum nominal volume of 0.3 mL).

TABLE 27
Composition of Quadrivalent Drug Product

| | | | | | Nominal |
|---|---|---|---|---|---|
| | | | | | Amount or |
| | | | | Filled | Net |
| | | | Unit | Amount | Quantity |
| | Grade/Quality | | Formula | (Total | (Net |
| Name of Ingredient | Standard | Function | (mg/mL) | mg/vial) | mg/vial) |
| PF-07829855 Drug | In-house | Active | 0.025 | 0.013 | 0.008 |
| Substance (Wisconsin) | specification | ingredient | | | |
| PF-07836258 Drug | In-house | Active | 0.025 | 0.013 | 0.008 |
| Substance (Cambodia) | specification | ingredient | | | |
| PF-07836259 Drug | In-house | Active | 0.025 | 0.013 | 0.008 |
| Substance (Phuket) | specification | ingredient | | | |
| PF-07836261 Drug | In-house | Active | 0.025 | 0.013 | 0.008 |
| Substance | specification | ingredient | | | |
| (Washington) | | | | | |
| ALC-0315^{a} | In-house | Functional | 1.43 | 0.72 | 0.43 |
| | specification | lipid | | | |
| ALC-0159^{b} | In-house | Functional | 0.18 | 0.09 | 0.05 |
| | specification | lipid | | | |
| DSPC^{c} | In-house | Structural | 0.31 | 0.16 | 0.09 |
| | specification | lipid | | | |
| Cholesterol | Ph. Eur., NF | Structural | 0.62 | 0.31 | 0.2 |
| | | lipid | | | |
| Sucrose | Ph. Eur., NF | Cryoprotectant | 102.69 | 51.35 | 30.81 |
| Tromethamine (Tris | Ph. Eur., USP | Buffer | 0.20 | 0.10 | 0.06 |
| base) | | component | | | |
| Tris (hydroxymethyl) | In-house | Buffer | 1.32 | 0.66 | 0.40 |
| aminomethane | specification | component | | | |
| hydrochloride (Tris HCl) | | | | | |
| Water for Injection | Ph. Eur., USP, | Solvent | q.s. ^{d} to | q.s. ^{d} to | q.s. ^{d} to |
| | JP | | 1.00 mL | 0.50 mL | 0.30 mL |

^{a}ALC-0315 = ((4-hydroxybutyl)azanediyl)bis(hexane-6,1-diyl)bis(2-hexyldecanoate)
^{b}ALC-0159 = 2-[(polyethylene glycol)-2000]-N, N-ditetradecylacetamide
^{c}DSPC = 1,2-Distearoyl-sn-glycero-3-phosphocholine
^{d}q.s. is an abbreviation for quantum satis meaning as much as is sufficient.

The recommended storage temperature of the FIH drug substance is −20±5° C.

The recommended long term storage temperature of the FIH drug product is −60 to −90° C.

The drug product may be stored at 2-8° C. at Point of Use.

TABLE 28
Batch Analyses for Quadrivalent Clinical Drug Product

| | | |
|---|---|---|
| Analytical | | |
| Procedure | Quality Attributes | Acceptance Criteria |

Composition and Strength

| | | |
|---|---|---|
| Appearance | Appearance | White to off-white |
| (Visual) | | suspension |
| Appearance | Appearance (Visible | May contain white to off- |
| (Particles) | Particulates) | white opaque, amorphous |
| | | particles |
| Subvisible | Subvisible particles | Particles ≥10 μm: ≤6000 |
| particulate | | per container |
| matter | | Particles ≥25 μm: ≤600 |
| | | per container |
| Potentiometry | pH | 7.4 ± 0.5 |
| Osmometry | Osmolality | 240-400 mOsmol/kg |
| Dynamic Light | LNP Size | 40-120 nm |
| Scattering (DLS) | LNP Polydispersity | ≤0.3 |
| Fluorescence | RNA Content | 0.074-0.126 mg/mL |
| Assay | RNA Encapsulation | ≥80% |
| HPLC-CAD | ALC-0315 Content | 0.90-1.85 mg/mL |
| | ALC-0159 Content | 0.11-0.24 mg/mL |
| | DSPC Content | 0.18-0.41 mg/mL |
| | Cholesterol Content | 0.36-0.78 mg/ml |
| Container | Vial content (volume) | Not less than 0.30 mL |
| content | | |

Identity

| | | |
|---|---|---|
| HPLC-CAD | Lipid identities | Retention times consistent |
| | | with references |
| | | (ALC-0315, ALC-0159, |
| | | Cholesterol, DSPC) |
| RT-PCR | Identity of encoded | Identity confirmed |
| | RNA sequence(s) | |

Purity

| | | |
|---|---|---|
| Capillary Gel | RNA Integrity | ≥55% intact |
| Electrophoresis | | RNA (release) |
| | | ≥50% intact |
| | | RNA (stability) |

Safety

| | | |
|---|---|---|
| Endotoxin (LAL) | Endotoxin (LAL) | ≤12.5 EU/mL |
| Sterility | Sterility | No growth detected |
| Dye incursion | Container Closure | Pass |
| | Integrity | |

TABLE 29
Batch Analyses for Quadrivalent Clinical Drug Product

| | | |
|---|---|---|
| Analytical | | |
| Procedure | Quality Attributes | Acceptance Criteria |

Composition and Strength

| | | |
|---|---|---|
| Appearance | Appearance | White to off-white |
| (Visual) | | suspension |
| Appearance | Appearance (Visible | May contain white to off- |
| (Particles) | Particulates) | white opaque, amorphous |
| | | particles |
| Subvisible | Subvisible particles | Particles ≥10 μm: ≤6000 |
| particulate | | per container |
| matter | | Particles ≥25 μm: ≤600 |
| | | per container |
| Potentiometry | pH | 7.4 ± 0.5 |
| Osmometry | Osmolality | 240-400 mOsmol/kg |
| Dynamic Light | LNP Size | 40-120 nm |
| Scattering (DLS) | LNP Polydispersity | ≤0.3 |
| Fluorescence | RNA Content | 0.074-0.126 mg/mL |
| Assay | RNA Encapsulation | ≥80% |
| HPLC-CAD | ALC-0315 Content | 0.90-1.85 mg/mL |
| | ALC-0159 Content | 0.11-0.24 mg/mL |
| | DSPC Content | 0.18-0.41 mg/mL |
| | Cholesterol Content | 0.36-0.78 mg/mL |
| Container content | Vial content (volume) | Not less than 0.30 mL |

Identity

| | | |
|---|---|---|
| HPLC-CAD | Lipid identities | Retention times consistent |
| | | with references (ALC- |
| | | 0315, ALC-0159, |
| | | Cholesterol, DSPC) |
| RT-PCR | Identity of encoded | Identity confirmed |
| | RNA sequence(s) | |

Purity

| | | |
|---|---|---|
| Capillary Gel | RNA Integrity | ≥55% intact RNA |
| Electrophoresis | | (release) |
| | | ≥50% intact RNA |
| | | (stability) |

Safety

| | | |
|---|---|---|
| Endotoxin (LAL) | Endotoxin (LAL) | ≤12.5 EU/mL |
| Sterility | Sterility | No growth detected |
| Dye incursion | Container Closure Integrity | Pass |

Primary Efficacy Analysis for Participants 18 Through 64 Years of Age (Northern Hemisphere): A Phase 3, Randomized, Observer-blinded Study to Evaluate the Efficacy, Safety, Tolerability, and Immunogenicity of a Modified RNA Vaccine Against Influenza (described above) Compared to Licensed Inactivated Influenza Vaccine in Healthy Adults 18 Years of Age or Older

Influenza modRNA vaccine candidates encoding HA antigen targeting a single, two, or four influenza strains were studied in the dose and schedule finding trial, Phase 1/2 study C4781001, and quadrivalent influenza modRNA vaccines (as selected during Phase 1/2) were then studied in an ongoing pivotal efficacy trial, Phase 3 study C4781004.

### Methods

We conducted a Phase 3, randomized, observer-blinded study to evaluate the safety, tolerability, immunogenicity, and efficacy of qIRV described above compared to a licensed influenza vaccine (QIV) against laboratory-confirmed (RT-PCR) influenza (LCI) in healthy adults 18 years and older (stratified by age: 18 through 64 and >65 years). Participants 18-64-years were enrolled during the 2022-2023 northern hemisphere influenza season. Immunogenicity was evaluated by hemagglutination inhibition (HAI) assay and quantification of hemagglutinin-specific CD4+ and CD8+ T cells. Reactogenicity was collected in a subset population and other adverse events were collected from all participants. An objective of the study aimed to demonstrate, among other things, that the efficacy of qIRV is noninferior to that of QIV against LCI associated with per-protocol ILI (ILI refers to influenza-like illness), in participants 18 through 64 years of age. An estimand involved in participants 18 through 64 years of age complying with the key protocol criteria (evaluable participants) at least 14 days after study intervention: RVE, defined as the relative reduction of the proportion of participants reporting LCI cases with associated per-protocol ILI in the qIRV group compared to the QIV group. A secondary estimand involved comparisons made using HAIs based on egg-derived virus for the influenza strains that are present in the study interventions: (i) GMR of HAI titers for each strain in qIRV recipients compared to QIV recipients 4 weeks after vaccination; and (ii) The difference in percentage of participants achieving seroconversion for each strain at 4 weeks after vaccination in qIRV recipients compared to QIV recipients.

This was a Phase 3, randomized, observer-blinded study to evaluate the efficacy, safety, tolerability, and immunogenicity of qIRV encoding HA of 4 seasonally recommended strains (2 A strains and 2 B strains) in healthy individuals ≥18 years of age. The strain composition of qIRV (2022-2023 influenza season) recommended by the WHO was: A/H1N1/Wisconsin/588/2019, A/H3N2/Darwin/6/2021, B/Victoria/Austria/1359417/2021, and B/Yamagata/Phuket/3073/2013.

The licensed QIV administered as a control for both age strata in Study C4781004 was Fluzone® Standard-Dose Quadrivalent (Sanofi Pasteur).

The strain composition of QIV (2022-2023 influenza season) recommended by the WHO was: A/H1N1/Victoria/2570/2019; A/H3N2/Darwin/9/2021; B/Victoria/Austria/1359417/2021; and B/Yamagata/Phuket/3073/2013.

Up to approximately 36,200 participants in the northern hemisphere were to be initially enrolled in this study and stratified by age as follows: Up to approximately 18,600 participants ≥65 years of age were to be enrolled and randomized 1:1 to receive 1 dose of either qIRV or seasonal QIV comparator. Up to approximately 17,600 participants 18 through 64 years of age were to be enrolled and randomized 1:1 to receive 1 dose of either qIRV or seasonal QIV comparator. In each age stratum: Approximately 6000 participants were to be included in a reactogenicity subset. For participants in the reactogenicity subset, a reactogenicity e-diary was completed by each participant for 7 days following vaccination. Approximately 4000 participants were to be included in an immunogenicity subset. Blood samples of approximately 15 mL were collected for immunogenicity assessments prior to vaccination and at 4 weeks and 6 months after vaccination. Efficacy was assessed in this study through surveillance for ILI.

The primary efficacy analysis was to be conducted when at least 130 first-episode evaluable LCI cases associated with per-protocol ILI, caused by any strain, accrued in a given age group. Descriptive statistics for binary variables (eg, proportions) are the percentage (%), the numerator (n) and the denominator (N) used in the percentage calculation, and the 95% Cls where applicable. The exact 95% CI for binary endpoints for each group will be computed using the F distribution (Clopper-Pearson). The 95% CI for the between-group difference for binary endpoints will be calculated using the Miettinen and Nurminen method. Unless otherwise stated, descriptive statistics for continuous variables are n, mean, median, standard deviation, minimum, and maximum. For the primary efficacy objectives, RVE was estimated along with the 2-sided 95% CI. RVE was defined as the relative risk reduction of the proportion of participants reporting first-episode LCI cases with associated per-protocol ILI caused by any strain, with symptom onset at least 14 days after vaccination, in the qIRV group compared to the QIV group. The analysis of efficacy used a conditional exact test based on the binomial distribution of the number of first-episode LCI cases in the qIRV group, given the total number of first-episode cases in both groups. The primary efficacy objectives were evaluated sequentially when ≥130 first-episode evaluable LCI cases associated with per-protocol ILI, caused by any strain. Noninferiority was declared if the lower bound of the 2-sided 95% CI for RVE was >-10%. If noninferiority was declared, superiority was assessed. Superiority was declared if the lower bound of the 2-sided 95% CI for RVE was >0%. All RVE estimations with any additional LCI cases collected after primary analysis were descriptively summarized with a 2-sided 95% C1. For the secondary and exploratory/tertiary efficacy objectives, RVE associated with different definitions of ILI was estimated along with the 2-sided 95% C1. HAI titers for the homologous (vaccine-encoded) strains were measured with assays based on the 2022-2023 northern hemisphere seasonal strains (2xA, 2xB). As recommended by WHO for egg-based influenza vaccines: A/H1N1/Victoria/2570/2019; A/H3N2/Darwin/9/2021; B/Victoria/Austria/1359417/2021; and B/Yamagata/Phuket/3073/2013. As recommended by WHO for recombinant or cell-based influenza vaccines: A/H1N1/Wisconsin/588/2019, A/H3N2/Darwin/6/2021, B/Victoria/Austria/1359417/2021, and B/Yamagata/Phuket/3073/2013. GMTs were calculated as the mean of assay results after logarithmic transformation and then exponentiating the mean to express results on the original scale. Two-sided 95% Cls were obtained by taking log transforms of assay results, calculating the 95% CI with reference to Student's t-distribution, and then exponentiating the confidence limits. GMFRs were defined as ratios of the HAI titer results after vaccination to the results before vaccination and were limited to participants with nonmissing values at both time points. GMFRs were calculated as the mean of the difference in logarithmically transformed assay results (later time point minus earlier time point) and exponentiating the mean, with the associated 2-sided 95% Cls obtained by constructing Cls using Student's t distribution for the mean difference on the logarithm scale and exponentiating the confidence limits. GMRs were calculated as the mean of the difference of logarithmically transformed HAI titer results and exponentiating the mean, with associated 2-sided Cls obtained by calculating Cls using Student's t-distribution for the mean difference of the logarithmically transformed assay results and exponentiating the confidence limits. Seroconversion was defined as an HAI titer. Noninferiority of the immune response to qIRV compared to QIV was assessed at 4 weeks after vaccination by examining: GMRs of HAI titers for each strain and the difference in the percentage of participants achieving seroconversion for each strain. Noninferiority was declared for a strain if both the lower bound of the 2-sided 95% CI for the GMR (qIRV/QIV) was >0.67 (1.5-fold margin) and the lower bound of the 2-sided 95% CI for the difference between vaccine groups (qIRV-QIV) in the percentage of participants with seroconversion was >-10% for each strain.

### Results

The primary analysis of relative vaccine efficacy (RVE) of qIRV versus QIV in participants 18 through 64 years of age was performed based on 144 total LCI cases. RVE in the evaluable efficacy population was 34.5% (95% CI 7.4-53.9) which met both non-inferiority (NI) and superiority criteria. The LCI cases included A/H3N2 and A/H1N1 strains. NI of HAI antibody responses was shown for influenza A strains but not influenza B. IFNγ+CD4+ T-cell and CD8+ T-cell GMFRs were higher in the qIRV group for all 4 strains. Primarily mild or moderate reactogenicity was observed in both vaccine groups but reported more frequently among qIRV recipients. AE profile was similar between both vaccine groups.

TABLE 8
HAI GMTs and GMRs for Each Strain-Comparison of qIRV (30 μg)
to Licensed QIV at 4 Weeks After Vaccination-18 Through 64
Years of Age-Evaluable Immunogenicity (HAI Assays Based
on Egg-Derived Virus) Population

| | Vaccine Group | | |
|---|---|---|---|
| | (as Randomized) | | |
| | qIRV (30 μg) | Licensed QIV | GMR^{d,e} |
| | (N^{a} = 741) | (N^{a} = 714) | (95% CI^{e}) |
| | n^{b} | n^{b} | qIRV |
| | GMT^{c} | GMT^{c} | (30 μg)/ |
| Assay Strain | (95% CI^{c}) | (95% CI^{c}) | Licensed QIV |
| A/Darwin/9/2021 (H3N2) | 740 | 714 | 0.98 |
| | 45.8 | 46.5 | (0.95, 1.02) |
| | (44.6, 47,0) | (46.3, 47.8) | |
| A/Victoria/2570/2019 | 741 | 734 | 0.99 |
| (H1N1) | 52.6 | 52.9 | (0.96, 1.03) |
| | (51.3, 53.9) | (51.5, 54.3) | |
| B/Austria/1359417/2021 | 740 | 713 | 1.01 |
| (B/Victoria lineage) | 34.8 | 34.6 | (0.97, 1.04) |
| | (33.9, 35.7) | (33.7, 35.5) | |
| B/Phuket/3073/2013 | 741 | 714 | 0.99 |
| (B/Yamagata lineage) | 45.1 | 45.5 | (0.95, 1.03) |
| | (43.9, 46.3) | (44.3, 46.7) | |

Abbreviations: GMR = geometric mean; geometric mean titer; HAI = hemagglutination inhibition; LLOQ = lower limit of quantitation.
Note:
The LLOQ for each strain-specific HAI titer is 10. Assay results below the LLOQ were set to 9.5 × LLOQ for analysis.
^{a}N = number of participants in the vaccine group.
^{b}n = Number of participants with valid and determinate assay results for the specified assay at the sampling time point.
^{c}GMTs and 2-sided 95% CIs were calculated by exponentiating the mean logarithm of the concentrations and the corresponding CIs (based on the Student t distribution).
^{d}GMRs are estimated by the ratio of the GMTs between pairs of qIRV (30 μg) and Licensed QIV.
^{e}GMRs and the corresponding 2-sided 95% CIs were calculated by exponentiating the mean logarithm of qIRV (30 μg) and Licensed QIV difference and the corresponding CIs (based on the Student t distribution).

### CONCLUSIONS

qIRV is the first mRNA vaccine to demonstrate efficacy in prevention of influenza. Lack of influenza B cases limits confirmation of efficacy against both influenza A and B strains. qIRV has an acceptable safety profile.

### Example6: LNP Flu HA modRNA Quadrivalent Study

The following example describes a study of LNP Flu HA modRNA Quadrivalent, in which mice were administered with different LNP_Flu HA modRNA materials as detailed in the table below. Sera collected at Day 21 post prime and at Day 42 (14 days post boost) were evaluated by serology testing (HAI, and neutralization).

TABLE 30

| | | | Dose Vol/ | Vax | Bleed |
|---|---|---|---|---|---|
| Mice | RNA DP Description | Dose (μg) | Route | (Day) | (Day) |
| 10 | Saline | — | 50 μl/IM | 0, 28 | 21, |
| | | | | | 42 |
| 10 | Quadrivalent | 4 | 50 μl/IM | 0, 28 | 21, |
| | (modRNAs premixed & | | | | 42 |
| | coformulate) | | | | |
| 10 | Quadrivalent | 0.8 | 50 μl/IM | 0, 28 | 21, |
| | (modRNAs premixed & | | | | 42 |
| | coformulate) | | | | |
| 10 | Quadrivalent (LNPs | 4 (1 ug | 50 μl/IM | 0, 28 | 21 |
| | made separately & | each) | | | 42 |
| | mixed), “post-mixed” | | | | |
| 10 | Quadrivalent (LNPs | 0.8 (0.2 | 50 μl/IM | 0, 28 | 21, |
| | made separately & | ug each) | | | 42 |
| | mixed) “post-mixed” | | | | |

HAI Titers were Comparable Between Pre-mixed Versus Post-mixed Drug Product at D21, see following tables 31-35.

TABLE 31
GMTs 3 weeks post-dose 1 (Wisconsin)

| | | | | | |
|---|---|---|---|---|---|
| GMT: | 10 | 686 | 343 | 485 | 299 |
| Sample: | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

TABLE 32
GMTs 3 weeks post-dose 1 (Cambodia)

| | | | | | |
|---|---|---|---|---|---|
| GMT: | 10 | 686 | 343 | 485 | 299 |
| Sample: | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

TABLE 33
GMTs 3 weeks post-dose 1 (Cambodia)

| | | | | | |
|---|---|---|---|---|---|
| GMT: | 28 | 40 | 49 | 46 | 53 |
| Sample: | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

TABLE 34
GMTs 3 weeks post-dose 1 (Washington)

| | GMT: | | | | |
|---|---|---|---|---|---|
| | 14 | 26 | 21 | 30 | 23 |
| | Sample: | | | | |
| | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

TABLE 35
GMTs 3 weeks post-dose 1 (Phuket)

| | GMT: | | | | |
|---|---|---|---|---|---|
| | 10 | 61 | 36 | — | 53 |
| | Sample: | | | | |
| | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

H1N1 A/Wisconsin: Comparable 50% Neutralization Titers Between Pre-mix and Post-Mix were observed. H3N2 A/Cambodia: Comparable 50% Neutralization Titers Between Pre-mix and Post-Mix were observed. By/Phuket: Comparable 50% Neutralization Titers Between Pre-mix and Post-Mix were observed. Bv/Washington: Comparable 50% Neutralization Titers Between Pre-mix and Post-Mix were also observed.

### Example7: Immunogenicity Data in Mice of a Multivalent Influenza modRNA Vaccine, cont'd

To evaluate the feasibility of a multivalent formulation of the modRNA influenza vaccine, modRNAs encoding 4 different HA proteins and 4 different neuraminidase (NA) proteins were generated. Immune responses elicited by mice vaccinated with LNP-formulated modRNA encoding a single strain-specific HA or NA were compared to groups vaccinated with an octavalent HA/NA modRNA formulation. Octavalent formulation methods were compared by separately formulating each modRNA expressing HA or NA in LNPs and then mixing the eight LNPs together in equal ratios, or by pre-mixing the eight modRNAs followed by a single co-formulation in LNPs.

BALB/c mice were immunized IM with 2 μg of each HA and NA-expressing modRNA either as a monovalent or octavalent vaccine formulation in LNPs on Days 0 and 28. Robust antibody and T cell responses were elicited by LNP-formulated modRNA to all HA and NA components, at levels similar to or greater than the licensed vaccine comparator. Similar HAI and neutralizing responses were observed on Day 49 (21 days after the second boost) for individual HA and octavalent formulations for influenza A strains. Antibodies measured against NA showed a similar trend as HA (data not shown). Immunogenicity studies in mice, benchmarked against a licensed, adjuvanted inactivated influenza vaccine, support the potential use of a multivalent influenza modRNA vaccine formulation to target at least four different influenza virus strains. Initial immunogenicity studies in mice of an octavalent HA/NA modRNA vaccine indicated no interference for influenza A strains and exhibited antibody responses for influenza B strains in comparison to monovalent control vaccines. These initial mouse immunogenicity data support the use of a multivalent modRNA formulation.

### Example8: ModRNA Flu Quadrivalent Feasibility Study

This study was performed to evaluate the immunogenicity of a quadrivalent modRNA vaccine candidate encoding influenza hemagglutinin (HA) from the four strains recommended for the Northern Hemisphere 21-22 season (H1N1 A/Wisconsin/588/2019, H3N2 B/Cambodia/e0826360/2020, By/Phuket/3073/2013, Bv/Washington/02/2019) compared to a monovalent modRNA-HA vaccine of each strain. Historically, lower titers have been induced against the less immunogenic Flu B strains when mixed in a multivalent formulation. This study investigated whether that interference can be rescued if Flu B doses in the vaccine were increased 2× or 4× relative to Flu A doses. Intramuscular immunization of Balb/c mice with LNP-formulated modRNA-HA monovalent or quadrivalent vaccines induced functional antibody responses as measured by the Hemagglutination Inhibition Assay (HAI) and 1-day Microneutralization Assay (MNT) at Day 21 (3 weeks post dose 1) and Day 42 (2 weeks post dose 2). Monovalent and quadrivalent vaccines were similarly immunogenic against the two A strains at D21, with a robust boost effect observed 2 weeks after the second dose. Interference was observed in titers elicited by the quadrivalent modRNA vaccine only against B/Washington at D21. After the second dose, a modest increase in titers was observed for the B strains. At D42, interference was observed for both B/Phuket and B/Washington. This interference was rescued at the low dose for both strains by increasing the Flu B HA concentration 2× or 4× compared to the Flu A HA. An effective quadrivalent modRNA Influenza vaccine may potentially include an adjusted Flu B dose.

The purpose of this study was to evaluate the feasibility of a quadrivalent modRNA-HA influenza vaccine. The objectives were two-fold: 1) to compare the immunogenicity of a quadrivalent vs. monovalent modRNA formulation in mice to assess levels of interference and 2) to determine if altering the dose composition of Flu B can “rescue” any interference. The influenza modRNA composition comprises up to 4 nucleoside-modified mRNAs that encodes the full-length hemagglutinin (HA)glycoprotein derived from a seasonal human influenza strain. The modRNA is formulated with two functional and two structural lipids, which protect the modRNA from degradation and enable transfection of the modRNA into host cells after intramuscular (IM) injection. The modRNA in the quadrivalent vaccine and the monovalent comparators studied herein encode HA proteins from the four strains recommended for the 2021-2022 Northern Hemisphere Influenza season. These strains are A/Wisconsin/588/2019 (H1N1) pdm09; A/Cambodia/e0826360/2020 (H3N2); B/Phuket/3073/2013 (B/Yamagata/16/88 lineage); and B/Washington/02/2019 (B/Victoria/2/87 lineage).

Balb/c mice were immunized on Days 0 and 28 with either a monovalent modRNA-HA vaccine for one of the four recommended strains or a quadrivalent composition. Quadrivalent vaccines were mixed either as modRNA drug substances then coformulated into LNPs (pre-mix) or as LNPs after formulation of each drug substance (post-mix), as described in earlier Examples. Increased relative Flu B doses (either 2× or 4× higher than Flu A doses) were tested to determine optimal dose for the less immunogenic B strains. Serum was collected 21 days post prime and 14 days post boost. Anti-HA antibodies were measured by the Hemagglutination Inhibition Assay (HAI) and 1-day Microneutralization Assay (MNT) to determine immunogenicity.

This study was designed with 17 groups as shown in Table 36, each containing a total of 10 female mice (strain of mice: Balb/c). The modRNA drug products were evaluated at 0.05 mL dose volume.

TABLE 36
Study design

| | | | | Dose Vol / | Vax | Bleed |
|---|---|---|---|---|---|---|
| Gp # | Mice | RNA DP Description | Dose (μg) | Route | (Day) | (Day) |
| 1 | 10 | Saline | — | 50 μL / IM | 0, 28 | 21, 42 |
| 2 | 10 | A/Wisconsin (H1N1) modRNA HA | 1 | 50 μL / IM | 0, 28 | 21, 42 |
| | | monovalent | | | | |
| 3 | 10 | A/Wisconsin (H1N1) modRNA HA | 0.2 | 50 μL / IM | 0, 28 | 21, 42 |
| | | monovalent | | | | |
| 4 | 10 | A/Cambodia (H3N2) modRNA HA | 1 | 50 μL / IM | 0, 28 | 21, 42 |
| | | monovalent | | | | |
| 5 | 10 | A/Cambodia (H3N2) modRNA HA | 0.2 | 50 μL / IM | 0, 28 | 21, 42 |
| | | monovalent | | | | |
| 6 | 10 | B/Phuket (By) modRNA HA | 1 | 50 μL / IM | 0, 28 | 21, 42 |
| | | monovalent | | | | |
| 7 | 10 | B/Phuket (By) modRNA HA | 0.2 | 50 μL / IM | 0, 28 | 21, 42 |
| | | monovalent | | | | |
| 8 | 10 | B/Washington (Bv) modRNA HA | 1 | 50 μL / IM | 0, 28 | 21, 42 |
| | | monovalent | | | | |
| 9 | 10 | B/Washington (Bv) modRNA HA | 0.2 | 50 μL / IM | 0, 28 | 21, 42 |
| | | monovalent | | | | |
| 10 | 10 | Quadrivalent (modRNAs premixed & | 4 | 50 μL / IM | 0, 28 | 21, 42 |
| | | coformulate) | | | | |
| 11 | 10 | Quadrivalent (modRNAs premixed & | 0.8 | 50 μL / IM | 0, 28 | 21, 42 |
| | | coformulate) | | | | |
| 12 | 10 | Quadrivalent (LNPs made separately | 4 (1 μg each) | 50 μL / IM | 0, 28 | 21, 42 |
| | | & mixed) | | | | |
| 13 | 10 | Quadrivalent (LNPs made separately | 0.8 (0.2 μg each) | 50 μL / IM | 0, 28 | 21, 42 |
| | | & mixed) | | | | |
| 14 | 10 | Quadrivalent (LNPs made separately | 4 (0.66 μg H1, H3 | 50 μL / IM | 0, 28 | 21, 42 |
| | | & mixed) - 2x Flu B dose | & 1.32 μg Bv, By) | | | |
| 15 | 10 | Quadrivalent (LNPs made separately | 1.2 (0.2 μg H1, H3 | 50 μL / IM | 0, 28 | 21, 42 |
| | | & mixed) - 2x Flu B dose | & 0.4 μg Bv, By) | | | |
| 16 | 10 | Quadrivalent (LNPs made separately | 4 (0.4 μg H1, H3 | 50 μL / IM | 0, 28 | 21, 42 |
| | | & mixed) - 4x Flu B dose | & 1.6 μg Bv, By) | | | |
| 17 | 10 | Quadrivalent (LNPs made separately | 2 (0.2 μg H1, H3 | 50 μL / IM | 0, 28 | 21, 42 |
| | | & mixed) - 4x Flu B dose | & 0.8 μg Bv, By) | | | |

One 0.3 m1 syringe was filled to 0.05 mL, and vaccine was administered via the intramuscular route for each animal. Procedure was repeated on day 28 for the booster vaccination.

TABLE 37
Test Article and Diluent

| Item | | Formulation Matrix and | | |
|---|---|---|---|---|
| # | Test articles/Diluent | information | Gps to be used | Vials #/Storage |
| 1 | Saline | 0.9% NaCl in water | 1 and as diluent for | 2 bottles |
| | | | groups 2-17 | RT |
| 2 | LNP HA mono-valent | 0.086 mg/mL of modRNA | 2-3 and 12-17 | 5 × 0.5 mL |
| | plasmid | LNP in 10 mM Tris/300 mM | | vials −80° C. |
| | (Wisconsin) | Sucrose, pH 7.4 | | (2) prime, (2) boost, |
| | | | | and 1 extra |
| 3 | LNP HA mono-valent | 0.117 mg/mL of modRNA | 4-5 and 12-17 | 5 × 0.5 mL |
| | plasmid | LNP in 10 mM Tris/300 mM | | vials −80° C. |
| | (Cambodia) | Sucrose, pH 7.4 | | (2) prime, (2) boost, |
| | | | | and 1 extra |
| 4 | LNP HA mono-valent | 0.093 mg/mL of modRNA | 6-7 and 12-17 | 7 × 0.5 mL |
| | plasmid | LNP in 10 mM Tris/300 mM | | vials −80° C. |
| | (Phuket) | Sucrose, pH 7.4 | | (3) prime, (3) boost, |
| | | | | and 1 extra |
| 5 | LNP HA mono-valent | 0.085 mg/mL of modRNA | 8-9 and 12-17 | 9 × 0.5 mL |
| | plasmid | LNP in 10 mM Tris/300 mM | | vials −80° C. |
| | (Washington) | Sucrose, pH 7.4 | | (4) prime, (4) boost, |
| | | | | and 1 extra |
| 6 | LNP HA quad-valent | 0.100 mg/mL of modRNA | 10 and 11 | 6 × 0.5 mL |
| | plasmid pre-mix DS | LNP in 10 mM Tris/300 mM | | vials −80° C. |
| | (Wisconsin, Phuket, | Sucrose, pH 7.4 | | (2) prime, (2) boost, |
| | Cambodia, Washington) | | | and 2 extra |

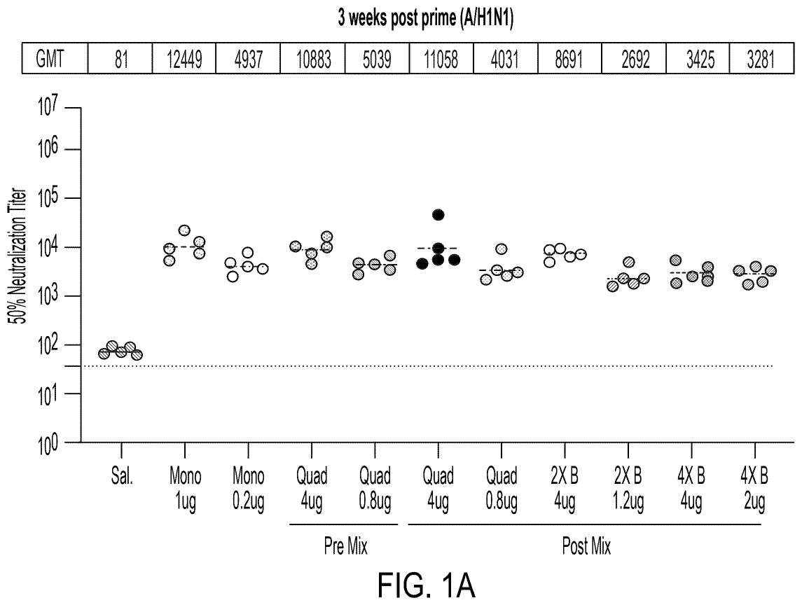

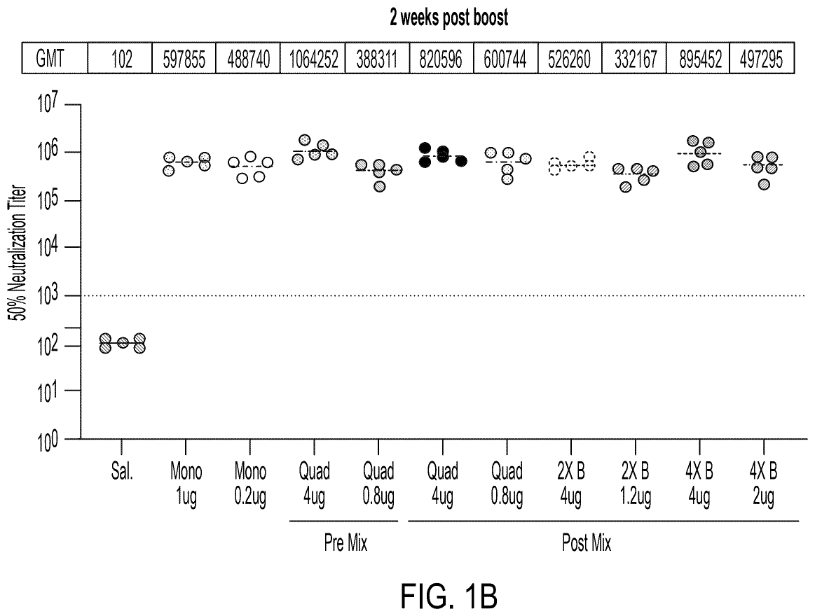

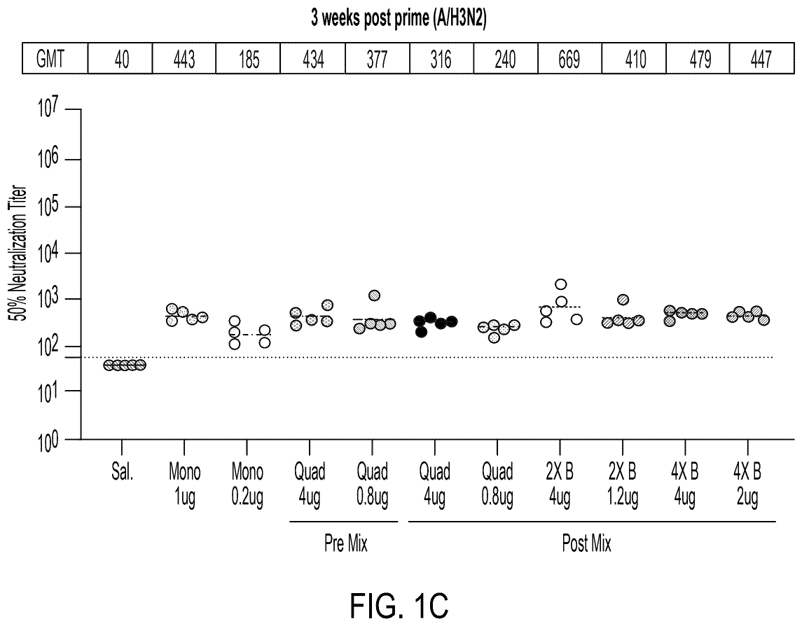

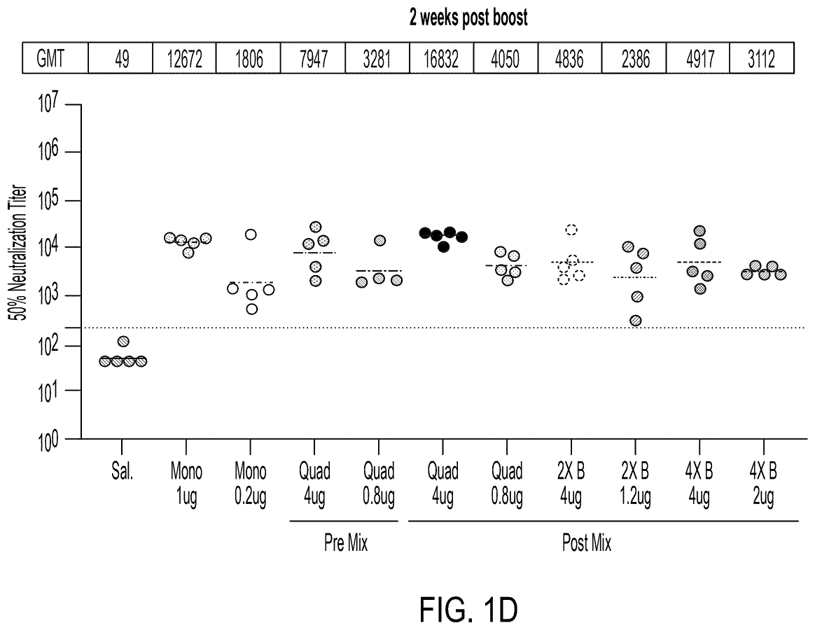

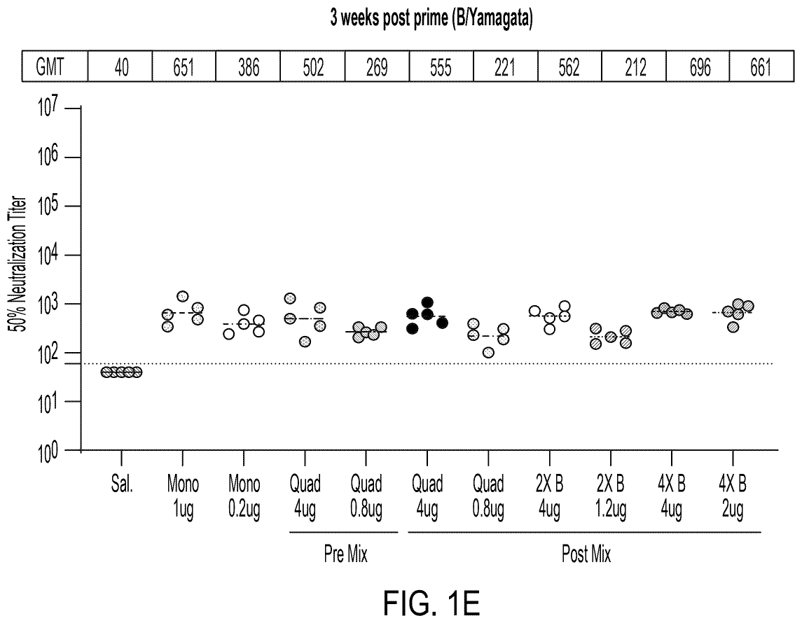

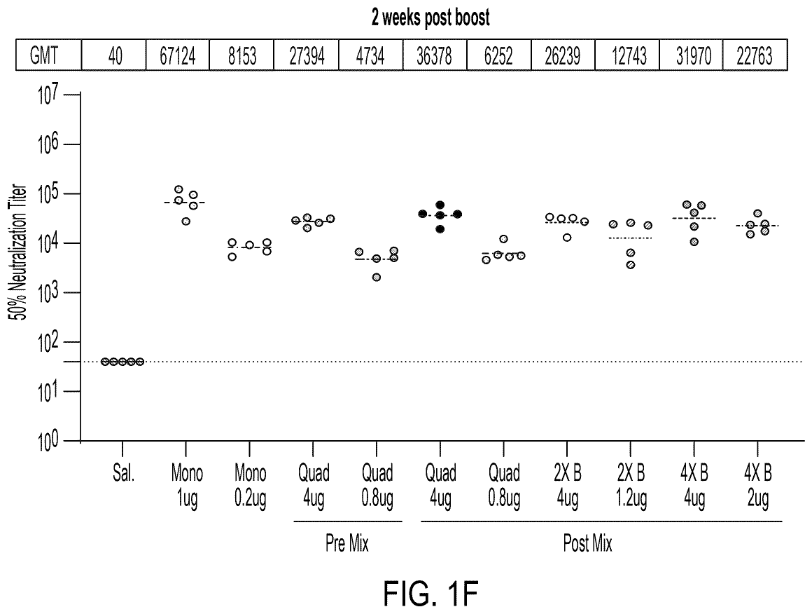

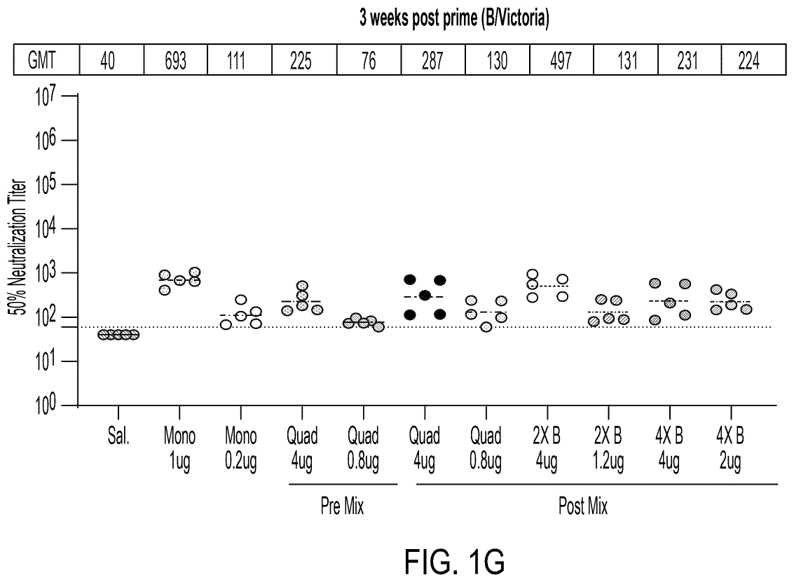

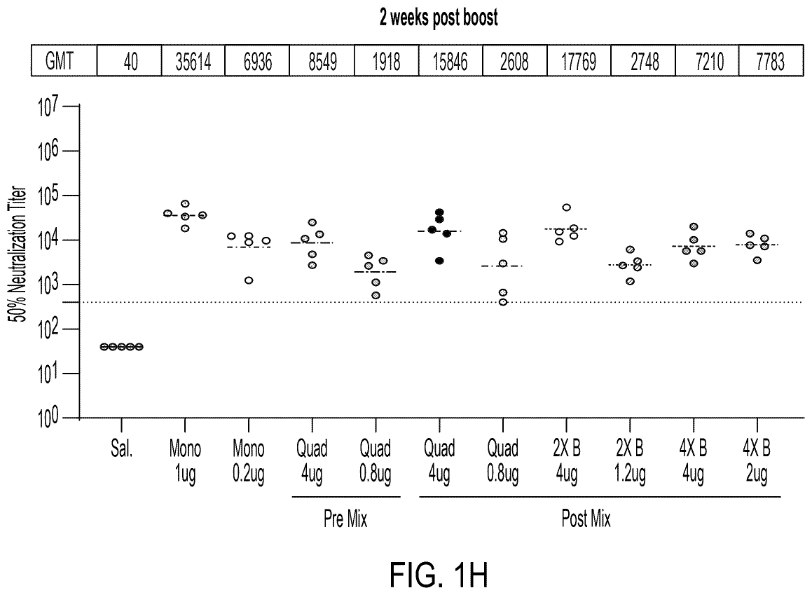

Intramuscular immunization of Balb/c mice with LNP formulated modRNA monovalent or quadrivalent vaccines encoding HA antigens from the four recommended NH 21-22 season strains (H1N1 A/Wisconsin/588/2019, H3N2 A/Cambodia/e0826360/2020, B/Phuket/3073/2013 (Yamagata), and B/Washington/02/2019 (Victoria)) induced functional antibody responses as measured by MNT (FIG. 1). HAI was performed but overall titers were low except for H1N1 A/Wisconsin, which made data interpretation challenging and therefore will not be included in this report.

MNT titers were induced by all the vaccine groups against the four strains with a robust boosting effect at Day 42. Comparable MNT titers were elicited by the quadrivalent modRNA mixes compared to each monovalent modRNA encoding H1N1 A/Wisconsin HA and H3N2 A/Cambodia HA at Day 21 and Day 42 (FIG. 1). Minimal difference in titers was observed between the pre-mix and post-mix quadrivalent formulations, although post mix formulations generally trended slightly higher at Day 42. In contrast to the Flu A strains, modest interference was observed for the Flu B strains, although the level of interference was dependent on dose level and time point. For example, MNT titers induced by the quadrivalent vaccine against B/Phuket were comparable to those elicited by the corresponding monovalent modRNA at Day 21, but trended lower at Day 42. Against B/Washington, MNT titers for the high dose (4 μg total) quadrivalent vaccines were reduced almost 3-fold compared to the 1 μg monovalent modRNA-HA control on Day 21, and slight interference for both the low and high dose groups were also observed on Day 42. Quadrivalent titers could be rescued when Flu B HA concentrations were increased 2× and 4× relative to Flu A HA, but mostly for the low dose vaccine groups.

FIG. 1—Female Balb/c mice were immunized IM on Day 0 and Day 28 with a high (1 μg or 4 μg) or low (0.2 μg or 0.8 μg) dose of either a monovalent LNP-formulated modRNA encoding HA from one of the four vaccine strains (H1N1 A/Wisconsin/588/2019, H3N2 A/Cambodia/e0826360/2020, By/Phuket/3073/2013, Bv/Washington/02/2019) or a modRNA (pre) or LNP (post) quadrivalent mix. Also tested were quadrivalent mixes with increased relative B strain concentrations at either 2× (4 μg=0.66 μg H1, H3/1.32 μg By, Bv; 1.2 μg=0.2 μg H1, H3/0.4 μg By, Bv) or 4× (4 μg=0.4 μg H1, H3/1.6 μg By, Bv; 2 μg=0.2 μg H1, H3/0.8 μg By, Bv) doses. Functional antibody responses against all four strains were measured by 1-day MNT on Day 21 (3 weeks post prime) and Day 42 (2 weeks post boost). 50% Neutralization titers are reported.

Functional antibody responses were produced against all four NH 21-22 strains (H1N1 A/Wisconsin/588/2019, H3N2 A/Cambodia/e0826360/2020, By/Phuket/3073/2013, Bv/Washington/02/2019) when Balb/c mice were vaccinated with a high (1 μg/4 μg) or low (0.2 μg/0.8 μg) dose of an LNP-formulated modRNA-HA monovalent or quadrivalent vaccine. Pre-mixed (modRNA) and post-mixed (LNPs) quad-valent constructs were similarly immunogenic for all four strains. At Day 21, MNT titers against H3N2 A/Cambodia, B/Phuket and B/Washington were lower than those against H1N1 A/Wisconsin, but a robust boost effect was observed for all four strains at two weeks after the second dose. Modest interference was detected in the quadrivalent titers against both B strains. However, this interference was counteracted at the low dose when concentrations of the B modRNA-HA were increased to 2 or 4 times the Flu A H1/H3 concentration. Importantly, the immunogenicity against the A strains was maintained at these modified doses. An effective quadrivalent modRNA Influenza vaccine may potentially include an adjusted Flu B dose.

### Example9: In Vitro Expression in Human Cells-HA Variants of Influenza B Virus with CT modifications, including truncation, addition, and cysteine mutations

### In Vitro Expression (IVE) Imaging Assay:

To characterize and evaluate the mutant constructs described herein, an in vitro Expression (IVE) Imaging assay has been developed in Hela cells: Cells are transfected with mRNA encoding wild type or mutant influenza B HA as DS (‘drug substance’)+Lipofectamine or DP (‘drug product’) in LNPs (Lipid Nanoparticles), then HA expression is examined at 24-48 hours by immunofluorescence (data not shown, wherein an 11-pt concentration response (in duplicate; 384 Well) showed increased HA expression for a HA variant compared to WT [anti-HA primary antibody/AlexaFluor-488 secondary antibody].).

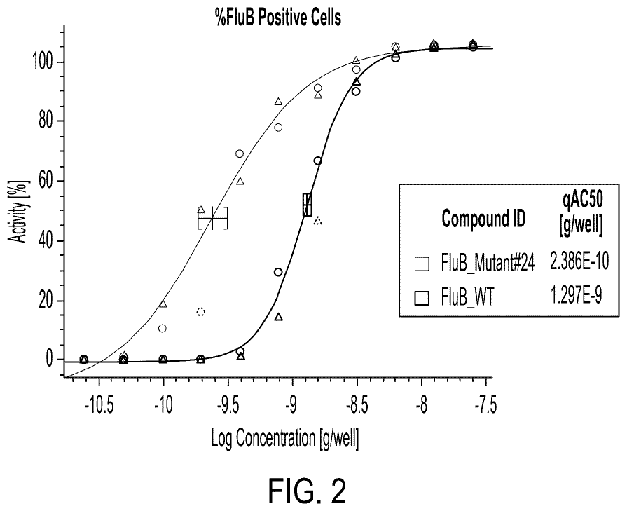

Several primary antibodies that bind to different portions of the HA ‘head’ or ‘stem’ region are used to evaluate/characterize influenza B HA expression. The images are analyzed with Signals Image Artist software, and multiple endpoints are calculated including MFI (mean fluorescence intensity), cell count (as a measure of toxicity/cell death) and % FluB positive cells. FIG. 2 shows an example of a mutant with improved IVE.

After analysis, Genedata Screener software is used to generate curves and calculate EC50s with WT B/Austria/1359417/2021 HA serving as the benchmark.

TABLE 38

| | | | DS IVE | DS IVE | DS IVE |
|---|---|---|---|---|---|
| | | | EC₅₀ | EC₅₀ | EC₅₀ |
| | | | (ng/well) | (ng/well) | (ng/well) |
| | | | in HeLa | In HeLa | In HeLa |
| Construct | Design | | based on | based on | based on |
| No. | Category | Description | B295 | CR8071 | CR9114 |
| WT (SEQ ID | Reference | B/Austria/1359417/2021- | 8.39E−10 | 1.21E−09 | 1.89E−09 |
| NO: 9) | | HA \| WT | | | |
| pBv-001 | CT | Δ(572-582) | 1.64E−09 | 1.91E−09 | 2.75E−09 |
| (SEQ ID NO: | modification | | | | |
| 10) | | | | | |
| pBv-002 | | V571C | 5.92E−10 | 5.99E−10 | 1.57E−09 |
| (SEQ ID NO: | | | | | |
| 11) | | | | | |
| pBv-003 | | S577C | 8.25E−10 | 9.73E−10 | 1.96E−09 |
| (SEQ ID NO: | | | | | |
| 12) | | | | | |
| pBv-004 | | S579C | 1.01E−10 | 1.65E−10 | 3.63E−10 |
| (SEQ ID NO: | | | | | |
| 13) | | | | | |
| pBv-005 | | L582C | 6.32E−10 | 6.47E−10 | 1.83E−09 |
| (SEQ ID NO: | | | | | |
| 14) | | | | | |
| pBv-006 | | V571C, S572C | 2.07E−10 | 3.68E−10 | 4.64E−10 |
| (SEQ ID NO: | | | | | |
| 15) | | | | | |
| pBv-007 | | V571C, S579C | 6.82E−10 | 8.10E−10 | 1.82E−09 |
| (SEQ ID NO: | | | | | |
| 16) | | | | | |
| pBv-008 | | N575C, V576C | 1.01E−09 | 1.17E−09 | 2.71E−09 |
| (SEQ ID NO: | | | | | |
| 17) | | | | | |
| pBv-009 | | V571C, S572C, S577C | 1.02E−09 | 1.33E−09 | 2.10E−09 |
| (SEQ ID NO: | | | | | |
| 18) | | | | | |
| pBv-010 | | V571C, S572C, S579C | 3.25E−10 | 3.87E−10 | 6.60E−10 |
| (SEQ ID NO: | | | | | |
| 19) | | | | | |
| pBv-011 | | R573C, D574C, S579C | 1.71E−10 | 2.24E−10 | 3.94E−10 |
| (SEQ ID NO: | | | | | |
| 20) | | | | | |
| pBv-012 | | R573C, D574C, | 1.87E−10 | 3.33E−10 | 5.41E−10 |
| (SEQ ID NO: | | S579C, +KGCCSCGSCC | | | |
| 21) | | | | | |

### Example10: In Vitro Expression in Human Cells-HA Variants of Influenza B Virus with deletion close to the cleavage site

TABLE 39

| | | | DS IVE | DS IVE | DS IVE |
|---|---|---|---|---|---|
| | | | EC₅₀ | EC₅₀ | EC₅₀ |
| | | | (ng/well) | (ng/well) | (ng/well) |
| | | | in HeLa | In HeLa | In HeLa |
| Construct | Design | | based on | based on | based on |
| No. | Category | Description | B295 | CR8071 | CR9114 |
| WT (SEQ ID | Reference | B/Austria/1359417/2021- | 8.39E−10 | 1.21E−09 | 1.89E−09 |
| NO: 9) | | HA \| WT | | | |
| pBv-013 | Deletion Near | D361, D378 | 1.82E−10 | 1.67E−10 | 4.43E−10 |
| (SEQ ID NO: | the Cleavage | | | | |
| 22) | Site | | | | |
| pBv-014 | | Δ(352-357) | 1.11E−09 | 1.36E−09 | 2.21E−09 |
| (SEQ ID NO: | | | | | |
| 23) | | | | | |
| pBv-015 | | Δ(352-359) | 8.63E−10 | 9.47E−10 | 1.88E−09 |
| (SEQ ID NO: | | | | | |
| 24) | | | | | |
| pBv-016 | | Δ(352-362) | 4.40E−10 | 5.72E−10 | 1.52E−09 |
| (SEQ ID NO: | | | | | |
| 25) | | | | | |
| pBv-017 | | Δ(352-365) | 1.21E−09 | 1.18E−09 | 1.83E−09 |
| (SEQ ID NO: | | | | | |
| 26) | | | | | |
| pBv-018 | | Δ(352-368) | 1.36E−09 | 1.41E−09 | 2.85E−09 |
| (SEQ ID NO: | | | | | |
| 27) | | | | | |
| pBv-019 | | Δ(352-370) | 1.34E−09 | 1.53E−09 | 2.73E−09 |
| (SEQ ID NO: | | | | | |
| 28) | | | | | |
| pBv-020 | | Δ(354-365) | 7.40E−11 | 7.71E−11 | 2.50E−10 |
| (SEQ ID NO: | | | | | |
| 29) | | | | | |
| pBv-021 | | Δ(354-366) | 1.32E−10 | 1.00E−10 | 3.10E−10 |
| (SEQ ID NO: | | | | | |
| 30) | | | | | |
| pBv-022 | | Δ(355-358) | 1.92E−09 | 2.43E−09 | 3.71E−09 |
| (SEQ ID NO: | | | | | |
| 31) | | | | | |
| pBv-023 | | Δ(355-361) | 1.38E−10 | 2.17E−10 | 3.28E−10 |
| (SEQ ID NO: | | | | | |
| 32) | | | | | |
| pBv-024 | | Δ(355-363) | 9.65E−11 | 1.13E−10 | 2.53E−10 |
| (SEQ ID NO: | | | | | |
| 33) | | | | | |
| pBv-025 | | Δ(355-364) | 1.47E−10 | 1.58E−10 | 4.41E−10 |
| (SEQ ID NO: | | | | | |
| 34) | | | | | |
| pBv-026 | | Δ(355-367) | 1.30E−10 | 1.59E−10 | 3.68E−10 |
| (SEQ ID NO: | | | | | |
| 35) | | | | | |
| WT (SEQ ID | Reference | B/Austria/1359417/2021- | 8.39E−10 | 1.21E−09 | 1.89E−09 |
| NO: 9) | | HA \| WT | | | |
| pBv-027 | Deletion Near | D(356-363) | 1.90E−10 | 2.07E−10 | 6.35E−10 |
| (SEQ ID NO: | the Cleavage | | | | |
| 36) | Site | | | | |
| pBv-028 | | D(358-362) | 5.23E−10 | 6.63E−10 | 1.22E−09 |
| (SEQ ID NO: | | | | | |
| 37) | | | | | |
| pBv-029 | | Δ(359-361) | 1.13E−09 | 1.16E−09 | 1.95E−09 |
| (SEQ ID NO: | | | | | |
| 38) | | | | | |
| pBv-030 | | Δ(359-363) | 4.89E−10 | 5.71E−10 | 1.24E−09 |
| (SEQ ID NO: | | | | | |
| 39) | | | | | |
| pBv-031(SEQ | | D(359-364) | 1.45E−10 | 1.49E−10 | 3.51E−10 |
| ID NO: 40) | | | | | |
| pBv-032 | | Δ(361-364) | 1.47E−10 | 1.87E−10 | 3.32E−10 |
| (SEQ ID NO: | | | | | |
| 41) | | | | | |
| pBv-033 | | Δ(361-367) | 2.10E−10 | 2.58E−10 | 4.67E−10 |
| (SEQ ID NO: | | | | | |
| 42) | | | | | |
| pBv-034 | | Δ(364-367) | 1.46E−10 | 1.66E−10 | 2.89E−10 |
| (SEQ ID NO: | | | | | |
| 43) | | | | | |
| pBv-035 | | D(364-369) | 5.31E−11 | 8.38E−11 | 1.97E−10 |
| (SEQ ID NO: | | | | | |
| 44) | | | | | |
| pBv-036 | | Δ(364-370) | 2.12E−10 | 2.75E−10 | 4.45E−10 |
| (SEQ ID NO: | | | | | |
| 45) | | | | | |
| pBv-037 | | Δ(366-373) | 1.27E−10 | 1.69E−10 | 3.78E−10 |
| (SEQ ID NO: | | | | | |
| 46) | | | | | |
| pBv-038 | | Δ(369-373) | 1.70E−10 | 1.84E−10 | 3.83E−10 |
| (SEQ ID NO: | | | | | |
| 47) | | | | | |

### Example11: In Vitro Expression in Human Cells-HA Variants of Influenza B Virus with hydrophobic mutations close to the cleavage site

TABLE 40

| | | | DS IVE EC₅₀ | DS IVE EC₅₀ | DS IVE EC₅₀ |
|---|---|---|---|---|---|
| | | | (ng/well) | (ng/well) | (ng/well) |
| | | | in HeLa | In HeLa | In HeLa |
| Construct | | | based on | based on | based on |
| No. | Design Category | Description | B295 | CR8071 | CR9114 |
| WT (SEQ ID | Reference | B/Austria/1359417/2021- | 8.39E−10 | 1.21E−09 | 1.89E−09 |
| NO: 9) | | HA \| WT | | | |
| pBv-039 | Hydrophobic | R359A, G360A | 2.92E−10 | 4.58E−10 | 1.17E−09 |
| (SEQ ID NO: | Mutations Near the | | | | |
| 48) | Cleavage Site | | | | |
| pBv-040 | | F361A | 2.86E−10 | 2.38E−10 | 5.70E−10 |
| (SEQ ID NO: | | | | | |
| 49) | | | | | |
| pBv-041 | | F362A | 2.49E−10 | 2.98E−10 | 6.97E−10 |
| (SEQ ID NO: | | | | | |
| 50) | | | | | |
| pBv-042 | | I365A | 2.30E−10 | 2.09E−10 | 5.48E−10 |
| (SEQ ID NO: | | | | | |
| 51) | | | | | |
| pBv-043 | | F368A | 1.34E−10 | 1.30E−10 | 4.82E−10 |
| (SEQ ID NO: | | | | | |
| 52) | | | | | |
| pBv-044 | | L369A | 1.74E−10 | 2.33E−10 | 5.88E−10 |
| (SEQ ID NO: | | | | | |
| 53) | | | | | |
| pBv-045 | | G360P, F361P, I365G | 1.49E−10 | 2.36E−10 | 3.28E−10 |
| (SEQ ID NO: | | | | | |
| 54) | | | | | |

### Example12: In Vitro Expression in Human Cells-HA Variants of Influenza B Virus with combination of CT modifications and deletions close to the cleavage site

TABLE 41

| | | | DS IVE EC₅₀ | DS IVE EC₅₀ | DS IVE EC₅₀ |
|---|---|---|---|---|---|
| | | | (ng/well) | (ng/well) | (ng/well) |
| | | | in HeLa | In HeLa | In HeLa |
| Construct | | | based on | based on | based on |
| No. | Design Category | Description | B295 | CR8071 | CR9114 |
| WT (SEQ ID | Reference | B/Austria/1359417/2021- | 8.39E−10 | 1.21E−09 | 1.89E−09 |
| NO: 9) | | HA \| WT | | | |
| pBv-046 (SEQ | Combination of | D(355-361), S579C | 2.20E−10 | 3.09E−10 | 4.77E−10 |
| ID NO: 55) | CT modification | | | | |
| pBv-047 (SEQ | and deletion | D(361-364), S579C | 2.66E−10 | 2.72E−10 | 4.19E−10 |
| ID NO: 56) | close to the | | | | |
| pBv-048 (SEQ | cleavage site | D(361-367), S579C | 2.19E−10 | 2.35E−10 | 3.90E−10 |
| ID NO: 57) | | | | | |
| pBv-049 (SEQ | | D(364-367), S579C | 4.03E−10 | 4.82E−10 | 6.54E−10 |
| ID NO: 58) | | | | | |
| pBv-050 (SEQ | | D(366-373), S579C | 1.22E−10 | 1.31E−10 | 2.98E−10 |
| ID NO: 59) | | | | | |
| pBv-051 (SEQ | | D(369-373), S579C | 1.41E−10 | 1.37E−10 | 3.46E−10 |
| ID NO: 60) | | | | | |
| pBv-052 (SEQ | | D(369-373), V571C, S572C | 1.09E−10 | 1.78E−10 | 5.13E−10 |
| ID NO: 61) | | | | | |
| pBv-053 (SEQ | | D(369-373), L582C | 9.15E−11 | 1.28E−10 | 2.47E−10 |
| ID NO: 62) | | | | | |
| pBv-054 (SEQ | | D(355-361), R573C, D574C, | 6.21E−11 | 1.30E−10 | 2.35E−10 |
| ID NO: 63) | | S579C | | | |
| pBv-055 (SEQ | | D(361-364), R573C, D574C, | 7.44E−10 | 6.50E−10 | 1.31E−09 |
| ID NO: 64) | | S579C | | | |
| pBv-056 (SEQ | | D(361-367), R573C, D574C, | 2.44E−10 | 2.88E−10 | 6.21E−10 |
| ID NO: 65) | | S579C | | | |
| pBv-057 (SEQ | | D(364-367), R573C, D574C, | 5.82E−10 | 6.90E−10 | 1.30E−09 |
| ID NO: 66) | | S579C | | | |
| pBv-058 (SEQ | | D(366-373), R573C, D574C, | 2.45E−10 | 1.94E−10 | 6.66E−10 |
| ID NO: 67) | | S579C | | | |
| pBv-059 (SEQ | | D(369-373), R573C, D574C, | 1.77E−10 | 2.24E−10 | 4.49E−10 |
| ID NO: 68) | | S579C | | | |

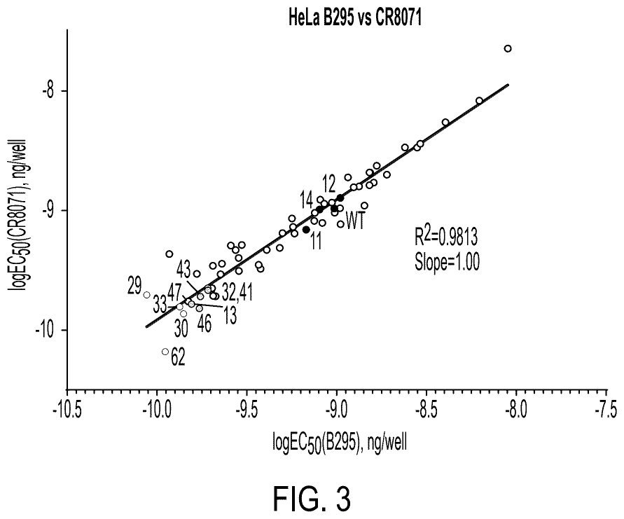

Examples 13. Correlation of In Vitro Expression in Human Hela Cells determined using monoclonal antibody CR8071 vs polycolonal antibody B295-HA variants of Influenza virus. Selected variants are labeled with their SEQ NO IDs. See FIG. 3.

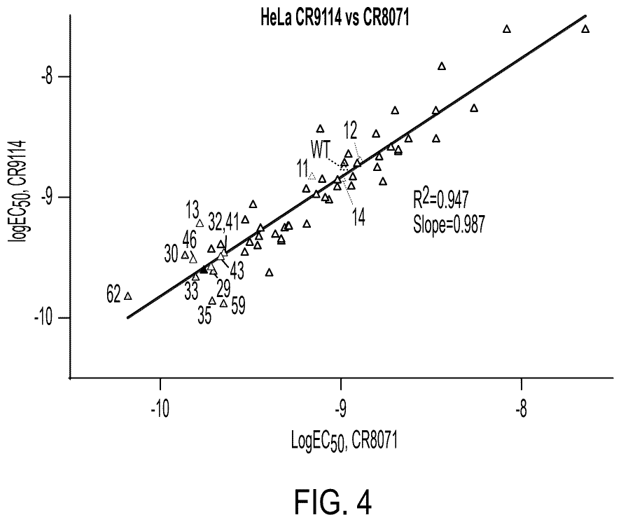

Examples 14. Correlation of In Vitro Expression in Human Hela Cells measured using monoclonal antibodies CR8071 and CR9114 of HA variants of Influenza virus. Selected variants are labeled with their SEQ NO IDs. CR8071 and CR9114 recognize different epitopes of the HA. See FIG. 4.

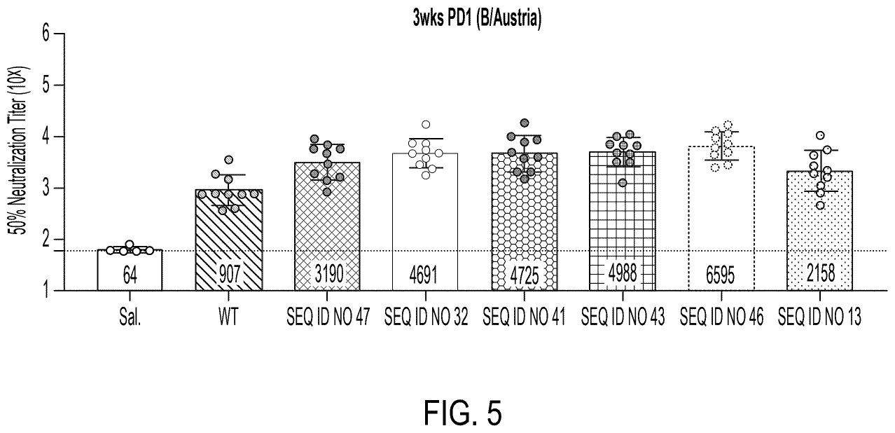

Example 15. Functional Anti-HA Antibodies Elicited by Immunization of Mice With Monovalent or Quadrivalent LNP-Formulated modRNA Encoding Influenza HA as Measured By MNT In vivo immunogenicity elicitated by HA variants of infleunza B virus determined by neutralization antibody titer, 3 weeks post dose 1. See FIG. 5.

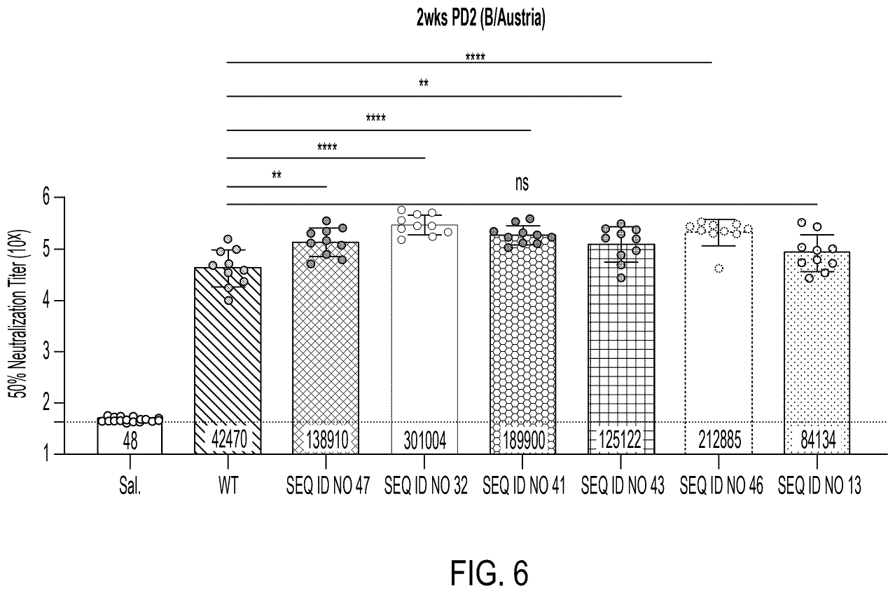

Example 16. In vivo immunogenicity eicitated by HA variants of infleunza B virus measured by neutralization antibody titer, 2 weeks post dose 2. See FIG. 6

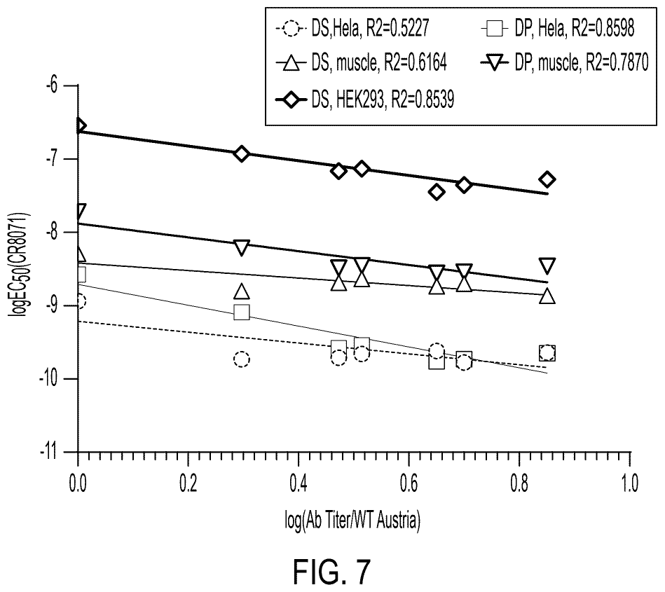

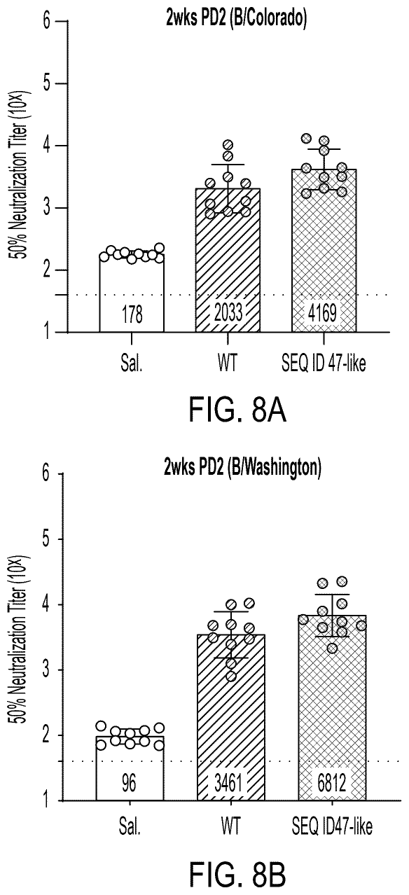

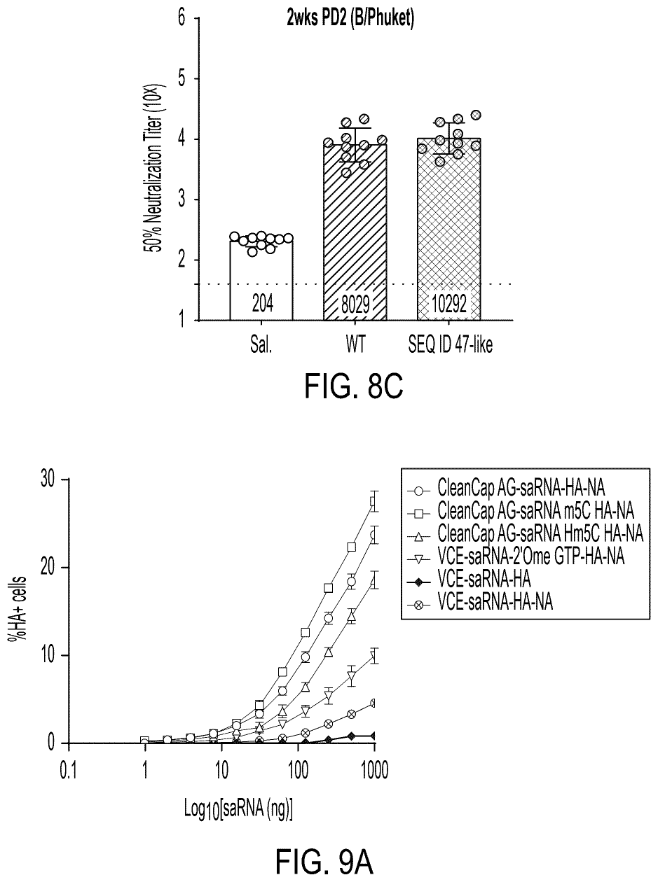

Example 17. Correlation of In vivo immunogenicity elicitated by HA variants of infleunza B virus measured by neutralization antibody titer, 2 weeks post dose 2, and their in vitro expression in heman cells. DS, drug substrances; DR, Drug product. See FIG. 7 Example 18. Same design principle can be applied to HA of other influenza B virus strains to improve the immunogenicity against those virus strains. In this example, the HA variant with the mutationa equivalent to SEQ ID NO: 47 was engineered in Washington and Colorado sublineage of Victory strain of influenza B virus, and Phuket sublineage of Yamagata strain. Improvements on immunogenicity measured by neutralization antibody titers were observed. See FIG. 8A-C.

### Example19: In Vitro Expression of Hemagglutinin (HA) from LNPs Formulated with alternative cholesterols

The objective of this Example was to assess in vitro expression (IVE) of GFP (reporter) from lipid nanoparticles (LNPs) formulated with alternative sterols and/or N/P ratios. Percentage of encapsulation efficiency (% EE, as used herein throughout), polydispersity index (PDI), and % of intact mRNA as measured by fragment analyzer (FA) are shown in Table 41.

TABLE 41

| | Formulation | % Cationic | % Helper | % | N/P | Flow | | | | FA % |
|---|---|---|---|---|---|---|---|---|---|---|
| # | description | Lipid | Lipid | Cholesterol | Ratio | Rate | % EE | Size | PDI | intact |
| 1 | Chol | ALC315 47.5 | DSPC 10 | Chol 40.7 | 6 | 30/10 | 97.2 | 66.2 | 0.109 | 85 |
| | benchmark-NP6 | | | | | | | | | |
| 2 | Chol-NP10 | ALC315 47.5 | DSPC 10 | Chol 40.7 | 10 | | 98.2 | 74.9 | 0.077 | 85 |
| 3 | sito/chol mix | ALC315 47.5 | DSPC 10 | Sito 24.4/ | 6 | | 94.6 | 95.8 | 0.039 | 86 |
| | (6:4)-NP6 | | | Chol 16.3 | | | | | | |
| 4 | sito/chol mix | ALC315 47.5 | DSPC 10 | Sito 24.4/ | 10 | | 94.4 | 95.0 | 0.012 | 84 |
| | (6:4)-NP10 | | | Chol 16.3 | | | | | | |
| 5 | sito/chol mix | ALC315 33.5 | DSPC 17.5 | Sito 28.3/ | 6 | | 98.9 | 104.2 | 0.036 | 84 |
| | Ic3-NP6 | | | Chol 18.9 | | | | | | |
| 6 | sito/chol mix | ALC315 33.5 | DSPC 17.5 | Sito 28.3/ | 10 | | 97.3 | 91.5 | 0.018 | 83 |
| | (6:4) Ic3-NP10 | | | Chol 18.9 | | | | | | |

N/P10 LNPs (i.e., LNPs having an N: P ratio of 10) performed better than N/P6 LNPs (i.e., LNPs having an N: P ratio of 6) in IVE in HEK293T cells, with LC3 mix formulation showing the largest enhancement and cholesterol showing the slightest improvement.

The following is an exemplary method to assess the presence and determine the in-vitro expression of mRNA-encoded target protein. Human embryonic kidney (HEK293T) cells are seeded on one to two 12-well culture plates per assay instance and transfected with control and drug product (DP) test samples across two assay instances. After 21-24 hours, cells are harvested from the 12-well plates and transferred to 96-well assay plates. The cells are stained with fixable aqua viability dye before being permeabilized and fixed. After the fixative is washed from the cells, a fluorophore-conjugated HA antibody cocktail is added which binds to influenza HA antigens. The cells are then analyzed for HA expression via flow cytometry, which detects the fluorescent signal of the fluorophore conjugated to the strain-specific anti-HA antibodies. The in-vitro expression of the HA antigen is determined from the average percent of viable, single cells bound with fluorophore-conjugated HA antibody.

NP6 and NP10 LNPs with FluB/Austria mod-HA had similar in DP analytics and IVE. In Table 42, various sterol mix ratios are tested. The LNPs encapsulated modified mRNA encoding GFP. Decreasing sitosterol percentage in the sterol mixture appeared to result in slightly higher % EE and smaller LNP size. Sito/chol 1:1 mixture showed slightly better EC50 than the other two ratios.

TABLE 42

| | Formulation | % Cationic | % Helper | % | N/P | Flow | % | | | FA % |
|---|---|---|---|---|---|---|---|---|---|---|
| # | description | Lipid | Lipid | Cholesterol | Ratio | Rate | EE | Size | PDI | intact |
| 1 | sito/chol mix | ALC315 47.5 | DSPC 10 | Sito 24.4/ | 10 | 30/10 | 94.4 | 95.0 | 0.012 | 84 |
| | (6:4) std-fast | | | Chol 16.3 | | | | | | |
| | rate-NP10 | | | | | | | | | |
| 2 | sito/chol mix | ALC315 47.5 | DSPC 10 | Sito 20.4/ | 10 | 30/10 | 96.8 | 91.1 | 0.026 | 80 |
| | (5:5) std-fast | | | Chol 20.4 | | | | | | |
| | rate-NP10 | | | | | | | | | |
| 3 | sito/chol mix | ALC315 47.5 | DSPC 10 | Sito 16.3/ | 10 | 30/10 | 97.3 | 87.1 | 0.038 | 84 |
| | (4:6) std-fast | | | Chol 24.4 | | | | | | |
| | rate-NP10 | | | | | | | | | |

In other studies, cholesterol derivatives were observed to increase IVE (˜5-10 times) of LNPs encapsulated with RMM72 NA/Wisconsin modRNA. See Table 43.

TABLE 43

| | | | | | IVE | |
|---|---|---|---|---|---|---|
| | | | | | IVE % | |
| | FA | | DLS | | Cells | IVE |
| | % | | Size | EE | positive | EC50 |
| Description | Integrity | LMS | (nm) | % | (125 ng) | (ng/well) |
| β-Sitosterol | 88 | 3 | 152 | 56 | 89 | 14 |
| Campesterol | 87 | 3 | 94 | 87 | 87 | 23 |
| Fucosterol (Delta | 90 | 3 | 144 | 80 | 94 | 15 |
| 5-avenasterol) | | | | | | |
| Stigmastanol | NT | NT | 124 | 36 | NT | NT |
| (sitostanol) | | | | | | |
| Stigmasterol | NT | NT | 82 | 7 | NT | NT |
| CholPC | NT | NT | 110 | 17 | NT | NT |
| Cholesterol | 88 | 3 | 77 | 94 | 50 | 131 |

See also Table 44 for data showing IVE of various sterol combinations, wherein the LNP encapsulated RMM72 NA/Wisconsin modRNA. In some preferred embodiments, the sterol comprises Cholesterol: 3-Sitosterol 4:6 LNP.

TABLE 44

| | FA | | DLS | | IVE | |
|---|---|---|---|---|---|---|
| | % | | Size by DLS, | EE | % Positive | IVE EC50 |
| Description | Integrity | LMS | Z-Ave(nm) | % | @ 125 ng | (ng/well) |
| Cholesterol:β- | 90 | 2 | 150 | 45 | 93 | 11 |
| Sitosterol 0:1 | | | | | | |
| Cholesterol:β- | 87 | 4 | 176 | 45 | 95 | 8 |
| Sitosterol 1:9 | | | | | | |
| Cholesterol:β- | 89 | 2 | 159 | 47 | 85 | 32 |
| Sitosterol 2:8 | | | | | | |
| Cholesterol:β- | 88 | 3 | 116 | 76 | 83 | 28 |
| Sitosterol 3:7 | | | | | | |
| Cholesterol:β- | 92 | 1 | 91 | 89 | 94 | 8 |
| Sitosterol 4:6 | | | | | | |
| Cholesterol:β- | 91 | 2 | 91 | 86 | 83 | 63 |
| Sitosterol 10:0 | | | | | | |

LNP encapsulated HA/California modRNA comprising Cholesterol: 3-Sitosterol 4:6 showed the excellent combination of integrity, EE and IVE compared to other formulations. See Table 45, wherein Lipid ratio tested was 47.5:40.7:10:1.8 (ALC-0315: Cholesterol/cholesterol analog: DSPC: ALC-0159); N/P=6; LNP Matrix had 10 mM Tris, 300 mM Sucrose, PH 7.4. LNP encapsulated HA/California modRNA using Cholesterol: β-Sitosterol of 4:6 showed a preferred embodiment of integrity, EE and IVE compared to other tested formulations.

TABLE 45

| | | | Size by | | | | IVE |
|---|---|---|---|---|---|---|---|
| | % | LMS | DLS, Z- | | EE | % Positive | EC50 |
| Cholesterol type | Integrity | NMT | Ave(nm) | PDI | % | @ 125 ng | (ng/well) |
| β-Sitosterol | 79 | 3% | 165 | 0.11 | 26 | 81.3 | 4 |
| Cholesterol:β- | 60 | 3% | 166 | 0.08 | 30 | 84.4 | 3 |
| Sitosterol 1:9 | | | | | | | |
| Cholesterol:β- | 83 | 3% | 137 | 0.08 | 54 | 82.8 | 4 |
| Sitosterol 2:8 | | | | | | | |
| Cholesterol:β- | 79 | 3% | 108 | 0.08 | 86 | 77.5 | 6 |
| Sitosterol 3:7 | | | | | | | |
| Cholesterol:β- | 85 | 3% | 106 | 0.07 | 86 | 85.1 | 4 |
| Sitosterol 4:6 | | | | | | | |
| Cholesterol:β- | 85 | 3% | 104 | 0.1 | 90 | 79.8 | 6 |
| Sitosterol 5:5 | | | | | | | |
| Cholesterol:β- | 80 | 3% | 107 | 0.12 | 75 | 81.1 | 6 |
| Sitosterol 6:4 | | | | | | | |
| Cholesterol:β- | 84 | 3% | 99 | 0.11 | 88 | 79.1 | 5 |
| Sitosterol 7:3 | | | | | | | |
| Campesterol | 86 | 3% | 106 | 0.07 | 87 | 72.6 | 7 |
| (batch 1) | | | | | | | |
| Campesterol | 76 | 3% | 111 | 0.13 | 80 | 81.2 | 4 |
| (batch 2) | | | | | | | |
| Fucosterol | 80 | 3% | 157 | 0.1 | 79 | 86.3 | 3 |
| LNP control (pure | 84 | 3% | 94 | 0.17 | 94 | 84.7 | 5 |
| cholesterol) | | | | | | | |

LNP encapsulated GFP modRNA comprising Cholesterol: β-Sitosterol 4:6 in 293T and HeLa cells were tested and EC50 observed. See Table 46, wherein Lipid ratio tested was 47.5:40. 7:10:1.8 (ALC-0315: Cholesterol/cholesterol analog: DSPC: ALC-0159); N/P=6; LNP Matrix had 10 mM Tris, 300 mM Sucrose, pH 7.4. LNP encapsulated modGFP using Cholesterol: β-Sitosterol of 4:6 showed a preferred embodiment of about 10 times improvement of EC50 as compared to, for example, the cholesterol formulation.

TABLE 46

| | % | % | % | Z-Ave | | EC50 in | EC50 in |
|---|---|---|---|---|---|---|---|
| Description | Cholesterol | EE | FA | (d · nm) | PDI | 293T | Hela |
| Cholesterol | Chol 40.7 | 94 | 88 | 77 | 0.054 | 6.1 | 9.1 |
| Campesterol | Cam 40.7 | 59 | 88 | 97 | 0.033 | 1.4 | 1.4 |
| β-Sitosterol | Sito 40.7 | 49 | 87 | 154 | 0.07 | 1.5 | 1.9 |
| Cholesterol:β- | Chol 16.3/ | 76 | 88 | 96 | 0.069 | 0.63 | 0.67 |
| Sitosterol = 0.4:0.6 | Sito 24.4 | | | | | | |

### Example20: SaRNA Synthesized with Cap Analogs, m7G-R/Ym-G Improves Self-Amplification of nucleoside-(un) modified, gene of therapeutic interest-encoding saRNA

We investigated whether incorporation of cap analogs, in a configuration of m7G (7-methylguanine)-Xm-G into saRNA alone and/or in combination with modified nucleotides can result in saRNA with enhanced biological properties. We found that a full-length saRNA can be synthesized and co-transcriptionally capped in the presence of the cap analogs, m7G-R/Ym-G (Table 47 and Table 48) and that the in-vitro transcribed saRNA has a higher replication capacity and concomitant increase in gene/antigen expression than saRNA capped with vaccinia capping enzymatic system when tested in human immune cells (Figure). We also tested a modified RNA polymerase promoter for saRNA synthesis to increase the co-transcriptional capping efficiency. saRNA transcription under the promoter is initiated by R/Ym-G docking onto the +1 and +2 template nucleotides and results in saRNA transcripts with >90% capping efficiency (Table 49). The co-transcriptional saRNA capping can also be done with modified nucleotides.
Current T7 RNA polymerase promoter for saRNA synthesis: TACGACTCACTATAG
   saRNA sequence: m7G-GmAUA . . .
Cap analogs tested: CLEANCAP AG; CLEANCAP AG (3′ Ome); CLEANCAP m6AG (3′ Ome)
Capping efficiency: 76-88%
Modified T7 RNA polymerase promoter for saRNA synthesis: TACGACTCACTATAAG
   saRNA sequence: m7G-AmGAUA . . .
Cap analogs tested: CLEANCAP AG; CLEANCAP AG (3′ Ome); CLEANCAP m6AG (3′ Ome)
Capping efficiency: 91-100%

Functional saRNA can be synthesized using cap analogs using any RNA polymerase promoter ending with TACGACTCACTATAG as opposed to TACGACTCACTATAAG as the former is compatible with any cap analog ending with G (i.e, much broader IP coverage independent of penultimate nucleotide next m7G).

### Methods:

In vitro transcription of saRNA and characterization

Capped saRNAs encoding the Hemagglutinin (HA) and Neuraminidase (NA) from A/Wisconsin/588/2019/H1N1 were in-vitro transcribed, purified, and analyzed by RNaseH digestion followed by LC-MS as previously described. In brief, transcriptions were performed at 33° C. for 2 hours using 9 mmol/l trinucleotide cap1 analogs, CLEANCAP AG (TriLink), T7 RNA polymerase, and nucleotide triphosphates at 11.25 mmol/l final concentration. To obtain saRNAs with modified nucleosides, the transcription reaction was assembled with the replacement of one or two nucleotide triphosphate with the corresponding triphosphate derivative of the following modified nucleosides: 5-methylcytidine (m5C), 5-hydroxymethylcytidine (Hm5C), 2′-O-methylguanosine (2′Ome-G) or N1-methylpseudouridine (m14′) (TriLink). Purification of the transcripts were performed by Turbo DNase digestion followed by LiCI precipitation. The quantity and quality of the saRNAs were determined with a Nanodrop spectrophotometer. All RNA samples were analyzed by LC-MS for capping efficiency.

Cell-based assays for assessing saRNA performance

THP-1 cells were seeded at 250,000 cells per well in 24-well plates and placed in an incubator at 37° C., with 5% CO₂ prior to saRNA transfections. RNA was diluted to the targeted working concentrations in water (DNase/RNase free) prior to complexation with the lipid-based transfection reagent, Lipofectamine RNA MessengerMax (MMax), in Opti-MEM, at a ratio of 1 μg saRNA per 2.25 μl MMax. Following lipoplex formation, THP-1 cells were transfected with the respective saRNA materials in a 2-fold, 11-point dilution series, starting with 1000 ng saRNA/250,000 cells as the highest dose. Following transfection, cells were allowed to rest for 22 hrs, before processing the samples for data acquisition via flow cytometry. Data regarding the percentage of antigen (HA) positive cells, relative HA expression (geometric mean fluorescence intensity (GMFI)), and cell viability were recorded. All samples were run in biological triplicate and data plotted is represented as mean, +/−SD.

THP-1 cells were transfected with either saRNA-TC83-A/Wisc/588/19 HA-40A or bicistronic saRNA-TC83-A/Wisc/588/19 HA-NA-80A either with no nucleoside modifications, m5C, Hm5C, or 2′Ome-G incorporation (11-point, 2-fold dilution series starting from 1000 ng). Number of HA expressing cells (% HA+ cells) (A), total HA expression per HA positive cell (Geometric mean fluorescence intensity (GMFI)) (B), and number of live cells (% live cells) (C), were determined by flow cytometry at 22 hrs post transfection. Results are presented as mean±standard deviation for each group from a representative experiment.

### Results:

saRNA synthesis and capping efficiency

In a series of transcription reactions using GOI-encoding plasmids and an RNA polymerase, we obtained full-length transcripts containing either no nucleoside modifications, m1Y′, m5C, Hm5C, or 2′Ome-G nucleoside modifications. All in-vitro transcribed transcripts had capping efficiency of ˜80%. When a DNA template with a modified T7 RNA polymerase promoter to accommodate the penultimate nucleoside of the trimer cap analog, >90% capping efficiency was achieved.

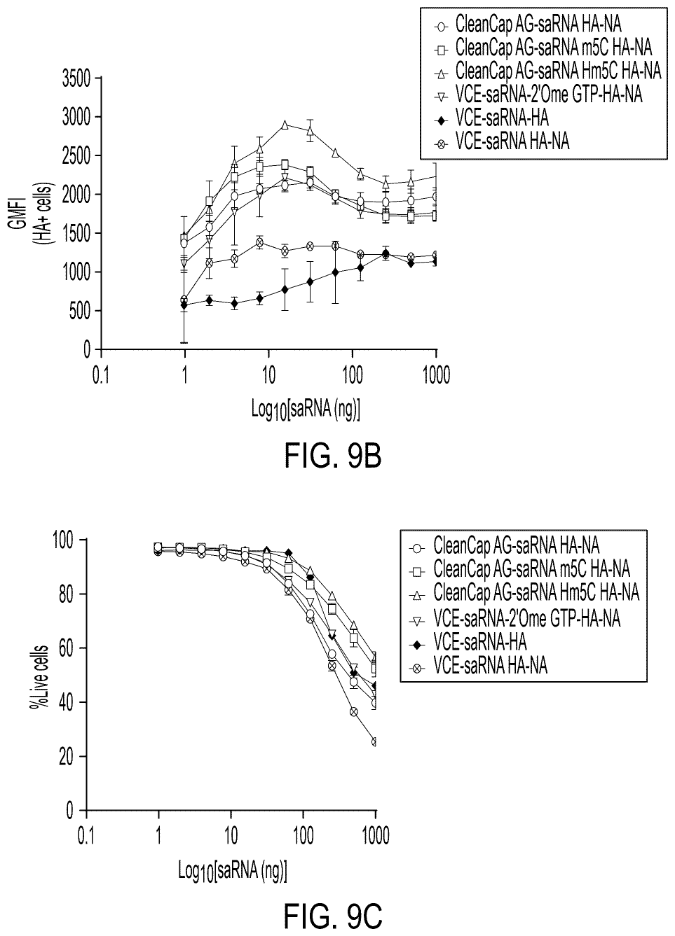

Translational efficacy of saRNA with CLEANCAP AG and/or nucleoside modification Across all saRNA test conditions, those in vitro transcribed using co-transcriptional capping, exhibited higher percentages of total cells expressing the vaccine gene of interest (GOI), when compared to those capped enzymatically (VCE). saRNA generated with CLEANCAP AG and m5C modified nucleoside had the highest overall transfection efficiency; however, the CLEANCAP AG construct containing Hm5C exhibited the highest overall GMFI from HA immunostaining, across all but the lowest dilutions. The unmodified saRNA (both mono and bicistronic) exhibited the lowest overall transfection efficiencies as well as GMFIs and had similar or inferior viability when compared to the CLEANCAP counterparts. See FIG. 9A-B. Overall, these data indicate that saRNA generated using a co-transcriptional capping process with CLEANCAP AG increases vaccine GOI expression in human monocytic cells.

TABLE 47
Bicistronic saRNA synthesis with CleanCap AG and/or modified nucleotides

| Sample | | Cap | Modified | Concentration | % |
|---|---|---|---|---|---|
| ID | DNA Template Description | analog | nucleoside | (ng/ml) | Capping |
| 00804232- | (TC83_No_Kozak_HA_Wisc_SGP_NA_ | AG | n/a | 6954 | 80% |
| 0016A | Wisc_80A) | | | | |
| 00804232- | T7 RNA polymerase promoter: | AG | 100% | 5828 | 83% |
| 0016B | TACGACTCACTATAG | | m5C | | |
| 00804232- | | AG | 100% | 5592 | 83% |
| 0016C | | | Hm5C | | |
| 00804232- | | n/a | 75% | 2359 | 98% |
| 0003 | | (VCE) | 2′Om-G | | |

TABLE 48
Bicistronic saRNA synthesis with alternative CleanCap AGs

| Sample | | Cap | Modified | Concentration | % |
|---|---|---|---|---|---|
| ID | DNA Template Description | analog | nucleoside | (ng/ml) | Capping |
| 00806702- | (TC83_No_Kozak_HA_Wisc_SGP_ | AG | n/a | 2886.5 | 81% |
| 0002- | NA_Wisc_80A) | | | | |
| 01 | T7 RNA polymerase promoter: | | | | |
| 00806702- | TACGACTCACTATAG | AG | n/a | 3577 | 76% |
| 0002- | | (3′Ome) | | | |
| 02 | | | | | |
| 00806702- | | m6AG | n/a | 642.7 | 88% |
| 0002- | | (3′Ome) | | | |
| 03 | | | | | |

TABLE 49
Bicistronic saRNA synthesis with alternative CleanCap AGs and modified
nucleotides

| Sample | | Cap | Modified | Concentration | % |
|---|---|---|---|---|---|
| ID | DNA Template Description | analog | nucleoside | (ng/ml) | Capping |
| 00806702- | (TC83_No_Kozak_HA_Wisc_SGP_ | AG | n/a | 2186 | 98% |
| 0003- | NA_Wisc_80A) | | | | |
| 01 | T7 RNA polymerase promoter: | | | | |
| 00806702- | TACGACTCACTATAAG | | 50% | 2008 | 100% |
| 0003- | | | m1Y | | |
| 02 | | | | | |
| 00806702- | | | 100% | 1653 | 98% |
| 0003- | | | m5C | | |
| 03 | | | | | |
| 00806702- | | AG | n/a | 4094 | 94% |
| 0003- | | (3′OMe) | | | |
| 04 | | | | | |
| 00806702- | | | 50% | 2052 | 96% |
| 0003- | | | m1Y | | |
| 05 | | | | | |
| 00806702- | | | 100% | 1930 | 91% |
| 0003- | | | m5C | | |
| 06 | | | | | |
| 00806702- | | | 75% | 802 | 96% |
| 0003- | | | 2′OMe- | | |
| 07 | | | G | | |
| 00806702- | | m6AG | n/a | 322 | 95% |
| 0003- | | (3′OMe) | | | |
| 08 | | | | | |
| 00806702- | | | 50% | 835 | 98% |
| 0003- | | | m1Y | | |
| 09 | | | | | |

### Example21: Flu B/Austria DP w/6:4 b-Sito: Cholesterol; Freeze/Thaw after Incorporation of sodium oleate

LNP formulations with b-sitosterol may be improved to tolerate freeze/thaw stresses and lyophilization. An experiment was performed to assess whether sodium oleate could mitigate potential impact from freeze/thaw stresses and lyophilization, with a focus on particle size change.

A pre-made Flu modRNA-encapsulated LNP formulation comprising b-sitosterol: cholesterol (6:4 molar ratio) was further re-formulated to incorporate sodium oleate at 800 mcg/mL. This formulation along with a control formulation underwent 1×, 3× freeze/thaw (F/T) cycles and lyophilization.

“Control LNP” in the present example refers to a pre-made Flu modRNA-encapsulated LNP formulation comprising b-sitosterol: cholesterol (6:4 molar ratio), as described in Example 19. Results show that sodium oleate appears to reduce impact of the freeze/thaw-induced stress.

TABLE 50
Results

| Sample Name | Z-Ave (d · nm) | PDI |
|---|---|---|
| Control LNP (never frozen) | 87.44 | 0.041 |
| Control LNP (1X F/T) | 102.1 | 0.091 |
| Control LNP (3X F/T) | 108.7 | 0.11 |
| LNP w/sodium oleate (never frozen) | 92.06 | 0.069 |
| LNP w/sodium oleate (1X F/T) | 91.97 | 0.065 |
| LNP w/sodium oleate (3X F/T) | 92.75 | 0.053 |

### Example22: Flu B/Austria DP w/6:4 b-Sito: Cholesterol; Lyophilization after incorporation of sodium oleate

LNP formulations with b-sitosterol may be improved to tolerate freeze/thaw stresses and lyophilization. An experiment was performed to assess whether sodium oleate could mitigate potential impact from lyophilization, with a focus on particle size change.

A pre-made Flu modRNA-encapsulated LNP formulation comprising b-sitosterol: cholesterol (6:4 molar ratio) was further re-formulated to incorporate sodium oleate at 800 mcg/mL and was lyophilized.

“Control LNP” in the present example refers to a pre-made Flu modRNA-encapsulated LNP formulation comprising b-sitosterol: cholesterol (6:4 molar ratio), as described in Example 19. Results show that sodium oleate appears to reduce particle size changes from lyophilization.

TABLE 51
Results

| Sample Name | Z-Ave (d · nm) | PDI |
|---|---|---|
| Control LNP (never frozen) | 87.44 | 0.041 |
| Lyophilized Control LNP | 149.3 | 0.151 |
| LNP w/sodium oleate (never frozen) | 92.06 | 0.069 |
| Lyophilized LNP w/sodium oleate | 98.96 | 0.1 |

### Example23: MRNA-LNP with Sitosterol and Sphingomyelin for Enhanced Efficacy

mRNA-LNPs were formulated by combining an mRNA-containing aqueous phase and a lipid containing organic phase using a T-mixer. The organic phase was prepared by solubilizing a mixture of ionizable lipid, sphingomyelin, polyethylene glycolipid, and cholesterol analogue at a pre-determined ratio in ethanol. The organic phase and aqueous phase were mixed by syringe pumps. The resulting solution was dialyzed against 10 mM Tris buffer (pH 7.4) or 1×DPBS (pH 7.4) for 18-20 h. Post-dialysis solution was concentrated and filtered to a final mRNA-LNP

### Solution

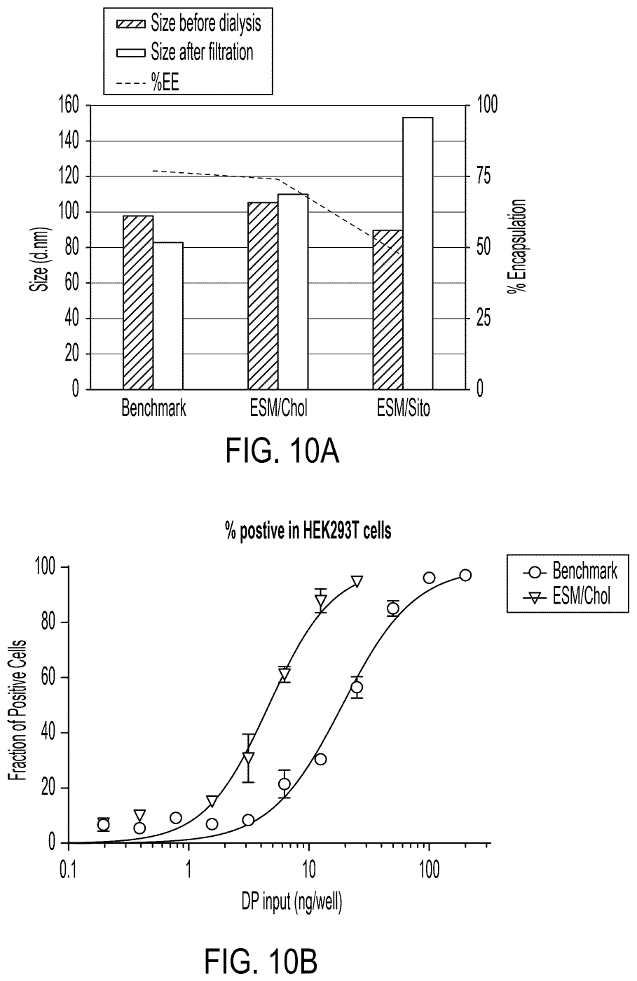

Formulations shown in table were tested for in vitro expression of GFP and assessed for LNP size before dialysis in the LNP formation process and assessed for size after filtration in the LNP formation process. See FIG. 10A. modGFP-LNP with high level of sphingomyelin (SM) exhibited improved IVE. See FIG. 10B.

TABLE 52

| Formulation | Cat % | Helper lipid % | Chol % | Note |
|---|---|---|---|---|
| Benchmark | 47.5 | DSPC 10 | Cholesterol | |
| | | | 40.7 | |
| DSPC/Chol | 30 | DSPC 40 | Cholesterol | Lipid mix remained |
| | | | 28.2 | partially undissolved |
| DSPC/Sito | 30 | DSPC 40 | Sitosterol 28.2 | |
| egg | 30 | sphingomyelin | Cholesterol | |
| sphingomyelin | | (SM) 40 | 28.2 | |
| (ESM)/Chol | | | | |
| ESM/Sito | 30 | SM 40 | Sitosterol 28.2 | |
| ESM/Stig | 30 | SM 40 | Stigmasterol | Lipid mix remained |
| | | | 28.2 | partially undissolved |

TABLE 53
Exemplary Sphingomyelin compounds

| Abbre- | Sphingo- | |
|---|---|---|
| viation | myelin | Molecule Structure |
| SM-01 | 12:0 (d18:1/ 12:0) | 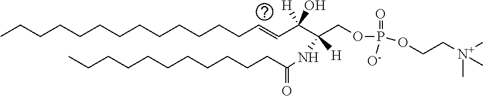 [SMILES: CCCCCCCCCCCCC/C=C/[C@@H](O)[C@H](COP(=O)([O-])OCC[N+](C)(C)C)NC(=O)CCCCCCCCCCC] |
| SM-02 | 14:0 (d18:1/ 14:0) | 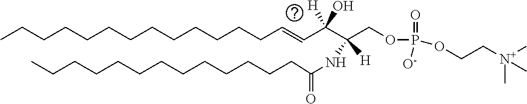 [SMILES: CCCCCCCCCCCCC/C=C/[C@@H](O)[C@H](COP(=O)([O-])OCC[N+](C)(C)C)NC(=O)CCCCCCCCCCCCC] |
| SM-03 (or SM) | 16:0 (d18:1/ 16:0) | 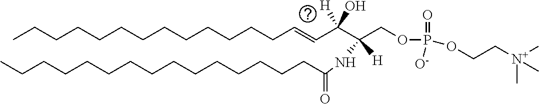 [SMILES: CCCCCCCCCCCCC/C=C/[C@@H](O)[C@H](COP(=O)([O-])OCC[N+](C)(C)C)NC(=O)CCCCCCCCCCCCCCC] |
| SM-04 | 16:1 (d18:1/ 16:1 (9Z)) | 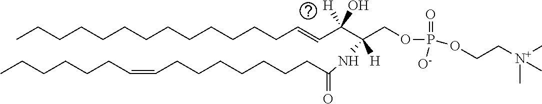 [SMILES: CCCCCC/C=C\CCCCCCCC(=O)N[C@@H](COP(=O)([O-])OCC[N+](C)(C)C)[C@H](O)/C=C/CCCCCCCCCCCCC] |
| SM-05 | 18:1 (d18:1/ 18:1 (9Z)) | 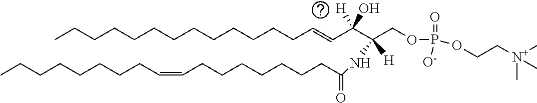 [SMILES: CCCCCCCC/C=C\CCCCCCCC(=O)N[C@@H](COP(=O)([O-])OCC[N+](C)(C)C)[C@H](O)/C=C/CCCCCCCCCCCCC] |
| SM-06 | 24:0 (d18:1/ 24:0) | 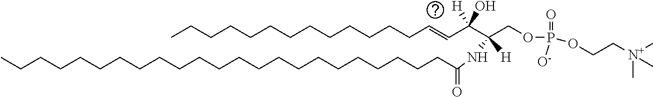 [SMILES: CCCCCCCCCCCCC/C=C/[C@@H](O)[C@H](COP(=O)([O-])OCC[N+](C)(C)C)NC(=O)CCCCCCCCCCCCCCCCCCCCCCC] |
| SM-07 | 18:0 (d18:1/ 18:0) | 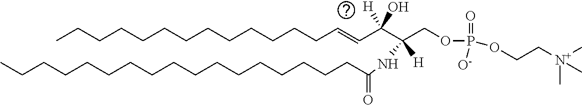 [SMILES: CCCCCCCCCCCCC/C=C/[C@@H](O)[C@H](COP(=O)([O-])OCC[N+](C)(C)C)NC(=O)CCCCCCCCCCCCCCCCC] |

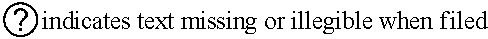

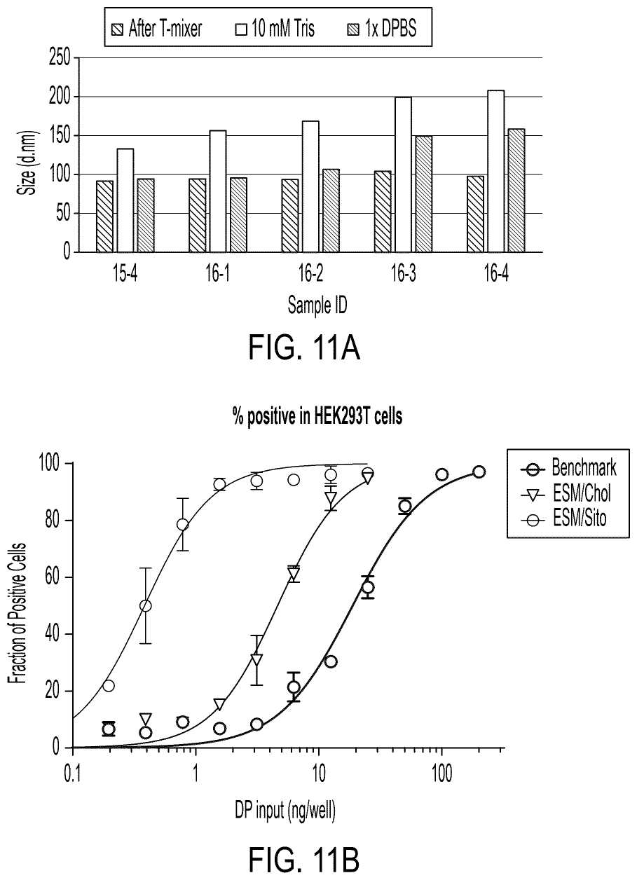

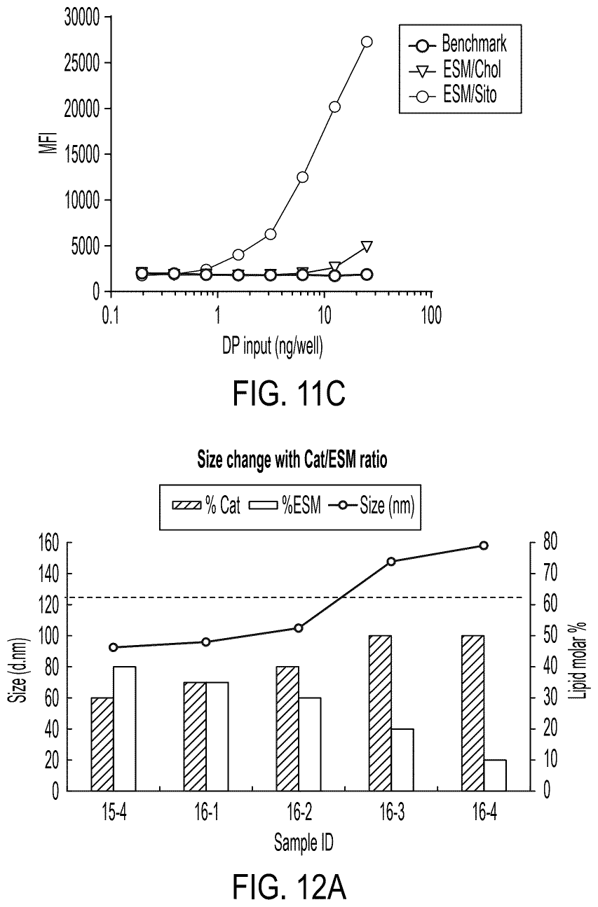

Assessing buffers in LNP formation process. Successful combination of sitosterol and SM can be achieved using PBS as buffer. See FIG. 11A-C.

TABLE 54

| Formulation | Cationic lipid % | Helper lipid % |
|---|---|---|
| 15-4 | 30 | SM 40 |
| 16-1 | 35 | SM 35 |
| 16-2 | 40 | SM 30 |
| 16-3 | 50 | SM 20 |
| 16-4 | 50 | SM 10 |

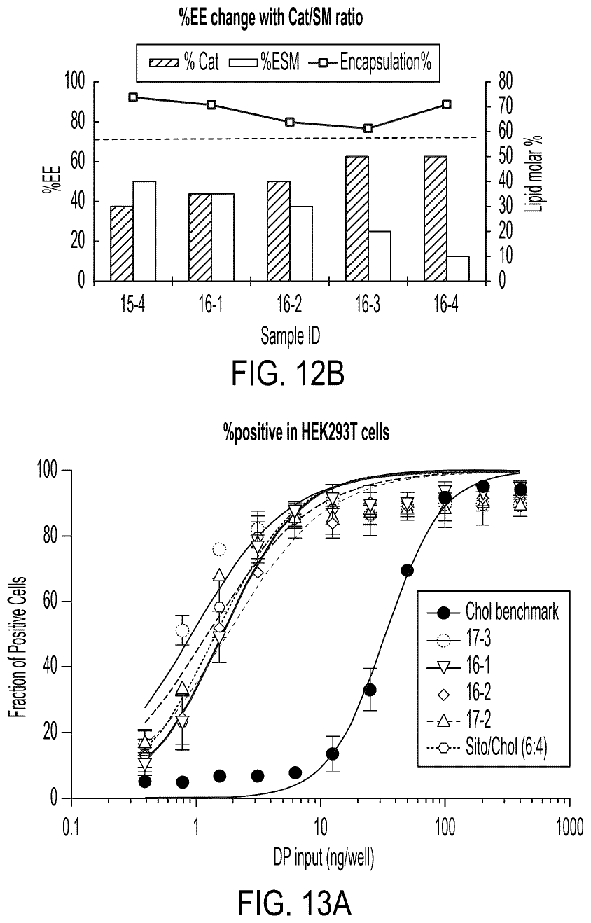

LNP size and % EE (encapsulation) appears to be associated with the cationic/SM ratio. See FIG. 12A-B.

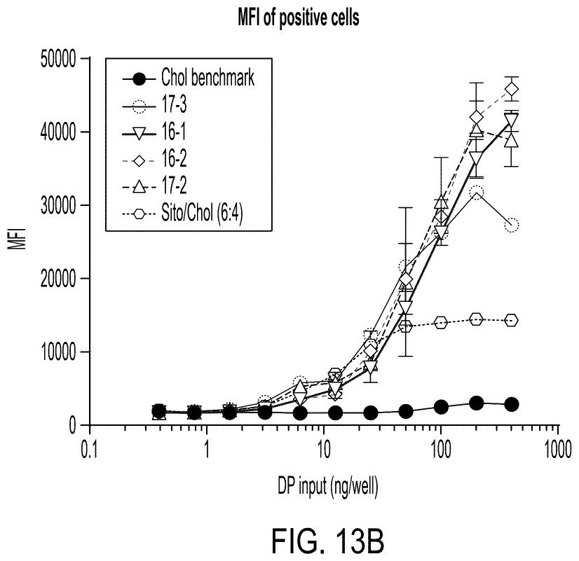

Significant increase in MFI was observed in sito/SM formulations comparing to sitosterol/chol (6:4). See FIG. 13A-B and Table 55.

TABLE 55

| | | Helper | | Size | | % |
|---|---|---|---|---|---|---|
| Formulation | Cat % | lipid % | PEG % | (nm) | PDI | EE |
| Chol benchmark | 47.5 | DSPC 10 | ALC-159 1.8 | 64.6 | 0.082 | 98 |
| 17-3 | 30 | SM 40 | ALC-159 1.8 | 100.5 | 0.084 | 96 |
| 16-1 | 35 | SM 35 | ALC-159 1.8 | 95.95 | 0.058 | 88 |
| 16-2 | 40 | SM 30 | ALC-159 1.8 | 104.9 | 0.083 | 80 |
| 17-2 | 35 | SM 35 | PC11K 0.4 | 120.3 | 0.080 | 90 |
| Sito/Chol (6:4) | 47.5 | DSPC 10 | ALC-159 1.8 | 95.8 | 0.040 | 95 |

### Example24: Preclinical Immunogenicity and Safety of Hemagglutinin-Encoding modRNA influenza vaccines

We describe the preclinical immunogenicity and safety of influenza modRNA vaccines (IRV), encoding full-length HA from seasonal human influenza strains, that utilize the modRNA technology and LNP formulation. Formulations of monovalent (mIRV), trivalent (tIRV), and quadrivalent (qIRV) modRNA-HA vaccines were tested for in vitro expression and immunogenicity in multiple animal species (mice, rhesus and cynomolgus macaques). Additionally, safety of both mIRV and qIRV was assessed in Wistar Han rats.

### Materials and Methods

Formulation of the Monovalent, Trivalent, and Quadrivalent modRNA-HA Vaccines

ModRNA-HA vaccines encoding full-length HA proteins of WHO-recommended strains for cell culture- or recombinant-based vaccines for use in either the 2021-2022 (A/Wisconsin/588/2019 (H1N1), A/Cambodia/e0826360/2020 (H3N2), B/Washington/02/2019 (B/Victoria), and B/Phuket/3073/2013 (B/Yamagata)) or 2022-2023 (A/Wisconsin/588/2019 (H1N1), A/Darwin/6/2021 (H3N2), B/Austria/1359417/2021 (B/Victoria), and B/Phuket/3073/2013 (B/Yamagata)) northern hemisphere influenza seasons were individually formulated and prepared as either monovalent, trivalent, or quadrivalent formulations as previously described for the BNT162b2 modRNA vaccine. Stock concentrations of modRNA-HA were diluted in saline to achieve the desired dose for vaccine administration in animals.

In Vitro Expression of HA from the Influenza modRNA-HA Vaccine

Individually LNP-formulated modRNAs encoding full-length HA from H1N1, H3N2, B/Victoria, or B/Yamagata strains were diluted in Opti-MEM (Thermo Fisher, Cat #31985062) and directly added to a HEK-293T (CRL-3216, ATCC) cell monolayer at four RNA dose levels (62.5, 31.1, 15.6, and 7.8 ng/well). The input amount of mRNA encoding the strain-specific HA was the same between modRNA formulations (e.g., different amounts of mIRV and qIRV were applied to cells to achieve a final concentration of 62.5 ng/well of mRNA encoding the B/Phuket/3073/2013 HA). Opti-MEM media alone was used as a negative control. Protein expression was measured with a flow cytometer (BD FACS Fortessa) using in-house generated rabbit polyclonal antibodies raised against each of the following strains: A/Wisconsin/588/2019 (H1N1), A/Darwin/6/2021 (H3N2), B/Austria/1359417/2021 (B/Victoria), or B/Phuket/3073/2013 (B/Yamagata), followed by a secondary anti-rabbit antibody conjugated to Alexa-Fluor 488 (Invitrogen, Cat #A-11008). The percentage of live cells expressing the strain-specific HA protein was enumerated and expression was measured by quantifying the number of live cells that had a positive signal for bound anti-HA antibody.

### Animals

Mouse immunogenicity studies utilized female BALB/c mice (The Jackson Laboratory) that were first immunized between 7-13 weeks of age. NHP immunization studies utilized female rhesus macaques (Macaca mulatta) and cynomolgus macaques (Macaca fasicularis) that were co-housed in standard quad caging. Rhesus and cynomolgus macaques were immunized at 5.5 and 13.5-15.5 years of age, respectively. For mIRV and qIRV toxicology studies, male and female Wistar Han rats, 11 weeks of age at the dosing study start.

### Mouse Immunization Study Design

Experimental vaccine groups each contained 10 female BALB/c mice. Control groups consisted of 5 or 10 female BALB/c mice that were administered 50 μL of saline intramuscularly (IM). For all studies, mice were immunized IM twice, on Days 0 and 28. Groups receiving modRNA-HA vaccines received 0.2 μg of each mRNA HA construct. Thus, mIRV vaccines were administered at a 0.2 μg dose in a 50 μL volume; tIRV was administered at a 0.6 μg dose (0.2 μg/HA) in a 50 μL volume; and qIRV vaccines were administered at a 0.8 μg dose (0.2 μg/HA) in 50 μL. An undiluted comparator QIV (Fluad®) was administered IM at a final concentration of 2.4 μg in a 20 μL dose volume (1/25th of the human dose), also on Days 0 and 28. Whole blood was collected on Day 21 (3 weeks post-dose 1) and Day 42 (2 weeks post-dose 2) and evaluated for levels of functional anti-HA antibodies by serology. Splenocytes were isolated on Day 42 (study end) to measure the T cell-mediated immune response following immunization.

### NHP Immunization Study Design

Rhesus and cynomolgus macaques (3 animals/group) were each immunized IM with a 30 μg dose of mIRV (A/Wisconsin/588/2019) in a 0.5 mL total volume. Whole blood was collected on Days-7 (pre-vaccination), 7, 21, 28, 35, 42, 77, 105, 133, and 168 and evaluated for levels of functional anti-HA antibodies by serology. PBMCs were isolated on Days-7 (pre-vaccination), 7, 35, 42, 77, 105, 133 and 168 to measure T cell responses following immunization.

### Toxicology Study Design

For both toxicology studies, male and female Wistar Han rats (15/sex/group) were randomly assigned to Groups 1 or 2, and doses were administered IM by 2 separate injections on Days 1 and 15. Group 1 was administered sterile saline. Group 2 was administered with either 34 μg mIRV or 30 μg qIRV (7.5 μg/HA). A subset of animals (10/sex/group) was euthanized 2 days after the second dose (Day 17), while the remaining animals (5/sex/group) underwent an approximate 3-week recovery and then were euthanized (Day 38-39). Serum samples were collected from each animal prior to dose initiation and on Day 17 (dosing phase) and Day 21 (recovery phase) for analysis of hemagglutination inhibition, to confirm in parallel functional immunogenicity of the mIRV and qIRV under toxicological observation.

Samples for clinical pathology analysis in toxicology studies (hematology, coagulation, clinical chemistry, acute phase proteins and urinalysis) were collected on Days 3 (nonterminal; hematology, clinical chemistry, and acute phase proteins only), 17 (terminal), and 39 (terminal), from overnight fasted animals. For non-terminal collections, hematology was assessed in the first 7 animals/sex/group and clinical chemistry was assessed in the last 8 animals/sex/group. Phlebotomy sites included the jugular vein (non-terminal bleed) or aorta under isoflurane anesthesia followed by exsanguination (terminal bleed).

### Animal Blood Collection and Splenocyte Isolation

For mouse immunization studies, the interim bleed was conducted using a submandibular bleeding technique. At the study end, blood was collected via cardiac puncture (terminal bleed). Whole blood tubes remained at room temperature (RT) for at least 30 minutes prior to centrifuging at 10,000 RPM for 3 minutes for sera collection. Samples for HAI and MNT assays were treated using a receptor destroying enzyme (RDE) kit (Accurate Chemical), heat inactivated and pre-adsorbed with turkey red blood cells (RBCs). Samples were stored at −80° C. until testing. At the study end, spleens were collected from 5 mice per group and separately placed in a 70 μm cell strainer (Fisher) immersed in 7 mL of complete RPMI (cRPMI: 10% FBS/RPMI; Pen-Strep; Sodium pyruvate; HEPES; MEM-NEAA; Amphotericin B) per well of a 6-well plate. Plates were maintained on ice during transit and before processing for single cell suspension. Spleens were homogenized, subjected to RBC lysis, and passed through a cell strainer to remove RBCs and clumps.

For NHP immunization studies, blood for serum and PBMC isolation was collected in BD Vacutainer® SST™ tubes and K2 EDTA 5.4 mg tubes, respectively, while animals were safely restrained. Whole blood samples were centrifuged at 3000 RPM for 10 minutes and sera were collected and stored at −80° C. until testing. Blood for PBMC isolation were retained at RT until processed. Diluted whole blood was layered on 90% Ficoll (GE Healthcare) to isolate PBMCs via density gradient centrifugation at 800×g. PBMCs were then frozen in Gibco Recovery™ Cell Culture Freezing Medium at 5×10⁶ cells/vial and stored in liquid nitrogen until analysis.

### Viruses

All viruses used for testing were rescued using a reverse genetics system. In brief, an eight-plasmid system was applied for virus rescue. Each bidirectional plasmid encoded one of the eight segmented genes of influenza virus. The sequences for HA and NA genes were strain-specific and the six influenza backbone genes were subtype-specific (IAV or IBV). For IAV, sequences for the backbone genes of PA, PB2, NP, NS, and M were from the A/Puerto Rico/8/1934 (H1N1) (PR8) strain while PB1 sequence was from the A/California/07/2009 (H1N1) strain. For IBV, all six backbone genes were from B/Brisbane/60/2008 (B/Vic). The pool of 2 μg of each plasmid in OptiMEM medium (Gibco #31985) was co-transfected into a co-culture of HEK-293T and MDCK cells (1:1 ratio) with Lipofectamine 2000 (Invitrogen) for 4 hours at 37° C. followed by replacement of media with OptiMEM supplemented with 1 μg/mL of TPCK-treated trypsin. The viruses were harvested at 72-hour post-transfection and propagated twice in MDCK cells with multiplicity of infection (MOI) of 0.001-0.01 for passage 1 and MOI of 0.0001-0.001 for passage 2. Passage 1 viruses served as virus seeds and passage 2 viruses served as viral stocks for HAI and MNT assay testing, described below.

### Hemagglutination Inhibition Assay (HAI)

modRNA-HA vaccine-induced functional anti-HA antibodies that prevent HA-mediated agglutination of RBCs were measured using the hemagglutination inhibition assay (HAI). All sera were pre-treated with RDE, heat-inactivated, and then pre-adsorbed with appropriate RBCs to remove any non-specific agglutinins. 2-fold serial dilutions of mouse or NHP sera, tested in duplicate, in PBS were mixed with the vaccine matched influenza virus strain on a shaker for 5 minutes then left to incubate for 30 minutes at RT. The neutralization reaction was then mixed with either turkey or guinea pig RBCs (Lampire Biological Laboratories) and incubated an additional 30 or 60 minutes, respectively, at RT. Assay plates were imaged on a FluHema (SciRobotics). The HAI titer was reported as the reciprocal of the highest serum dilution resulting in loss of HA activity, visualized as a full smear reaching the bottom of the well with substantial footing when the microtiter plate was tilted 60° for 30 seconds, when using turkey RBCs. If guinea pig RBCs were used, loss of HA activity was observed as a pellet on the microtiter plate without tilting. All samples were run in duplicate.

For the toxicity studies, the HAI assay was conducted by VisMederi (Siena, Italy). In the VisMederi assay, sera collected from mIRV and qIRV immunized rats were pre-treated with RDE, heat-inactivated and then pre-adsorbed with appropriate RBCs to remove any non-specific agglutinins. The treated serum, tested in duplicate per sample, was serially titrated two-fold in a dilution plate starting at a 1:10 dilution. An equal volume of standardized influenza antigen, obtained from Francis Crick Institute (London, UK) and propagated by VisMederi Research (Siena, Italy), was added to the serum samples and the plates were incubated 60 minutes at RT. RBCs were then added to all wells, and plates were incubated further for 60 minutes at RT. Following the last incubation, the plates were tilted, and the titer was determined as the reciprocal of the highest serum dilution in which agglutination was still completely inhibited. The geometric mean of four titers per sample (two analysts+two readers) was reported for each influenza vaccine antigen.

### Microneutralization Test (MNT)

Anti-HA neutralizing antibody responses following vaccination were measured using a 1-day microneutralization assay (MNT). Sera were pre-treated with RDE and heat-activated prior to use in the MNT assay. Serial dilutions of either mouse or NHP sera were incubated in a flat bottom 96-well plate with the vaccine matched influenza virus strain for 1 hour at 37° C./5% CO₂. Adherent MDCK cells (MDCK NBL-2, ATCC CCL-34) were added in suspension on top of the neutralization reaction and incubated at 37° C./5% CO₂ for 18-20 hours. Cells were then fixed with methanol and stained with either Polyclonal Rabbit IgG Anti-Influenza A NP or Anti-Influenza B NP (Invitrogen) primary antibody followed by AlexaFluor 488 goat anti-rabbit IgG H+L (Life Technologies) secondary antibody. Infected cells were counted using a CTL ImmunoSpot S6 Universal-V Analyzer with ImmunoCapture Software (Cellular Technology Ltd). MNT titers were reported as the reciprocal of the dilution that resulted in 50% reduction in infection when compared to a no serum control. All samples were run in duplicate.

### Intracellular Cytokine Staining Assay

Vaccine-induced T cell responses to influenza were measured by flow cytometry-based intracellular cytokine staining assay (ICS). In mouse studies, freshly-isolated splenocytes (2×10⁶ cells/well) were cultured in cRPMI with media containing DMSO only (unstimulated) or a specific peptide pool representing HA sequences of the A/Wisconsin/588/2019 (H1N1) influenza virus strain (Mimotopes) for 5 hours at 37° C. in the presence of protein transport inhibitors, GolgiPlug and GolgiStop. Following stimulation, cells were stained for surface and intracellular markers to identify activated and/or cytokine-expressing T cell types (CD3+ cells for CD4 vs CD8), activation markers (CD154/CD40L), and cytokines (IFN-γ, IL-2, IL-4, TNF-α, CD154, and CD107a). The eBioscience™ fixable viability dye eFluor 506 (Invitrogen) was used prior to surface staining, per manufacturer's instructions, to exclude dead cells. After staining, the cells were washed and resuspended in flow cytometry buffer (2% FBS/PBS). Cells were acquired on a BD LSR Fortessa and data were analyzed using BD FlowJo™ software. Results are background (media-DMSO) subtracted and shown as a percentage of CD4+ T cells or CD8+ T cells.

In NHP studies, the ex vivo stimulation with HA peptides was performed, as described above, using PBMCs collected at different timepoints in place of splenocytes. Frozen PBMCs were thawed and rested for the ICS assay. Following stimulation, PBMCs were stained for surface and intracellular markers to identify IFN-γ-expressing T cells for both species (rhesus and cynomolgus macaques). Acquired data were analyzed as described above . . .

### Toxicology Observations and Measurements

For in-life assessments, during the Dosing Phase all animals were weighed twice prior to the initiation of dosing (PID) on Days 1 and 7, prior to dosing on Days 1 and 15, and on non-dosing Days 4, 8, and 11, and a fasted weight was collected just prior to scheduled necropsy. Body weights were collected on Recovery Phase Days 1, 8, 15, and 21. Clinical observations occurred at least once daily prior to the initiation of dosing, at least twice daily on non-dosing days and during the recovery phase, and prior to and after each dose on dosing days. Body temperatures were collected on all animals on Dosing Phase Days 1 and 15 prior to dosing and at approximately 4- and 24-hours post-dose. Injection sites were observed on Dosing Phase Days 1 and 15 prior to dosing and approximately 4- and 24-hours post-dose on all animals. Local reactions were assessed using the Draize scoring method in both studies.

Hematology was evaluated using a Siemens Advia 2120i analyzer (Siemens Healthineers Tarrytown, NY, USA). Fibrinogen activated partial thromboplastin time, and prothrombin time was evaluated on the Diagnostic Stago STA-R evaluation coagulation analyzer (Diagnostic Stago, Parsippany, NJ, USA). Blood smears were prepared for the first 7 animals on Day 3 and all animals on Day 17 and Day 39. Blood cell morphology was evaluated microscopically on 5 animals of each sex from all groups at both scheduled necropsies (i.e., at dosing and recovery phases). Routine clinical chemistry parameters were evaluated using a Siemens Advia 1800 clinical chemistry analyzer (Siemens Healthineers, Tarrytown, NY, USA). Acute phase proteins alpha-2 macroglobulin (A2M) and alpha-1-acid glycoprotein (A1AGP) were measured using the rat MSD Acute Phase Protein Panel 1 on the MSD SECTOR S 600 Analyzer (Meso Scale Design). Routine urinalysis parameters were measured, and a microscopic examination of sediment for formed elements was performed on 5 animals of each sex from all dose groups at both scheduled necropsies (i.e., dosing and recovery phases).

For post-mortem analysis, complete necropsies, tissue collection, organ weights, and macroscopic tissue evaluation were performed on all animals. Animals were euthanized by gas anesthesia (isoflurane) followed by exsanguination on Dosing Phase Day 17 (2 days after the last dose) or on Recovery Phase Day 22, the last day of the Recovery Phase (surviving animals). Necropsy, tissue collection, organ weights, macroscopic tissue evaluation, and microscopic examination were performed.

### Statistical Analysis

Animal immunogenicity data was analyzed using GraphPad PRISM software. GMTs of the immune responses for vaccine groups and strains were calculated and are displayed in each bar chart. An independent two sample t-test was performed to compare immune responses of two groups between mIRV or qIRV and QIV (Fluad®). An analysis of variance (ANOVA) was conducted to compare immune responses among mIRV, tIRV, qIRV, and QIV (Fluad®). All pairwise comparisons of the four groups were performed and Tukey's test was applied to adjust for multiple comparisons. All tests were two-tailed. A p-value less than 0.01 was considered statistically significant and is marked with asterisk(s) in the bar charts. Statistical analysis of body weight, body weight change, food consumption, body temperature, hematology and coagulation, clinical chemistry, acute phase proteins, urinalysis, relative and absolute organ weights, and injection site reactions were conducted. Descriptive statistics were generated for each parameter and group at each scheduled sampling time or each time interval. Statistical tests were conducted at the 5% and 1% significance levels. Analysis of body temperature was based on the maximum body temperature post injection for each animal. Analysis of injection site score was based on the average irritation score post-injection for each animal. A nonparametric (rank-transform) one-way ANOVA was conducted, with a 2-sided pairwise comparisons of each dose group to the reference group (saline control) using Dunnett' test. Average ranks were assigned to ties.

### Results

Monovalent, Trivalent, and Quadrivalent modRNA-HA Efficiently Express IAV and IBV HA Proteins In Vitro

modRNA constructs encoding codon-optimized, full-length HA proteins derived from A/Wisconsin/588/2019 (H1N1), A/Darwin/6/2021 (H3N2), B/Austria/1359417/2021 (B/Victoria), and B/Phuket/3073/2013 (B/Yamagata) were designed, synthesized, and encapsulated in LNPs. Expression of IAV and IBV HA from LNP-formulated mIRV, tIRV, and qIRV was confirmed in vitro. Influenza HA protein was expressed in HEK-293T cells in a dose-dependent manner from modRNA encoding the strain-specific HA antigen, as measured by flow cytometry. The data demonstrate that the HA protein can be efficiently expressed from modRNA-HA vaccines regardless of valency (mIRV, tIRV, qIRV) or HA type (IAV or IBV).

Mice Immunized with mIRV Exhibit Higher Functional Antibody Responses and Polyfunctional T Cell Responses Post-Boost as Compared to an Adjuvanted Inactivated QIV

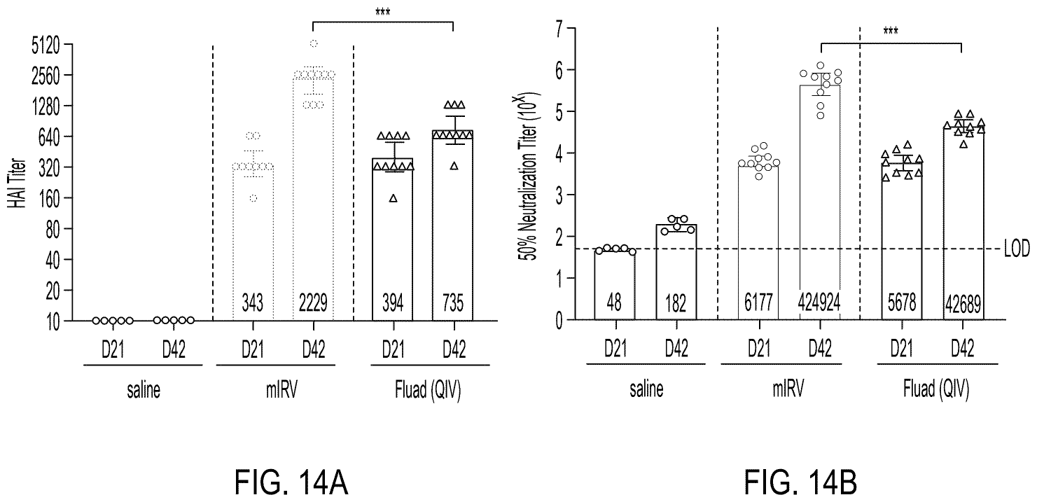

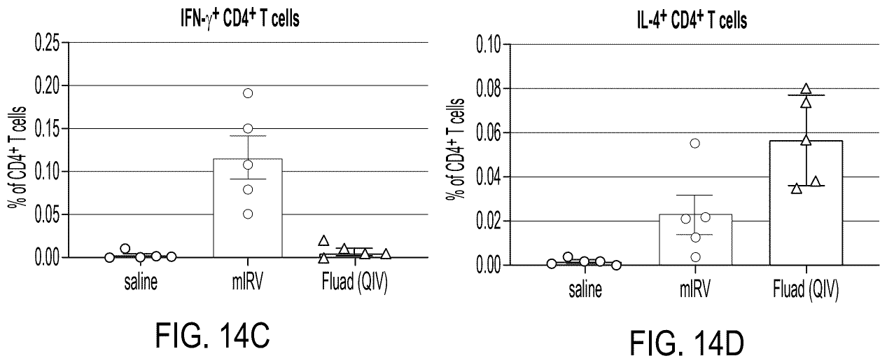

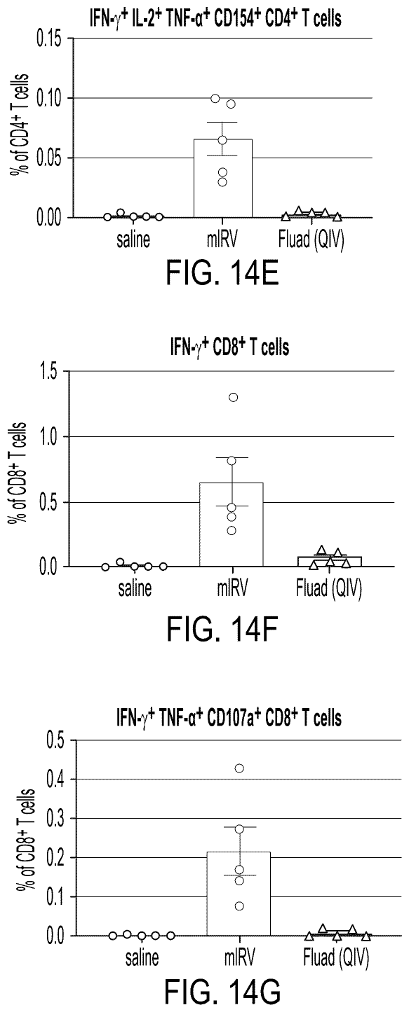

To evaluate immunogenicity of a monovalent modRNA-HA vaccine, mice (10 animals/group) were immunized with two doses of mIRV encoding the HA antigen from A/Wisconsin/588/2019 (H1N1) or a licensed adjuvanted QIV comparator (Fluad®) delivered 28 days apart. Sera were collected at immunologically relevant timepoints for evaluation of the magnitude and functionality of humoral responses. HA-specific antibody titers were measured by a hemagglutination inhibition assay (HAI) (FIG. 14A) and a microneutralization assay test (MNT) (FIG. 14B). Three weeks after the first dose (Day 21), mIRV and the QIV comparator induced similar HAI and MNT titers against A/Wisconsin/588/2019 (H1N1), however, mIRV induced statistically significantly higher HAI and MNT titers after the second dose compared to QIV. Two weeks after the second dose of mIRV (Day 42), HAI titers increased more than 6-fold (FIG. 14A) and MNT titers increased 68-fold (FIG. 14B) above titers obtained after the first dose (Day 21).

To evaluate HA-specific T cell responses, splenocytes were harvested two weeks after the second immunization, stimulated with peptides spanning the H1N1 HA protein from the vaccine strain (A/Wisconsin/588/2019), and assessed by intracellular cytokine staining for CD4+ T cells expressing IFN-γ, IL-4, IL-2, TNF-α and/or CD154, and CD8+ T cells expressing IFN-γ, TNF-α and/or CD107a. Immunization with two doses of mIRV induced a higher percentage of IFN-g-producing CD4+ T cells than IL-4-producing CD4+ T cells (FIG. 14C), indicative of a Th1-biased response, whereas two doses of QIV induced a higher percentage of IL-4+CD4+ T cells than IFN-γ+CD4+ T cells (FIG. 14D), indicative of a Th2-biased response. A strong polyfunctional (IFN-g⁺, IL-2⁺, TNF-α⁺, CD154⁺) CD4⁺ T cell response was also observed with the mIRV vaccine, but not with QIV (FIG. 14E). In addition, immunization with mIRV induced higher levels of IFN-g⁺CD8⁺ T cells (FIG. 14F) and polyfunctional (IFN-g⁺, TNF-α⁺, CD107a⁺) CD8⁺ T cells (FIG. 14G) compared to QIV.

Rhesus and Cynomolgus Macaques Immunized with mIRV Exhibit Durable Functional Antibody Responses and Polyfunctional T Cell Responses

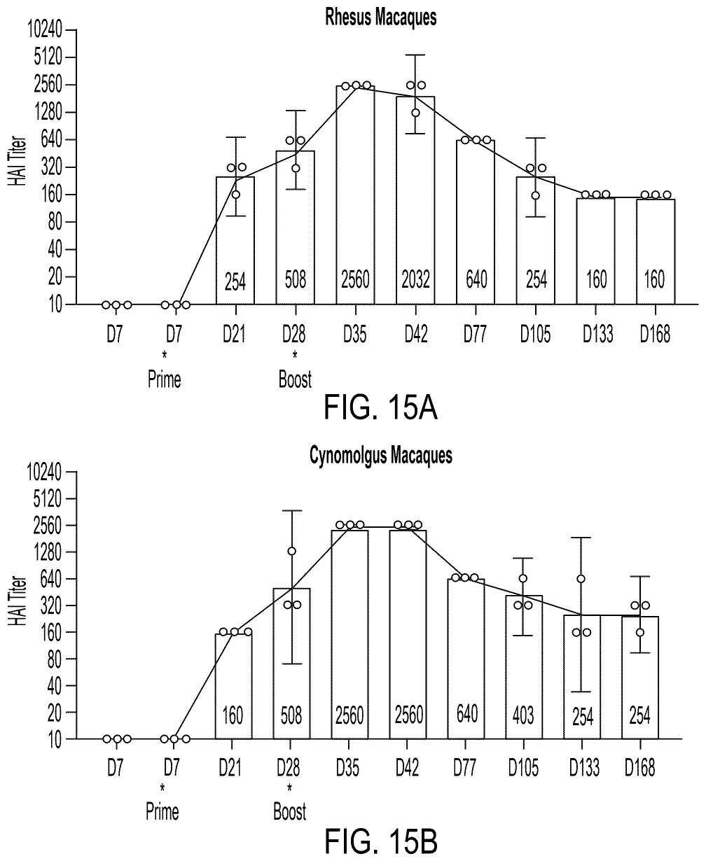

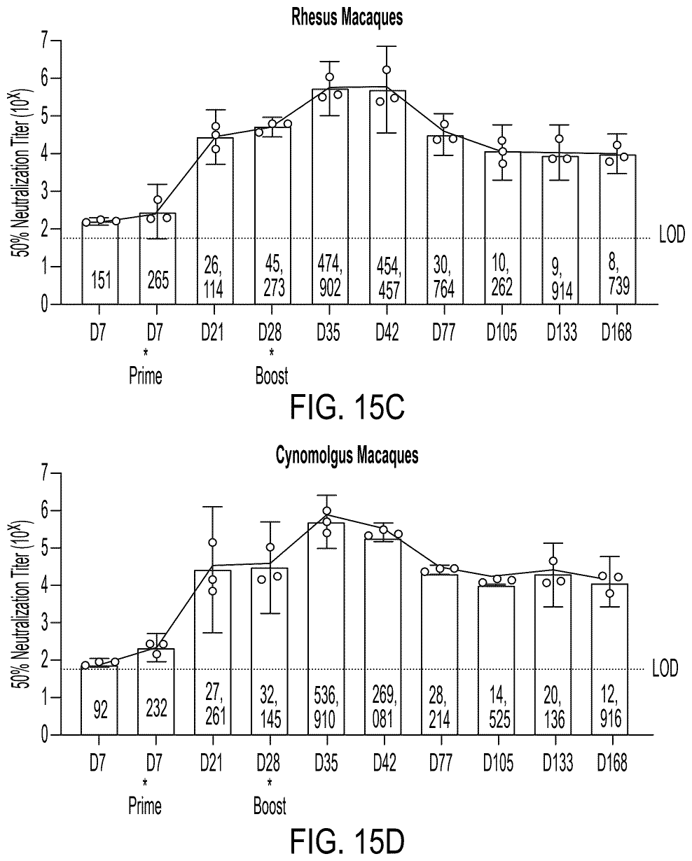

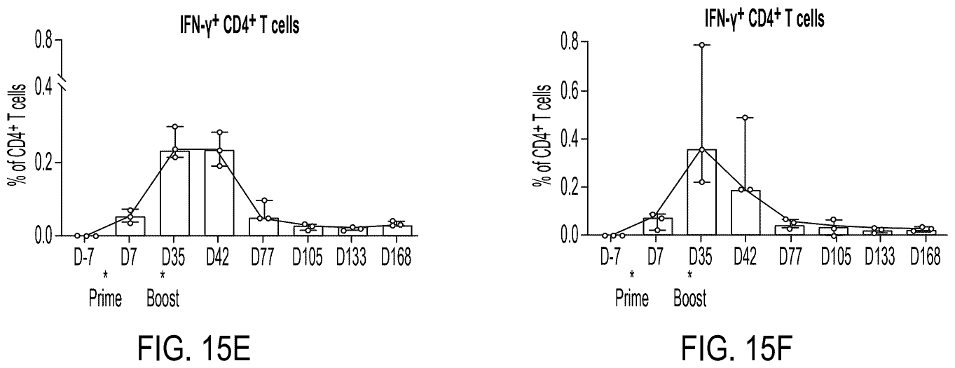

To evaluate durability of the modRNA platform, immunogenicity of mIRV was evaluated in two species of NHPs, rhesus and cynomolgus macaques, for approximately 5 months following vaccination. A total of three animals per group were immunized with two doses of mIRV (H1) 28 days apart. HA-specific antibodies were measured by HAI (FIG. 15A-B) and MNT (FIG. 15C-D). Vaccination with one dose of mIRV elicited a consistent pattern of HA-specific antibody responses against the homologous vaccine strain (A/Wisconsin/588/2019), inducing both HAI and neutralizing antibodies at three and four weeks after the first dose (Day 21 and Day 28) (FIG. 15A-D). Following the second immunization, HA-specific antibody levels peaked at one week after the second dose (Day 35) and then waned over the measured period of 19 weeks but stayed above baseline levels (Day-7). HAI and MNT titers plateaued from Day 105 through Day 168 of the study, maintaining levels comparable to those measured after the first dose (Day 21). T cell immunity was quantified by measuring cytokine-expressing peripheral CD4+ and CD8+ T cells after ex vivo stimulation of peripheral blood mononuclear cells (PBMCs) with HA peptide pools derived from the H1N1 vaccine strain (FIG. 15E-F). Immunization with mIRV induced IFN-g-expressing CD4+ T cells with responses peaking approximately one week after the second dose (Day 35) and returning to baseline levels by Day 105 for both species. No change in peripheral CD8+ T cells was detected in either species after immunization (data not shown). Overall, the data provide evidence that an HA-containing modRNA vaccine elicits robust and durable humoral and cellular immune responses.

Immunization with Two Doses of qIRV in Mice Elicited Functional Antibody Responses Against IAV and IBV Strains that were Greater than or Comparable to an Adjuvanted Inactivated QIV

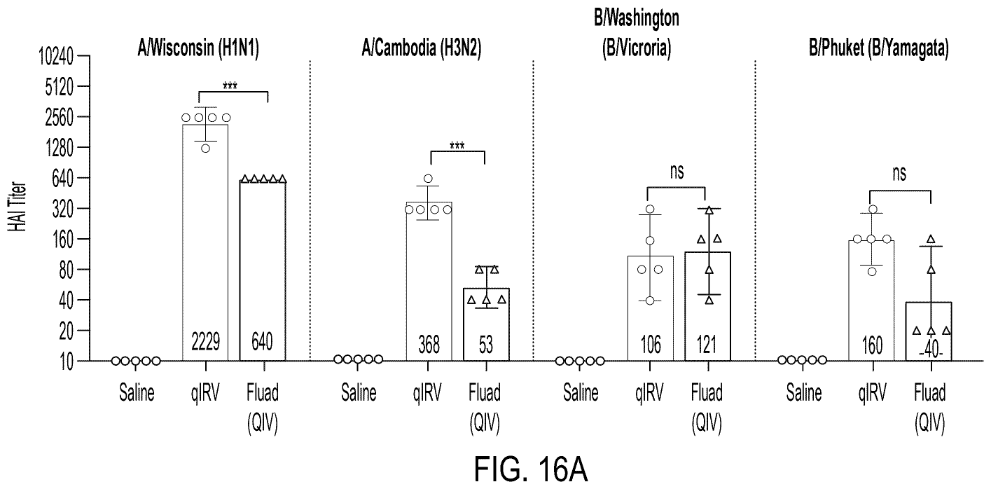

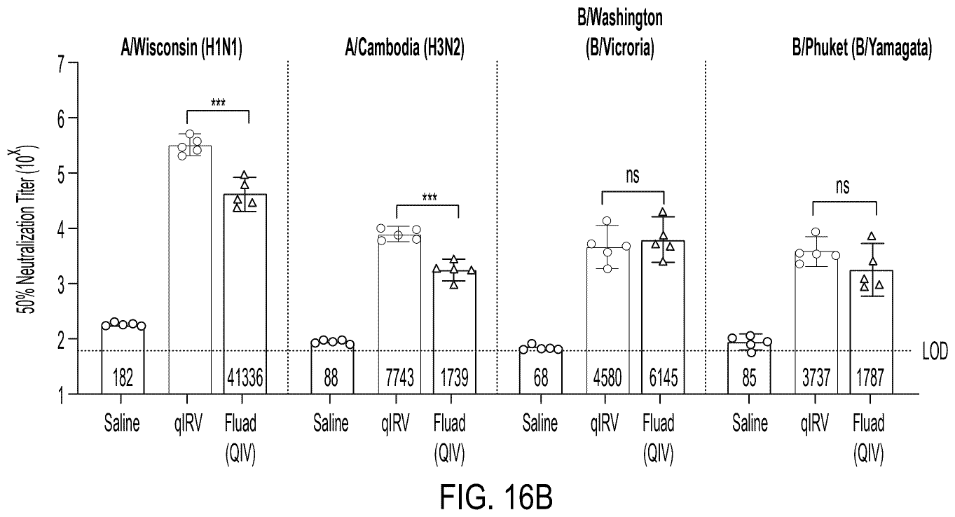

As current licensed seasonal influenza vaccines are designed to protect against up to four different influenza viruses (H1N1, H3N2, B/Victoria, B/Yamagata), we next evaluated the immunogenicity of a quadrivalent modRNA-HA vaccine in mice. The qIRV encoded HAs from the WHO-recommended influenza strains for cell culture- or recombinant-based vaccines for use in the 2021-2022 northern hemisphere influenza season: A/Wisconsin/588/2019 (H1N1), A/Cambodia/e0826360/2020 (H3N2), B/Washington/02/2019 (B/Victoria), and B/Phuket/3073/2013 (B/Yamagata). Mice were immunized with two doses of qIRV or licensed adjuvanted QIV 28 days apart. Two weeks after the second immunization, functional antibodies against each of the four strains encoded by the vaccines were measured by HAI (FIG. 16A) and MNT (FIG. 16B). Immunization with qIRV induced statistically significant higher HAI and MNT titers against H1N1 and H3N2, and similar HAI and MNT titers against B/Victoria and B/Yamagata compared to QIV (FIGS. 16A and B).

Immunization with Two Doses of tIRV or qIRV in Mice Elicited Similar IAV and IBV Neutralizing Antibody Responses as mIRV

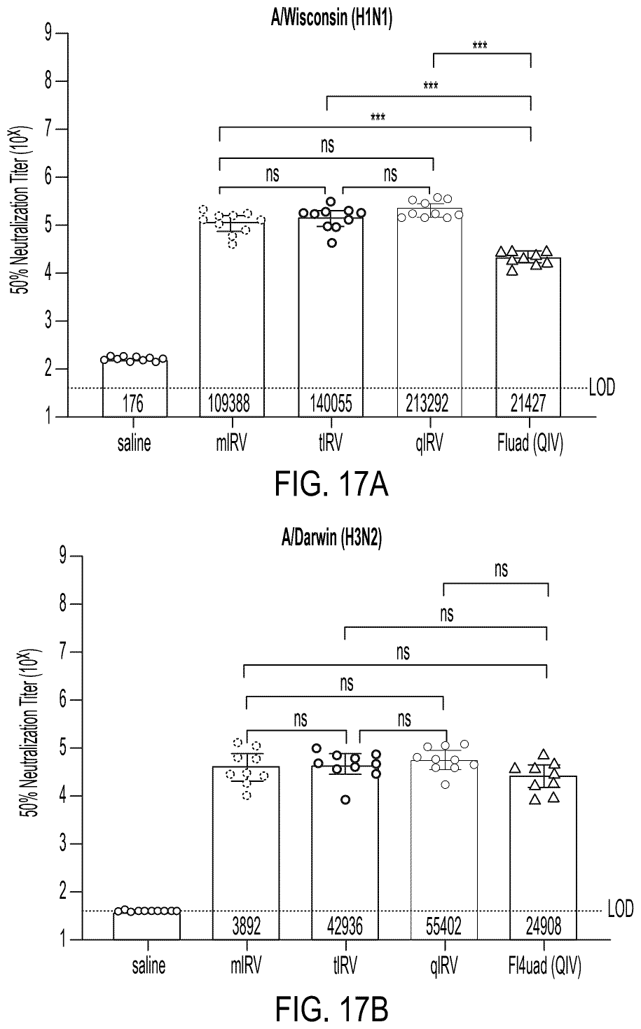

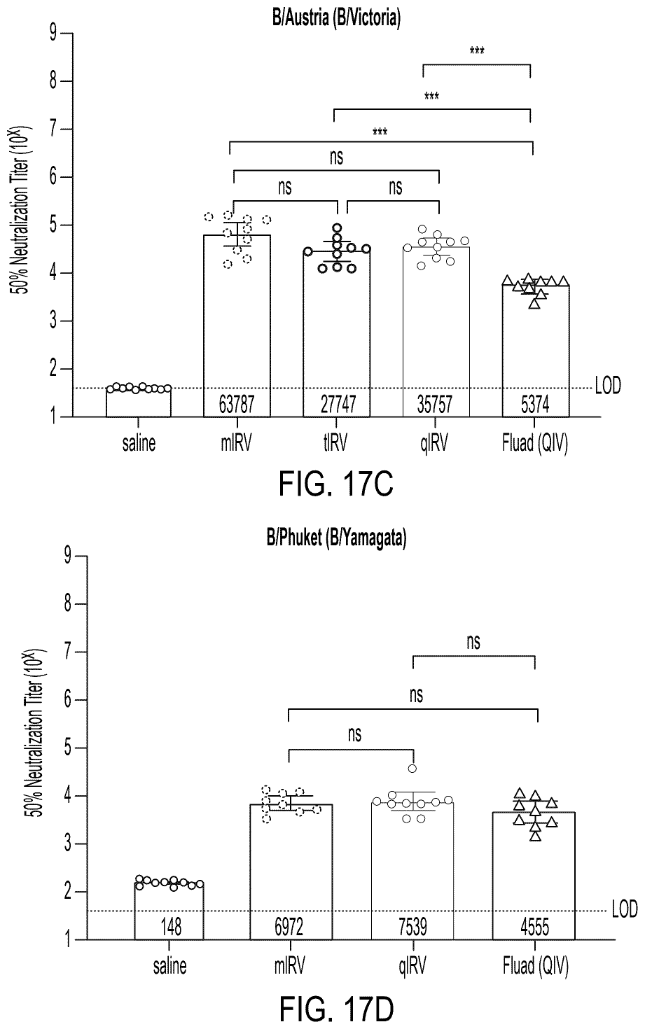

Due to the absence of WHO-confirmed detection of naturally occurring B/Yamagata lineage influenza viruses since March 2020, the WHO and VRBPAC have recommended the use of trivalent vaccines without the B/Yamagata component for the 2024-2025 northern hemisphere influenza season. To assess if a trivalent (tIRV) modRNA vaccine could perform as well as a qIRV modRNA vaccine for the three shared vaccine strains, we compared the immunogenicity of tIRV and qIRV to one another and to licensed adjuvanted QIV in mice. mIRVs targeting the individual vaccine strains were evaluated in parallel to assess any possible interference between IAV and IBV components of the multivalent IRVs that could result in lower immunogenicity. In this study, the modRNA-HA vaccines encoded HAs from the WHO-recommended influenza strains for cell culture- or recombinant-based vaccines for use in the 2022-2023 northern hemisphere influenza season: A/Wisconsin/588/2019 (H1N1), A/Darwin/6/2021 (H3N2), B/Austria/1359417/2021 (B/Victoria), B/Phuket/3073/2013 (B/Yamagata). Two weeks after the second immunization, virus neutralization titers against the vaccine-matched strains were measured by MNT (FIG. 17). Neutralization titers elicited by mIRV, tIRV, or qIRV against the shared vaccine strains (H1N1, H3N2, and B/Vic) were not statistically different (FIG. 17A-C) indicating an absence of interference. Neutralization titers against B/Yamagata elicited by mIRV and qIRV were also not statistically different (FIG. 17D). Notably, all three modRNA-HA vaccine formulations induced statistically significantly higher virus neutralizing titers against H1N1 (FIG. 17A) and B/Victoria (FIG. 17C) than the licensed QIV comparator. Neutralization titers against H3N2 (FIG. 17B) and B/Yamagata (FIG. 17D) were similar between the modRNA-HA vaccine formulations and the QIV control.

Toxicological Evaluation of mIRV and qIRV in Wistar Han Rats

To evaluate the nonclinical safety of modRNA-HA influenza vaccines, repeat-dose toxicity studies were conducted with mIRV encoding the HA antigen from A/Wisconsin/588/2019 (H1N1) and qIRV encoding HA antigens from the WHO-recommended influenza strains for cell culture- or recombinant-based vaccines for use in the 2021-2022 northern hemisphere influenza season. Briefly, male and female Wistar Han rats were administered 2 doses of mIRV or qIRV two weeks apart (Days 1 and 15). Dosing phase animals were euthanized two days after the second dose (Day 17) while recovery phase animals were euthanized approximately three weeks following the second dose (Day 38-39).

### In-Life Findings

In the repeat-dose toxicity studies, administration of mIRV and qIRV was tolerated without evidence of systemic toxicity. There were no vaccine-related mortalities, clinical signs, or effects on injection site dermal scores or ophthalmoscopic parameters. Vaccine-related effects on mean food consumption were observed for both mIRV (males only) and qIRV (both sexes) and animals recovered by Day 8 in both studies. There was no effect on mean food consumption in the recovery phase for either vaccine. For both vaccines, the mean body weight in the males was lower than controls on Day 4 (0.96×), with recovery observed by Day 8. There was no effect on mean body weight in the recovery phase for either vaccine. Transiently higher mean body temperature was seen in male and female animals in the mIRV group compared with the saline control group on Day 1 (1.03×) and Day 15 (1.02×) and for male animals in the qIRV group on Day 1 (1.01× saline group) and Day 15 (1.01×saline group).

### Serology

To confirm immunogenicity of both vaccine formulations administered in rats, functional antibodies titers were measured by HAI. An HAI response was detected in rats at the end of the dosing and recovery phases for all strains encoded by mIRV and qIRV. Hemagglutinin inhibition was not observed in the saline control animals.

### Clinical Pathology

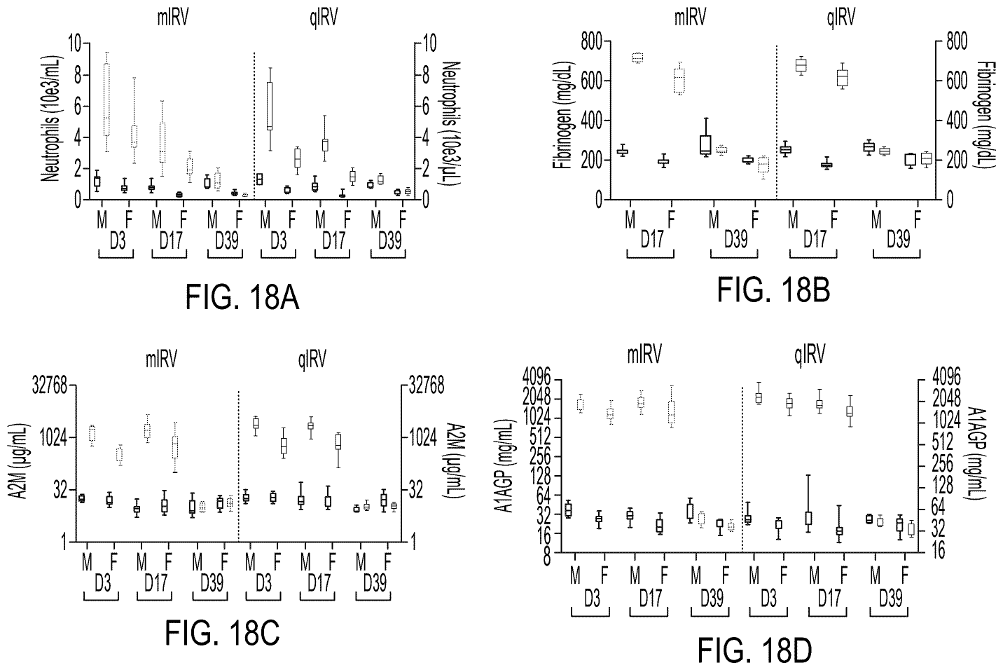

Key clinical pathology observations in rats were similar for both mIRV and qIRV and reflected the expected immune response following vaccination. Hematology findings were consistent with an inflammatory leukogram and included higher neutrophil counts on Days 3 and 17; a higher incidence of hyper-segmented neutrophils on Day 17; and higher monocytes, eosinophils, and/or large unstained cells on Days 3 and 17 (FIG. 18A). Findings consistent with an acute phase response were also noted for both vaccines and included higher fibrinogen on Day 17; higher globulin and/or lower albumin on Days 3 and 17; and higher alpha-2 macroglobulin (A2M) and alpha-1-acid glycoprotein (A1AGP) on Days 3 and 17 (FIG. 18B-D). All clinical pathology changes recovered by the end of the recovery phase except for higher globulin and/or lower albumin. In addition, transient, slightly lower reticulocyte and/or platelet counts on Day 3 and a nominal prolongation in prothrombin time (PT) on Day 17 were observed for both vaccines, a spectrum of findings consistent with immune activation and similar to observations with COVID-19 modRNA vaccines.

### Anatomic Pathology

Histopathologic features were comparable between mIRV and qIRV vaccines, with similar non-adverse vaccine-related microscopic findings at the injection site and in the draining (iliac) and inguinal lymph nodes, spleen, bone marrow, thymus (mIRV only), and liver. Vaccine-related cell inflammation and edema ranging from minimal to mild were observed at the injection site. These findings are typically associated with the IM administration of LNP-encapsulated mRNA vaccines and correlated with macroscopic observations of abnormal color (dark). At the end of the recovery phase, full recovery of edema and dark injection site and partial recovery of inflammation (minimal) at the injection site was observed.

Vaccine-related findings in the draining lymph nodes (increased cellularity of plasma cells and germinal centers), spleen (increased cellularity of hematopoietic cells and germinal centers), and bone marrow (increased cellularity of hematopoietic cells) were secondary to immune activation and/or inflammation at the injection site. The presence of plasma cells in the draining and inguinal lymph nodes was interpreted to reflect a robust immunological response to the vaccines and correlated with enlarged draining lymph nodes after vaccine administration. Microscopic observations in the spleen correlated with increased splenic weights and macroscopically enlarged spleens. At the end of the recovery phase, full recovery occurred for enlarged spleen, higher spleen weights, increased cellularity of hematopoietic cells in the spleen and bone marrow, and increased cellularity of germinal centers in the spleen. Partial recovery occurred for enlarged draining lymph nodes and increased cellularity of plasma cells and germinal centers in the draining and inguinal lymph nodes. An infiltration of macrophages in the draining and inguinal lymph nodes was observed with mIRV at the end of the recovery phase and was considered indicative of a reparative process (phagocytosis of material draining to the lymph node from the injection site).

A microscopic finding of minimal decreased lymphocyte cellularity in the thymus was observed for mIRV and was considered secondary to stress (indicated by a slight decrease in body weight or food consumption or a slight increase in body temperature) and not directly related to the vaccine. Decreased lymphocyte cellularity correlated with lower thymic weights. At the end of recovery phase, these findings were completely recovered.

Microscopic findings of minimal periportal hepatocyte vacuolation were observed for both vaccines at the end of the dosing phase. This was not associated with microscopic or biochemical evidence of hepatocyte damage (no increases in ALT or AST) and was interpreted to reflect hepatocyte uptake of the LNP lipids, as observed previously. At the end of the recovery phase, this finding was completely recovered.

### Discussion

Our studies described here provide further evidence that modRNA-HA vaccines targeting seasonal influenza strains are immunogenic. Here, a modRNA-LNP platform was leveraged to develop seasonal influenza vaccines. HA antigens administered in monovalent and multivalent (tIRV, qIRV) formulations in equal doses per antigen exhibited consistent antibody responses specific to each HA subtype in mice, demonstrating that potency of modRNA-HA vaccines is preserved when multiple antigens are targeted. Studies in both mice and NHPs demonstrate that influenza modRNA-HA vaccines elicit a multi-faceted immune response characterized by strong HA-specific functional and neutralizing antibody responses as well as cell-mediated immune responses. Unadjuvanted monovalent and quadrivalent modRNA-HA vaccines induced similar or better HAI titers compared to an adjuvanted licensed QIV vaccine. An HAI titer of ≥1:40 is an accepted immunological correlate of protection against influenza infection in humans. Influenza modRNA-HA vaccines induced robust Th1-type CD4+ T cell responses in mice and NHPs. IFNg+CD8+ T cell responses following vaccination were also observed in mice. The lack of CD8+ T cell activity observed in NHPs is similar to observations from NHP studies of a COVID-19 mRNA vaccine; however, vaccination of humans with COVID-19 mRNA vaccines has been shown to elicit durable CD8+ T cell responses.

The immune response elicited by the influenza modRNA-HA vaccines in preclinical animal studies aligns with clinical data from both SARS-COV-2 and influenza modRNA vaccines. Data from a Phase 1/2 clinical trial of the BNT162b2 COVID-19 modRNA vaccine (NCT04368728) demonstrated that vaccination induced high levels of SARS-COV-2 neutralizing antibody titers as well as antigen-specific CD8+ and Th1-type CD4+ T cell responses. Preliminary data from a Phase 2 trial of a quadrivalent influenza modRNA-HA vaccine (NCT05052697) also demonstrated the ability of modRNA vaccines to induce both neutralizing antibody responses and strain-specific CD4+ and CD8+ T cell responses. Thus, the modRNA vaccine may provide benefits over currently licensed seasonal influenza vaccines which induce limited T cell immunity. Altogether, the data presented here demonstrate the ability of modRNA vaccines to induce robust, balanced humoral and cellular immune responses to influenza with a tolerable nonclinical safety profile.

### Example25: Human Challenge Data

The seasonal influenza modRNA vaccine platform was evaluated (performed under the authority of the UK's MHRA by hVIVO). Study PIR-CSV-001 was a randomized double-blind, parallel design, single vaccination human influenza challenge study evaluating a monovalent modRNA vaccine encoding HA in reducing the incidence of infection or disease burden due to A/H1N1 (A/Delaware/55/2019) virus challenge, compared to placebo. The challenge virus used was antigenically similar to the A/Wisconsin/588/2019 HA encoded by the modRNA vaccine described herein. Approximately 50 participants ages 18 through 49 per group received a single vaccination at approximately 30 days prior to inoculation with the A/H1N1 influenza challenge virus. The goal was to evaluate the effect of the seasonal influenza modRNA vaccine in reduction in one or more of the following endpoints, when compared to placebo: (1) Area under the viral load-time curve as determined by qRT-PCR; (2) Peak viral load as defined by the maximum viral load determined by quantifiable qRT-PCR measurements; and (3) RT-PCR confirmed infection plus symptoms.

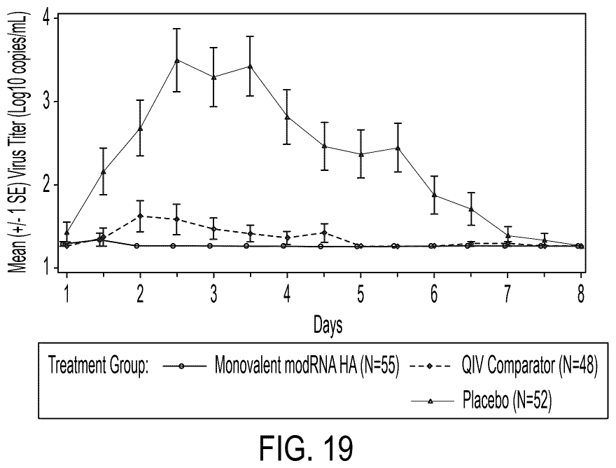

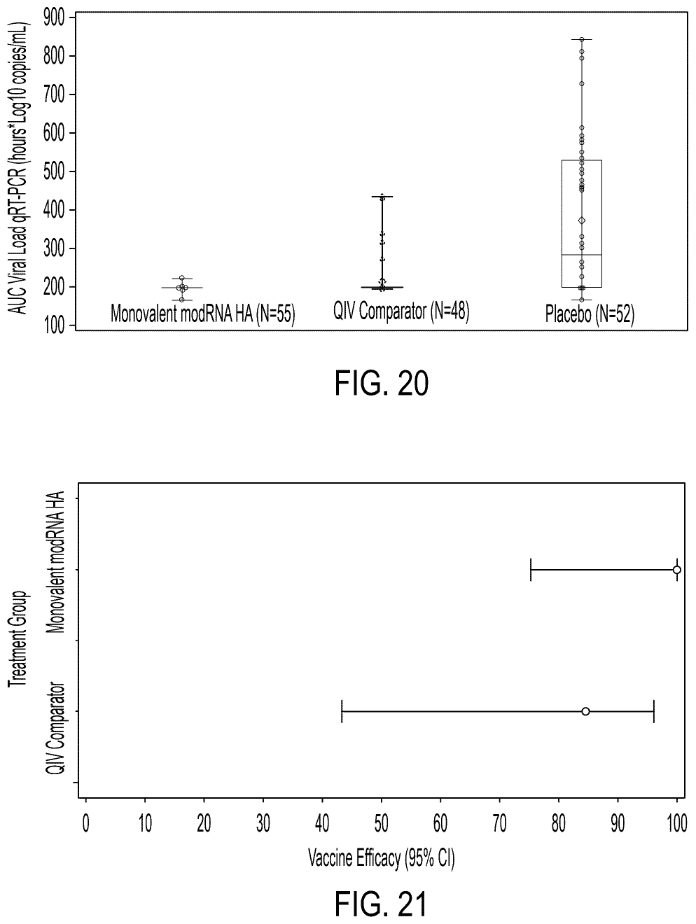

Data shown in FIG. 19 and FIG. 20 demonstrate that participants received the monovalent H1N1 modRNA vaccine had statistically significant lower viral load as measured by quantifiable qRT-PCR than those received placebo. As shown in FIG. 21, the modRNA vaccine also demonstrated 100% VE for qRT-PCR confirmed moderately severe and febrile infection. Although the study was not powered for a direct comparison between modRNA and the licensed QIV group, the data also suggested that viral load measurements trended lower and vaccine efficacy trended higher for the modRNA vaccine group.

The observation of reduced shedding in the modRNA vaccine group in side-by-side comparison with QIV also parallels the findings that both groups demonstrated high rates of seroconversion and HAI titer ≥1:40 (“seroprotection”) following vaccination (Table 56).

TABLE 56
Immunogenicity Analysis: HAI Seroconversion and Seroprotection
in Immunogenicity Population at Day 28 Post-Vaccination

| | Monovalent | |
|---|---|---|
| | modRNA HA | QIV |
| | % (95% confidence | % (95% confidence |
| | interval) | interval) |
| | (N = 73) | (N = 73) |
| Post Vaccination | 97.0% | 92.6% |
| Seroconversion* | (89.63, 99.64) | (83.67, 97.57) |
| Post Vaccination | 100% | 94.1% |
| “Seroprotection”*, ** | (94.64, 100.00) | (85.62, 98.37) |

*source tables: study timeframe: Pre-Vaccination (Day −30) to Admission (Day −2)
**seroprotection defined as HAI titer ≥ 1:40

### Example26: Co-Lyophilization of a Subunit and modRNA-LNP (PRL-RSV-Ms-2024-07)

The aim of this study is to evaluate the immunogenicity of a co-lyophilized drug product composed of RSV subunit (A+B) and modRNA-LNP (lipid nanoparticle) encapsulating influenza HA/California mRNA versus frozen liquid controls. This study will also investigate the effect of sodium oleate on the immunogenicity of the combined drug product compared to combinations without sodium oleate. Mice were immunized with RSV subunit and HA/California modRNA LNP drug product materials either alone or in combination. Sera collected on Day 21 (post prime) and at Day 42 (14 days post boost) will be evaluated by serology testing (assays specific for RSV and Flu). RSV Subunit and HA/California modRNA LNPs will be formulated using increasing amounts of sodium oleate to yield target formulations (−80C). These co-formulated RSV and LNPs will then be lyophilized and reconstituted with water such that the pre-lyophilization composition is obtained. The combined DPs will be dosed to assess formulation and lyophilization impact on immunogenicity.

TABLE 57
Test Articles: PRL-RSV-Ms-2024-07: RSV subunit and modCali
HA LNP w/sodium oleate co-lyophilization study

| Grp | Description | Final DP | Condition |
|---|---|---|---|
| 1 | Saline | N/A | N/A |
| 2 | RSV Subunit DP (Lyophilized | 0.240 mg/mL RSV (A + B) in 15 mM Tris, | 5° C. |
| | DP) | 2.25% Sucrose, 4.5% Mannitol, 0.015% | (Lyo) |
| | | PS80, pH 7.4 | |
| 3 | Cali HA modRNA | 0.13 mg/mL modRNA Cali HA in 20 mM | −80° C. |
| | | Tris, 300 mM Sucrose, 0.02% PS80, | (Liquid) |
| | | pH 7.4 | |
| 4 | RSV Subunit DP (Lyophilized | Mixed DP Gp 2 and 3 | Mixed DP |
| | DP) + | | |
| | Cali HA modRNA | | |
| 5 | Cali HA modRNA LNP + | 0.13 mg/mL modRNA Cali HA + | −80° C. |
| | RSV Sub unit Pre-Lyo | 0.52 mg/mL RSV (A + B) in 20 mM Tris, | (Liquid) |
| | | 300 mM Sucrose, 0.02% PS80, pH 7.4 | |
| 6 | Cali HA modRNA LNP + | 0.13 mg/mL modRNA Cali HA + 0.52 | −80° C. |
| | RSV Sub unit Pre-Lyo w/ | mg/mL RSV (A + B) in 20 mM Tris, 300 mM | (Liquid) |
| | 200 mcg/mL sodium oleate | Sucrose, 0.02% PS80, pH 7.4 w/200 | |
| | | ug/mL sodium oleate | |
| 7 | Cali HA modRNA LNP + | 0.13 mg/mL modRNA Cali HA + | −80° C. |
| | RSV Sub unit Pre-Lyo w/ | 0.52 mg/mL RSV (A + B) in 20 mM Tris, | (Liquid) |
| | 400 mcg/mL sodium oleate | 300 mM Sucrose, 0.02% PS80, pH 7.4 w/ | |
| | | 400 ug/mL sodium oleate | |
| 8 | Cali HA modRNA LNP + | 0.13 mg/mL modRNA Cali HA + | −80° C. |
| | RSV Sub unit Pre-Lyo w/ | 0.52 mg/mL RSV (A + B) in 20 mM Tris, | (Liquid) |
| | 800 mcg/mL sodium oleate | 300 mM Sucrose, 0.02% PS80, pH 7.4 w/ | |
| | | 800 ug/mL sodium oleate | |
| 9 | Cali HA modRNA LNP + | 0.0431 mg/mL modRNA Cali HA, pH 7.4 + | 5° C. |
| | RSV Sub unit Co-Lyo | 0.1724 mg/mL RSV (A + B) | (Lyophilized) |
| 10 | Cali HA modRNA LNP + | 0.0431 mg/mL modRNA Cali HA, pH 7.4 + | 5° C. |
| | RSV Sub unit Co-Lyo w/~66 | 0.1724 mg/mL RSV (A + B) w/~66 | (Lyophilized) |
| | mcg/mL sodium oleate | ug/mL sodium oleate | |
| 11 | Cali HA modRNA LNP + | 0.0431 mg/mL modRNA Cali HA, pH 7.4 + | 5° C. |
| | RSV Sub unit Co-Lyo w/~132 | 0.1724 mg/mL RSV (A + B) w/~132 | (Lyophilized) |
| | mcg/mL sodium oleate | ug/mL sodium oleate | |
| 12 | Cali HA modRNA LNP + | 0.0431 mg/mL modRNA Cali HA, pH 7.4 + | 5° C. |
| | RSV Sub unit Co-Lyo w/~264 | 0.1724 mg/mL RSV (A + B) w/~264 | (Lyophilized) |
| | mcg/mL sodium oleate | ug/mL sodium oleate | |

TABLE 58
PRL-RSV-Ms-2024-07: RSV subunit and modCali HA
LNP w/sodium oleate co-lyophilization study

| | | | | Dose | | | | |
|---|---|---|---|---|---|---|---|---|
| | | | | modCali/ | Vol/ | Vax | Bleed | |
| Grp | N | Description | Final DP | RSV | Route | (Day) | (Day) | Condition |
| 1 | 10 | Saline | N/A | N/A | 50 μL/IM | 0, 28 | 21, 42 | N/A |
| 2 | 10 | RSV Subunit | 0.240 mg/mL | 1.6 | 50 μL/IM | 0, 28 | 21, 42 | 5° C. |
| | | (A + B) DP | RSV (A + B) | | | | | (Lyo) |
| | | (Lyophilized DP) | in 15 mM | | | | | |
| | | | Tris, 2.25% | | | | | |
| | | | Sucrose, 4.5% | | | | | |
| | | | Mannitol, 0.015% | | | | | |
| | | | PS80, pH 7.4 | | | | | |
| 3 | 10 | HA/California | 0.13 mg/mL | 0.4 | 50 μL/IM | 0, 28 | 21, 42 | −80° C. |
| | | modRNA | modRNA Cali | | | | | (Liquid) |
| | | | HA, pH 7.4 | | | | | |
| 4 | 10 | RSV Subunit DP | Mixed RSV | 0.4/1.6 | 50 μL/IM | 0, 28 | 21, 42 | Mixed DP |
| | | (Lyophilized DP) + | DP and | | | | | (bedside) |
| | | HA/California | ModCali HA | | | | | |
| | | modRNA | DP | | | | | |
| 5 | 10 | HA/California | 0.13 mg/mL | 0.4/1.6 | 50 μL/IM | 0, 28 | 21, 42 | −80° C. |
| | | modRNA + | modRNA Cali | | | | | (Liquid) |
| | | RSV Subunit | HA, pH 7.4 + | | | | | |
| | | (A + B) Pre-Lyo | 0.52 mg/mL | | | | | |
| | | | RSV (A + B) | | | | | |
| 6 | 10 | HA/California | 0.13 mg/mL | 0.4/1.6 | 50 μL/IM | 0, 28 | 21, 42 | −80° C. |
| | | modRNA LNP + | modRNA Cali | | | | | (Liquid) |
| | | RSV Subunit | HA, pH 7.4 + | | | | | |
| | | (A + B) Pre-Lyo w/ | 0.52 mg/mL | | | | | |
| | | 200 μg/mL | RSV (A + B) | | | | | |
| | | sodium oleate | w/200 ug/mL | | | | | |
| | | | sodium oleate | | | | | |
| 7 | 10 | HA/California | 0.13 mg/mL | 0.4/1.6 | 50 μL/IM | 0, 28 | 21, 42 | −80° C. |
| | | modRNA + RSV | modRNA Cali | | | | | (Liquid) |
| | | Subunit (A + B) | HA, pH 7.4 + | | | | | |
| | | Pre-Lyo w/400 | 0.52 mg/mL | | | | | |
| | | μg/mL sodium | RSV (A + B) | | | | | |
| | | oleate | w/400 ug/mL | | | | | |
| | | | sodium oleate | | | | | |
| 8 | 10 | HA/California | 0.13 mg/mL | 0.4/1.6 | 50 μL/IM | 0, 28 | 21, 42 | −80° C. |
| | | modRNA + RSV | modRNA Cali | | | | | (Liquid) |
| | | Subunit (A + B) | HA, pH 7.4 + | | | | | |
| | | Pre-Lyo w/800 | 0.52 mg/mL | | | | | |
| | | μg/mL sodium | RSV (A + B) | | | | | |
| | | oleate | w/800 ug/mL | | | | | |
| | | | sodium oleate | | | | | |
| 9 | 10 | HA/California | 0.0431 mg/mL | 0.4/1.6 | 50 μL/IM | 0, 28 | 21, 42 | 5° C. |
| | | modRNA LNP + | modRNA Cali | | | | | (Lyo) |
| | | RSV Subunit | HA, pH 7.4 + | | | | | |
| | | (A + B) Co-Lyo | 0.1724 mg/mL | | | | | |
| | | | RSV (A + B) | | | | | |
| 10 | 10 | HA/California | 0.0431 mg/mL | 0.4/1.6 | 50 μL/IM | 0, 28 | 21, 42 | 5° C. |
| | | modRNA LNP + | modRNA Cali | | | | | (Lyo) |
| | | RSV Subunit | HA, pH 7.4 + | | | | | |
| | | (A + B) Co-Lyo | 0.1724 mg/mL | | | | | |
| | | w/~66 μg/mL | RSV (A + B) | | | | | |
| | | sodium oleate | w/~66 ug/mL | | | | | |
| | | | sodium oleate | | | | | |
| 11 | 10 | HA/California | 0.0431 mg/mL | 0.4/1.6 | 50 μL/IM | 0, 28 | 21, 42 | 5° C. |
| | | modRNA LNP + | modRNA Cali | | | | | (Lyo) |
| | | RSV Subunit | HA, pH 7.4 + | | | | | |
| | | (A + B) Co-Lyo | 0.1724 mg/mL | | | | | |
| | | w/~132 μg/mL | RSV (A + B) | | | | | |
| | | sodium oleate | w/~132 ug/mL | | | | | |
| | | | sodium oleate | | | | | |
| 12 | 10 | HA/California | 0.0431 mg/mL | 0.4/1.6 | 50 μL/IM | 0, 28 | 21, 42 | 5° C. |
| | | modRNA LNP + | modRNA Cali | | | | | (Lyo) |
| | | RSV Subunit | HA, pH 7.4 + | | | | | |
| | | (A + B) Co-Lyo | 0.1724 mg/mL | | | | | |
| | | w/~264 μg/mL | RSV (A + B) | | | | | |
| | | sodium oleate | w/~264 ug/mL | | | | | |
| | | | sodium oleate | | | | | |

TABLE 59
Data

| | | | | | | | IVE (%+/ | | | |
|---|---|---|---|---|---|---|---|---|---|---|
| | | RG | EE* | Size | | FA | EC50, 125 | Prefusion | Endotoxin | AA |
| Gp# | DP Description | (μg/mL) | (%) | (nm) | PDI | (%) | ng/well) | ELISA | (EU/mL) | Result |
| 1 | Saline | N/A | N/A | N/A | N/A | N/A | N/A | N/A | N/A | N/A |
| 2 | RSV Subunit DP | N/A | N/A | N/A | N/A | N/A | N/A | 81 | <0.050 | Negative |
| | (Lyophilized DP) | | | | | | | | | |
| 3 | Cali HA modRNA | 111 | 93 | 72 | 0.09 | 91/NMT 3% | 73/6** | N/A | <0.050 | Negative |
| 5 | Cali HA modRNA | 122 | 95 | 70 | 0.08 | 90/NMT 3% | 84/18 | 56 | <0.050 | Negative |
| | LNP + RSV Sub | | | | | | | | | |
| | unit Pre-Lyo | | | | | | | | | |
| 6 | Cali HA modRNA | 112 | 85 | 73 | 0.09 | 88/NMT 3% | 82/18 | TBD | <0.050 | Negative |
| | LNP + RSV Sub | | | | | | | | | |
| | unit Pre-Lyo w/ | | | | | | | | | |
| | 200 mcg/mL | | | | | | | | | |
| | sodium oleate | | | | | | | | | |
| 7 | Cali HA modRNA | 107 | 86 | 73 | 0.08 | 90/NMT 3% | 89/13 | TBD | <0.050 | Negative |
| | LNP + RSV Sub | | | | | | | | | |
| | unit Pre-Lyo w/ | | | | | | | | | |
| | 400 mcg/mL | | | | | | | | | |
| | sodium oleate | | | | | | | | | |
| 8 | Cali HA modRNA | 103 | 46 | 78 | 0.07 | 86/NMT 3% | 92/14 | TBD | <0.147 | Negative |
| | LNP + RSV Sub | | | | | | | | | |
| | unit Pre-Lyo w/ | | | | | | | | | |
| | 800 mcg/mL | | | | | | | | | |
| | sodium oleate | | | | | | | | | |
| 9 | Cali HA modRNA | 41 | 72 | 83 | 0.10 | 51/NMT 3% | 74/19 | 68 | <0.050 | Negative |
| | LNP + RSV Sub | | | | | | | | | |
| | unit Co-Lyo | | | | | | | | | |
| 10 | Cali HA modRNA | 39 | 44 | 85 | 0.10 | 52/NMT 3% | 74/21 | TBD | <0.055 | Negative |
| | LNP + RSV Sub | | | | | | | | | |
| | unit Co-Lyo | | | | | | | | | |
| | w/~66 mcg/mL | | | | | | | | | |
| | sodium oleate | | | | | | | | | |
| 11 | Cali HA modRNA | 38 | 51 | 83 | 0.09 | 69/NMT 3% | 88/13 | TBD | <0.050 | Negative |
| | LNP + RSV Sub | | | | | | | | | |
| | unit Co-Lyo | | | | | | | | | |
| | w/~132 mcg/mL | | | | | | | | | |
| | sodium oleate | | | | | | | | | |
| 12 | Cali HA modRNA | 37 | 34 | 91 | 0.09 | 72/NMT 3% | 87/14 | 71 | <0.050 | Negative |
| | LNP + RSV Sub | | | | | | | | | |
| | unit Co-Lyo | | | | | | | | | |
| | w/~264 mcg/mL | | | | | | | | | |
| | sodium oleate | | | | | | | | | |

*Low % EE by RG is expected for modRNA formulation with Sodium Oleate. The impact of low measured EE % by RG is to be determined
**IVE results for LNP alone reported at 63 ng/well (125 ng/well saturating level for this lot)
Oleate was observed to increase immunogenicity in both the pre-lyophilization and post-lyophilization modRNA-LNP materials. Although each individual formulation shows a loss in immunogenicity on lyophilization, oleate is able to recover the immunogenicity lost on lyophilization of the modRNA component (A/California for this study) - non-oleate pre-lyo vs oleate-containing post-lyo (~264 ug/mL).

The RSV subunit post 1^{st} dose bleeds may not yield appreciable titers at the dose used in the study (i.e., 1.6 μg) but the formulations containing oleate (specifically the ˜264 μg/mL group) yielded titers that appear to significantly boost titers above the non-oleate formulation (RSV/B pre-lyo 49 vs 1721 and post-lyo 174 vs 1566). Increased immunogenicity for the oleate-containing drug product appears to be higher than the adjuvanting effect observed when including LNPs (RSV/B compared to RSV/B+modRNA LNP (34 and 160, respectively) when compared against RSV/B+modRNA LNP w/oleate. In addition, increased immunogenicity for the oleate-containing drug product appears to be higher than the adjuvanting effect observed when including LNPs (RSV/B compared to RSV/B+modRNA LNP (34 and 160, respectively) when compared against lyophilized RSV/B+modRNA LNP w/oleate (1721 and 1566, respectively)).

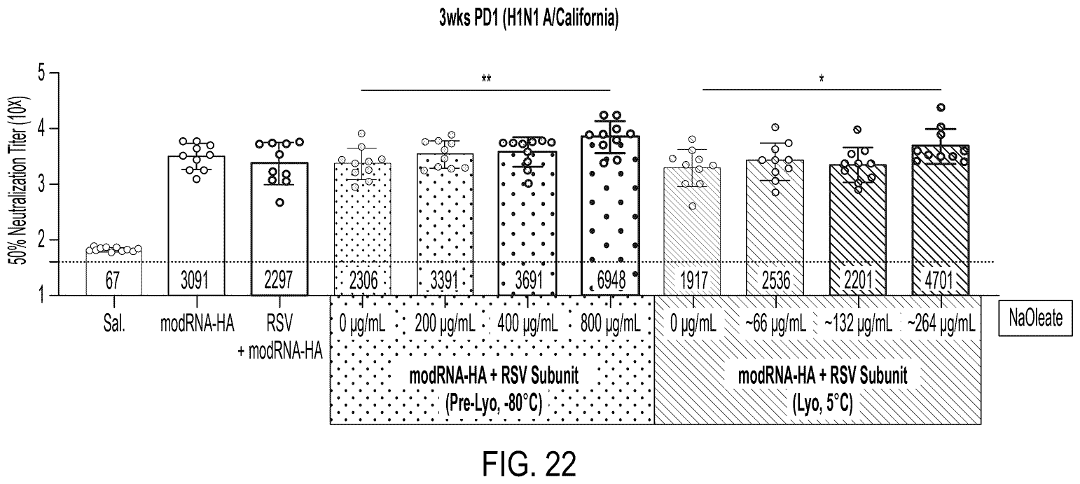

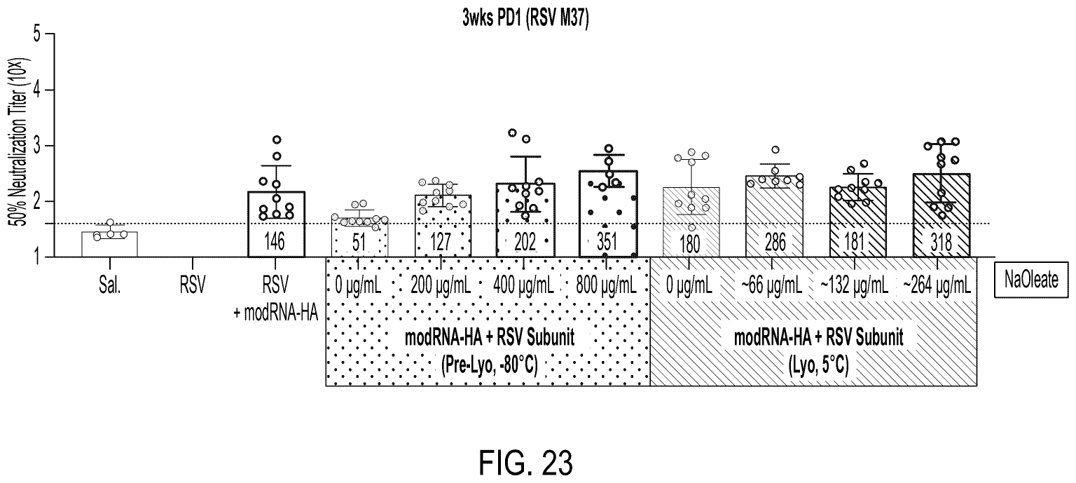

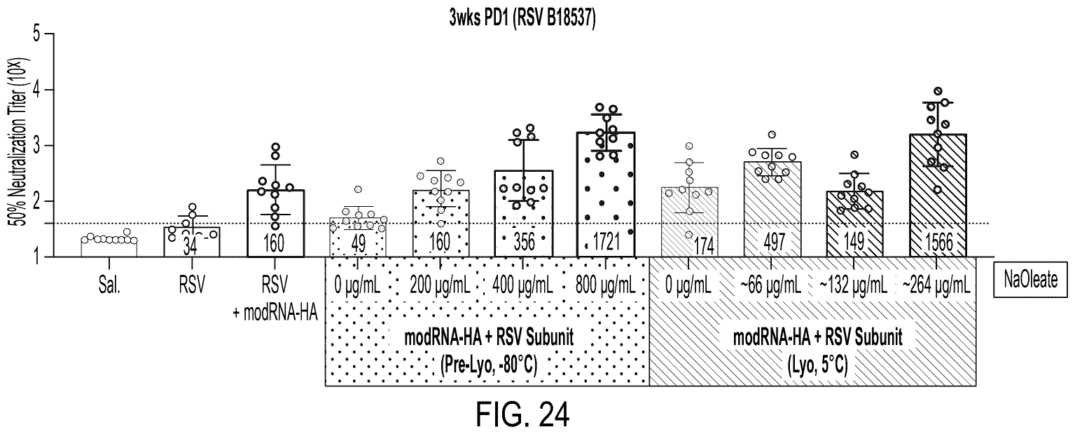

Co-formulated modRNA-HA+RSV subunit (pre-lyo) induced neutralizing Ab titers against A/California comparable to mixed DP and modRNA-HA alone. See FIG. 22. Titers tend to increase with increasing concentrations of sodium oleate for both Pre-Lyo and Lyo DPs DPs formulated with the highest Na Oleate concentration induced significantly higher titers than co-formulated DPs without Na Oleate under both Pre-Lyo (3-fold increase) and Lyo (2.5-fold increase) conditions. See FIG. 22. FIG. 23 depicts the resulting RSV Subunit Immunogenicity Data, Memphis 37 (wt RSV-A). FIG. 24 depicts the resulting RSV Subunit Immunogenicity Data, RSV/B/18537.

Post dose 1 summary with respect to modRNA Flu-No interference observed on immune responses to Flu modRNA-HA when mixed or co-formulated with RSV Subunit DP (pre-lyo). Slight negative impact on modRNA-HA immunogenicity when lyophilized, which was recovered with addition of sodium oleate. Incorporation of sodium oleate improved the immunogenicity of both pre-lyo and lyo drug products compared to co-formulations without sodium oleate. Three-fold improvement on pre-lyo titers was observed at the highest concentration tested; 2.5-fold improvement on lyo titers was observed at the highest concentration tested.

Post dose 1 summary with respect to RSV subunit-Slight interference on immune responses to RSV/A and RSV/B when co-formulated vs mixed with modCali LNP DP (pre-lyo), which was recovered and improved immunogenicity with the addition of sodium oleate. Slight improvement to no negative impact on RSV/A and RSV/B immunogenicity when lyophilized. Improved immunogenicity with addition of sodium oleate. Incorporation of sodium oleate improved the immunogenicity of both pre-lyo and lyo DPs compared to co-formulations without sodium oleate.

### Example27: PRL-Flu-Ms-2023-45: In-Vivo Results-Improving Flu B modRNA immunogenicity through rational antigen design: FP mutants

Objective: To determine the utility of the HA fusion peptide (FP) deletion A (369-373) with additional strains to increase Flu B modRNA HA immunogenicity compared to their corresponding wild-type (WT) codon optimized benchmark controls. And to test the utility of additional FP deletion mutants and the incorporation of another palmitoylation site at the HA CT at increasing Flu B HA immunogenicity, compared to the corresponding WT codon optimized benchmark control.

TABLE 60

| | | | Dose | Dose/ | Vaccination | Bleed |
|---|---|---|---|---|---|---|
| Gp | N | Test Article Description | (μg) | route | (Day) | (Day) |
| 1 | 10 | Saline | — | 50 μL/ | 0 & 27 | 19 & 41 |
| 2 | 10 | Bv/Colorado/06/2017-HA_WT | 0.2 | IM | | |
| 3 | 10 | Bv/Colorado/06/2017-HA-Δ370-374 | | | | |
| 4 | 10 | Bv/Washington/02/2019-HA-WT | | | | |
| 5 | 10 | Bv/Washington/02/2019-HA-Δ369-373 | | | | |
| 6 | 10 | By/Phuket/3073/2013-HA WT | | | | |
| 7 | 10 | By/Phuket/3073/2013-HA-Δ371-375 | | | | |
| 8 | 10 | Bv/Austria/1359417/2021-HA-WT | | | | |
| 9 | 10 | Bv/Austria/1359417/2021-HA-Δ369-373 | | | | |
| 10 | 10 | Bv/Austria/1359417/2021-HA-Δ355-361 | | | | |
| 11 | 10 | Bv/Austria/1359417/2021-HA-Δ361-364 | | | | |
| 12 | 10 | Bv/Austria/1359417/2021-HA-Δ363-366 | | | | |
| 13 | 10 | Bv/Austria/1359417/2021-HA-Δ366-373 | | | | |
| 14 | 10 | Bv/Austria/1359417-HA-S579C | | | | |

TABLE 61
PRL-Flu-Ms-2023-45: Improving Flu B modRNA Immunogenicity
through Rational Antigen Design (DS/DP material Summary)

| | | | | FACS DS | | FACS DP | | Cyt5 DP | | Cyt 5 DP | |
|---|---|---|---|---|---|---|---|---|---|---|---|
| | DS | DS | DP | Hek293 IVE | | Hek293 IVE | | Hek293 IVE | | Hela IVE | |
| | FA | Cap | FA | | Ave. | | Ave. | | Ave. | | Ave. |
| Construct | % | % | % | EC50 | MFI | EC50 | MFI | EC50 | MFI | EC50 | MFI |
| Bv/Colorado/06/2017-HA_WT | 92 | 94 | 87 | 81 | 683 | 59 | 529 | 33 | 3271 | >1000 | 2371 |
| Bv/Colorado/06/2017-HA-Δ370-374 | 91 | 94 | 84 | 68 | 825 | 35 | 772 | 17 | 6143 | 70 | 2613 |
| Bv/Washington/02/2019-HA-WT | 82 | 94 | 75 | 103 | 688 | 52 | 549 | 32 | 3608 | >1000 | 2369 |
| Bv/Washington/02/2019-HA-Δ369-373 | 91 | 94 | 87 | 84 | 808 | 34 | 897 | 12 | 7813 | 55 | 2785 |
| By/Phuket/3073/2013-HA WT | 92 | 92 | 87 | 193 | 703 | 80 | 505 | 33 | 4438 | >1000 | 2378 |
| By/Phuket/3073/2013-HA-Δ371-375 | 91 | 93 | 87 | 94 | 774 | 32 | 993 | 13 | 7491 | 50 | 2822 |
| Bv/Austria/1359417/2021-HA-WT | 91 | 93 | 88 | 206 | 660 | 69 | 564 | 29 | 2857 | >1000 | 3133 |
| Bv/Austria/1359417/2021-HA-Δ369-373 | 72 | 92 | 70 | 260 | 697 | 25 | 959 | 7.4 | 7294 | 46 | 3716 |
| Bv/Austria/1359417/2021-HA-Δ355-361 | 93 | 94 | 90 | 200 | 737 | 13 | 1282 | 5.3 | 9403 | 37 | 4263 |
| Bv/Austria/1359417/2021-HA-Δ361-364 | 93 | 94 | 89 | 204 | 782 | 9.2 | 1574 | 3.6 | 10822 | 30 | 4666 |
| Bv/Austria/1359417/2021-HA-Δ363-366 | 93 | 95 | 89 | 171 | 819 | 15 | 1282 | 6.9 | 8277 | 43 | 3699 |
| Bv/Austria/1359417/2021-HA-Δ366-373 | 92 | 94 | 89 | 184 | 703 | 11 | 1466 | 4.4 | 9370 | 29 | 4369 |
| Bv/Austria/1359417-HA-S579C | 93 | 94 | 88 | 159 | 639 | 26 | 846 | 12 | 3160 | >1000 | 3141 |

TABLE 62
PRL-Flu-Ms-2023-45: Improving Flu B modRNA Immunogenicity
through Rational Antigen Design (DP Material Summary)

| | DP | DP | DP | DP size | | DP | DP |
|---|---|---|---|---|---|---|---|
| Construct | (ng/ul) | FA % | EE | (d · nM) | DP | End | AA |
| Bv/Colorado/06/2017-HA_WT | 139 | 87 | 98 | 63 | 0.09 | Neg | Neg |
| Bv/Colorado/06/2017-HA-Δ370-374 | 143 | 84 | 99 | 67 | 0.12 | Neg | Neg |
| Bv/Washington/02/2019-HA-WT | 132 | 75 | 98 | 63 | 0.07 | Neg | Neg |
| Bv/Washington/02/2019-HA-Δ369-373 | 138 | 87 | 98 | 65 | 0.11 | Neg | Neg |
| By/Phuket/3073/2013-HA WT | 144 | 87 | 99 | 63 | 0.08 | Neg | Neg |
| By/Phuket/3073/2013-HA-Δ371-375 | 133 | 87 | 99 | 63 | 0.09 | Neg | Neg |
| Bv/Austria/1359417/2021-HA-WT | 148 | 88 | 99 | 62 | 0.07 | Neg | Neg |
| Bv/Austria/1359417/2021-HA-Δ369-373 | 126 | 70 | 98 | 67 | 0.13 | Neg | Neg |
| Bv/Austria/1359417/2021-HA-Δ355-361 | 122 | 90 | 98 | 63 | 0.09 | Neg | Neg |
| Bv/Austria/1359417/2021-HA-Δ361-364 | 124 | 89 | 98 | 67 | 0.11 | Neg | Neg |
| Bv/Austria/1359417/2021-HA-Δ363-366 | 137 | 89 | 98 | 62 | 0.07 | Neg | Neg |
| Bv/Austria/1359417/2021-HA-Δ366-373 | 132 | 89 | 98 | 64 | 0.1 | Neg | Neg |
| Bv/Austria/1359417-HA-S579C | 138 | 88 | 98 | 68 | 0.1 | Neg | Neg |

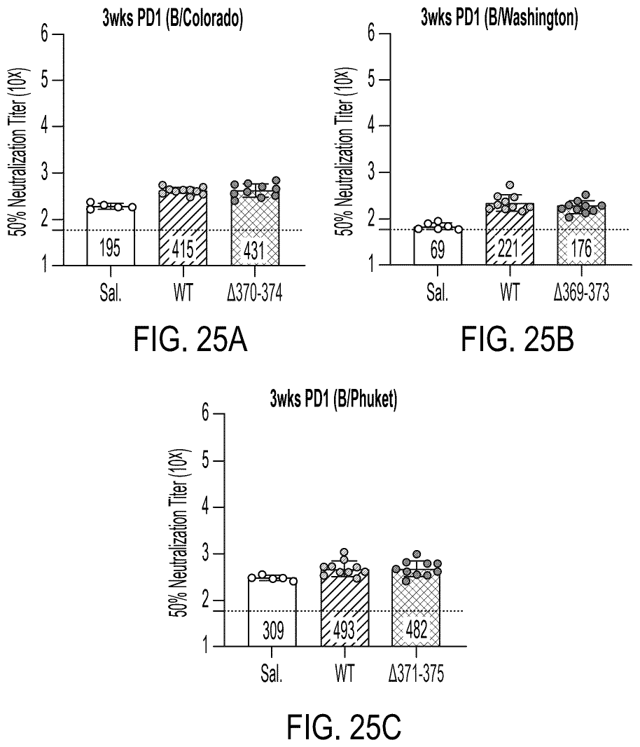

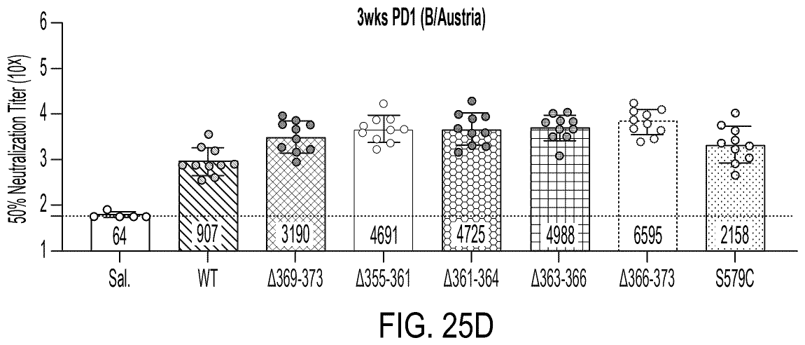

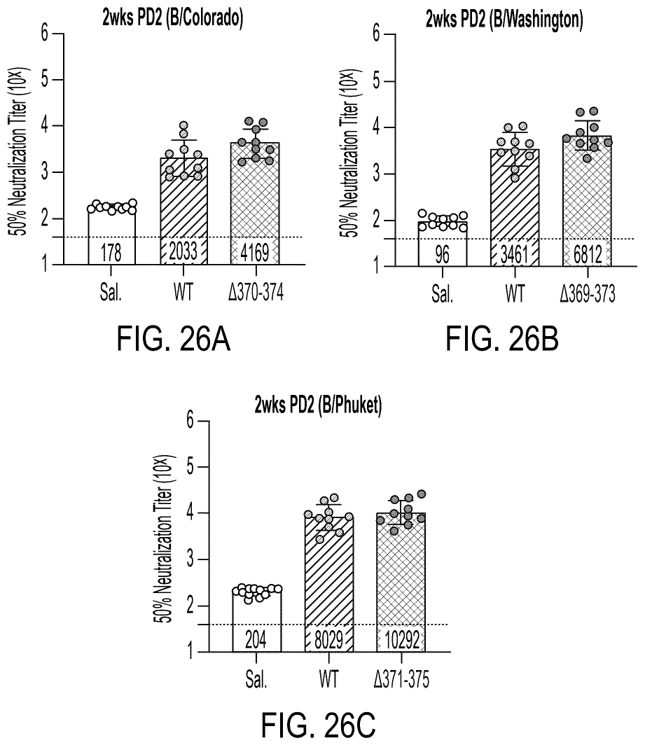

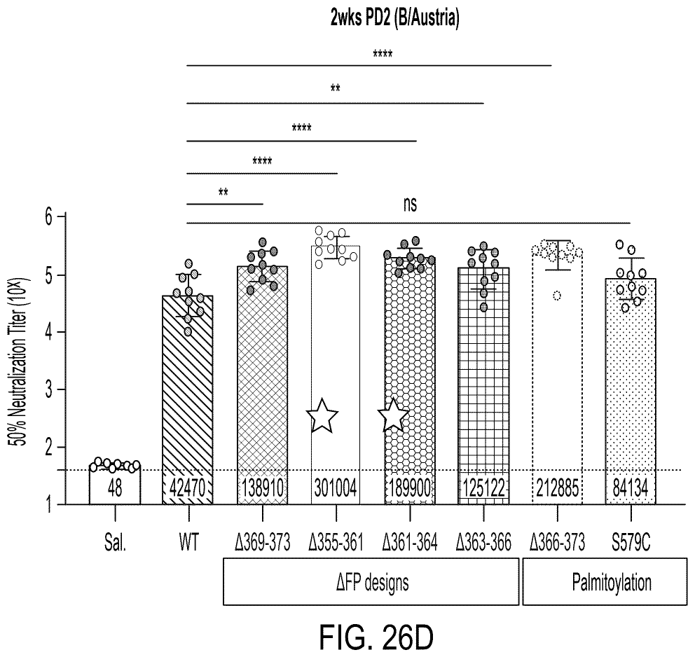

Results. All FP mutant candidates increased B/Austria titers 5-7-fold over WT HA. S579C mutation improves immunogenicity by 2.5-fold. See FIG. 25A-D. All FP mutant candidates increased B/Austria titers 3-7-fold over WT HA. S579C mutation improves immunogenicity by 2-fold. See FIG. 26A-D.

### Example28: PRL-Flu-Ms-2024-03: Enhancing Flu B modRNA Immunogenicity in tIRV through rational antigen design-combo mutants

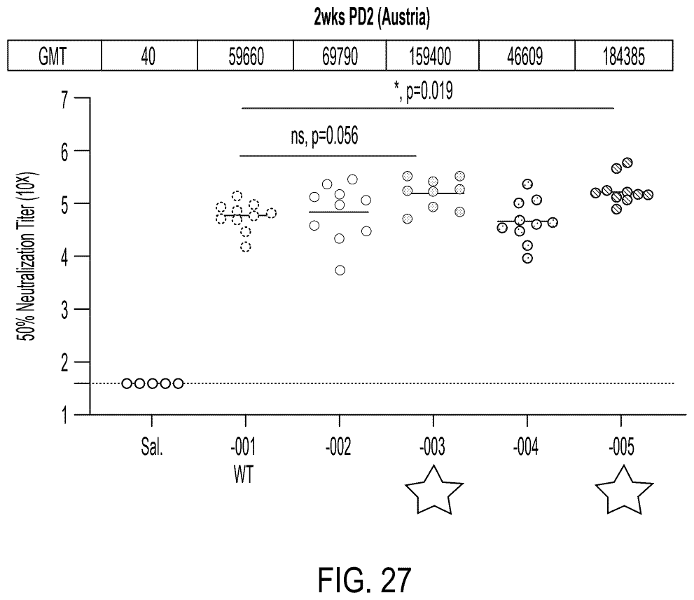

Rational antigen design 1st set: Thermostability & prefusion HA stability. ˜Up to 3-fold improvement in neutralization titers was observed with two designs sharing a common deletion in the HA fusion peptide A (369-373). See FIG. 27.

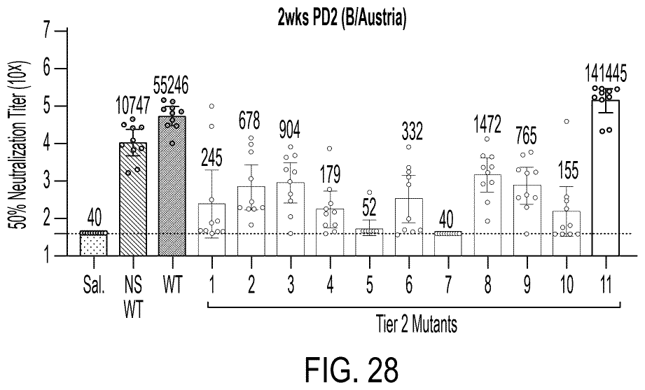

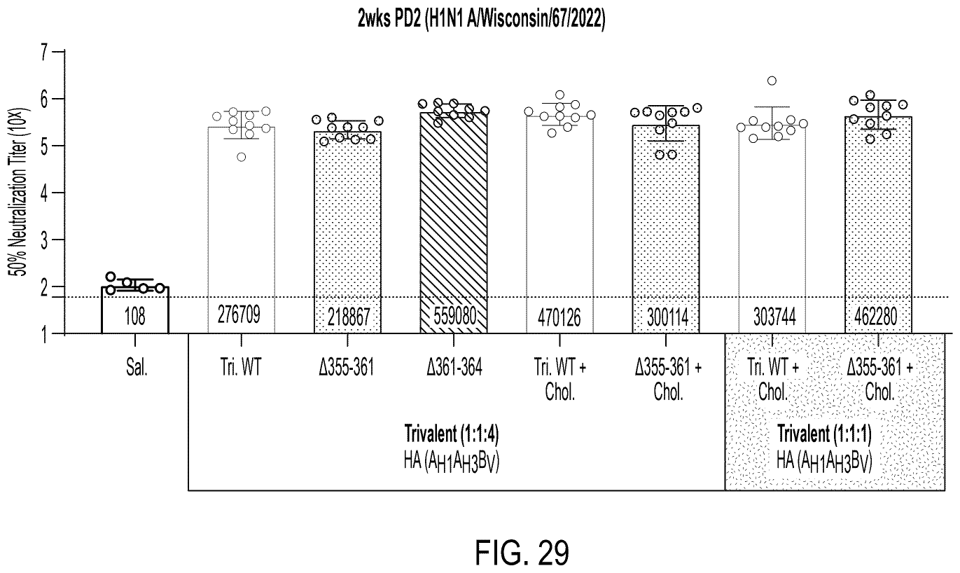

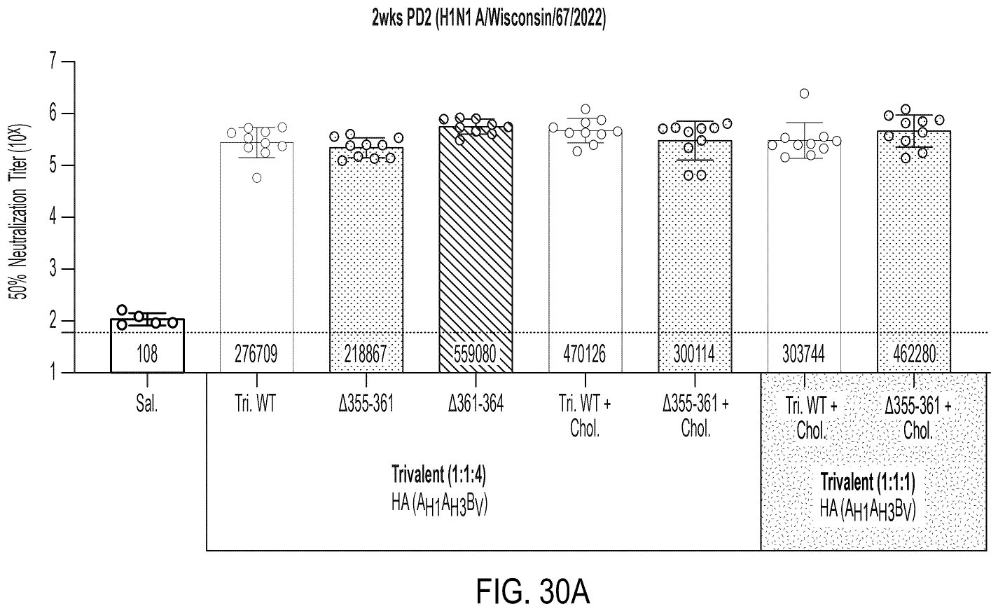

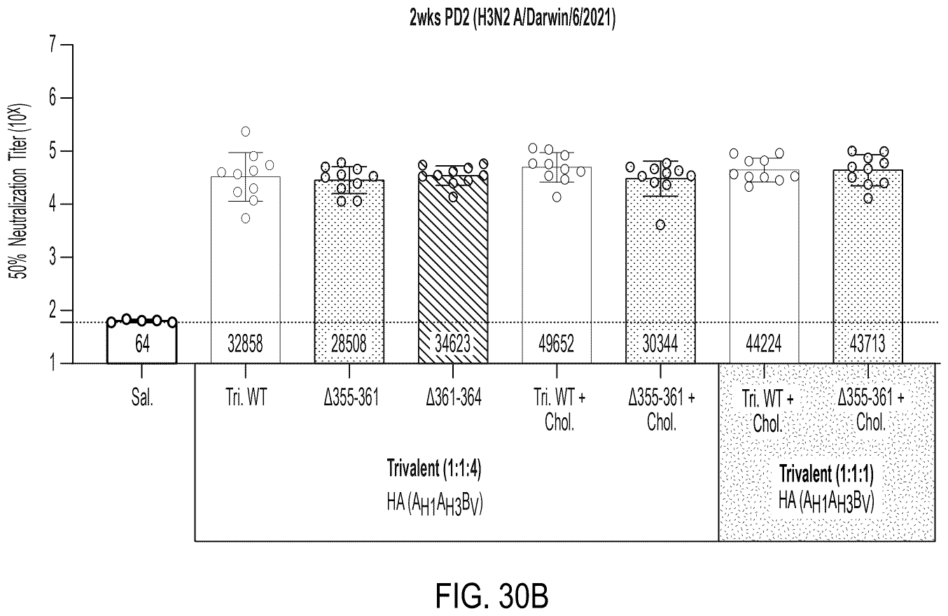

Rational antigen design 2nd set: Increase trimeric prefusion HA expression+A (369-373). Up to 2.5-fold increase in neutralization titer was observed when compared to the benchmark for isolated FP deletion. See FIG. 28. Rational antigen design 3rd set: A (369-373), AFP PFE designs & palmitoylation mutant. AFP mutants improve neutralization titers by 3 to 7-fold and palmitoylation mutant by 2-fold. See FIG. 26D. Lead Monovalent Flu B HA Candidate Showed 5.3-fold Higher In vitro Expression and 7-fold Higher Immunogenicity in Mice Compared to WT. See FIG. 26D, namely A355-361 compared to WT. Rational antigen design+improved LNP in tIRV: Top two AFP PFE candidates+cholesterol analogs. AFP improve Flu B titers in tIRV by ˜2-fold Δ (355-361) & ˜4.4-fold Δ (361-364); AFP improves Flu B titers in 1:1:1 tIRV by ˜3-fold Δ (355-361); AFP+cholesterol analogs improve titers in tIRV by ˜4-fold Δ (355-361). See FIG. 29. H1 in trivalent formulations containing Flu B mutant in the context of improved LNP shows slight (not significant) reduction in titer at the 1:1:4, but not at the 1:1:1 ratio. H3 in trivalent formulations containing Flu B mutant in the context of improved LNP shows slight (not significant) reduction in titer at the 1:1:4, but not at the 1:1:1 ratio. See FIG. 30. We also conducted a study to determine the ability of partial HA fusion peptide deletions (AFP) in combination with palmitoylation mutations to further increase Flu B HA immunogenicity in tIRV, compared to their corresponding WT and AFP single mutant controls. We generated a panel of Flu B and Flu A HA antigens, LNP formulated them as mIRV or tIRV, and evaluated their immunogenicity in BALB/c mice.

TABLE 63
study materials

| | | | Dose | Dose/ | Vax | Bleed |
|---|---|---|---|---|---|---|
| Gp# | Mice | Description | (μg) | route | (Day) | (Day) |
| 1 | 10 | Saline | — | 50 μl/IM | | |
| 2 | 10 | Bv/Austria/1359417/2021-HA-WT | 0.2 | 50 μl/IM | 0, 28 | 21, 42 |
| 3 | 10 | Bv/Austria/1359417/2021-HA-Δ355-361 + L582C | 0.2 | 50 μl/IM | 0, 28 | 21, 42 |
| 4 | 10 | Bv/Austria/1359417/2021-HA-Δ361-364 + L582C | 0.2 | 50 μl/IM | 0, 28 | 21, 42 |
| 5 | 10 | Bv/Austria/1359417/2021-HA-Δ355-363 + L582C | 0.2 | 50 μl/IM | 0, 28 | 21, 42 |
| 6 | 10 | Bv/Austria/1359417/2021-HA-Δ354-366 + L582C | 0.2 | 50 μl/IM | 0, 28 | 21, 42 |
| 7 | 10 | Trivalent modRNA HA (A_{H1}A_{H3}B_{V}) (1:1:4) | 1.2 (0.2A_{H1}, | 50 μl/IM | 0, 28 | 21, 42 |
| | | A/Wisc, A/Darwin, B/Austria-WT | 0.2A_{H3}, 0.8B_{V}) | | | |
| 8 | 10 | Trivalent modRNA HA (A_{H1}A_{H3}B_{V}) (1:1:4) | 1.2 (0.2A_{H1}, | 50 μl/IM | 0, 28 | 21, 42 |
| | | A/Wisc, A/Darwin, B/Austria (Δ355-361) Mut 1 | 0.2A_{H3}, 0.8B_{V}) | | | |
| 9 | 10 | Trivalent modRNA HA (A_{H1}A_{H3}B_{V}) (1:1:4) | 1.2 (0.2A_{H1}, | 50 μl/IM | 0, 28 | 21, 42 |
| | | A/Wisc, A/Darwin, B/Austria (Δ355-361) Mut 1 + L582C | 0.2A_{H3}, 0.8B_{V}) | | | |
| 10 | 10 | Trivalent modRNA HA (A_{H1}A_{H3}B_{V}) (1:1:4) | 1.2 (0.2A_{H1}, | 50 μl/IM | 0, 28 | 21, 42 |
| | | A/Wisc, A/Darwin, B/Austria (Δ361-364) Mut 2 | 0.2A_{H3}, 0.8B_{V}) | | | |
| 11 | 10 | Trivalent modRNA HA (A_{H1}A_{H3}B_{V}) (1:1:4) | 1.2 (0.2A_{H1}, | 50 μl/IM | 0, 28 | 21, 42 |
| | | A/Wisc, A/Darwin, B/Austria (Δ361-364) Mut 2 + L582C | 0.2A_{H3}, 0.8B_{V}) | | | |
| 12 | 10 | Trivalent modRNA HA (A_{H1}A_{H3}B_{V}) (1:1:4) | 1.2 (0.2A_{H1}, | 50 μl/IM | 0, 28 | 21, 42 |
| | | A/Wisc, A/Darwin, B/Austria (Δ355-363) Mut 3 + L582C | 0.2A_{H3}, 0.8B_{V}) | | | |
| 13 | 10 | Trivalent modRNA HA (A_{H1}A_{H3}B_{V}) (1:1:4) | 1.2 (0.2A_{H1}, | 50 μl/IM | 0, 28 | 21, 42 |
| | | A/Wisc, A/Darwin, B/Austria (Δ354-366) Mut 4 + L582C | 0.2A_{H3}, 0.8B_{V}) | | | |

TABLE 64
DS analytics Summary of the study materials from Table 63:

| | | | DS |
|---|---|---|---|
| | | DS | Capping |
| pVV # | Construct | FA % | % |
| pVV-01549 | A/Wisconsin/67/21-HA WT | 90 | 95% |
| pVV-00221 | A/Darwin/06/21-WT | 91 | 95% |
| pVV-00213 | Bv/Austria/1359417/2021-HA-WT | 92 | 93% |
| pVV-01779 | Bv/Austria/1359417-HA-Δ355-361 | 92 | 91% |
| pVV-02231 | Bv/Austria/1359417-HA-Δ355-361 + L582C | 92 | 92% |
| pVV-01780 | Bv/Austria/1359417-HA-Δ361-364 | 91 | 91% |
| pVV-02230 | Bv/Austria/1359417-HA-Δ361-364 + L582C | 91 | 92% |
| pVV-02228 | Bv/Austria/1359417-HA-Δ354-366 + L582C | 90 | 91% |
| pVV-02229 | Bv/Austria/1359417-HA-Δ355-363 + L582C | 88 | 92% |

TABLE 65
B/Austria-HA Mutant Monovalent HEK293T/Hela DP IVE
Summary Pre-screening for tIRV candidate selection:

| | | | HEK293T | | |
|---|---|---|---|---|---|
| | | HEK293T | IVE | Hela | Hela |
| | | IVE | MFI | IVE | IVE |
| pVV# | Construct | EC50 | average | MFI | EC50 |
| pVV- | Bv/Austria/1359417/2021-HA-WT | 20 | 2,640 | 2,640 | >1000 |
| 00213 | | | | | |
| pVV- | Bv/Austria/1359417-HA-Δ355-361* | 9.8 | 4,791 | 4,791 | 45 |
| pVV- | Bv/Austria/1359417-HA-Δ355-361 + | 3.9 | 7,289 | 7,289 | 12 |
| 02231 | L582C | | | | |
| pVV- | Bv/Austria/1359417-HA-Δ355-361 + | 8.2 | 4,922 | 4,922 | 60 |
| 02232 | S579C | | | | |
| pVV- | Bv/Austria/1359417-HA-Δ355-361 + | 7.3 | 5,293 | 5,293 | 41 |
| 02233 | V571C | | | | |
| pVV- | Bv/Austria/1359417-HA-Δ361-364* | 7.0 | 5,046 | 5,046 | 48 |
| 01780 | | | | | |
| pVV- | Bv/Austria/1359417-HA-Δ361-364 + | 7.7 | 5,524 | 5,524 | 41 |
| 02230 | L582C | | | | |
| pVV- | Bv/Austria/1359417-HA-Δ354-366 + | 7.0 | 5,386 | 5,386 | 29 |
| 02228 | L582C | | | | |
| pVV- | Bv/Austria/1359417-HA-Δ355-363 + | 7.5 | 5,531 | 5,531 | 23 |
| 02229 | L582C | | | | |

TABLE 66
mIRV & tIRV HEK293T DP IVE Summary

| | | DP HEK293 IVE | | | | | |
|---|---|---|---|---|---|---|---|
| Grp# | Construct | EC50 | | | Avg. MFI | | |
| 2 | Bv/Austria/1359417/2021-HA-WT | 2.8 | | | 7,784 | | |
| 3 | Bv/Austria/1359417/2021-HA-Δ355-361 + L582C | 0.6 | | | 10,161 | | |
| 4 | Bv/Austria/1359417/2021-HA-Δ361-364 + L582C | 2.0 | | | 10,695 | | |
| 5 | Bv/Austria/1359417/2021-HA-Δ355-363 + L582C | 1.2 | | | 13,160 | | |
| 6 | Bv/Austria/1359417/2021-HA-Δ354-366 + L582C | 1.3 | | | 12,351 | | |
| | | H1 | H3 | Bv | H1 | H3 | Bv |
| 7 | Trivalent modRNA HA (A_{H1}A_{H3}B_{V}) (1:1:4) | 1.3 | 0.7 | 2.0 | 4439 | 5109 | 7200 |
| | A/Wisc, A/Darwin, B/Austria-WT | | | | | | |
| 8 | Trivalent modRNA HA (A_{H1}A_{H3}B_{V}) (1:1:4) | 1.5 | 0.8 | 1.6 | 4602 | 5052 | 10496 |
| | A/Wisc, A/Darwin, B/Austria (Δ355-361) Mut 1 | | | | | | |
| 9 | Trivalent modRNA HA (A_{H1}A_{H3}B_{V}) (1:1:4) | 1.3 | 0.7 | 1.7 | 4871 | 5307 | 10926 |
| | A/Wisc, A/Darwin, B/Austria (Δ355-361) Mut 1 + L582C | | | | | | |
| 10 | Trivalent modRNA HA (A_{H1}A_{H3}B_{V}) (1:1:4) | 1.3 | 0.6 | 1.7 | 4775 | 5300 | 10590 |
| | A/Wisc, A/Darwin, B/Austria (Δ361-364) Mut 2 | | | | | | |
| 11 | Trivalent modRNA HA (A_{H1}A_{H3}B_{V}) (1:1:4) | 1.4 | 0.8 | 1.8 | 4605 | 5203 | 10215 |
| | A/Wisc, A/Darwin, B/Austria (Δ361-364) Mut 2 + L582C | | | | | | |
| 12 | Trivalent modRNA HA (A_{H1}A_{H3}B_{V}) (1:1:4) | 1.2 | 0.6 | 1.2 | 4789 | 5211 | 11574 |
| | A/Wisc, A/Darwin, B/Austria (Δ355-363) Mut 3 + L582C | | | | | | |
| 13 | Trivalent modRNA HA (A_{H1}A_{H3}B_{V}) (1:1:4) | 1.8 | 0.8 | 1.6 | 4263 | 4649 | 10736 |
| | A/Wisc, A/Darwin, B/Austria (Δ354-366) Mut 4 + L582C | | | | | | |

TABLE 67
PRL-Flu-Ms-2024-03: Enhancing Flu B immunogenicity in tIRV
with rational antigen design - DP analytics Summary

| | | Conc. | EE | Size | | DP | AA |
|---|---|---|---|---|---|---|---|
| Gp# | Description | (ug/mL) | % | (d · Nm) | PDI | FA % | Result |
| 1 | Saline | | | | | | Negative |
| 2 | Bv/Austria/1359417/2021-HA-WT | 108 | 98 | 59 | 0.07 | 186 | Negative |
| 3 | Bv/Austria/1359417/2021-HA-Δ355-361 + L582C | 107 | 98 | 72 | 0.08 | 85 | Negative |
| 4 | Bv/Austria/1359417/2021-HA-Δ361-364 + L582C | 104 | 98 | 60 | 0.09 | 87 | Negative |
| 5 | Bv/Austria/1359417/2021-HA-Δ355-363 + L582C | 111 | 98 | 60 | 0.07 | 80 | Negative |
| 6 | Bv/Austria/1359417/2021-HA-Δ354-366 + L582C | 126 | 98 | 60 | 0.09 | 88 | Negative |
| 7 | Trivalent modRNA HA (A_{H1}A_{H3}B_{V}) (1:1:4) | 114 | 98 | 61 | 0.11 | 86 | Negative |
| | A/Wisc, A/Darwin, B/Austria-WT | | | | | | |
| 8 | Trivalent modRNA HA (A_{H1}A_{H3}B_{V}) (1:1:4) | 133 | 98 | 64 | 0.09 | 87 | Negative |
| | A/Wisc, A/Darwin, B/Austria (Δ355-361) Mut 1 | | | | | | |
| 9 | Trivalent modRNA HA (A_{H1}A_{H3}B_{V}) (1:1:4) | 138 | 98 | 65 | 0.14 | 87 | Negative |
| | A/Wisc, A/Darwin, B/Austria (Δ355-361) Mut 1 + L582C | | | | | | |
| 10 | Trivalent modRNA HA (A_{H1}A_{H3}B_{V}) (1:1:4) | 110 | 98 | 64 | 0.14 | 86 | Negative |
| | A/Wisc, A/Darwin, B/Austria (Δ361-364) Mut 2 | | | | | | |
| 11 | Trivalent modRNA HA (A_{H1}A_{H3}B_{V}) (1:1:4) | 121 | 98 | 62 | 0.11 | 88 | Negative |
| | A/Wisc, A/Darwin, B/Austria (Δ361-364) Mut 2 + L582C | | | | | | |
| 12 | Trivalent modRNA HA (A_{H1}A_{H3}B_{V}) (1:1:4) | 116 | 98 | 62 | 0.1 | 87 | Negative |
| | A/Wisc, A/Darwin, B/Austria (Δ355-363) Mut 3 + L582C | | | | | | |
| 13 | Trivalent modRNA HA (A_{H1}A_{H3}B_{V}) (1:1:4) | 135 | 99 | 61 | 0.11 | 87 | Negative |
| | A/Wisc, A/Darwin, B/Austria (Δ354-366) Mut 4 + L582C | | | | | | |

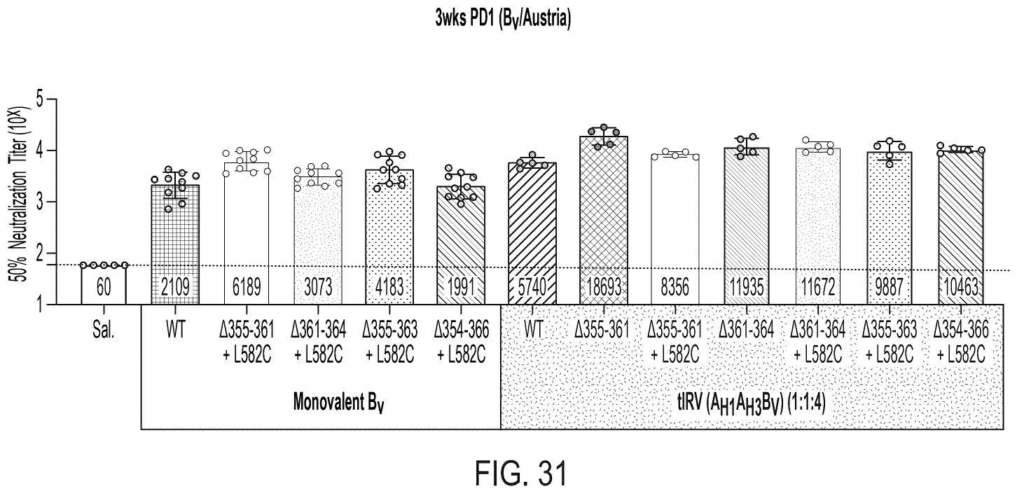

Based on the above results, FP mutant 1 (A355-361) (SEQ ID NO: 32) alone provided the largest enhancement of the immunogenicity of B/Austria HA in a trivalent formulation, inducing titers ˜3-fold higher than WT. Combining with the palmitoylation mutation negatively impacted the immunogenicity of mutant 1 (A355-361). Mutant 2 (otherwise referred to herein as 4361-364 (SEQ ID NO: 41) (with or without the palmitoylation mutation), mutant 3 (otherwise referred to herein as A355-363 (SEQ ID NO: 33)) and mutant 4 (otherwise referred to herein as A354-366 (SEQ ID NO: 30)) were similarly immunogenic in the tIRV, eliciting titers ˜2-fold higher than WT. See FIG. 31.

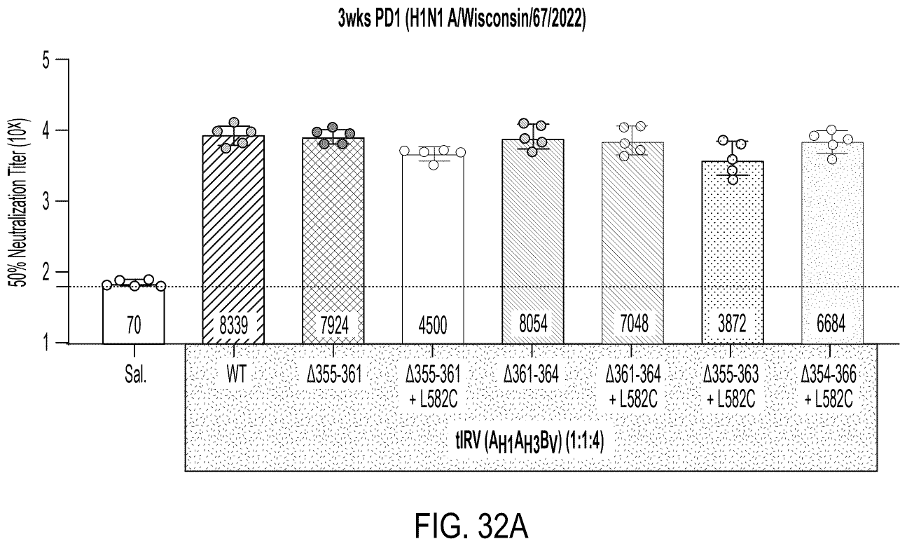

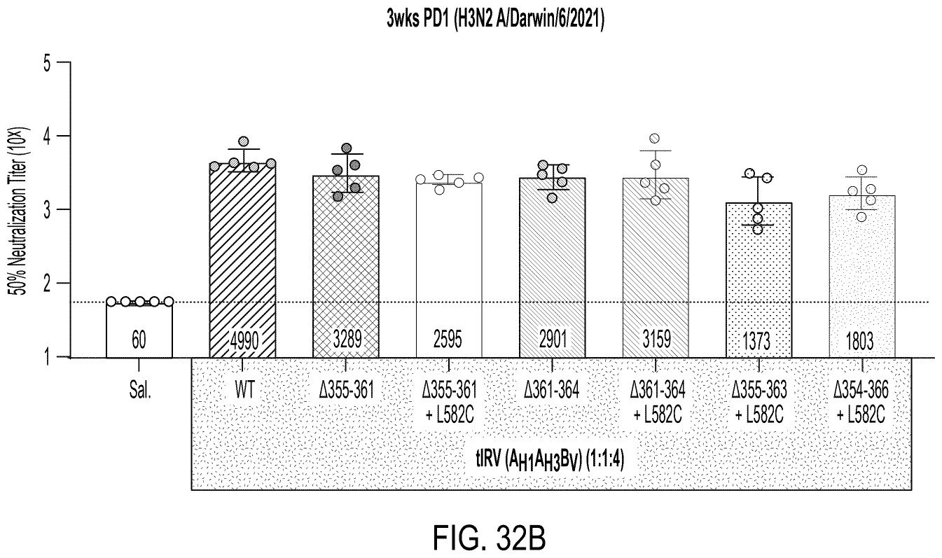

B/Austria combo mutants 1 (A355-361)+L582C and 3 (A355-363)+L582C in a tIRV formulation resulted in interference in H1N1 titers (˜2-fold reduction) whereas FP mutant 1 alone did not. tIRV formulations with B/Austria combo mutants 3 (A355-363)+L582C and 4 (A354-366)+L582C induced ˜3 to 4-fold lower titers against H3N2 compared to WT. See FIG. 32A-B.

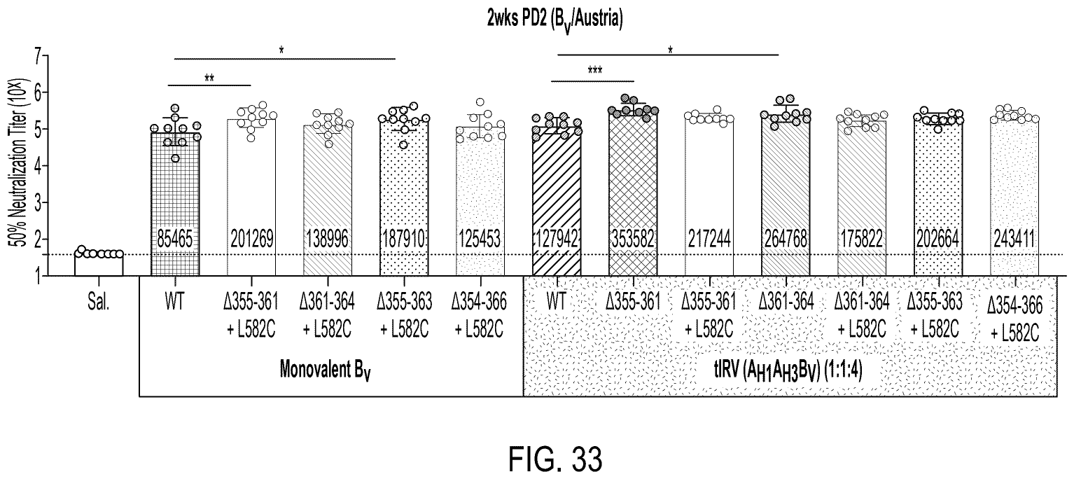

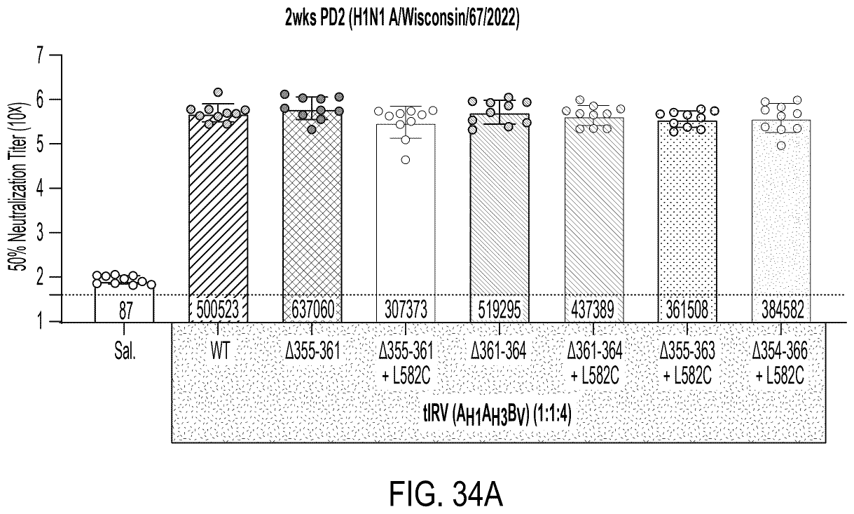

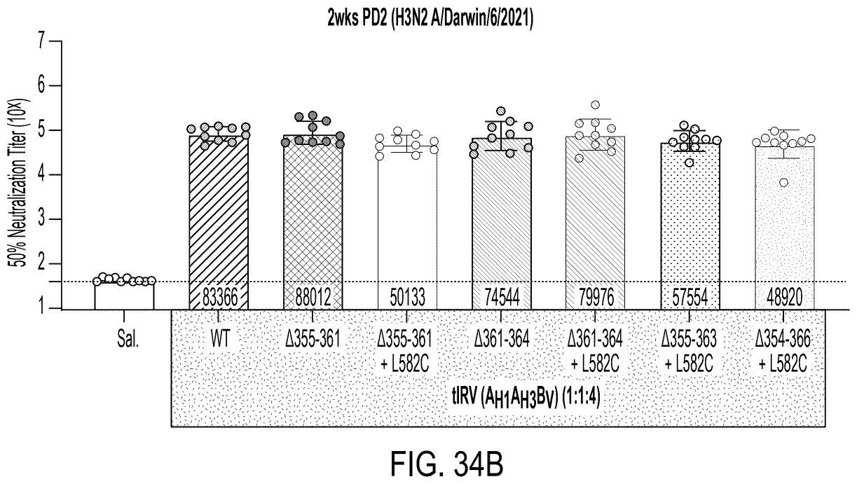

Combining a palmitoylation mutation with FP deletion does not provide any further boost to immunogenicity of B/Austria-HA in a trivalent modRNA formulation. A355-361 (mutant 1) continues to perform the best in vivo, eliciting titers 2.8-fold higher than WT HA in the trivalent. See FIG. 33. The Trivalent Vaccine Containing Lead Flu B HA Candidate A355-361 Showed 2.7-fold Higher Immunogenicity in Mice Compared to WT. See FIG. 33, FIG. 34A, and FIG. 34B. B/Austria combo mutants 1 (A355-361)+L582C, 3 (A355-363)+L582C and 4 (A354-366)+L582C in a tIRV formulation resulted in slight interference in H1N1 and H3N2 titers, whereas FP mutant A355-361 on its own did not. See FIG. 34.

### CLAUSES

1. An influenza virus vaccine, comprising: at least one ribonucleic acid (RNA) polynucleotide having an open reading frame encoding at least one influenza virus antigenic polypeptide or an immunogenic fragment thereof, formulated in a lipid nanoparticle.

2. The influenza vaccine of clause 1, wherein the RNA further comprises a 5′ cap analog.

3. The influenza vaccine of clause 2, wherein the 5′ cap analog comprises m27,3′-OGppp (m12-0) ApG.

4. The influenza vaccine of clause 1, wherein the RNA further comprises a modified nucleotide.

5. The influenza vaccine of clause 4, wherein the modified nucleotide comprises N1-Methylpseudourodine-5′-triphosphate (m1Y′TP).

6. The influenza vaccine of clause 1, wherein the at least one antigenic polypeptide is influenza hemagglutinin 1 (HA1), hemagglutinin 2 (HA2), an immunogenic fragment of HA1 or HA2, or a combination of any two or more of the foregoing.

7. The influenza vaccine of clause 1, wherein at least one antigenic polypeptide is HA1, HA2, or a combination of HA1 and HA2, and at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2).

8. The influenza vaccine of clause 1, wherein at least one antigenic polypeptide is HA1, HA2, or a combination of HA1 and HA2, and at least one antigenic polypeptide is neuraminidase (NA).

9. The influenza vaccine of clause 1, wherein the composition comprises a) at least one ribonucleic acid (RNA) polynucleotide having an open reading frame encoding influenza hemagglutinin 1 (HA1); b) at least one ribonucleic acid (RNA) polynucleotide having an open reading frame encoding hemagglutinin 2 (HA2); c) at least one ribonucleic acid (RNA) polynucleotide having an open reading frame encoding at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2); and d) at least one ribonucleic acid (RNA) polynucleotide having an open reading frame encoding at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2).

10. The influenza vaccine according to clause 5, wherein the open reading frame is codon-optimized.

11. The influenza vaccine of clause 1, wherein the composition further comprises a cationic lipid.

12. The influenza vaccine of clause 1, wherein the composition comprises a lipid nanoparticle encompassing the mRNA molecule.

13. The influenza vaccine of clause 1, wherein the composition comprises a) a lipid nanoparticle encompassing at least one ribonucleic acid (RNA) polynucleotide having an open reading frame encoding influenza hemagglutinin 1 (HA1); b) a lipid nanoparticle encompassing at least one ribonucleic acid (RNA) polynucleotide having an open reading frame encoding hemagglutinin 2 (HA2); c) a lipid nanoparticle encompassing at least one ribonucleic acid (RNA) polynucleotide having an open reading frame encoding at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2); and d) a lipid nanoparticle encompassing at least one ribonucleic acid (RNA) polynucleotide having an open reading frame encoding at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2).

14. The influenza vaccine of clause 13, wherein the lipid nanoparticle size is at least 40 nm.

15. The influenza vaccine of clause 13, wherein the lipid nanoparticle size is at most 180 nm.

16. The influenza vaccine of clause 13, wherein at least 80% of the total RNA in the composition is encapsulated.

17. The influenza vaccine of clause 1, wherein the composition comprises

18. The influenza vaccine of clause 1, wherein the composition comprises ALC-0315 (4-hydroxybutyl) azanediyl)bis(hexane-6,1-diyl)bis(2-hexyldecanoate).

19. The influenza vaccine of clause 1, wherein the composition comprises ALC-0159 (2-[(polyethylene glycol)-2000]-N, N-ditetradecylacetamide).

20. The influenza vaccine of clause 1, wherein the composition comprises 1,2-Distearoyl-sn-glycero-3-phosphocholine (DSPC).

21. The influenza vaccine of clause 1, wherein the composition comprises cholesterol.

22. The influenza vaccine of clause 1, wherein the composition comprises 0.9-1.85 mg/mL ALC-0315; 0.11-0.24 mg/mL ALC-0159; 0.18-0.41 mg/mL DSPC; and 0.36-0.78 mg/mL cholesterol.

23. The influenza vaccine of clause 1, wherein the composition comprises Tris.

24. The influenza vaccine of clause 1, wherein the composition comprises sucrose.

25. The influenza vaccine of clause 1, wherein the composition does not further comprise sodium chloride.

26. The influenza vaccine of clause 1, wherein the composition comprises 10 mM Tris.

27. The influenza vaccine of clause 1, wherein the composition comprises 300 mM sucrose.

28. The influenza vaccine of clause 1, wherein the composition has a pH 7.4.

29. The influenza vaccine of clause 1, wherein the composition has less than or equal to 12.5 EU/mL of bacterial endotoxins.

30. The influenza vaccine of clause 1, wherein the RNA polynucleotide comprises a 5′ cap, 5′ UTR, 3′ UTR, histone stem-loop and poly-A tail.

31. The influenza vaccine of clause 30, wherein the 5′ UTR comprises the sequence

(SEQ ID NO: 4)
AATAAACTAGTATTCTTCTGGTCCCCACAGACTCAGAGAGAACCC (5′
WHO UTR1).

32. The influenza vaccine of clause 30, wherein the 5′ UTR comprises the sequence

(SEQ ID NO: 5)
GAGAAΨAAACΨAGΨAΨΨCΨΨ CΨGGΨCCCCA CAGACΨCAGA
GAGAACCCGCCACC

33. The influenza vaccine of clause 30, wherein the 5′ UTR comprises the sequence

(SEQ ID NO: 6)
AGAATAAACTAGTATTCTTCTGGTCCCCACAGACTCAGAGAGAACCC
(5′ WHO UTR1).

34. The influenza vaccine of clause 30, wherein the 3′ UTR comprises the sequence

(SEQ ID NO: 7)
CUCGAGCUGGUACUGCAUGCACGCAAUGCUAGCUGCCCCUUUCCCGUCCU
GGGUACCCCGAGUCUCCCCCGACCUCGGGUCCCAGGUAUGCUCCCACCUC
CACCUGCCCCACUCACCACCUCUGCUAGUUCCAGACACCUCCCAAGCACG
CAGCAAUGCAGCUCAAAACGCUUAGCCUAGCCACACCCCCACGGGAAACA
GCAGUGAUUAACCUUUAGCAAUAAACGAAAGUUUAACUAAGCUAUACUAA
CCCCAGGGUUGGUCAAUUUCGUGCCAGCCACACCCUGGAGCUAGC (3′
WHO UTR2).

35. The influenza vaccine of clause 30, wherein the 3′ UTR comprises the sequence

(SEQ ID NO: 8)
CΨCGAGCΨGGΨACΨGCAΨGCACGCAAΨGCΨAGCΨGCCCCΨΨΨCCCGΨ
CCΨGGGΨACCCCGAGΨCΨCCCCCGACCΨCGGGΨCCCAGGΨAΨGCΨCC
CACCΨCCACCΨGCCCCACΨCACCACCΨCΨGCΨAGΨΨCCAGACACCΨC
CCAAGCACGCAGCAAΨGCAGCΨCAAAACGCΨΨAGCCΨAGCCACACCC
CCACGGGAAACAGCAGΨGAΨAACCΨΨΨAGCAAΨAACGAAAGΨΨΨAA
CΨAAGCΨAΨACΨAACCCCAGGGΨΨGGΨCAAΨΨΨCGΨGCCAGCCACAC
CCΨGGAGCΨAGC (3′ WHO ΨTR2)..

36. An immunogenic composition comprising: (i) a first ribonucleic acid (RNA) polynucleotide having an open reading frame encoding a first antigen, said antigen comprising at least one influenza virus antigenic polypeptide or an immunogenic fragment thereof, and (ii) a second RNA polynucleotide having an open reading frame encoding a second antigen, said second antigen comprising at least one influenza virus antigenic polypeptide or an immunogenic fragment thereof, wherein the first and second RNA polynucleotides are formulated in a lipid nanoparticle (LNP).

37. The immunogenic composition of clause 36, wherein the first and second antigens comprise hemagglutinin (HA), or an immunogenic fragment or variant thereof.

38. The immunogenic composition of clause 36 or 37 wherein the first antigen comprises an HA from a different subtype of influenza virus to the influenza virus antigenic polypeptide or an immunogenic fragment thereof of the second antigen.

39. The immunogenic composition of any of clause 36-38, wherein the first and second RNA polynucleotides are formulated in a single lipid nanoparticle.

40. The immunogenic composition of any preceding clause further comprising: (iii) a third antigen comprising at least one influenza virus antigenic polypeptide or an immunogenic fragment thereof, wherein the third antigen is from influenza virus but is from a different strain of influenza virus to both the first and second antigens.

41. The immunogenic composition of clause 40, wherein the first, second and third RNA polynucleotides are formulated in a lipid nanoparticle.

42. The immunogenic composition of clause 41, wherein the first, second and third RNA polynucleotides are formulated in a single lipid nanoparticle.

43. The immunogenic composition of any preceding clause further comprising: (iv) a fourth RNA polynucleotide having an open reading frame encoding a fourth antigen, said antigen comprising at least one influenza virus antigenic polypeptide or an immunogenic fragment thereof, wherein the fourth antigen is from influenza virus but is from a different strain of influenza virus to the first, second and third antigens.

44. The immunogenic composition of clause 43, wherein the first, second, third, and fourth RNA polynucleotides are formulated in a lipid nanoparticle.

45. The immunogenic composition of clause 44, wherein the first, second, third, and fourth RNA polynucleotides are formulated in a single lipid nanoparticle.

46. The immunogenic composition of any preceding clause further comprising: (v) a fifth RNA polynucleotide having an open reading frame encoding a fifth antigen, said antigen comprising at least one influenza virus antigenic polypeptide or an immunogenic fragment thereof, wherein the fifth antigen is from influenza virus but is from a different strain of influenza virus to the first, second, third, and fourth antigens.

47. The immunogenic composition of clause 46, wherein the first, second, third, fourth, and fifth RNA polynucleotides are formulated in a lipid nanoparticle.

48. The immunogenic composition of clause 47, wherein the first, second, third, fourth, and fifth RNA polynucleotides are formulated in a single lipid nanoparticle.

49. The immunogenic composition of any preceding clause further comprising: (vi) a sixth RNA polynucleotide having an open reading frame encoding a sixth antigen, said antigen comprising at least one influenza virus antigenic polypeptide or an immunogenic fragment thereof, wherein the sixth antigen is from influenza virus but is from a different strain of influenza virus to the first, second, third, fourth, and fifth antigens.

50. The immunogenic composition of clause 49, wherein the first, second, third, fourth, and fifth RNA polynucleotides are formulated in a lipid nanoparticle.

51. The immunogenic composition of clause 50, wherein the first, second, third, fourth, and fifth RNA polynucleotides are formulated in a single lipid nanoparticle.

52. The immunogenic composition of any preceding clause further comprising: (vii) a seventh RNA polynucleotide having an open reading frame encoding a seventh antigen, said antigen comprising at least one influenza virus antigenic polypeptide or an immunogenic fragment thereof, wherein the seventh antigen is from influenza virus but is from a different strain of influenza virus to the first, second, third, fourth, fifth, and sixth antigens.

53. The immunogenic composition of clause 52, wherein the first, second, third, fourth, fifth, sixth and seventh RNA polynucleotides are formulated in a lipid nanoparticle.

54. The immunogenic composition of clause 53, wherein the first, second, third, fourth, fifth, sixth and seventh RNA polynucleotides are formulated in a single lipid nanoparticle.

55. The immunogenic composition of any preceding clause further comprising: (viii) an eighth RNA polynucleotide having an open reading frame encoding an eighth antigen, said antigen comprising at least one influenza virus antigenic polypeptide or an immunogenic fragment thereof, wherein the eighth antigen is from influenza virus but is from a different strain of influenza virus to the first, second, third, fourth, fifth, sixth and seventh antigens.

56. The immunogenic composition of clause 55, wherein the first, second, third, fourth, fifth, sixth, seventh and eighth RNA polynucleotides are formulated in a lipid nanoparticle.

57. The immunogenic composition of clause 56, wherein the first, second, third, fourth, fifth, sixth, seventh and eighth RNA polynucleotides are formulated in a single lipid nanoparticle.

58. The immunogenic composition of any preceding clause further comprising: (v) a fifth RNA polynucleotide having an open reading frame encoding a fifth antigen, said antigen comprising at least one influenza virus antigenic polypeptide or an immunogenic fragment thereof, wherein the fifth antigen is from influenza virus but is from a different strain of influenza virus to the first, second, third, and fourth antigens.

59. The immunogenic composition of clause 58, wherein the first, second, third, fourth, and fifth RNA polynucleotides are formulated in a lipid nanoparticle.

60. The immunogenic composition of clause 59, wherein the RNA polynucleotides are present in about equal ratios.

61. The immunogenic composition of any preceding clause, wherein each RNA polynucleotide comprises a modified nucleotide.

62. The immunogenic composition of clause 61, wherein the modified nucleotide is selected from the group consisting of pseudouridine, 1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

63. The immunogenic composition of any preceding clause, wherein each RNA polynucleotide comprises a 5′ terminal cap, a 5′ UTR, a 3′UTR, and a 3′ polyadenylation tail.

64. The immunogenic composition of clause 63, wherein the 5′ terminal cap comprises:

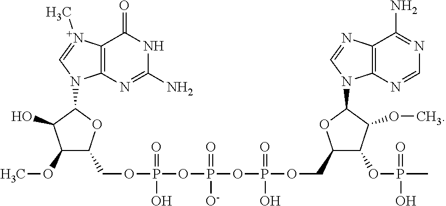

65. The immunogenic composition of clause 63, wherein the 5′ UTR comprises SEQ ID NO: 1.

66. The immunogenic composition of clause 63, wherein the 3′ UTR comprises SEQ ID NO: 2.

67. The immunogenic composition of clause 63, wherein the 3′ polyadenylation tail comprises SEQ ID NO: 3.

68. The immunogenic composition of any preceding clause, wherein the RNA polynucleotide has an integrity greater than 85%.

69. The immunogenic composition of any preceding clause, wherein the RNA polynucleotide has a purity of greater than 85%.

70. The immunogenic composition of any preceding clause, wherein the lipid nanoparticle comprises 20-60 mol % ionizable cationic lipid, 5-25 mol % neutral lipid, 25-55 mol % cholesterol, and 0.5-5 mol % PEG-modified lipid.

71. The immunogenic composition of any preceding clause, wherein the cationic lipid comprises:

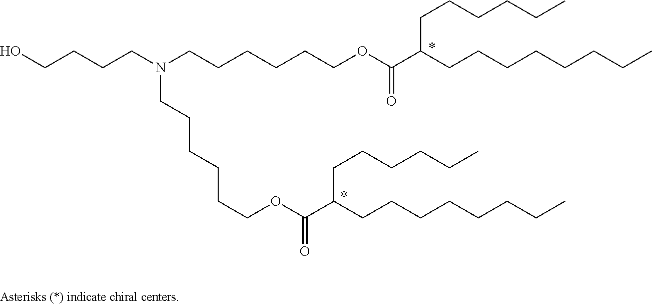 [SMILES: CCCCCCCCC(CCCCCC)C(=O)OCCCCCCN(CCCCO)CCCCCCOC(=O)C(CCCCCC)CCCCCCCC]

72. The immunogenic composition of any preceding clause, wherein the PEG-modified lipid comprises:

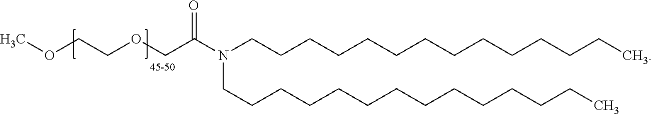

73. The immunogenic composition of any preceding clause, wherein the first antigen is HA from influenza A subtype H1 or an immunogenic fragment or variant thereof and the second antigen is HA from a different H1 strain to the first antigen or an immunogenic fragment or variant thereof.

74. The immunogenic composition of any preceding clause, wherein the first and second antigens are HA from influenza A subtype H3 or an immunogenic fragment or variant thereof and wherein both antigens are derived from different strains of H3 influenza virus.

75. The immunogenic composition of any preceding clause, wherein the first and second antigens are HA from influenza A subtype H1 or an immunogenic fragment or variant thereof and the third and fourth antigens are from influenza A subtype H3 or an immunogenic fragment or variant thereof and wherein the first and second antigens are derived from different strains of H1 virus and the third and fourth antigens are from different strains of H3 influenza virus.

76. The immunogenic composition of any preceding clause, wherein at least the first and second RNA polynucleotides are formulated in a single lipid nanoparticle.

77. The immunogenic composition of any preceding clause, wherein the first and second RNA polynucleotides are formulated in a single lipid nanoparticle.

78. The immunogenic composition of any preceding clause, wherein the first, second, and third RNA polynucleotides are formulated in a single lipid nanoparticle.

79. The immunogenic composition of any preceding clause, wherein the first, second, third, and fourth RNA polynucleotides are formulated in a single LNP.

80. The immunogenic composition of any one of clauses 36-75, wherein each of the RNA polynucleotides is formulated in a single LNP, wherein each single LNP encapsulates the RNA polynucleotide encoding one antigen.

81. The immunogenic composition of clause 80, wherein the first RNA polynucleotide is formulated in a first LNP; and the second RNA polynucleotide is formulated in a second LNP.

82. The immunogenic composition of clause 80, wherein the first RNA polynucleotide is formulated in a first LNP; the second RNA polynucleotide is formulated in a second LNP; and the third RNA polynucleotide is formulated in a third LNP.

83. The immunogenic composition of clause 80, wherein the first RNA polynucleotide is formulated in a first LNP; the second RNA polynucleotide is formulated in a second LNP; the third RNA polynucleotide is formulated in a third LNP; and the fourth RNA polynucleotide is formulated in a fourth LNP.

84. The immunogenic composition of clause 80, wherein the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; and the fifth RNA polynucleotide is formulated in a fifth LNP.

85. The immunogenic composition of clause 80, wherein the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; and the sixth RNA polynucleotide is formulated in a sixth LNP.

86. The immunogenic composition of clause 80, wherein the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; the sixth RNA polynucleotide is formulated in a sixth LNP; and the seventh RNA polynucleotide is formulated in a seventh LNP.

87. The immunogenic composition of clause 80, wherein the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; the sixth RNA polynucleotide is formulated in a sixth LNP; the seventh RNA polynucleotide is formulated in a seventh LNP; and the eighth RNA polynucleotide is formulated in an eighth LNP.

88. The immunogenic composition of any preceding clause, for use in the eliciting an immune response against influenza.

89. A method of eliciting an immune response against influenza disease, comprising administering an effective amount of an immunogenic composition according to any one of clauses 36-79.

90. A method of purifying an RNA polynucleotide synthesized by in vitro transcription, comprising ultrafiltration and diafiltration.

91. The method according to clause 90, wherein the method does not comprise a chromatography step.

92. The method according to clause 90, wherein the purified RNA polynucleotide is substantially free of contaminants comprising short abortive RNA species, long abortive RNA species, double-stranded RNA (dsRNA), residual plasmid DNA, residual in vitro transcription enzymes, residual solvent and/or residual salt.

93. The method according to clause 90, wherein the residual plasmid DNA is ≤500 ng DNA/mg RNA.

94. The method according to clause 90, wherein purity of the purified mRNA is between about 60% and about 100%.

95. The method according to clause 90, further comprising encapsulating the RNA polynucleotide in a lipid nanoparticle.

96. The method according to clause 95, wherein the LNPs are buffer exchanged and concentrated via flat sheet cassette membranes.

97. A composition according to any one of clause 1-88, wherein the lipid nanoparticle comprises a sphingomyelin (SM).

98. A composition according to any one of clause 1-88, wherein the lipid nanoparticle comprises a egg sphingomyelin (ESM).

99. A mutant influenza polypeptide comprising at least 80% identity to any one of amino acid sequences SEQ ID NO: 10-SEQ ID NO: 68.

100. A polynucleotide encoding a mutant influenza polypeptide comprising at least 80% identity to any one of amino acid sequences SEQ ID NO: 10-SEQ ID NO: 68.

101. A composition comprising: (i) a first ribonucleic acid (RNA) polynucleotide comprising an open reading frame encoding a first polypeptide, said polypeptide comprising a polypeptide derived from influenza B virus or an immunogenic fragment thereof, wherein the polypeptide comprises at least 80% identity to any one of amino acid sequences SEQ ID NO: 10-SEQ ID NO: 68.

102. The composition according to clause 3, further comprising (ii) a second RNA polynucleotide comprising an open reading frame encoding a second antigen, wherein the first and second RNA polynucleotides are formulated in a lipid nanoparticle (LNP), wherein the amount of second RNA polynucleotide is greater than the amount of the first RNA polynucleotide.

103. The composition of clause 3, wherein the first and second antigens comprise hemagglutinin (HA), or an immunogenic fragment or variant thereof.

104. The immunogenic composition of clause 3 or 4 wherein the first and second antigens each comprise an HA, or an immunogenic fragment thereof, that are from different subtypes of influenza virus.

105. The composition of any one of clauses 101-104, wherein the ratio of the first RNA polynucleotide to the second RNA polynucleotide is 1: greater than 1.

106. The composition of any one of clauses 101-105, wherein the ratio of the first RNA polynucleotide to the second RNA polynucleotide is 1:2.

107. The composition of any one of clauses 101-106, wherein the ratio of the first RNA polynucleotide to the second RNA polynucleotide is 1:4.

108. The composition of any one of clauses 101-107, further comprising: (iii) a third RNA polynucleotide comprising an open reading frame encoding an antigen comprising at least one influenza virus antigenic polypeptide or an immunogenic fragment thereof, wherein the third antigen is from an influenza virus different from the strain of influenza virus of both the first and second antigens.

109. The composition of clause 108, wherein the first, second and third RNA polynucleotides are formulated in a lipid nanoparticle.

110. The composition of clause 109, further comprising: (iv) a fourth RNA polynucleotide comprising an open reading frame encoding a fourth antigen, said antigen comprising at least one influenza virus antigenic polypeptide or an immunogenic fragment thereof, wherein the fourth antigen is from influenza virus but is from a different strain of influenza virus to the first, second and third antigens.

111. The composition of clause 110, wherein the first, second, third, and fourth RNA polynucleotides are formulated in a lipid nanoparticle.

112. The composition of any one of clauses 101-111, wherein the RNA polynucleotides are not present in equal ratios.

113. The composition of any one of clauses 101-112, wherein each RNA polynucleotide comprises a modified nucleotide.

114. The composition of clause 114, wherein the modified nucleotide is selected from the group consisting of pseudouridine, 1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

115. The composition of any one of clauses 101-114, wherein each RNA polynucleotide comprises a 5′ terminal cap, a 5′ UTR, a 3′UTR, and a 3′ polyadenylation tail.

116. The composition of clause 115, wherein the 5′ terminal cap comprises:

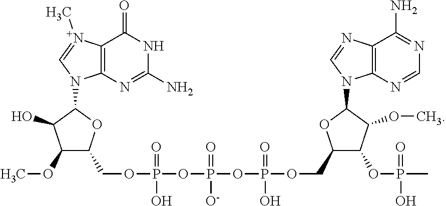

117. The composition of clause 115, wherein the 5′ UTR comprises SEQ ID NO: 1. 118. The composition of clause 115, wherein the 3′ UTR comprises SEQ ID NO: 2.

119. The composition of clause 115, wherein the 3′ polyadenylation tail comprises SEQ ID NO: 3.

120. The composition of any one of clauses 101-119, wherein the RNA polynucleotide has an integrity greater than 85%.

121. The composition of any one of clauses 101-120, wherein the RNA polynucleotide has a purity of greater than 85%.

122. The composition of any one of clauses 101-121, wherein the lipid nanoparticle comprises 20-60 mol % ionizable cationic lipid, 5-25 mol % neutral lipid, 25-55 mol % cholesterol, and 0.5-5 mol % PEG-modified lipid.

123. The composition of any one of clauses 101-122, wherein the cationic lipid comprises:

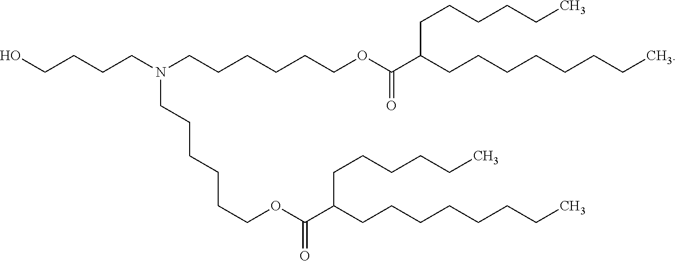 [SMILES: CCCCCCCCC(CCCCCC)C(=O)OCCCCCCN(CCCCO)CCCCCCOC(=O)C(CCCCCC)CCCCCCCC]

124. The composition of any one of clauses 101-123, wherein the PEG-modified lipid comprises:

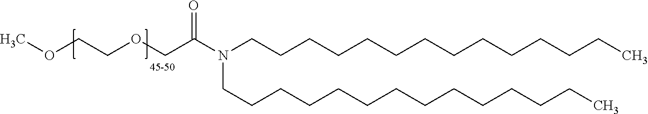

125. The composition of any one of clauses 101-124, wherein the first antigen is HA from influenza A subtype H1 or an immunogenic fragment or variant thereof and the second antigen is HA from a different H1 strain to the first antigen or an immunogenic fragment or variant thereof.

126. The composition of any one of clauses 101-125, wherein the first and second antigens are HA from influenza A subtype H3 or an immunogenic fragment or variant thereof and wherein both antigens are derived from different strains of H3 influenza virus.

127. The composition of any one of clauses 101-126, wherein the first and second antigens are HA from influenza A subtype H1 or an immunogenic fragment or variant thereof and the third and fourth antigens are from influenza A subtype H3 or an immunogenic fragment or variant thereof and wherein the first and second antigens are derived from different strains of H1 virus and the third and fourth antigens are from different strains of H3 influenza virus.

128. The composition of any one of clauses 101-127, wherein at least the first and second RNA polynucleotides are formulated in a single lipid nanoparticle.

129. The composition of any one of clauses 101-128, wherein the first and second RNA polynucleotides are formulated in a single lipid nanoparticle.

130. The composition of any one of clauses 101-129, wherein the first, second, and third RNA polynucleotides are formulated in a single lipid nanoparticle.

131. The composition of any preceding clause, wherein the first, second, third, and fourth RNA polynucleotides are formulated in a single LNP.

132. The composition of any one of clauses 101-131, wherein each of the RNA polynucleotides is formulated in a single LNP, wherein each single LNP encapsulates the RNA polynucleotide encoding one antigen.

133. The composition of clause 132, wherein the first RNA polynucleotide is formulated in a first LNP; and the second RNA polynucleotide is formulated in a second LNP.

134. The composition of clause 132, wherein the first RNA polynucleotide is formulated in a first LNP; the second RNA polynucleotide is formulated in a second LNP; and the third RNA polynucleotide is formulated in a third LNP.

135. The composition of clause 132, wherein the first RNA polynucleotide is formulated in a first LNP; the second RNA polynucleotide is formulated in a second LNP; the third RNA polynucleotide is formulated in a third LNP; and the fourth RNA polynucleotide is formulated in a fourth LNP.

136. The composition of any one of clauses 101-135, for use in the eliciting an immune response against influenza in a subject.

137. A method of eliciting an immune response against influenza disease in a subject, comprising administering an effective amount of a composition according to any one of clauses 99-136.

138. A method of producing an RNA polynucleotide-encapsulated lipid nanoparticle (LNP), the method comprises purifying an RNA polynucleotide comprising an open reading frame encoding a first antigen, said antigen comprising at least one influenza virus antigenic polypeptide or an immunogenic fragment thereof through ultrafiltration and diafiltration; formulating the purified RNA polynucleotide in an LNP, wherein the LNP is buffer exchanged and concentrated via flat sheet cassette membranes.

139. The method according to clause 138, wherein the method does not comprise a chromatography step or hollow fiber membranes.

140. The method according to clause 139, wherein the purified RNA polynucleotide is substantially free of contaminants comprising short abortive RNA species, long abortive RNA species, double-stranded RNA (dsRNA), residual plasmid DNA, residual in vitro transcription enzymes, residual solvent and/or residual salt.

1. A polypeptide comprising an amino acid sequence of a hemagglutinin of an influenza virus strain, wherein the amino acid sequence of the polypeptide comprises a deletion of three to seven consecutive amino acid residues within a region when compared to the amino acid sequence of a hemagglutinin of a respective wild-type influenza virus strain, wherein the region comprises a sequence having 100% identity to the amino acid sequence set forth in positions 352 to 382 of SEQ ID NO: 9.

2. The polypeptide according to claim 1, comprising an amino acid sequence having at least 80% identity to SEQ ID NO: 10-SEQ ID NO: 115.

3. The polypeptide according to claim 1, comprising an amino acid sequence having at least 80% identity to any one of SEQ ID NO: 30; SEQ ID NO: 32; SEQ ID NO: 33; SEQ ID NO: 41; and SEQ ID NO: 47.

4. (canceled)

5. (canceled)

6. (canceled)

7. A polynucleotide comprising an open reading frame encoding a polypeptide comprising an amino acid sequence of a hemagglutinin of an influenza virus strain, wherein the amino acid sequence of the polypeptide comprises a deletion of three to seven consecutive amino acid residues within a region when compared to the amino acid sequence of a hemagglutinin of a respective wild-type influenza virus strain, wherein the region comprises a sequence having 100% identity to the amino acid sequence set forth in positions 352 to 382 of SEQ ID NO: 9.

8. The polynucleotide according to claim 7, wherein the polynucleotide is RNA.

9. The polynucleotide according to claim 8, further comprising at least one poly(A) sequence comprising 30 to 200 adenosine nucleotides.

10. The polynucleotide according to claim 8, wherein the RNA comprises at least one untranslated region selected from at least one heterologous 5′-UTR and at least one heterologous 3′-UTR.

11. The polynucleotide according to claim 8, wherein the RNA comprises a nucleotide analog.

12. The polynucleotide according to claim 8, wherein the RNA comprises a 1-methylpseudouridine substitution.

13. The polynucleotide according to claim 8, wherein the RNA comprises a 5′-cap structure, which comprises a structure selected from the group consisting of m7G, cap0, cap1, cap2, a modified cap0, and a modified cap1 structure.

14. A composition comprising an RNA polynucleotide comprising at least one open reading frame encoding a polypeptide comprising an amino acid sequence of a hemagglutinin of an influenza virus strain, wherein the amino acid sequence of the polypeptide comprises a deletion of three to seven consecutive amino acid residues within a region when compared to the amino acid sequence of a hemagglutinin of a respective wild-type influenza virus strain, wherein the region comprises a sequence having 100% identity to the amino acid sequence set forth in positions 352 to 382 of SEQ ID NO: 9.

15. The composition according to claim 14, further comprising at least one heterologous untranslated region (UTR); and (c) at least one pharmaceutically acceptable carrier, wherein the RNA is complexed or associated with lipids, wherein the lipids comprise a cationic lipid, a neutral lipid, a steroid, and a PEG-lipid.

16. The composition according to claim 14, further comprising a second RNA polynucleotide comprising an open reading frame encoding a second antigen, wherein the first and second RNA polynucleotides are complexed or associated with lipids.

17. The composition according to claim 16, wherein the first and second antigens comprise hemagglutinin (HA) or an immunogenic fragment or variant thereof.

18. The composition according to claim 17, wherein the first and second antigens each comprise an HA, or an immunogenic fragment thereof, from different subtypes of influenza virus.

19. The composition according to claim 14, wherein the polypeptide comprises an amino acid sequence having at least 80% identity to any one of amino acid sequences SEQ ID NO: 10-SEQ ID NO: 115.

20. The composition according to claim 14, wherein the polypeptide comprises an amino acid sequence having at least 80% identity to any one of SEQ ID NO: 30; SEQ ID NO: 32; SEQ ID NO: 33; SEQ ID NO: 41; and SEQ ID NO: 47.

21. (canceled)

22. (canceled)

23. (canceled)

24. (canceled)

25. (canceled)

26. The composition according to claim 14, wherein the RNA polynucleotide further comprises at least one subgenomic promoter.

27. The polynucleotide according to claim 8, further comprising at least one subgenomic promoter.


SEQUENCE LISTING (118 sequences)

SEQ ID NO: 1 - RNA, 42 nt, synthetic construct
  source 1..42: mol_type = other RNA
gaaaaacagaccggccccacagaccagagagaacccgccacc

SEQ ID NO: 2 - RNA, 237 nt, synthetic construct
  source 1..237: mol_type = other RNA
ccgagcggacgcagcacgcaagcagcgcccccccgccgggaccccgagccccccgacccgggcccaggagccccaccccaccgccccaccaccacccgcagccagacacccccaagcacgcagcaagcagccaaaacgcagccagccacacccccacgggaaacagcaggaaaccagcaaaaacgaaagaacaagcaacaaccccagggggcaacggccagccacacccggagcagc

SEQ ID NO: 3 - RNA, 107 nt, synthetic construct
  source 1..107: mol_type = other RNA
aaaaaaaaaaaaaaaaaaaaaaaaaaaaaagcaagacaaaaaaaaaaaaaaaaaaaaaaaaaaaaaaaaaaaaaaaaaaaaaaaaaaaaaaaaaaaaaaaaaaaaaa

SEQ ID NO: 4 - RNA, 45 nt, synthetic construct
  source 1..45: mol_type = other RNA
aataaactagtattcttctggtccccacagactcagagagaaccc

SEQ ID NO: 5 - RNA, 44 nt, synthetic construct
  source 1..44: mol_type = other RNA
gagaaaaacagaccggccccacagaccagagagaacccgccacc

SEQ ID NO: 6 - RNA, 47 nt, synthetic construct
  source 1..47: mol_type = other RNA
agaataaactagtattcttctggtccccacagactcagagagaaccc

SEQ ID NO: 7 - RNA, 295 nt, synthetic construct
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 8 - RNA, 237 nt, synthetic construct; identical to SEQ ID NO: 2
  source 1..237: mol_type = other RNA

SEQ ID NO: 9 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 10 - protein, 571 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMV

SEQ ID NO: 11 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMCSRDNVSCSICL

SEQ ID NO: 12 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVCCSICL

SEQ ID NO: 13 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCCICL

SEQ ID NO: 14 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 15 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMCCRDNVSCSICL

SEQ ID NO: 16 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMCSRDNVSCCICL

SEQ ID NO: 17 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDCCSCSICL

SEQ ID NO: 18 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMCCRDNVCCSICL

SEQ ID NO: 19 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMCCRDNVSCCICL

SEQ ID NO: 20 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSCCNVSCCICL

SEQ ID NO: 21 - protein, 592 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSCCNVSCCICLKGCCSCGSCC

SEQ ID NO: 22 - protein, 580 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFGAIAGFLEGGWEGMIGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 23 - protein, 576 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 24 - protein, 574 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 25 - protein, 571 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 26 - protein, 567 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 27 - protein, 565 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 28 - protein, 563 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 29 - protein, 570 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 30 - protein, 569 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 31 - protein, 578 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKRGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 32 - protein, 575 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 33 - protein, 573 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 34 - protein, 572 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 35 - protein, 569 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 36 - protein, 574 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 37 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 38 - protein, 579 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKEFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 39 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKEAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 40 - protein, 576 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKEIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 41 - protein, 578 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 42 - protein, 575 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 43 - protein, 578 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 44 - protein, 576 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 45 - protein, 575 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 46 - protein, 574 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 47 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 48 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKEAAFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 49 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGAFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 50 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFAGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 51 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAAAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 52 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGALEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 53 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFAEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 54 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERPPFGAGAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 55 - protein, 575 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCCICL

SEQ ID NO: 56 - protein, 578 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCCICL

SEQ ID NO: 57 - protein, 575 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCCICL

SEQ ID NO: 58 - protein, 578 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCCICL

SEQ ID NO: 59 - protein, 574 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCCICL

SEQ ID NO: 60 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCCICL

SEQ ID NO: 61 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMCCRDNVSCSICL

SEQ ID NO: 62 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 63 - protein, 575 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSCCNVSCCICL

SEQ ID NO: 64 - protein, 578 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSCCNVSCCICL

SEQ ID NO: 65 - protein, 575 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSCCNVSCCICL

SEQ ID NO: 66 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSCCNVSCCIC

SEQ ID NO: 67 - protein, 574 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSCCNVSCCICL

SEQ ID NO: 68 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSCCNVSCCICL

SEQ ID NO: 69 - protein, 578 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 70 - protein, 578 aa, synthetic construct; identical to SEQ ID NO: 56

SEQ ID NO: 71 - protein, 575 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 72 - protein, 575 aa, synthetic construct; identical to SEQ ID NO: 55

SEQ ID NO: 73 - protein, 575 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMCSRDNVSCSICL

SEQ ID NO: 74 - protein, 573 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMCSRDNVSCSICL

SEQ ID NO: 75 - protein, 569 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 76 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCCICC

SEQ ID NO: 77 - protein, 564 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 78 - protein, 574 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 79 - protein, 573 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 80 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERPPFGAGAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 81 - protein, 580 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFGAIAGFLEGGWEGMIGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 82 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGALEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICC

SEQ ID NO: 83 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGALEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCCICL

SEQ ID NO: 84 - protein, 581 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERPPFGAGAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSIC

SEQ ID NO: 85 - protein, 580 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERPPFGAGAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSI

SEQ ID NO: 86 - protein, 581 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSIC

SEQ ID NO: 87 - protein, 580 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSI

SEQ ID NO: 88 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSESEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 89 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEDLLKLERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 90 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPFYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTWSYQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 91 - protein, 582 aa, synthetic construct
MKAIIVLLMVVTSNADRICTGITSSNSPHVVKTACQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINCITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 92 - protein, 582 aa, synthetic construct
MKAIIVLLMVVTSNADRICTGITSSNSPHVVKTATQGEVNVTGVIPLTTTPTKSHFANLKGTKTRGKLCPKCLNCTDLDVALGRPKCTGKIPSARVSILHEVRPVTSGCFPIMHDRTKIRQLPNLLRGYEHVRLSTRNVINTEKAPGGPYEIGTSGSCLNITNGKGFFATMAWAVPKNKTATNPLTIEVPYICTKEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 93 - protein, 582 aa, synthetic construct
MKAIIVLLMVVTSNADRICTGITSSNSPHVVKTATQGEVNVTGVIPLTTTPTKSCFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLCGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 94 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLFRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 95 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHPPILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 96 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVCNLQRLSGAMDELHNEILELDEKVDDLRACTISSQIELAVLLSNEGIINSEDEHLLALERYLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 97 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVCNLQRLSGAMDELHPPILELDEKVDDLRACTISSQIELAVLLSNEGIINSEDEHLLALERYLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 98 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTWSYQIELAVLLSNEGIINSEDEHLLALERYLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 99 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTIYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHPPILELDEKVDDLRADTISSQIELAVLLSNEGIINSEDEDLLALERKLKKMLGPSAVEIGNGCFETKFKCNQTCLDRIAAGTFDAGEFSLPTFDSLNITAASLNDDGLDNHTILLYYSTAASSLAVTLMIAIFVVYMVSRDNVSCSICL

SEQ ID NO: 100 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTIYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVCNLQRLSGAMDELHPPILELDEKVDDLRACTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 101 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTIYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVCNLQRLSGAMDELHNEILELDEKVDDLRACTISSQIELIVLLSNEGIINSEDEHLLALERKLKKMLGPSAVEIGNGCFETKFKCNQTCLDRIAAGTFDAGEFSLPTFDSLNITAASLNDDGLDNHTILLYYSTAASSLAVTLMIAIFVVYMVSRDNVSCSICL

SEQ ID NO: 102 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQQFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHPPILELDEKVDDLRADTISSQIELIVLLSNEGIINSEDEHLLALERKLKKMLGPSAVEIGNGCFETKFKCNQTCLDRIAAGTFDAGEFSLPTFDSLNITAASLNDDGLDNHTILLYYSTAASSLAVTLMIAIFVVYMVSRDNVSCSICL

SEQ ID NO: 103 - protein, 581 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTIYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTWSYQIELAVLLSNEGIINSEDEDLLALERKLKKMLGPSAVEIGNGCFETKFKCNQTCLDRIAAGTFDAGEFSLPTFDSLNITAASLNDDGLDNHTILLYYSTAASSLAVTLMIAIFVVYMVSRDNVSCSIC

SEQ ID NO: 104 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWCGYTSHGAHGVAVAADLKCTQEAINKITKNLNSLSELEVKNLQRLSGAMDELHPPILELDEKVDDLRADTISSQIELAVLLSNEGIINSEDEDLLALERYLKKMLGPSAVEIGNGCFETKFKCNQTCLDRIAAGTFDAGEFSLPTFDSLNITAASLNDDGLDNHTILLYYSTAASSLAVTLMIAIFVVYMVSRDNVSCSICL

SEQ ID NO: 105 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGESAVEIGNGCFETKHKCNQTCLDRIAAGTFDAGEFSLETFDSRNITAASLNDDGLDNHTILLYYSTAASSLAVTLMIAIFVVYMVSRDNVSCSICL

SEQ ID NO: 106 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLDTFDSLNITAASLNDDGLDNHTILLYYSTAASSLAVTLMIAIFVVYMVSRDNVSCSICL

SEQ ID NO: 107 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRCLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGACDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 108 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTCIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDECHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 109 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSACGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDCLHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 110 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVDTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 111 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPKWVKQNTLKLANGKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERYLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 112 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASCLAVCLMIAIFVVYMCSRDCVSCSICL

SEQ ID NO: 113 - protein, 582 aa, synthetic construct; identical to SEQ ID NO: 112

SEQ ID NO: 114 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASVLAVCLMIAIFVVYMCSRDNVSCSICL

SEQ ID NO: 115 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASCLAVCLMIAIFVVYMCSRDNVSCSICL

SEQ ID NO: 116 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 117 - protein, 578 aa, synthetic construct; identical to SEQ ID NO: 43

SEQ ID NO: 118 - protein, 577 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL